(19) United States
(12) Patent Application Publication
(10) Pub. No.: US 2006/0182685 A1
(43) Pub. Date: Aug. 17, 2006

(54) Hollow fiber technique for in vivo study of cell populations

(76) Inventors: William R. Bishai, Baltimore, MD (US); Petros C. Karakousis, Lutherville, MD (US)

Correspondence Address: MAYER, BROWN, ROWE & MAW LLP, P.O. BOX 2828, CHICAGO, IL, 60690-2828, US

(21) Appl. No.: 11/221,294

(22) Filed: Sep. 6, 2005

Related U.S. Application Data
(60) Provisional application No. 60/606,939, filed on Sep. 4, 2004.

(51) Int. Cl. A61K 49/00; C12Q 1/00

(52) U.S. Cl. 424/9.2; 435/4

(57) ABSTRACT

The present invention relates to a method of using hollow fibers to evaluate cellular changes in vivo. A method for evaluating cellular changes in vivo in response to administration of a drug or drugs of interest is provided. The hollow fiber technique is also used to evaluate cellular changes in a microorganism in vivo. The technique is further used to evaluate cellular changes in a microorganism in vivo in response to administration of a drug or drugs of interest. A vaccine and method of vaccination are also provided.

### CROSS-REFERENCE TO RELATED APPLICATIONS

This application claims priority to U.S. Provisional patent application 60/606,939, filed Sep. 3, 2004, incorporated herein by reference in its entirety.

### STATEMENT REGARDING FEDERALLY SPONSORED RESEARCH OR DEVELOPMENT

Embodiments of this invention were made with Government support under AI-37856, AI-43846 and AI-07608 awarded by the PHS. The Government may have certain rights in the invention.

### BACKGROUND OF THE INVENTION

M. tuberculosis infects approximately one third of the world's population, resulting in 3 million deaths annually. Cegielski, J. P., et al. Infect Dis Clin North Am 16:1-58 (2002). Soon after inhalation of tubercle bacilli, the organisms are phagocytosed by alveolar macrophages, resulting in potent cell-mediated immune responses and the formation of granulomas, which consist primarily of T cells and M. tuberculosis-infected macrophages. Flynn, J. L., and J. Chan. Infect Immun 69:4195-4201 (2001); Kaplan, G., et al. Infect Immun 71:7099-7108 (2003). Six to eight weeks after infection in humans, and coincident with the development of a delayed-type hypersensitivity response manifested by tuberculin skin test positivity, these granulomas undergo caseous necrosis, resulting in the death of the majority of tubercle bacilli and destruction of surrounding host tissue. Grosset, J. Antimicrob Agents Chemother 47:833-836 (2003). The small proportion of surviving bacilli are thought to exist in a nonreplicating hypometabolic state, as an adaptation to the unfavorable milieu in the solid caseous material. Id. This altered physiologic state, termed latent tuberculosis infection, can endure for the lifetime of the infected individual, but in approximately 10% of cases, through unknown mechanisms, these dormant bacilli reactivate many years to decades later to produce disease.

Efforts to gain insight into the adaptive mechanisms by which M. tuberculosis persists in the host have been impeded by the inability to recover sufficient quantities of M. tuberculosis RNA from host lesions consistent with contained latent tuberculosis infection. Talaat, A. M., et al. Proc Natl Acad Sci USA 101:4602-4607 (2004). Consequently, several groups have turned to in vitro models which may reflect the persistent state, and have defined the gene expression profile of M. tuberculosis under conditions of hypoxia (Sherman, D. R., et al. Proc Natl Acad Sci USA 98:7534-7539 (2001), Rosenkrands, I., et al. J Bacteriol 184:3485-3491 (2002)), nutrient starvation (Betts, J. C., et al. Mol Microbiol 43:717-731 (2002)), low pH (Fisher, M. A., et al. J Bacteriol 184:4025-4032 (2002)), low concentrations of nitric oxide (Voskuil, M. I., et al. J Exp Med 198:705-713 (2003)), and in the phagosomal compartment of murine macrophages (Schnappinger, D., et al. J Exp Med 198:693-704 (2003)). Current work has focused on the role of the two-component response regulator dormancy survival regulator (dosR), which initially was found to be the primary mediator of the hypoxic response in M. tuberculosis (Sherman, supra, Park, H. D., et al. Mol Microbiol 48:833-843 (2003)). Bacilli exposed to low, nontoxic concentrations of nitric oxide in vitro enter a nonreplicating persistent state marked by the induction of a 48-gene regulon under the control of dosR, suggesting that the dosR regulon may mediate the transition of these bacilli into dormancy. Voskuil, M. I., et al. J Exp Med 198:705-713 (2003). Consistent with these findings, several dosR regulon genes, including acr (Rv2031c), Rv2623c, and Rv2626, are upregulated in infected mouse tissues after the onset of Th1 immunity. Voskuil, supra, Shi, L., et al. Proc Natl Acad Sci USA 100:241-246 (2003).

Because evaluation of cellular changes in vivo is typically difficult, if not impossible, the results of the studies of latent tuberculosis infection discussed above have been limited to in vitro models. Thus, there remains a need for a fast, effective in vivo technique to investigate changes in defined populations of prokaryotic and eukaryotic cells within an animal.

### SUMMARY OF THE INVENTION

The present invention is directed to a method of using hollow fibers to evaluate cellular changes in vivo. The hollow fiber technique can be used to study the behavior of microorganisms or other cells of interest under various conditions in animals, such as, for example, in response to a specific drug or drugs of interest. Thus, a method for evaluating cellular changes in vivo in response to administration of a drug or drugs of interest is provided. In another embodiment, the hollow fiber technique is used to evaluate cellular changes in a microorganism in vivo. In one embodiment, the technique is used to evaluate cellular changes in a microorganism in vivo in response to administration of a drug or drugs of interest. A vaccine and a method of vaccinating an animal are also provided.

Other systems, methods, features, and advantages of the present invention will be or become apparent to one with skill in the art upon examination of the following drawings and detailed description. It is intended that all such additional systems, methods, features, and advantages be included within this description and be within the scope of the present invention.

### BRIEF DESCRIPTION OF THE DRAWINGS

Many aspects of the invention can be better understood with reference to the following drawings.

FIG. 1 is a photograph of an SKH1 mouse with a subcutaneously-implanted hollow fiber containing M. tuberculosis.

FIG. 2 is a graph of the reduced growth of bacilli within hollow fibers in vivo. Colony-forming unit (CFU) counts per fiber of hollow fiber-encapsulated M. tuberculosis implanted into mice (HF in vivo) are compared to those of hollow fiber-encapsulated M. tuberculosis incubated in vitro (HF in vitro).

FIG. 3 is a photograph of hollow-fiber encapsulated bacilli that remain viable in vivo. As a control, in vitro-grown cultures of M. tuberculosis H37Rv-lux were treated with 70% ethanol for 3 hours in order to promote bacillary death (FIG. 3a). Live bacilli exhibit green fluorescence while dead bacilli fluoresce red. Approximately half of all in vivo hollow fiber-encapsulated organisms on days 21 (FIG. 3b) and 28 (FIG. 3c) after fiber implantation were determined to be viable based on their staining properties.

FIG. 4 is a graph of the reduced metabolic activity of encapsulated bacilli in vivo. FIG. 4a is a graph of the relationship of relative light units (RLU) to colony-forming units (CFU) in mid-log phase M. tuberculosis H37Rv-lux grown in vitro. FIG. 4b is a graph of luciferase activity of hollow fiber-encapsulated M. tuberculosis implanted into mice (HF in vivo) vs. hollow fiber-encapsulated M. tuberculosis incubated in vitro (HF in vitro).

FIG. 5 is a graph demonstrating that hollow fiber-encapsulated bacilli are more susceptible to rifampin than to isoniazid. The activities of isoniazid 0.05% in the diet (INH) and rifampin 0.02% in the diet (RIF) against hollow fiber-encapsulated bacilli in vivo are compared to no treatment (Control).

FIG. 6 is a photograph of the formation of granuloma-like lesions surrounding M. tuberculosis-containing hollow fibers. Gross skin lesions surrounding hollow fibers containing liquid broth alone at day 1 (FIG. 6a), day 14 (FIG. 6b), and day 28 (FIG. 6c), and those containing M. tuberculosis H37Rv-lux at day 1 (FIG. 6d), day 14 (FIG. 6e), and day 28 (FIG. 6f), following hollow fiber implantation. Histopathology of tissues surrounding hollow fibers containing liquid broth is in FIGS. 6g-6i and those containing M. tuberculosis H37Rv-lux is shown in FIGS. 6j-6l) 28 days after hollow fiber implantation (hematoxylin-eosin stain). Arrows indicate hollow fiber membrane.

FIG. 7 is a graph demonstrating that containment of intra-fiber bacillary growth in vivo is immune-mediated and IFNγ-dependent. Mice implanted with hollow fibers containing M. tuberculosis (HF+M. tb) developed enlarged spleens as early as 14 days after implantation, as compared to mice implanted with fibers containing media (HF control) (FIG. 7a). Wild type Balb-C/J (WT) mice were able to contain the growth of hollow fiber-encapsulated M. tuberculosis to a greater extent than isogenic IFNγ-deficient (IFNg−/−) mice 28 days after hollow fiber implantation (FIG. 7b).

FIG. 8 is a graph of the absence of rel_{Mtb}-deficient mutant by PCR from a pool of mutants after 21 days of cultivation within mouse granulomas. PCR amplification of the transposon insertion junction reveals presence of the rel_{Mtb}::Tn mutant in both input (Day 1) and output (Day 21) pools in hollow fibers incubated in vitro (FIG. 8a) but absence of the mutant in the output pool (Day 21) in mouse-implanted hollow fibers (FIG. 8b), suggesting reduced survival of this mutant in vivo. 1=Rv1347 (hypothetical transcriptional regulator); 2 =Rv0250 (miscellaneous oxidoreductase); 3=Rv2583 (rel_{Mtb}); 4=Rv1069 (pra). Hollow fiber-encapsulated wild-type M. tuberculosis CDC 1551 (WT) and rel_{Mtb}::Tn mutant (RelMtb) grow equally when incubated in vitro (FIG. 8c), but the latter strain demonstrates significantly reduced survival when hollow fibers are implanted into mice (FIG. 8d).

FIG. 9 is a photograph of the colony size of hollow fiber-encapsulated M. tuberculosis incubated in vitro (left) versus implanted in mice (right) for 21 days. Photograph was obtained 17 days after plating.

FIG. 10 is a graph of the activity of moxifloxacin (MXF) against hollow fiber-encapsulated bacilli implanted into mice, as compared with no treatment (control).

### DETAILED DESCRIPTION

The present invention is directed to a method of using hollow fibers to evaluate cellular changes in vivo. The hollow fiber technique involving the use of semi-diffusible hollow fibers can be used to study the behavior of encapsulated microorganisms or other cells of interest under various conditions in animals.

The hollow fiber technique provides a unique method to study the behavior of a pure population of prokaryotic or eukaryotic cells in response to various conditions in an animal. In one embodiment, this technique is used to evaluate cellular changes in vivo in response to administration of a drug or drugs of interest. In one embodiment, the cells employed are eukaryotic cells such as human cells that are evaluated for potential toxicity or activity in response to administration of a drug or drugs of interest. In another embodiment, the hollow fiber technique is used to evaluate cellular changes in microorganisms in vivo, such as, e.g., during latency. In one embodiment, the technique is used to evaluate cellular changes in a microorganism in vivo in response to administration of a drug or drugs of interest. In another embodiment cellular changes of defined populations of prokaryotic or eukaryotic cells in the conditions described above including latency, exposure to drugs, exposure to immunomodulators, or exposure to gene therapy vectors may be monitored in animals that are genetically engineered with deficiencies or altered gene expression of specific animal genes. Thus, this hollow fiber technique can be used to evaluate such cellular changes as those developed during latent tuberculosis infection and can be used to characterize the human cellular pharmacogenomic expression profiles of various drugs against different human cell types.

In one embodiment, this technique is used to evaluate cellular changes in vivo in response to administration of a drug or drugs of interest. In one embodiment, the cells are evaluated for potential toxicity or activity in response to administration of a drug or drugs of interest. In one embodiment, this assay is used to screen novel drugs for desired activity or undesired toxicity.

Any suitable cells or cell lines can be evaluated, including, but not limited to, differentiated human cells. In one embodiment, the cells are non-transformed cells. In another embodiment, the cells are non-transformed cells isolated from a human. For example, in one embodiment, a drug or drugs are screened for potential toxicity against human cell types, for example, stem cells, peripheral blood cells (including peripheral blood mononuclear cells), lymphoid cells, hepatocytes, bone marrow-derived cells, skin biopsies, broncho-alveolar lavage washings, breast tissue cells, kidney cells, oral, urethral, vaginal, cervical, or gastric, or intestinal mucosal cells or mucosal biopsies, reproductive cells (ova or spermatocytes), adipose cells, nerve or stromal cells, bone or synovial cells, or other suitable human cell types, by studying the transcriptional profiles of these cells. It is envisioned that both normal and malignant cells from the tissues mentioned will be amenable to hollow-fiber testing. Global gene expression patterns of various human cell types with the use of microarrays, reverse transcriptase-polymerase chain reaction (RT-PCR), or other gene expression methods are correlated with specific cellular toxicity profiles. In one embodiment, different profiles are associated with cells isolated from different patients, which could allow for individualized medicine. In another embodiment, novel chemotherapeutic agents are tested for activity against specific human cancer cell lines, by quantification of cells prior to and following administration of the drug. In addition, the same drugs are then screened using the hollow fiber technique for potential toxicity against other human cell types. Therefore, the hollow fiber technique provides a rapid, inexpensive in vivo assay with which to screen promising new drugs for human activity and toxicity parameters prior to the investment of significant resources in human clinical trials.

In one embodiment, a method for evaluating cellular changes in vivo in response to administration of a drug or drugs of interest is provided. The method comprises encapsulating cells of interest in a hollow fiber; implanting the hollow fiber into an animal; administering the drug or drugs of interest to the animal; isolating the cells from the hollow fiber; and evaluating the transcriptional profiles of the cells. The animal can be any suitable animal, such as, for example, a mouse, a rat, a guinea pig, a rabbit, a sheep, a pig, a cow, a chicken, or a dog, or genetically engineered variant animals of the aforementioned species. Of course, more than one animal can be utilized. The cells can be any suitable cells, such as those described herein, such as, e.g., cells isolated from a human. The transcriptional profiles can be evaluated in any suitable manner, such as those known in the art, for example, by using microarray analysis. In one embodiment, the transcriptional profiles are correlated with specific cellular toxicity or activation profiles. In this embodiment, the correlation allows a determination of the effects of the administration of the drug or drugs on the cells.

The drug or drugs administered can be any suitable drug or combination of drugs, including, e.g., novel drugs and known drugs. The drug can be administered in any suitable manner or using any suitable dosing schedule, such as, e.g., once a day, twice a day, three times a day, bi-weekly, three times a week, four times a week, or any other suitable dosing schedule. If more than one drug is administered, they can be administered concurrently or sequentially or at different times or even different days altogether. In one embodiment, the drug or drugs is selected from the group consisting of: analgesics, anesthetics, anti-acne agents, antibiotics, anticholinergics, anticoagulants, anticonvulsants, antidiabetic agents, antidyskinetics, antifibrotic agents, antifungal agents, anti-glaucoma agents, anti-infectives, anti-inflammatory compounds, antimicrobial compounds, antineoplastics, antiParkinsonian agents, antirheumatic agents, antiosteoporotics, antiseptics, antisporatics, antithrombotics, antivirals, appetitite stimulants, bacteriostatics, biologicals, blood modifiers, bone metabolism regulators, calcium regulators, cardioprotective agents, cardiovascular agents, central nervous system stimulants, cholinesterase inhibitors, contraceptives, cystic fibrosis agents, deodorants, detoxifying agnets, diagnostics, disinfectants, dietary supplements, dopamine receptor agonists, enzymes, erectile dysfunction agents, fertility agents, gastrointestinal agents, gout agents, hormones, hypnotics, immunomodulators, immunosuppressives, keratolytics, mast cell stabilizers, migraine agents, motion sickness agents, multiple sclerosis treatments, muscle relaxants, nasal preparations, nucleoside analogs, obesity agents, opthalmic agents, osteoporosis agents, parasympatholytics, parasympathomimetics, prostaglandins, psychotherapeutic agents, respiratory agents, sclerosing and anti-sclerosing agents, sedatives, skin and mucous membrane agents, smoking cessation agents, sympatholytics, ultraviolet screening agents, urinary tract agents, vaginal agents, vasodilators, or combinations thereof. Suitable classes of drugs are described in, e.g., Physicians' Desk Reference, 56^{th} Ed., Medical Economics Company, Inc., Montvale, N.J., pages 201-202 (2002). In one embodiment, the drug is a novel drug with unknown or uncertain activity and/or toxicity.

The drug or drugs can be administered for any period of time suitable to give the desired result. In one embodiment, the drug or drugs is administered for a period of from about 1 hour to about 30 days, such as, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, and about 30 days. Of course, a suitable amount of time could also include more than about 30 days where appropriate.

In another embodiment, the hollow fiber technique is used to evaluate cellular changes in a microorganism in vivo, such as, e.g., during latency. In one embodiment, a method for evaluating cellular changes in a microorganism in vivo is provided. The method comprises: encapsulating one or more microorganisms in a hollow fiber; implanting the hollow fiber into an animal; isolating the microorganisms from the hollow fiber; and evaluating the transcriptional profiles of the microorganisms. Any suitable animal can be employed, such as, e.g., those described herein. The microorganisms can isolated from the hollow fiber after any period of time suitable for evaluating the cellular changes, such as, e.g., from about 1 hour to about 30 days, such as, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, and about 30 days. Of course, a suitable amount of time could also include more than about 30 days where appropriate.

The transcriptional profiles can be evaluated in any suitable manner, such as those known in the art, for example, by using microarray analysis. In one embodiment, the transcriptional profiles are used to design drugs that specifically target the microorganism. For example, in one embodiment, the method allows identification of genes required for persistence that can be specifically targeted in the design of drugs or vaccines. In one embodiment, such drugs or vaccines can be screened against the microorganism using the hollow fiber technique. In one embodiment, the microorganism is deficient in a specific gene, such as, e.g., a gene required for persistence.

Any suitable microorganism can be evaluated. In one embodiment, the microorganism is selected from the group consisting of: Mycobacterium species including M. tuberculosis, Staphylococcal species including Staphylococcus aureus, Streptococcus pneumoniae, Streptococcus pyogenes, other pathogenic Streptococcal species, including Enterococci, Haemophilus species, Moraxella species, Neisseria species, Legionella species, Listeria species, Chlamydia species, Mycoplasma species, Pseudomonas species, Escherichia coli, Klebsiella species, Enterobacter species, Serratia species, Acinetobacter species, Xanthomonas species, Stenotrophomonas, Borrelia species, Treponemal species, Nocardia species, Actinomycete species, Bacteroides species, Clostridial species including C. difficile, Peptostreptococci, Bacillus species, Francisella species, Yersinia species, Candida species, including Candida albicans, Histoplasma species, Cryptococcus species, Aspergillus species, Blastomycosis species, viruses within appropriate cellular carriers including HIV, smallpox virus (Variola), hepatitis A, B, C, D, and E viruses, influenza viruses, rhinoviruses, adenoviruses, coxsackie viruses, parainfluenza viruses, poliovirus, measles virus, Varicella virus, Herpesviruses including HSV-1 and HSV-2, Cytomegalovirus (CMV), Noroviruses, and parasitic species including Plasmodia species, Giardia species, Toxoplasma species, Schistosoma species, Trypanosoma species, and Leishmania species. In one embodiment, the hollow fiber technique is used to evaluate microorganisms other than M. tuberculosis, such as, e.g., aerobic and anaerobic bacteria, fungi and yeasts, parasites, and virus carried within appropriate defined cells.

In one embodiment, the technique is used to evaluate cellular changes in a microorganism in vivo in response to administration of a drug or drugs of interest. In one embodiment, a method for evaluating cellular changes in a microorganism in vivo in response to administration of a drug or drugs of interest, comprising: encapsulating one or more microorganisms in a hollow fiber; implanting the hollow fiber into an animal; administering the drug or drugs of interest to the animal; isolating the microorganisms from the hollow fiber; and evaluating the transcriptional profiles of the microorganisms. The animal can be any suitable animal, such as, for example, those described herein. The microorganism can be any suitable microorganism, such as those described herein. The transcriptional profiles can be evaluated in any suitable manner, such as those known in the art, for example, by using microarray analysis. In one embodiment, the transcriptional profiles are correlated with specific cellular toxicity or activation profiles. In this embodiment, the correlation allows a determination of the effects of the administration of the drug or drugs on the microorganism. In one embodiment, the microorganism is a microorganism other than M. tuberculosis, such as, e.g., such as, e.g., aerobic and anaerobic bacteria, fungi and yeasts, parasites, and virus carried within appropriate defined cells.

The drug or drugs administered can be any suitable drug or combination of drugs, such as, e.g, those described herein. In one embodiment, the drug or drugs can be an antibacterial antibiotic, an antiviral agent, antiparasitic agent, or anti-fungal agent or combination thereof.

The drug or drugs can be administered for any period of time suitable to give the desired result. In one embodiment, the drug or drugs is administered for a period of from about 1 hour to about 30 days, such as, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, and about 30 days. Of course, a suitable amount of time could also include more than about 30 days where appropriate.

In one embodiment, this technique is used to study the transcriptional profile of Mycobacterium tuberculosis during latency using microarray analysis, in order to identify upregulated genes whose products may be involved in metabolic pathways critical for mycobacterial survival and persistence within the host. This information provides the basis for rational design of drugs to specifically target the persistent stage of tuberculosis infection. M. tuberculosis mutants deficient in specific genes are tested individually or as members of a pool for the ability to persist in the hollow fiber mouse model. Genes required for persistence are specifically targeted in the rational design of attenuated vaccine strains. Finally, promising new anti-TB drugs are screened in the hollow fiber model of latent TB infection for activity against slowly-replicating, metabolically hypoactive (i.e., dormant) M. tuberculosis.

The hollow fiber technique is additionally useful for: screening drugs as antibiotics against M. tuberculosis; screening drugs as antibiotics against other bacteria, such as, for example, bacteria that cause osteomyelitis, or foreign-body (e.g., catheters, prostheses, etc.) infections, e.g., Staph. aureus, Enterococci, fungi, such as, e.g., Candida albicans; assessing the transcriptional and proteomic profile of bacteria within the fibers (“latent-like”) bacteria, a novel approach to vaccination (the bacteria appear to secrete small molecules outside of the fibers which elicit immune responses); or screening encapsulated human cells implanted in mice for transcriptional signatures associated with drug efficacy or drug toxicity.

Therefore, in one embodiment, a vaccine is provided. The vaccine comprises at least one hollow fiber comprising at least one microorganism. Any suitable microorganism can be utilized, such as, e.g., those set forth herein. In one embodiment, the microorganism is attenuated. In one embodiment, the microorganism is Mycobacterium tuberculosis. In another embodiment, the microorganism is mutant M. tuberculosis. Any suitable attenuated microorganism can be used, such as, e.g., microorganisms deficient in genes for persistence.

A method of vaccinating an animal is also provided. The method comprises encapsulating one or more microorganisms in a hollow fiber and implanting the hollow fiber into the animal. Any suitable animal can be vaccinated, such as, e.g., a human. In one embodiment, the microorganism is attenuated. In one embodiment, the microorganism is Mycobacterium tuberculosis. In another embodiment, the microorganism is mutant M. tuberculosis. Any suitable attenuated microorganism can be used, such as, e.g., microorganisms deficient in genes for persistence.

In one embodiment, the vaccine is prepared by encapsulating one or more microorganisms into at least one hollow fiber. The vaccine can be prepared using any suitable microorganism, including vaccines that are attenuated and/or mutated. Attenuation and/or mutation of the microorganism can be accomplished using any suitable techniques, such as, e.g., those described herein or otherwise known in the art.

The hollow fiber technique provides a rapid screening tool to test the activity of novel chemotherapeutic agents against encapsulated organisms or other cells of interest in animals. This technique can also be used to study the global transcriptional profile of encapsulated organisms or other cells of interest within animals. In addition, the hollow fiber technique can be used to study the ability of various bacterial mutants to persist within animals, thereby allowing the identification of virulence genes, or genes required for in vivo bacterial survival. Finally, the hollow fiber technique permits evaluation of the effects of soluble factors secreted by encapsulated organisms or other cells of interest on host immune response and can be used as a vaccination strategy.

In one embodiment, the hollow fiber encapsulation/implantation technique is used to study the behavior of microorganisms in the extracellular compartment of an animal. In one embodiment, the hollow fiber encapsulation/implanatation technique is used to encapsulate bacilli in semi-diffusible hollow fibers which are implanted subcutaneously into mice, creating an in vivo model of tuberculosis. This embodiment grants a unique opportunity to study the behavior of extracellular Mycobacterium tuberculosis in animals. Specifically, this embodiment provides insight into the adaptive mechanisms employed by M. tuberculosis during persistence in the host through global gene expression analysis, and evaluation of specific gene-deficient mutants for the ability to persist. In one embodiment, this model can be used to study the activity of various drugs against dormant bacilli in vivo.

Mycobacterium tuberculosis residing within pulmonary granulomas and cavities represents an important reservoir of persistent organisms during human latent tuberculosis infection. Granulomatous lesions develop around these hollow fibers, and in this microenvironment, the organisms demonstrate an altered physiologic state characterized by stationary-state colony-forning unit counts and decreased metabolic activity. Moreover, these organisms show an antimicrobial susceptibility pattern similar to persistent bacilli in current models of tuberculosis chemotherapy in that they are more susceptible to the sterilizing drug rifampin, than to the bactericidal drug isoniazid. This model of extracellular persistence within host granulomas can be used to study both gene expression patterns and mutant survival patterns.

In one embodiment the hollow-fiber method is used to cultivate microbial species which either are impossible or difficult to cultivate in vitro or in non-hollow-fiber animal models. These microbes could be cultivated in relatively large quantities within hollow fibers implanted with an appropriate animal host for research purposes, for purposes of developing anti-microbials, for the purposes of clinical diagnosis, for the purpose of deriving pure microbial products in relatively large quantities, for the purpose of developing improved diagnostics, for the purpose of developing vaccines, or for other suitable purposes. Such microbes include but are not limited to: Mycobacterium leprae (agent of leprosy), Treponema pallidum (agent of syphilis), and hepatitis C virus with appropriate carrier eukaryotic cells, or other suitable microbes described herein.

In one embodiment, the hollow fiber technique employs semi-diffusible fibers. In one embodiment, the fibers are commercially available fibers, such as, e.g., PVDF hollow fibers with 500 kDa molecular weight cutoff available from Spectrum Laboratories, Inc., 18617 Broadwick Street, Rancho Dominguez, Calif. 90220 USA. In one embodiment, the physical parameters of the hollow fibers can be altered to optimize their utility. For example, the fibers are composed of various synthetic compounds in order to reduce immunogenicity. The internal diameter of the fibers can be increased to accommodate a great volume of cells of interest. In addition, the pore size of the fiber membranes can be varied, according to the specific cell types or secreted factors studied. In one embodiment, the hollow fibers are implanted in the subcutaneous or intraperitoneal spaces of experimental mice, rats, guinea pigs, and rabbits. In one embodiment, for the study of lung-tropic pathogens, fibers may be deposited bronchoscopically in the airways of rabbits.

The present invention is further illustrated by the following examples, which should not be construed as limiting in any way. The practice of the present invention will employ, unless otherwise indicated, conventional techniques that are within the skill of the art.

### EXAMPLES

### Example 1

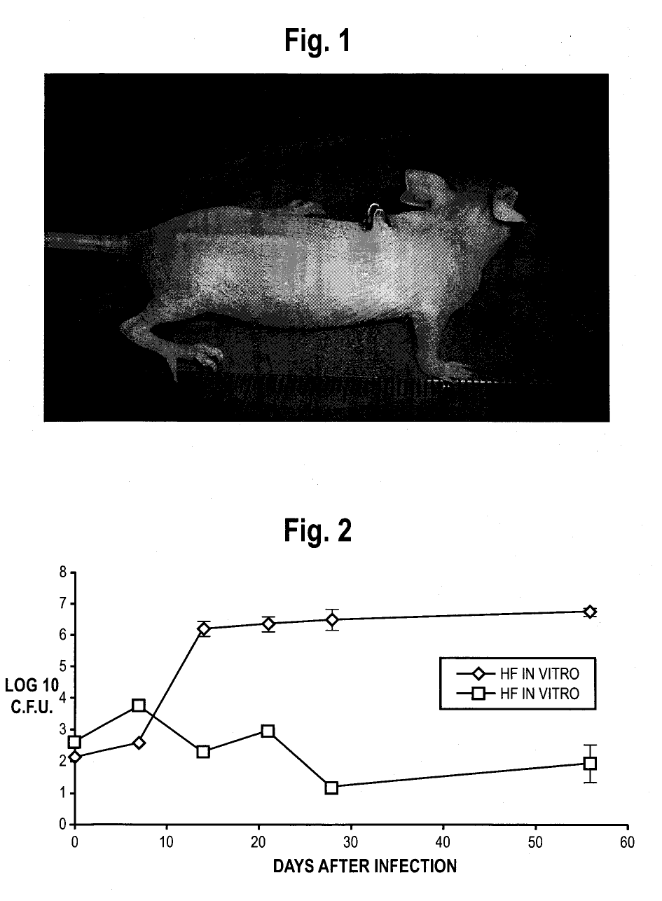

This example demonstrates the physical containment of extracellular M. tuberculosis within mice as a means of comparing in vivo-cultivated persistent bacteria with in vitro latency models. This example demonstrates the encapsulation of tubercle bacilli in polyvinylidene fluoride (PVDF) hollow fibers (Hollingshead, M. G., et al. Life Sci 57:131-141 (1995), Xu, Z. Q., et al. Bioorg Med Chem Lett 9:133-138 (1999)) and the implantation of these fibers into the subcutaneous space of mice (FIG. 1). The PVDF fibers have a molecular weight cutoff of 500 kDa, which allows for the diffusion of small soluble molecules, but prevents the entry of host immune cells and the exit of intact bacilli. The prevention of direct host-pathogen interactions imposed by the physical properties of the hollow fibers provides a unique opportunity to study the behavior of extracellular M. tuberculosis in the host.

Materials and Methods

Strains: M. tuberculosis H37Rv expressing firefly luciferase (H37Rv-lux) was passaged twice through mice and used for all infections. The organisms were grown in plastic roller bottles at 37° C. in Middlebrook 7H9 liquid broth (Difco Laboratories) supplemented with 10% OADC (Becton Dickinson), 0.5% glycerol and 0.05% Tween-80.

Hollow fiber assay: Infection with M. tuberculosis was achieved in 6-8 week old female hairless immunocompetent SKH1 mice (Charles River Laboratories) using the hollow fiber encapsulation/ implantation technique as described previously. Hollingshead, M. G., et al. Life Sci 57:131-141 (1995), Xu, Z. Q., et al. Bioorg Med Chem Lett 9:133-138 (1999). Briefly, liquid cultures of M. tuberculosis were inoculated into the lumen of polyvinylidene fluoride (PVDF) hollow fibers (molecular weight cutoff 500 kDa, Spectrum Laboratories) with a syringe and 20-gauge needle. The ends of the hollow fibers were heat-sealed and individual fibers were prepared by heat-sealing at 2-cm intervals. Mice were anesthetized by intraperitoneal injection of Avertin (2,2,2-tribromoethanol) 240 mg/kg (Sigma-Aldrich) and the dorsal skin surface was sterilized with 70% ethanol. A small incision was made at the nape of the neck and one fiber was deposited into the subcutaneous space of each flank with a tumor trocar (2 fibers/mouse). Incisions were closed with a small surgical clip (FIG. 1). For immunology studies, Balb-C/J and IFNγ−/−(B6-IFNg^{tmITSIJ}) were used (Jackson Labs). For experiments involving hollow fiber-encapsulated M. tuberculosis incubated in vitro, hollow fibers were incubated at 37° C. in 50-ml conical tubes containing 20 ml Middlebrook 7H9 liquid broth (Difco) supplemented with 10% OADC (Becton Dickinson), 0.5% glycerol and 0.05% Tween-80. For determination of colony-forming unit (CFU) counts, hollow fibers were recovered from mice at the time of sacrifice and their contents plated on 7H10 agar plates (Fischer Scientific). Log-transformed CFU values were used to calculate averages and standard errors for graphing purposes.

Luciferase assay: The luciferase reaction was initiated by the addition of 150 μl of luciferin (1 mM in 0.1 M Na citrate; Promega) to 50 μl of each undiluted sample. Luminescence was detected 20 seconds after the addition of substrate by a TD-20/20 luminometer (Turner Designs). Three successive measurements were made and the average relative light unit (RLU) values recorded. The log₁₀ of average RLU values multiplied by 1000 was represented graphically.

Microarrays: Hollow fibers each containing 10⁶ bacilli were implanted into 15-20 SKH1 mice (2 hollow fibers/mouse). Hollow fibers were retrieved from mice 10 days after hollow fiber encapsulation and fiber contents were recovered and snap-frozen. Pooled samples were suspended in TriZOL reagent (GIBCO/BRL), and M. tuberculosis membranes were disrupted using zirconia/silica beads in a bead beater. M. tuberculosis RNA was recovered by centrifugation, chloroform extraction, and isopropyl alcohol precipitation, and purified using RNeasy column (Qiagen) as previously described. Sherman, supra, Betts, J. C., et al. Mol Microbiol 43:717-731 (2002). The same steps were followed to extract and purify RNA from mid-logarithmic phase (A₆₀₀, 0.600-0.850) M. tuberculosis H37Rv-lux cultures grown in plastic roller bottles at 37° C. in supplemented liquid broth. Fluorescently labeled cDNA was generated using Powerscript (Clontech), using fluorescent dyes Cy3 and Cy5. These cDNA were competitively hybridized on microarray slides containing commercially available (Operon) M. tuberculosis 70-mer oligo-nucleotides representing all opening reading frames annotated in the H37Rv genome sequencing project (Cole, S. T., et al. Nature 393:537-544 (1998)), and fluorescence intensity data were collected with a GenePix 4000 scanner (Axon Instruments) with GenePix Pro 4.0 software. Data were normalized based on total intensity of good-quality spots above background for each channel, and ratios of in vivo hollow fiber to in vitro cDNA were calculated based on normalized data. In this assay, the ratio of the signal from in vivo hollow fiber samples to that of in vitro control samples for a given open reading frame (ORF) should represent the relative abundance of the transcripts of that ORF under the two conditions. Three biological replicates were performed, and microarray samples were reverse-labeled in one experiment. Significant differential regulation of genes was defined by ≧2-fold change in gene expression as compared to control samples and p<0.01.

Quantitative real-time RT-PCR: RNA samples were treated with DNA-free kit (Ambion) according to the instructions of the manufacturer, and DNA contamination was excluded by PCR amplification using primers for Rv2031 (acr) and absence of PCR product on gel electrophoresis. Reverse transcription of RNA samples (˜0.5 μg RNA/sample) was accomplished using random hexamer primers (Invitrogen; 0.5 μg/reaction) and Powerscript Reverse Transcriptase (Clontech). Real-time PCR was performed on cDNA using the SYBR-green assay and Premix D (Epicentre) for all samples, and fluorescence was measured by iCycler (Biorad). Gene expression was compared to that of sigA, a M. tuberculosis housekeeping gene.

Transposon mutants: Random insertion mutagenesis of M. tuberculosis CDC 1551 strain was performed using the Himar1 transposon as previously described. Rubin, E. J., et al. Proc Natl Acad Sci USA 96:1645-1650 (1999), Lamichhane, G., M. et al. Proc Natl Acad Sci USA 100:7213-7218 (2003). Transposon (Tn) insertion sites were identified by sequencing the insertion junction as previously described (Lamichhane, supra). One hundred different mutants, each with Tn insertion within the proximal 80% or proximal to the distal 100 base pairs of the ORF, were randomly selected from the library of mutants. Each of the selected mutants was separately grown in 37° C. in supplemented Middlebrook 7H9 liquid broth to an A₆₀₀ of 0.8-1.0, and 2 master pools, each consisting of 50 mutants, were prepared by mixing an equal volume of culture of each mutant. This mixture was diluted to an A₆₀₀ of 0.1 and the latter was used to inoculate the hollow fibers. In separate experiments, the rel_{Mtb}-disrupted Tn insertion mutant was grown separately in vitro using the conditions described above to an A₆₀₀ of ˜1.0, and diluted 1:10. The diluted culture was then added in a 1:1 ratio to a similarly grown and diluted culture of wild-type CDC1551. The Δrel_{Mtb}/wild-type culture suspension was then encapsulated in hollow fibers as described above and either implanted subcutaneously into mice or incubated in vitro. Recovered hollow fiber samples were plated on Middlebrook 7H10 plates (Difco) and on 7H10 plates containing kanamycin 20 μg/ml (the transposon insertion contains a kanamycin resistance gene. Lamichhane, supra).

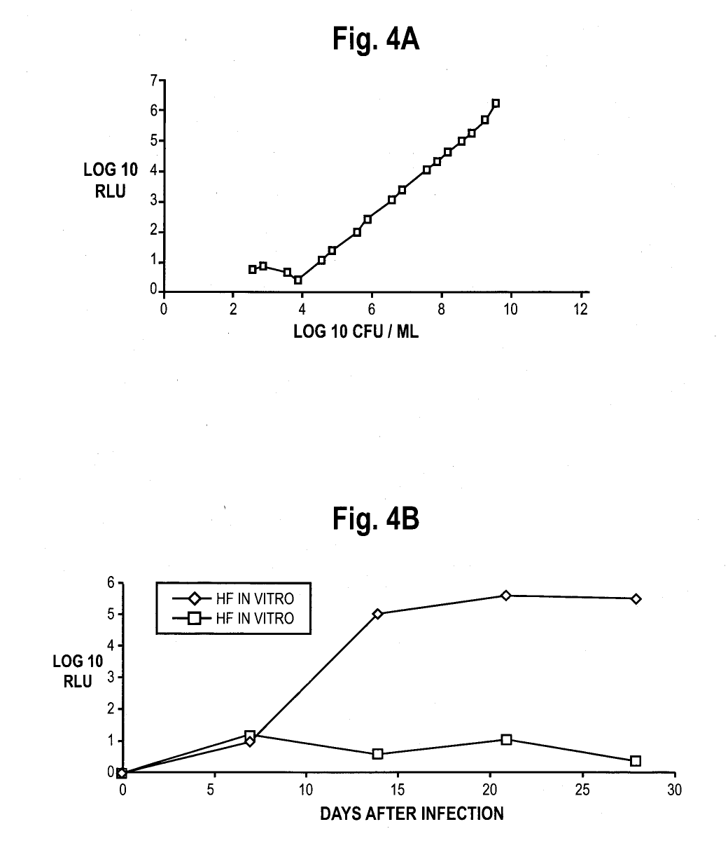

Viability assay: The LIVE/DEAD BacLight Bacterial Viability Kit (Molecular Probes) was used to assay for mycobacterial viability after removal from hollow fibers. Briefly, Component A (SYTO 9 green-fluorescent nucleic acid stain, 3.34 mM) and Component B (propidium iodide, 20 mM) were mixed in equal volumes. An equivalent of 3 μ1 of this 1:1 nucleic acid stain mixture was added to each 1 ml of sample, and samples were incubated in the dark for ≧15 min. As a control, in vitro-grown M. tuberculosis H37Rv-lux was treated for 3 hours with 70% ethanol in order to promote mycobacterial death and highlight differences in fluorescence staining between live and dead bacilli (FIG. 3a). Samples were treated with 20 μl of 4% paraformaldehyde (which does not alter cell permeability characteristics) for ≧15 min and samples were observed using epifluorescence microscopy (Nikon Eclipse E800). Images were obtained using a Nikon Digital Camera DXM1200 and processed using Spot Version 3.4 software.

Antibiotic studies: Mice received a powdered diet containing 1% sugar with either 0.02% (by weight) rifampin (Sigma) or 0.05% isoniazid (Sigma), beginning 14 days after implantation of hollow fibers. Untreated control mice received powdered diet containing 1% powdered sugar alone. In separate experiments, mice received diet containing 2.5% sugar alone (untreated controls) or with 0.25% moxifloxacin beginning on day 1 after hollow fiber implantation. Mouse dietary consumption was measured and recorded for all groups on a daily basis. At the time of sacrifice, blood was obtained from antibiotic-treated mice by cardiac puncture, mouse serum was separated, and serum samples were stored at −70° C. until the time of analysis. Mouse serum samples were evaluated for determination of serum antibiotic concentrations.

A complete list of all transposon (Tn) mutants used, including information on the gene mutated and the exact coordinate of Tn insertion, is presented in Table 2. DosR genes which fulfilled one but not both criteria for significant upregulation are also presented in Table 2. The complete lists of genes found to be significantly upregulated or downregulated by microarray analysis in hollow fiber-encapsulated bacilli in vivo, as well as all genes not found to be differentially regulated, are also presented in Table 2.

Results

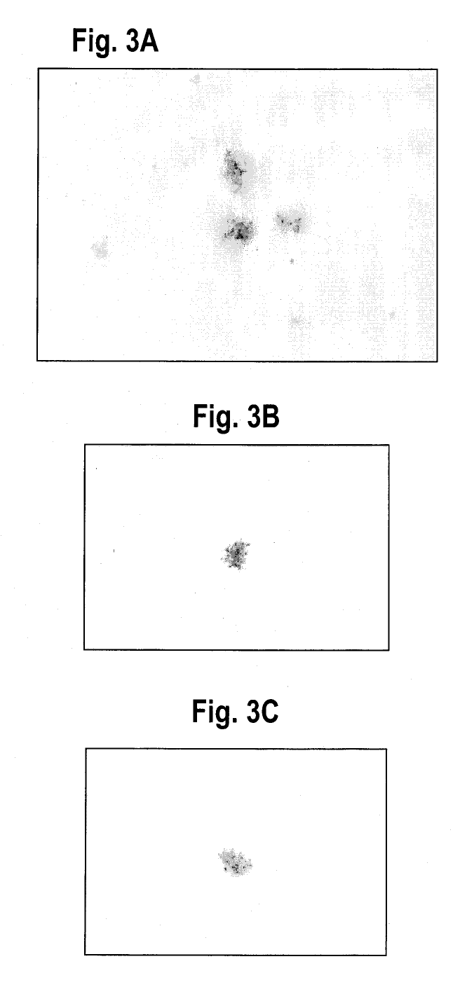

Inhibition of Growth of Encapsulated Tubercle Bacilli In Vivo (FIG. 2)

M. tuberculosis H37Rv expressing firefly luciferase (H37Rv-lux) was encapsulated in hollow fibers and implanted into the subcutaneous space of mice or incubated at 37° in supplemented Middlebrook 7H9 broth. Whereas hollow fiber-encapsulated bacilli incubated in vitro multiply exponentially for 14-21 days before reaching a plateau, bacilli encapsulated in hollow fibers and implanted into mice rapidly achieve stationary-state colony-forming unit (CFU) counts (FIG. 2). The continued growth of in vitro hollow fiber-encapsulated bacilli implies that the failure of in vivo hollow fiber-encapsulated bacilli to multiply is not simply a result of physical containment imposed by the internal dimensions of the hollow fibers.

Assessment of Bacillary Viability and Metabolic Activity

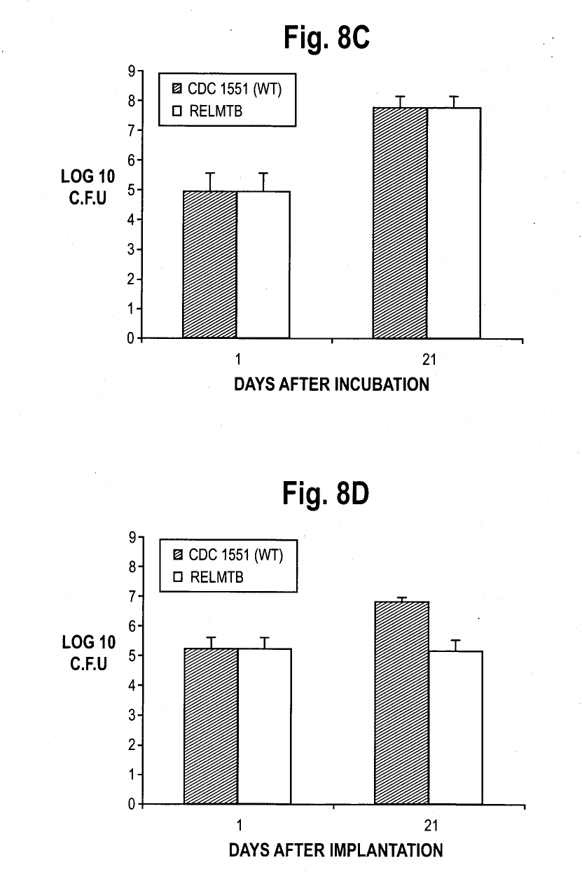

In order to further investigate the CFU equilibrium in hollow fiber-encapsulated bacilli in vivo, the bacilli recovered from fibers was examined using a bacterial viability assay in which live M. tuberculosis bacilli emit green fluorescence, while dead bacilli fluoresce red, based on differences in cell membrane permeability to the two nucleic acid stains used in the assay (FIG. 3). If the stationary-state CFU counts observed in hollow fiber-encapsulated bacilli in vivo represented an equilibrium between bacillary multiplication and death, one would expect that live-staining bacilli would comprise a very small proportion of all staining bacilli, due to the vast accumulation of dead bacilli over time. However, approximately half of all staining organisms on days 21 (FIG. 3b) and 28 (FIG. 3c) after hollow fiber implantation were determined to be alive by their staining properties, consistent with the hypothesis that these organisms are in a slowly replicating or non-replicating persistent state. In addition, a significant lag time in the appearance of colonies after plating the in vivo-cultivated bacilli was detected, as compared to hollow fiber-encapsulated bacilli incubated in vitro. At the day 21 time point, microcolonies were detected 10-11 days after plating in the in vitro hollow fiber samples, whereas these were not detectable in the in vivo hollow fiber samples until 14 days after plating. Furthermore, average colony size for in vivo hollow fiber samples was significantly smaller than corresponding in vitro hollow fiber samples when examined 17 days after plating (FIG. 9). These data are consistent with the hypothesis that hollow fiber-encapsulated M. tuberculosis implanted in mice rapidly enters a state of decreased replication.

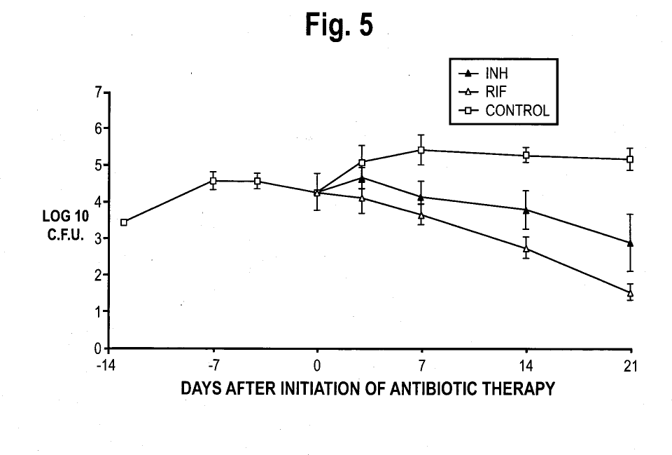

Because the oxidation of luciferin to oxyluciferin and photons (which can be detected by a luminometer at ˜560 nm) is an ATP-dependent process, the luciferase assay may be used as an indirect measure of the ATP content of cells. In log-phase in vitro cultures of M. tuberculosis (i.e., not encapsulated in fibers), the relationship between CFU and relative light units (RLU) is linear (FIG. 4a). Hollow fiber-encapsulated M. tuberculosis grown in vitro maintains baseline metabolic activity, as reflected by RLU values that closely parallel CFU counts at the corresponding time points (FIG. 4b). On the contrary, RLU values of in vivo-cultivated hollow fiber-encapsulated bacilli on days 14 and 21 after implantation (FIG. 4b) were 0.8 log₁₀ and 0.93 log₁₀, respectively, less than predicted based on the corresponding CFU values (FIG. 2) and the proportionality of CFU:RLU (FIG. 4a), suggesting reduced metabolic activity of these organisms, as measured by the luciferase assay. These data suggest that in vivo hollow fiber-encapsulated M. tuberculosis bacilli rapidly enter a state of decreased metabolic activity.

Antibiotic Susceptibility of Hollow Fiber-encapsulated Bacilli

Animal and human chemotherapy studies have pointed to several populations of M. tuberculosis in the mammalian host, including rapidly-multiplying, and non-replicating or sporadically-replicating persistent bacilli. Mitchison, D. A. J Antimicrob Chemother 29:477-493 (1992), McKinney, J. D. Nat Med 6:1330-1333 (2000). In keeping with this mixed bacterial population model, drug treatment studies have demonstrated that the sterilizing drug rifampin is more active against sporadically-multiplying and persistent bacilli than the bactericidal drug isoniazid, the former drug requiring a significantly shorter duration of therapy in order to achieve acceptable relapse rates. Lecoeur, H. F., et al. Am Rev Respir Dis 140:1189-1193 (1989); Am Rev Respir Dis 145:36-41 (1992); Dickinson, J. M., and D. A. Mitchison. Am Rev Respir Dis 123:367-371 (1981); Jasmer, R. M., et al. N Engl J Med 347:1860-1866 (2002); Fox, W., and D. A. Mitchison. Am Rev Respir Dis 111:325-353 (1975). Thus, rifampin likewise should be more effective than isoniazid against hollow fiber-encapsulated M. tuberculosis implanted into mice if these bacilli were truly in a slowly-replicating, metabolically quiescent state. In order to allow bacilli to enter this altered physiologic state, antibiotics were not initiated until 14 days after hollow fiber implantation. Experimental groups of mice received either powdered diet containing isoniazid 0.05% or rifampin 0.02% (by weight), while untreated mice received antibiotic-free diet. Serum concentrations of each antibiotic were measured in sacrificed mice at each timepoint, and were found to be at least 10 times greater than their respective MIC₉₀ (Table 1).

TABLE 1
Serum isoniazid and rifampin concentrations in mice
Isoniazid

| SKH1 mice (0.02% diet): | | Humans (5 mg/kg): | |
|---|---|---|---|
| Cmean (mg/L) | AUC (mg h/L) | Cmax (mg/L) | AUC (mg h/L) |
| 0.77 ± 0.21 | 18.5 ± 5.0 | 5.4 ± 2.0^{a} | 19.9 ± 6.1^{a} |
| | | 7.1 ± 1.9^{b} | 48.2 ± 1.5^{b} |

Rifampin

| SKH1 mice (0.05% diet): | | Humans (10 mg/kg): | |
|---|---|---|---|
| Cmean (mg/L) | AUC (mg h/L) | Cmax (mg/L) | AUC (mg h/L) |
| 3.09 ± 0.41 | 74.2 ± 9.8 | 14.91 | 117.93 |

^{a}= rapid acetylators
^{b}= slow acetylators

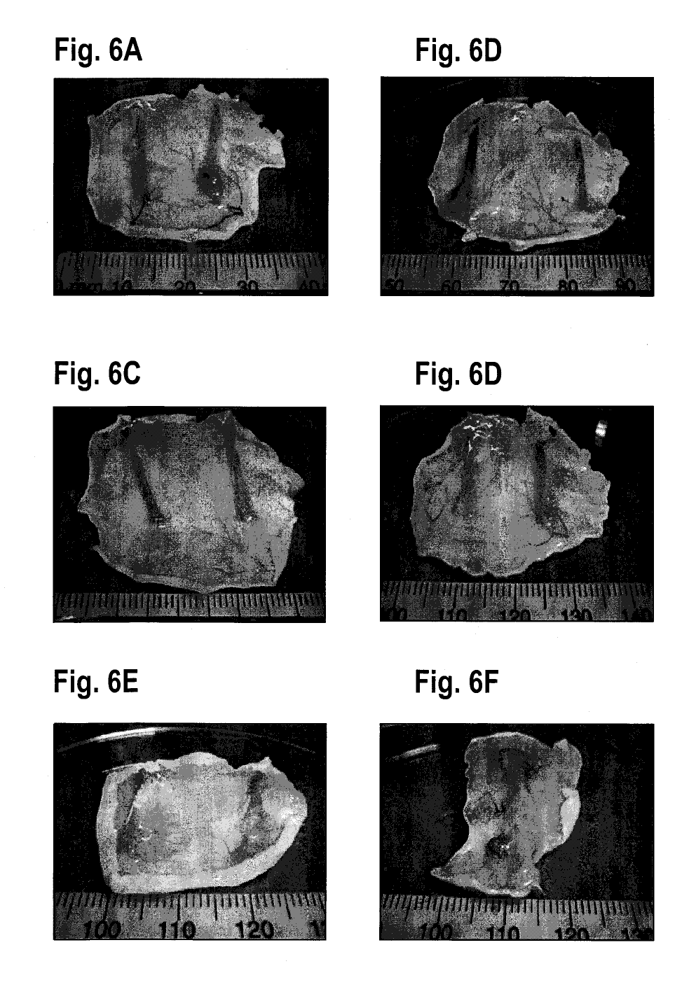

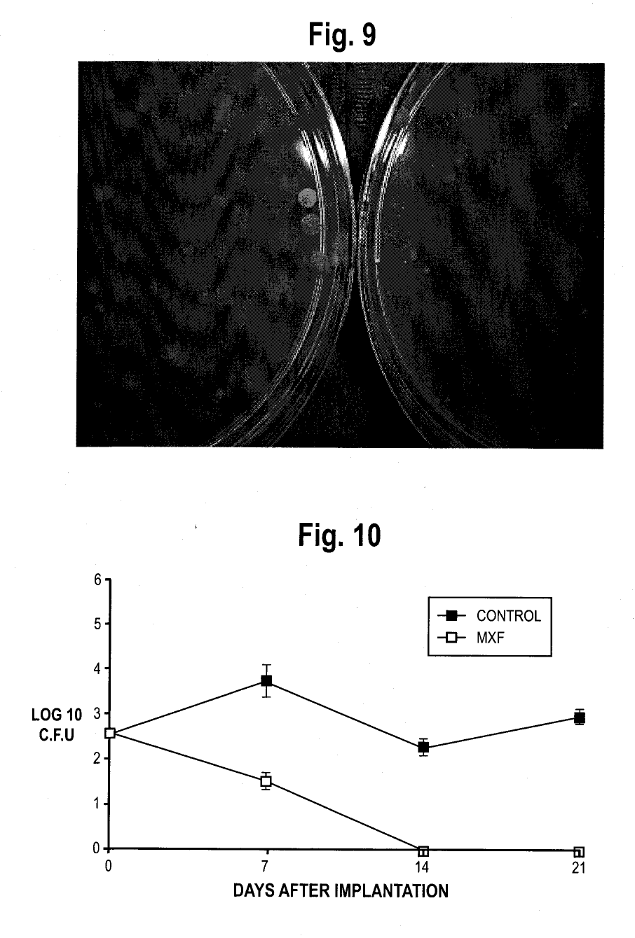

Daily area under the serum concentration-time curve (AUC) properties of each dietary regimen in mice were found to be similar to the AUC in humans after standard daily doses of each drug. Kim, Y. G., Clin Pharmacol Ther 54:612-620 (1993), Kenny, M. T., and B. Strates. Drug Metab Rev 12:159-218 (1981). Although isoniazid was somewhat effective against bacilli in the hollow fiber model in vivo, the activity of rifampin was more consistent and the magnitude of its effect greater than that of isoniazid at each time point, resulting in a 3-log kill by 21 days after initiation of therapy (FIG. 5). When mice were treated with the new 8-methoxyfluoroquinolone moxifloxacin, which has potent bactericidal activity against M. tuberculosis in mice (Nuermberger, E. L., et al. Am J Respir Crit Care Med. (2003)), beginning on day 1 after hollow fiber implantation, fibers were culture-negative by day 14 after daily therapy (FIG. 10). These data suggest that by 14 days after hollow fiber implantation, hollow fiber-encapsulated M. tuberculosis has entered an altered physiologic state in which bacilli are more resistant to the activity of anti-TB drugs, but more sensitive to rifampin than to isoniazid, consistent with the antibiotic susceptibility of persistent bacilli in human latent TB infection. Lecoeur, supra; Am Rev Respir Dis 145:36-41 (1992); Dickinson, J. M., supra; Jasmer, R. M., supra; Fox, W., supra.
Granuloma Formation Surrounding Bacilli-containing Hollow Fibers in Mice

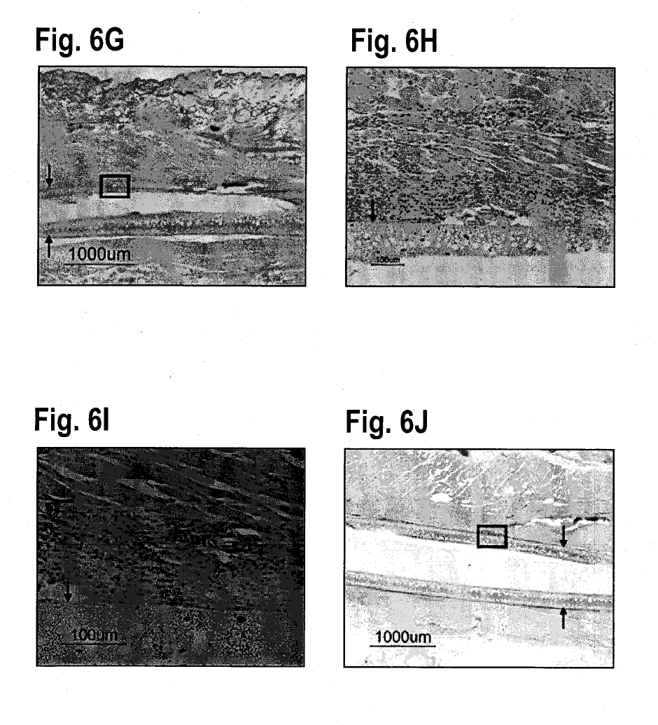

Interestingly, the progressive formation of thick, granuloma-like lesions encasing hollow fibers containing M. tuberculosis H37Rv-lux was observed (FIG. 6d-f), but such formation was not observed in those containing liquid broth alone (FIG. 6a-c). By day 28 after hollow fiber implantation, histologic analysis revealed much greater cellular infiltration of macrophages, lymphocytes, and fibroblasts in the tissues surrounding hollow fibers continuing tubercle bacilli (FIG. 6j-k), as compared to those containing liquid broth (FIG. 6g-i). Acid-fast staining of the surrounding tissue revealed no detectable bacilli, and inflammatory cells were not detected by microscopy within the hollow fibers. These results suggest the possibility that M. tuberculosis secretes soluble factors that diffuse from the fibers, leading to recruitment of host inflammatory cells. The formation of granulomatous lesions encasing hollow fibers may create a hostile microenvironment in which bacilli are forced to reduce their replication and metabolic activity, consequently becoming more susceptible to rifampin than to isoniazid.

Containment of Intra-fiber Bacillary Growth In Vivo is Immune-mediated and IFN-γ-dependent

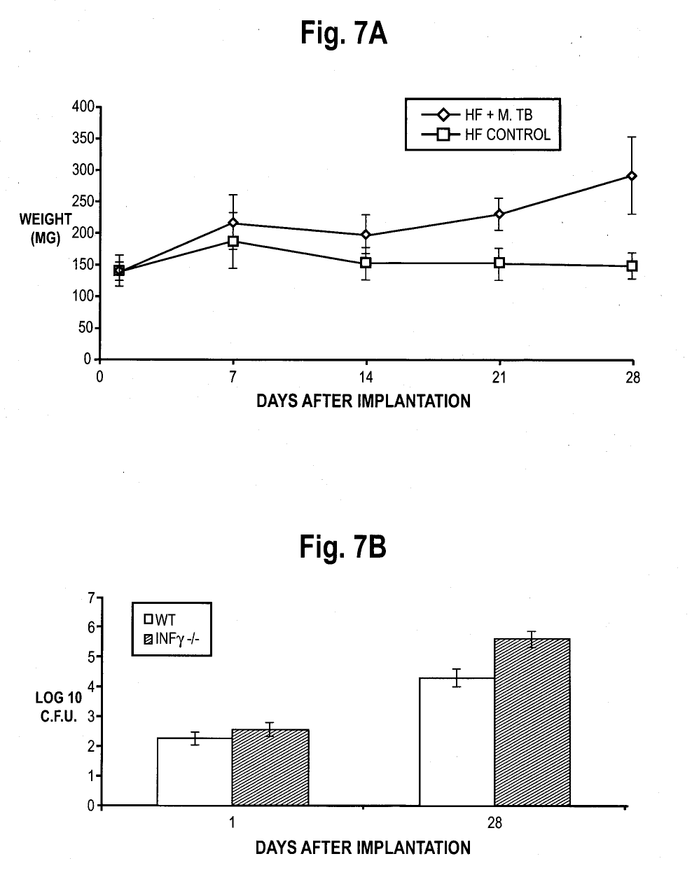

In addition to local granuloma formation, significant spleen enlargement was detected as early as 14 days after hollow fiber implantation in mice which had implanted fibers containing M. tuberculosis as compared to those which had implanted fibers containing media (FIG. 7a), suggesting a systemic immune response in the former group of mice. In order to further investigate the phenomena of granuloma formation and splenomegaly and to exclude the possibility that intra-fiber bacillary growth containment in vivo is mediated exclusively by nutrient starvation, the growth of hollow fiber-encapsulated M. tuberculosis in interferon gamma (IFNγ)-deficient mice was studied. Briefly, approximately 500 bacilli were encapsulated in each hollow fiber, and fibers were implanted subcutaneously either into wild type Balb-C/J mice or Balb-C/J IFNγ-deficient mice, and colony counts were measured 28 days after implantation. Interestingly, although there was some growth of bacilli in wild type mice over 28 days, colony-forming unit counts of hollow fiber-encapsulated M. tuberculosis in IFNγ-deficient mice were 1.3 log₁₀ higher at 28 days than those in wild type mice, suggesting that IFNγ plays a role in containment of intra-fiber bacillary growth in vivo (FIG. 7b).

Reduced Survival of rel_{Mtb}-deficient M. tuberculosis in Hollow Fibers Implanted into Mice

As proof-of-principle to determine whether this model could predict genes essential for extracellular persistence within granulomas, a library of mutants was generated by transposon mutagenesis of M. tuberculosis CDC 1551 strain using the Himar1 transposon as previously described. Lamichhane, supra. One hundred genetically-defined mutants, each deficient in a specific gene (as defined by transposon insertion within the proximal 80% or proximal to the distal 100 base pairs of the open reading frame of each particular gene (Id)), were selected from the library and divided into 2 master pools, each consisting of 50 mutants (Table 2).

TABLE 2
Transposon Insertion Mutant Pool 1

| MT No. | Rv No. | Open reading frame (ORF) description | ORF size | POI |
|---|---|---|---|---|
| 1850 | 1801 | PPE family protein (PPE) | 1271 | 210 |
| 1515 | 1469 | cation transporting ATPase, E1-E2 family ctpD | 1973 | 838 |
| 524 | 503 | cyclopropane-fatty-acyl-phospholipid synthase 2 | 965 | 746 |
| | | (cmaA2) | | |
| 2036 | 1983 | PE_PGRS | 1676 | 60 |
| 2660 | 2583 | GTP pyrophosphokinase (relMtb) | 2381 | 322 |
| 2005 | 1956 | DNA binding protein, putative | 449 | 319 |
| 1551 | 1502 | HP | 899 | 632 |
| 3936 | 3828 | IS1537, resolvase | 611 | 418 |
| 3068 | 2990 | HP | 860 | 62 |
| 154 | 146 | conserved HP | 941 | 126 |
| 3395 | 3296 | ATP-dependent helicase, putative (1 hr) | 4541 | 631 |
| 115 | 106 | conserved HP | 1196 | 17 |
| 364 | 349 | HP | 542 | 76 |
| 3399 | 3300 | RNA pseudouridylate synthase family | 917 | 30 |
| 3011 | 2941 | acyl-CoA synthase, FadD28 | 1742 | 74 |
| 1987 | 1937 | Ferredoxin reductase, electron transfer | 2519 | 459 |
| 3848 | 3740 | HP | 1346 | 1144 |
| 344 | 329 | MitM-related protein | 626 | 462 |
| 3472 | 3364 | Conserved HP | 392 | 253 |
| 2794 | 2721 | Conserved HP | 2099 | 279 |
| 3503 | 3395 | HP | 641 | 169 |
| 2414 | 2349 | Phospholipase C (plcC) | 1526 | 476 |
| 2787 | 2714 | Conserved HP | 974 | 240 |
| 315 | 302 | transcriptional regulator, TetR family | 632 | 336 |
| 3153 | 3068 | Phosphoglucomutase (pgmA) | 1643 | 76 |
| 262 | 248 | FAD flavoprotein oxidase | 1940 | 859 |
| 267 | 254 | Cobinamide/cobinamide phosphate | 524 | 85 |
| | | guanylyltransferase (cobU) | | |
| 1962 | 1911 | Phosphatidylethanolamine binding protein (lppC) | 617 | 65 |
| 953 | 926 | HP | 1076 | 829 |
| 917 | 893 | Conserved HP | 977 | 56 |
| 1780 | 1738 | HP (b/w Rv1737/1738) | 461 | 129 |
| 738 | 711 | arylsulfatase (atsA) | 2271 | 1895 |
| 183 | 174 | virulence factor mce protein | 1547 | 321 |
| 3433 | 3330 | penicillin binding protein | 1217 | 682 |
| 1628 | 1592 | Conserved HP | 1367 | 1072 |
| 77 | 71 | Conserved HP | 716 | 310 |
| 3580 | 3476 | sugar transporter family protein (kgtP) | 1349 | 103 |
| 3569 | 3463 | HP | 857 | 624 |
| 3497 | 3390 | lipoprotein, putative (lpqD) | 710 | 399 |
| 2829 | 2759 | Conservative HP | 395 | 111 |
| 40 | 35 | AMP-binding family protein (fadD34) | 1688 | 103 |
| 2410 | 2345 | Conserved HP | 1982 | 282 |
| 3806 | 3703 | Conservative HP | 1277 | 1116 |
| 1594 | 1542 | protozoan/cianobacterial globin family protein | 410 | 125 |
| | | (glbN) | | |
| 204 | 194 | ABC transporter, ATP-binding protein (HP) | 3725 | 1738 |
| 3510 | 3402 | Degt/DnrJ/EryC1 family protein (HP) | 1238 | 77 |
| 3583 | 3479 | HP | 3227 | 1514 |
| 1099 | 1069 | HP | 1811 | 1063 |
| 2545 | 2469 | HNH endonuclease family protein | 668 | 56 |
| 2315 | 2254 | HP | 455 | 226 |

Transposon Insertion Mutant Pool 2

| MT No. | Rv No. | ORF description | ORF size | POI |
|---|---|---|---|---|
| 2483 | 2410 | Conserved HP | 977 | 598 |
| 3557 | 3451 | serine exterase, cutinase family | 743 | 586 |
| 3838 | 3733 | HP | 500 | 350 |
| 1979 | 1927 | Conserved HP | 774 | 218 |
| 1501 | 1454 | Quinone oxidoreductase, putative (qor) | 1007 | 172 |
| 1533.2 | 1488 | SPFH domain/Band 7 family protein | 1145 | 877 |
| 1402 | 1358 | transcriptional regulator, LuxR family | 3479 | 896 |
| 2785 | 2712 | HP | 1058 | 727 |
| 2956 | 2888 | amidase family protein (amiC) | 1421 | 1123 |
| 1671 | 1635 | HP | 1697 | 1108 |
| 676 | 648 | glycosyl hydrolase | 4196 | 794 |
| 1109 | 1078 | proline-rich antigen (pra) | 722 | 312 |
| 618 | 589 | virulence factor (mce2) | 1214 | 867 |
| 2329 | 2267 | HP | 1166 | 531 |
| 3458 | 3350 | PPE | 11355 | 7007 |
| 1861 | 1813 | HP | 431 | 153 |
| 1956 | 1905 | D-amino acid oxidase | 962 | 64 |
| 1765 | 1724 | HP | 419 | 300 |
| 2749 | 2675 | Conserved HP | 752 | 564 |
| 2086 | 2027 | GAF family protein | 1721 | 273 |
| 2801.1 | 2730 | HP | 476 | 239 |
| 1608 | 1557 | membrane protein, mmpL family (mmpL6) | 1193 | 921 |
| 871 | 848 | cysteine synthase, putative (cysM3) | 1213 | 415 |
| 652 | 624 | Conserved HP | 395 | 112 |
| 2982 | 2914 | Ser/Thr protein kinase (pknI) | 1811 | 43 |
| 1172 | 1139 | Conserved HP | 374 | 235 |
| 2831 | 2761 | Type I restriction system specificity protein | 1094 | 66 |
| 48 | 42 | Transcriptional regulator, MarR family | 626 | 427 |
| 79 | 73 | ABC transporter, ATP binding protein | 992 | 36 |
| 2780 | 2707 | Conserved HP | 974 | 748 |
| 1172.1 | 1139 | HP (b/w Rv1139/1140) | 218 | 154 |
| 787 | 762 | HP | 545 | 123 |
| 1184 | 1147 | IS Mt 1 transposase (HP) | 927 | 387 |
| 1556 | 1508 | HP | 1799 | 488 |
| 2280 | 2222 | glutamine synthase (glnA2) | 1340 | 1093 |
| 202 | 192 | Conserved HP | 1100 | 787 |
| 1625 | 1590 | Conserved HP | 239 | 50 |
| 3005 | 2935 | polyketide synthase (ppsE) | 4464 | 566 |
| 2563 | 2488 | Transcriptional regulator, LuxR family (HP) | 3341 | 919 |
| 2883 | 2816 | Conserved HP | 341 | 67 |
| 2407 | 2342 | HP | 293 | 123 |
| 2239 | 2184 | Conserved HP | 1142 | 894 |
| 3438 | 3334 | transcriptional regulator, MerR family | 438 | 339 |
| 3337 | 3239 | Drug transporter | 3197 | 2589 |
| 105 | 96 | PPE | 1391 | 715 |
| 3176 | 3091 | HP | 1691 | 187 |
| 1834 | 1785 | P450 heme-thiolate protein | 1280 | 400 |
| 1868 | 1820 | TPP requiring enzyme ilvG (acetoactate | 1643 | 63 |
| | | synthase) | | |
| 2674 | 2599 | HP | 431 | 333 |
| 1223 | 1186 | HP | 1616 | 586 |

Other Up-regulated dosR Regulon Genes in the Hollow Fiber Model In Vivo

| Rv No. | Gene | Protein Function | Fold upregulation | P-value |
|---|---|---|---|---|
| 79 | | HP | 2.1 | 0.01 |
| ·81 | | Transcriptional regulator | 1.5 | 0.003 |
| 573c | | CHP | 1.9 | 0.007 |
| 574c | | CHP | 1.8 | 0.002 |
| 2003c | | CHP | 1.7 | 0.005 |
| 2028c | | CHP-USPA motif | 1.5 | 0.006 |
| 2032 | acg | CHP | 5.5 | 0.01 |
| 2624c | | CHP-USPA motif | 2.3 | 0.04 |
| 2625c | | CHP | 1.8 | 0.01 |
| 2630 | | HP | 2.3 | 0.02 |
| 3127 | | CHP | 9.5 | 0.02 |
| 3129 | | CHP | 2.1 | 0.04 |

| | | | |
|---|---|---|---|
| Rv No. | Gene | Fold | P value |

Complete List of Significantly Up-regulated M. tuberculosis Genes in the Hollow
Fiber Model In Vivo

| | | | |
|---|---|---|---|
| Rv0003 | recF | 3.3 | 0.006 |
| Rv0008c | HP | 2.3 | 0.002 |
| Rv0028 | HP | 2.2 | 0.005 |
| Rv0035 | fadD34 | 2.3 | 0.009 |
| Rv0064 | HP | 4.4 | 0.001 |
| Rv0080 | HP | 2.9 | 0.0009 |
| Rv0099 | fadD10 | 3.0 | 0.003 |
| Rv0101 | Nrp | 3.3 | 0.008 |
| Rv0102 | HP | 4.7 | 0.004 |
| Rv0105c | HP | 2.4 | 0.0003 |
| Rv0116c | HP | 2.6 | 0.004 |
| Rv0131c | fadE1 | 2.4 | 0.009 |
| Rv0136 | HP | 2.0 | 0.008 |
| Rv0188 | HP | 2.7 | 0.0002 |
| Rv0197 | HP | 3.1 | 0.006 |
| Rv0204c | HP | 2.8 | 0.002 |
| Rv0211 | pckA | 2.6 | 1.90E−06 |
| Rv0216 | HP | 2.2 | 0.005 |
| Rv0251c | Hsp | 3.9 | 0.004 |
| Rv0255c | cobQ | 2.2 | 9.40E−08 |
| Rv0260c | HP | 2.2 | 0.006 |
| Rv0261c | narK3 | 2.2 | 0.007 |
| Rv0282 | HP | 2.2 | 0.001 |
| Rv0284 | HP | 2.0 | 0.0008 |
| Rv0287 | HP | 2.2 | 0.0004 |
| Rv0288 | HP | 2.9 | 0.004 |
| Rv0309 | HP | 2.1 | 0.004 |
| Rv0324 | HP | 2.4 | 0.006 |
| Rv0336 | HP | 2.4 | 0.009 |
| Rv0355c | PPE | 11.6 | 0.008 |
| Rv0367c | HP | 2.1 | 0.002 |
| Rv0387c | HP | 8.1 | 0.003 |
| Rv0388c | PPE | 3.5 | 0.006 |
| Rv0399c | lpqK | 2.6 | 0.006 |
| Rv0406c | HP | 2.2 | 0.008 |
| Rv0412c | HP | 2.5 | 2.70E−05 |
| Rv0419 | lpqM | 3.3 | 0.004 |
| Rv0440 | groEL2 | 3.6 | 0.0005 |
| Rv0441c | HP | 2.9 | 0.0009 |
| Rv0465c | HP | 3.6 | 0.005 |
| Rv0483 | HP | 2.1 | 0.005 |
| Rv0489 | gpm | 2.4 | 0.0003 |
| Rv0515 | HP | 3.8 | 0.006 |
| Rv0537c | HP | 2.6 | 0.002 |
| Rv0538 | HP | 2.7 | 0.002 |
| Rv0549c | HP | 2.1 | 0.001 |
| Rv0569 | HP | 4.9 | 0.007 |
| Rv0572c | HP | 2.2 | 0.002 |
| Rv0588 | HP | 2.3 | 0.003 |
| Rv0592 | HP | 4.8 | 0.002 |
| Rv0623 | HP | 2.5 | 0.006 |
| Rv0648 | HP | 3.1 | 0.008 |
| Rv0687 | HP | 2.4 | 0.004 |
| Rv0727c | fucA | 2.6 | 0.008 |
| Rv0772 | purD | 2.6 | 0.003 |
| Rv0782 | ptrBa | 2.0 | 0.009 |
| Rv0790c | HP | 4.0 | 0.008 |
| Rv0791c | HP | 2.3 | 0.002 |
| Rv0794c | lpdB | 2.3 | 0.008 |
| Rv0828c | HP | 3.7 | 0.003 |
| Rv0836c | HP | 6.7 | 0.004 |
| Rv0846c | HP | 2.3 | 0.007 |
| Rv0873 | fadE10 | 2.4 | 0.007 |
| Rv0879c | HP | 2.2 | 0.003 |
| Rv0885 | HP | 3.8 | 4.80E−06 |
| Rv0887c | HP | 2.1 | 0.007 |
| Rv0892 | HP | 2.9 | 0.002 |
| Rv0897c | HP | 2.1 | 0.001 |
| Rv0923c | HP | 2.4 | 0.004 |
| Rv0925c | HP | 3.0 | 0.007 |
| Rv0942 | HP | 3.3 | 0.007 |
| Rv0962c | HP | 2.1 | 0.0001 |
| Rv0966c | HP | 4.6 | 0.002 |
| Rv0967 | HP | 2.5 | 0.006 |
| Rv0970 | HP | 22.7 | 0.009 |
| Rv0978c | PE_PGRS | 5.1 | 0.0005 |
| Rv0980c | PE_PGRS | 3.3 | 0.009 |
| Rv0982 | HP | 6.4 | 0.008 |
| Rv0988 | HP | 7.0 | 0.004 |
| Rv1018c | glmU | 8.0 | 0.002 |
| Rv1029 | kdpA | 4.6 | 0.0009 |
| Rv1031 | kdpC | 2.6 | 0.005 |
| Rv1032c | HP | 3.0 | 0.002 |
| Rv1039c | PPE | 3.2 | 0.004 |
| Rv1073 | HP | 2.6 | 9.70E−07 |
| Rv1075c | HP | 3.0 | 0.009 |
| Rv1128c | HP | 3.4 | 0.003 |
| Rv1131 | gltA1 | 4.5 | 0.004 |
| Rv1139c | HP | 3.9 | 0.003 |
| Rv1169c | PE | 2.8 | 0.004 |
| Rv1204c | HP | 4.2 | 0.005 |
| Rv1277 | HP | 2.1 | 0.007 |
| Rv1302 | rfe | 2.5 | 1.50E−07 |
| Rv1367c | HP | 2.4 | 0.002 |
| Rv1375 | HP | 2.3 | 0.0003 |
| Rv1393c | HP | 3.5 | 0.006 |
| Rv1394c | HP | 2.4 | 0.0006 |
| Rv1405c | HP | 2.9 | 0.0009 |
| Rv1406 | fmt | 3.0 | 0.004 |
| Rv1493 | mutB | 2.4 | 0.001 |
| Rv1499 | HP | 2.7 | 0.003 |
| Rv1513 | HP | 2.0 | 0.009 |
| Rv1517 | HP | 2.4 | 0.004 |
| Rv1538c | ansA | 3.3 | 0.003 |
| Rv1556 | HP | 2.4 | 0.007 |
| Rv1572c | HP | 2.9 | 0.009 |
| Rv1592c | HP | 2.4 | 0.0007 |
| Rv1638 | uvrA | 2.2 | 2.10E−08 |
| Rv1702c | HP | 2.7 | 0.002 |
| Rv1730c | HP | 3.5 | 0.003 |
| Rv1733c | HP | 3.0 | 0.004 |
| Rv1752 | HP | 5.7 | 0.004 |
| Rv1753c | PPE | 7.3 | 0.005 |
| Rv1759c | PE_PGRS | 3.0 | 0.006 |
| Rv1782 | HP | 2.3 | 0.0002 |
| Rv1801 | PPE | 2.8 | 0.005 |
| Rv1808 | PPE | 2.8 | 0.003 |
| Rv1809 | PPE | 2.4 | 0.001 |
| Rv1825 | HP | 3.8 | 0.002 |
| Rv1907c | HP | 5.6 | 0.004 |
| Rv1909c | furA | 2.8 | 0.004 |
| Rv1930c | HP | 2.1 | 0.0008 |
| Rv1936 | HP | 3.2 | 0.006 |
| Rv1954c | HP | 2.9 | 0.007 |
| Rv1956 | HP | 2.3 | 0.0009 |
| Rv1970 | lprM | 3.1 | 0.006 |
| Rv1972 | HP | 3.1 | 0.001 |
| Rv1981c | nrdF | 3.5 | 0.006 |
| Rv1984c | HP | 3.7 | 0.009 |
| Rv1986 | HP | 3.8 | 0.003 |
| Rv1988 | HP | 2.9 | 0.005 |
| Rv1990c | HP | 2.7 | 0.0001 |
| Rv1992c | ctpG | 2.3 | 1.70E−07 |
| Rv1995 | HP | 7.1 | 0.009 |
| Rv1997 | ctpF | 13.2 | 0.007 |
| Rv2005c | HP | 2.2 | 0.004 |
| Rv2015c | HP | 2.2 | 8.30E−06 |
| Rv2018 | HP | 2.3 | 1.30E−06 |
| Rv2026c | HP | 3.5 | 0.002 |
| Rv2030c | HP | 7.8 | 3.30E−05 |
| Rv2031c | hspX | 25.0 | 0.009 |
| Rv2055c | rpsR2 | 2.6 | 0.003 |
| Rv2056c | rpsN2 | 4.3 | 9.90E−07 |
| Rv2057c | rpmG | 5.9 | 0.001 |
| Rv2059 | HP | 2.6 | 0.0003 |
| Rv2068c | blaC | 2.0 | 0.001 |
| Rv2069 | sigC | 2.2 | 0.004 |
| Rv2072c | cobL | 2.1 | 0.004 |
| Rv2089c | pepE | 4.4 | 0.005 |
| Rv2113 | HP | 2.1 | 0.003 |
| Rv2114 | HP | 2.1 | 0.003 |
| Rv2122c | hisI | 3.4 | 0.003 |
| Rv2131c | cysQ | 2.4 | 0.006 |
| Rv2166c | HP | 3.0 | 0.003 |
| Rv2170 | HP | 3.9 | 0.003 |
| Rv2173 | idsA2 | 3.9 | 0.003 |
| Rv2225 | panB | 2.1 | 2.80E−05 |
| Rv2227 | HP | 2.5 | 0.004 |
| Rv2269c | HP | 3.1 | 0.003 |
| Rv2273 | HP | 5.1 | 0.009 |
| Rv2306c | HP | 3.3 | 0.002 |
| Rv2322c | rocD1 | 2.1 | 0.009 |
| Rv2330c | lppP | 0.0 | 0.004 |
| Rv2343c | dnaG | 2.7 | 0.0007 |
| Rv2370c | HP | 4.0 | 0.005 |
| Rv2378c | mbtG | 2.1 | 0.001 |
| Rv2380c | mbtE | 4.0 | 0.007 |
| Rv2411c | HP | 2.5 | 0.002 |
| Rv2429 | ahpD | 3.5 | 0.002 |
| Rv2437 | HP | 2.2 | 0.001 |
| Rv2455c | HP | 2.4 | 1.60E−10 |
| Rv2497c | pdhA | 2.0 | 0.0008 |
| Rv2512c | HP | 2.3 | 0.0005 |
| Rv2524c | Fas | 4.5 | 0.007 |
| Rv2531c | Adi | 5.3 | 0.005 |
| Rv2566 | HP | 2.4 | 0.003 |
| Rv2577 | HP | 3.4 | 0.008 |
| Rv2601 | speE | 2.4 | 0.001 |
| Rv2617c | HP | 3.1 | 0.0007 |
| Rv2621c | HP | 2.3 | 0.009 |
| Rv2623 | HP | 25.8 | 0.0002 |
| Rv2626c | HP | 14.6 | 0.0001 |
| Rv2627c | HP | 8.0 | 0.008 |
| Rv2628 | HP | 8.4 | 0.004 |
| Rv2631 | HP | 2.1 | 0.001 |
| Rv2643 | arsC | 3.5 | 0.001 |
| Rv2657c | HP | 2.8 | 0.008 |
| Rv2659c | HP | 3.3 | 0.003 |
| Rv2660c | HP | 3.3 | 0.006 |
| Rv2700 | HP | 2.2 | 0.009 |
| Rv2706c | HP | 2.2 | 0.001 |
| Rv2707 | HP | 3.1 | 0.003 |
| Rv2710 | sigB | 4.7 | 7.40E−06 |
| Rv2712c | HP | 2.4 | 0.006 |
| Rv2713 | HP | 2.7 | 0.008 |
| Rv2725c | hflX | 2.0 | 0.007 |
| Rv2744c | 35kd_ag | 3.3 | 0.0001 |
| Rv2757c | HP | 3.1 | 0.009 |
| Rv2780 | Ald | 2.4 | 0.001 |
| Rv2787 | HP | 2.2 | 0.008 |
| Rv2833c | ugpB | 3.8 | 0.007 |
| Rv2835c | ugpA | 2.4 | 0.003 |
| Rv2843 | HP | 2.2 | 0.004 |
| Rv2863 | HP | 2.0 | 0.008 |
| Rv2887 | HP | 2.8 | 0.009 |
| Rv2932 | ppsB | 2.3 | 0.008 |
| Rv2963 | HP | 3.5 | 0.008 |
| Rv2979c | HP | 2.8 | 0.001 |
| Rv3021c | PPE | 2.4 | 0.003 |
| Rv3047c | HP | 2.0 | 0.004 |
| Rv3048c | nrdG | 2.9 | 0.007 |
| Rv3074 | HP | 2.9 | 0.006 |
| Rv3076 | HP | 4.0 | 0.003 |
| Rv3080c | pknK | 2.5 | 0.0002 |
| Rv3082c | virS | 3.4 | 0.004 |
| Rv3088 | HP | 2.1 | 0.0009 |
| Rv3105c | prfB | 2.9 | 0.001 |
| Rv3126c | HP | 2.2 | 0.0001 |
| Rv3128c | HP | 3.2 | 0.004 |
| Rv3130c | HP | 15.4 | 0.0004 |
| Rv3131 | HP | 11.4 | 0.0008 |
| Rv3132c | HP | 4.8 | 0.001 |
| Rv3133c | HP | 2.1 | 0.007 |
| Rv3134c | HP | 7.1 | 0.007 |
| Rv3139 | fadE24 | 5.8 | 0.004 |
| Rv3189 | HP | 2.2 | 0.004 |
| Rv3223c | sigH | 3.4 | 0.004 |
| Rv3270 | ctpC | 2.4 | 0.0008 |
| Rv3287c | rsbW | 4.6 | 0.0005 |
| Rv3290c | lat | 5.6 | 0.009 |
| Rv3334 | HP | 3.3 | 0.0002 |
| Rv3335c | HP | 3.0 | 0.009 |
| Rv3340 | metC | 2.8 | 0.0007 |
| Rv3343c | PPE | 3.4 | 0.005 |
| Rv3468c | rmlB3 | 3.2 | 0.003 |
| Rv3515c | fadD19 | 3.1 | 0.009 |
| Rv3521 | HP | 2.9 | 0.009 |
| Rv3531c | HP | 2.4 | 0.003 |
| Rv3538 | ufaA2 | 2.1 | 0.003 |
| Rv3539 | PE | 7.6 | 0.006 |
| Rv3650 | PE | 4.6 | 0.008 |
| Rv3651 | HP | 3.2 | 0.004 |
| Rv3660c | HP | 2.0 | 0.0004 |
| Rv3669 | HP | 3.0 | 0.004 |
| Rv3698 | HP | 2.6 | 0.008 |
| Rv3811 | csp | 2.0 | 0.005 |
| Rv3839 | HP | 2.6 | 0.002 |
| Rv3848 | HP | 2.9 | 0.002 |
| Rv3862c | HP | 5.3 | 0.0003 |
| Rv3894c | HP | 5.2 | 0.002 |
| Rv3896c | HP | 2.2 | 0.007 |
| Rv3898c | HP | 2.2 | 0.0007 |
| Rv3900c | HP | 2.0 | 0.003 |
| Rv3912 | HP | 2.7 | 0.003 |

Significantly Down-regulated M. Tuberculosis Genes in the Hollow Fiber
Model In Vivo

| | | | |
|---|---|---|---|
| Rv0172 | HP | −2.5 | 2.0E−04 |
| Rv0682 | rpsL | −2.2 | 6.6E−05 |
| Rv0683 | rpsG | −2.5 | 1.2E−07 |
| Rv0692 | HP | −5.4 | 1.5E−09 |
| Rv0693 | pqqE | −2.2 | 4.3E−05 |
| Rv0694 | lldD1 | −2.1 | 4.2E−05 |
| Rv0696 | HP | −2.3 | 5.0E−04 |
| Rv0700 | rpsJ | −2.8 | 4.5E−06 |
| Rv0701 | rplC | −2.2 | 1.5E−05 |
| Rv0703 | rplW | −2.4 | 1.7E−07 |
| Rv0704 | rplB | −2.6 | 2.0E−04 |
| Rv0706 | rplV | −2.3 | 3.5E−05 |
| Rv0708 | rplP | −2.4 | 4.0E−04 |
| Rv0710 | rpsQ | −2.1 | 9.0E−04 |
| Rv0716 | rplE | −2.4 | 1.3E−10 |
| Rv0722 | rpmD | −2.2 | 3.5E−05 |
| Rv1037c | HP | −2.5 | 3.9E−11 |
| Rv1038c | HP | −2.5 | 1.4E−08 |
| Rv1196 | PPE | −2.6 | 1.5E−06 |
| Rv1197 | HP | −2.1 | 3.9E−08 |
| Rv1198 | HP | −2.4 | 2.1E−06 |
| Rv1304 | atpB | −3.3 | 4.8E−07 |
| Rv1306 | atpF | −3.3 | 9.5E−10 |
| Rv1308 | atpA | −2.2 | 6.6E−05 |
| Rv1310 | atpD | −3.8 | 1.1E−10 |
| Rv1311 | atpC | −2.7 | 6.5E−07 |
| Rv1312 | HP | −2.1 | 2.7E−06 |
| Rv1448c | tal | −2.9 | 1.1E−06 |
| Rv1871c | HP | −2.2 | 2.0E−04 |
| Rv1872c | lldD2 | −2.5 | 5.4E−08 |
| Rv1886c | fbpB | −2.3 | 5.2E−05 |
| Rv2347c | HP | −2.0 | 6.8E−07 |
| Rv2391 | nirA | −2.9 | 4.0E−06 |
| Rv2392 | cysH | −2.2 | 2.0E−03 |
| Rv2393 | HP | −2.1 | 5.0E−03 |
| Rv2986c | hupB | −2.3 | 5.0E−07 |
| Rv3148 | nuoD | −2.2 | 3.2E−05 |
| Rv3308 | pmmB | −2.2 | 3.1E−08 |
| Rv3456c | rplQ | −2.3 | 3.0E−10 |
| Rv3477 | PE | −4.1 | 5.2E−09 |
| Rv3478 | PPE | −2.9 | 2.3E−11 |
| Rv3584 | lpqE | −2.4 | 2.0E−06 |
| Rv3619c | HP | −2.1 | 7.9E−08 |
| Rv3620c | HP | −3.3 | 7.1E−07 |
| Rv3648c | cspA | −7.7 | 1.5E−09 |
| Rv3774 | echA21 | −2.2 | 6.2E−06 |
| Rv3804c | fbpA | −3.5 | 9.8E−08 |
| Rv3841 | bfrB | −2.4 | 9.9E−10 |
| Rv3874 | HP | −2.5 | 1.6E−05 |
| Rv3922c | HP | −2.0 | 3.9E−05 |

Complete Gene Expression Profile of M. tuberculosis in the Hollow Fiber
Model In Vivo

| | | | |
|---|---|---|---|
| Rv0001 | dnaA | 7.62 | 0.01408 |
| Rv0002 | dnaN | 1.51 | 0.04068 |
| Rv0003 | recF | 3.26 | 0.00577 |
| Rv0004 | hypothetical protein Rv0004 | 1.98 | 0.00205 |
| Rv0005 | gyrB | 0.74 | 0.00000 |
| Rv0006 | gyrA | 2.34 | 0.05837 |
| Rv0007 | hypothetical protein Rv0007 | 1.05 | 0.38482 |
| Rv0008c | hypothetical protein Rv0008c | 2.26 | 0.00151 |
| Rv0009 | ppiA | 0.87 | 0.22407 |
| Rv0010c | hypothetical protein Rv0010c | 1.59 | 0.00510 |
| Rv0011c | hypothetical protein Rv0011c | 1.56 | 0.07362 |
| Rv0012 | hypothetical protein Rv0012 | 1.31 | 0.00139 |
| Rv0013 | pabA | 1.44 | 0.12174 |
| Rv0014c | pknB | 1.32 | 0.11038 |
| Rv0015c | pknA | 0.74 | 0.02424 |
| Rv0016c | pbpA | 1.74 | 0.00266 |
| Rv0017c | rodA | 0.66 | 0.00142 |
| Rv0018c | ppp | 0.87 | 0.07012 |
| Rv0019c | hypothetical protein Rv0019c | 0.80 | 0.00889 |
| Rv0020c | hypothetical protein Rv0020c | 1.11 | 0.22439 |
| Rv0021c | hypothetical protein Rv0021c | 1.43 | 0.06305 |
| Rv0022c | hypothetical protein Rv0022c | 1.69 | 0.00366 |
| Rv0023 | hypothetical protein Rv0023 | 2.49 | 0.03514 |
| Rv0024 | hypothetical protein Rv0024 | 2.17 | 0.02775 |
| Rv0025 | hypothetical protein Rv0025 | 1.20 | 0.03772 |
| Rv0026 | hypothetical protein Rv0026 | 2.09 | 0.01115 |
| Rv0027 | hypothetical protein Rv0027 | 1.71 | 0.00524 |
| Rv0028 | hypothetical protein Rv0028 | 2.22 | 0.00536 |
| Rv0029 | hypothetical protein Rv0029 | 0.86 | 0.05482 |
| Rv0030 | hypothetical protein Rv0030 | 1.10 | 0.20629 |
| Rv0031 | hypothetical protein Rv0031 | 1.43 | 0.00627 |
| Rv0032 | bioF2 | 1.10 | 0.26952 |
| Rv0033 | hypothetical protein Rv0033 | 1.58 | 0.03617 |
| Rv0034 | hypothetical protein Rv0034 | 1.07 | 0.26452 |
| Rv0035 | fadD34 | 2.27 | 0.00921 |
| Rv0036c | hypothetical protein Rv0036c | 0.79 | 0.00146 |
| Rv0037c | hypothetical protein Rv0037c | 1.69 | 0.01294 |
| Rv0038 | hypothetical protein Rv0038 | 0.77 | 0.00599 |
| Rv0039c | hypothetical protein Rv0039c | 1.38 | 0.16834 |
| Rv0040c | hypothetical protein Rv0040c | 1.16 | 0.33581 |
| Rv0041 | leuS | 3.94 | 0.03657 |
| Rv0042c | hypothetical protein Rv0042c | 1.01 | 0.49169 |
| Rv0043c | hypothetical protein Rv0043c | 1.19 | 0.09607 |
| Rv0044c | hypothetical protein Rv0044c | 1.09 | 0.21109 |
| Rv0045c | hypothetical protein Rv0045c | 1.96 | 0.04308 |
| Rv0046c | hypothetical protein Rv0046c | 0.92 | 0.38568 |
| Rv0047c | hypothetical protein Rv0047c | 0.84 | 0.17734 |
| Rv0048c | hypothetical protein Rv0048c | 1.32 | 0.10253 |
| Rv0049 | hypothetical protein Rv0049 | 1.00 | 0.49535 |
| Rv0050 | ponA | 0.93 | 0.12565 |
| Rv0051 | hypothetical protein Rv0051 | 1.58 | 0.06821 |
| Rv0052 | hypothetical protein Rv0052 | 1.53 | 0.00015 |
| Rv0053 | rpsF | 0.67 | 0.00010 |
| Rv0054 | ssb | 1.08 | 0.13777 |
| Rv0055 | rpsR | 0.84 | 0.00071 |
| Rv0056 | rplI | 1.11 | 0.00179 |
| Rv0057 | hypothetical protein Rv0057 | 0.76 | 0.00029 |
| Rv0058 | dnaB | 1.46 | 0.01688 |
| Rv0059 | hypothetical protein Rv0059 | 0.66 | 0.00027 |
| Rv0060 | hypothetical protein Rv0060 | 1.51 | 0.00114 |
| Rv0061 | hypothetical protein Rv0061 | 1.40 | 0.09329 |
| Rv0062 | celA | 1.22 | 0.15083 |
| Rv0063 | hypothetical protein Rv0063 | 1.69 | 0.05013 |
| Rv0064 | hypothetical protein Rv0064 | 4.38 | 0.00147 |
| Rv0065 | hypothetical protein Rv0065 | 2.72 | 0.05184 |
| Rv0066c | icd2 | 0.78 | 0.00001 |
| Rv0067c | hypothetical protein Rv0067c | 2.60 | 0.10434 |
| Rv0068 | hypothetical protein Rv0068 | 1.23 | 0.13320 |
| Rv0069c | sdaA | 1.15 | 0.29172 |
| Rv0070c | glyA2 | 1.82 | 0.00943 |
| Rv0071 | hypothetical protein Rv0071 | 1.41 | 0.00732 |
| Rv0072 | hypothetical protein Rv0072 | 0.88 | 0.22075 |
| Rv0073 | hypothetical protein Rv0073 | 1.11 | 0.20733 |
| Rv0074 | hypothetical protein Rv0074 | 1.21 | 0.09829 |
| Rv0075 | hypothetical protein Rv0075 | 1.64 | 0.04670 |
| Rv0076c | hypothetical protein Rv0076c | 1.45 | 0.05273 |
| Rv0077c | hypothetical protein Rv0077c | 1.55 | 0.01544 |
| Rv0078 | hypothetical protein Rv0078 | 1.04 | 0.35604 |
| Rv0079 | hypothetical protein Rv0079 | 2.09 | 0.01241 |
| Rv0080 | hypothetical protein Rv0080 | 2.92 | 0.00092 |
| Rv0081 | hypothetical protein Rv0081 | 1.52 | 0.00302 |
| Rv0082 | hypothetical protein Rv0082 | 0.98 | 0.44245 |
| Rv0083 | hypothetical protein Rv0083 | 1.00 | 0.49823 |
| Rv0084 | hycD | 1.34 | 0.00281 |
| Rv0085 | hycP | 1.06 | 0.13935 |
| Rv0086 | hycQ | 1.03 | 0.41002 |
| Rv0087 | hycE | 1.37 | 0.04652 |
| Rv0088 | hypothetical protein Rv0088 | 1.17 | 0.11596 |
| Rv0089 | hypothetical protein Rv0089 | 2.37 | 0.02677 |
| Rv0090 | hypothetical protein Rv0090 | 1.56 | 0.00374 |
| Rv0091 | hypothetical protein Rv0091 | 2.30 | 0.01655 |
| Rv0092 | ctpA | 1.55 | 0.14462 |
| Rv0093c | hypothetical protein Rv0093c | 1.06 | 0.21870 |
| Rv0094c | hypothetical protein Rv0094c | 1.29 | 0.19103 |
| Rv0095c | hypothetical protein Rv0095c | 1.87 | 0.01271 |
| Rv0096 | PPE | 0.97 | 0.35955 |
| Rv0097 | hypothetical protein Rv0097 | 1.13 | 0.03765 |
| Rv0098 | hypothetical protein Rv0098 | 1.81 | 0.00000 |
| Rv0099 | fadD10 | 2.99 | 0.00304 |
| Rv0100 | hypothetical protein Rv0100 | 1.53 | 0.00000 |
| Rv0101 | nrp | 3.30 | 0.00776 |
| Rv0102 | hypothetical protein Rv0102 | 4.74 | 0.00406 |
| Rv0103c | ctpB | 1.48 | 0.00639 |
| Rv0104 | hypothetical protein Rv0104 | 4.61 | 0.01093 |
| Rv0105c | rpmB | 2.37 | 0.00038 |
| Rv0106 | hypothetical protein Rv0106 | 11.76 | 0.01758 |
| Rv0107c | ctpI | 1.93 | 0.00515 |
| Rv0108c | hypothetical protein Rv0108c | 0.54 | 0.00003 |
| Rv0109 | PE_PGRS | 1.65 | 0.06724 |
| Rv0110 | hypothetical protein Rv0110 | 0.85 | 0.03892 |
| Rv0111 | hypothetical protein Rv0111 | 1.01 | 0.46257 |
| Rv0112 | gca | 0.76 | 0.00839 |
| Rv0113 | gmhA | 0.76 | 0.01189 |
| Rv0114 | hypothetical protein Rv0114 | 0.70 | 0.00071 |
| Rv0115 | hypothetical protein Rv0115 | 0.61 | 0.00101 |
| Rv0116c | hypothetical protein Rv0116c | 2.56 | 0.00355 |
| Rv0117 | oxyS | 2.91 | 0.02590 |
| Rv0118c | oxcA | 1.01 | 0.46822 |
| Rv0119 | fadD7 | 2.95 | 0.02603 |
| Rv0120c | fusA2 | 0.72 | 0.00489 |
| Rv0121c | hypothetical protein Rv0121c | 1.51 | 0.12846 |
| Rv0122 | hypothetical protein Rv0122 | 0.91 | 0.20153 |
| Rv0123 | hypothetical protein Rv0123 | 1.19 | 0.14990 |
| Rv0124 | PE_PGRS | 0.89 | 0.23289 |
| Rv0125 | pepA | 1.10 | 0.23952 |
| Rv0126 | hypothetical protein Rv0126 | 2.54 | 0.04694 |
| Rv0127 | hypothetical protein Rv0127 | 1.25 | 0.01959 |
| Rv0128 | hypothetical protein Rv0128 | 0.73 | 0.00247 |
| Rv0129c | fbpC2 | 1.77 | 0.04255 |
| Rv0130 | hypothetical protein Rv0130 | 0.68 | 0.15876 |
| Rv0131c | fadE1 | 2.38 | 0.00940 |
| Rv0132c | hypothetical protein Rv0132c | 1.35 | 0.39242 |
| Rv0133 | hypothetical protein Rv0133 | 1.79 | 0.14979 |
| Rv0134 | ephF | 0.98 | 0.36570 |
| Rv0135c | hypothetical protein Rv0135c | 1.11 | 0.09391 |
| Rv0136 | hypothetical protein Rv0136 | 2.03 | 0.00761 |
| Rv0137c | hypothetical protein Rv0137c | 3.08 | 0.02914 |
| Rv0138 | hypothetical protein Rv0138 | 1.86 | 0.06308 |
| Rv0139 | hypothetical protein Rv0139 | 1.17 | 0.31042 |
| Rv0140 | hypothetical protein Rv0140 | 1.20 | 0.17407 |
| Rv0141c | hypothetical protein Rv0141c | 0.66 | 0.00003 |
| Rv0142 | hypothetical protein Rv0142 | 0.96 | 0.22149 |
| Rv0143c | hypothetical protein Rv0143c | 0.90 | 0.09759 |
| Rv0144 | hypothetical protein Rv0144 | 0.55 | 0.00001 |
| Rv0145 | hypothetical protein Rv0145 | 1.74 | 0.03616 |
| Rv0146 | hypothetical protein Rv0146 | 1.40 | 0.01320 |
| Rv0147 | hypothetical protein Rv0147 | 1.40 | 0.00144 |
| Rv0148 | hypothetical protein Rv0148 | 1.29 | 0.02580 |
| Rv0149 | hypothetical protein Rv0149 | 1.49 | 0.02632 |
| Rv0150c | hypothetical protein Rv0150c | 1.73 | 0.01893 |
| Rv0151c | PE | 2.65 | 0.06380 |
| Rv0152c | PE | 3.89 | 0.01010 |
| Rv0153c | hypothetical protein Rv0153c | 1.80 | 0.00454 |
| Rv0154c | fadE2 | 1.14 | 0.09008 |
| Rv0155 | pntAA | 1.69 | 0.00132 |
| Rv0156 | pntAB | 4.66 | 0.02338 |
| Rv0157 | pntB | 2.07 | 0.03314 |
| Rv0158 | hypothetical protein Rv0158 | 0.80 | 0.00007 |
| Rv0159c | PE | 1.33 | 0.04383 |
| Rv0161 | hypothetical protein Rv0161 | 1.41 | 0.12934 |
| Rv0162c | adhE | 0.83 | 0.06726 |
| Rv0163 | hypothetical protein Rv0163 | 0.96 | 0.36198 |
| Rv0164 | hypothetical protein Rv0164 | 0.81 | 0.07576 |
| Rv0165c | hypothetical protein Rv0165c | 2.56 | 0.01736 |
| Rv0166 | fadD5 | 0.64 | 0.00238 |
| Rv0167 | hypothetical protein Rv0167 | 0.77 | 0.02507 |
| Rv0168 | hypothetical protein Rv0168 | 0.90 | 0.08838 |
| Rv0169 | mce1 | 0.76 | 0.03396 |
| Rv0170 | hypothetical protein Rv0170 | 0.64 | 0.02296 |
| Rv0171 | hypothetical protein Rv0171 | 21.17 | 0.16935 |
| Rv0172 | hypothetical protein Rv0172 | 0.40 | 0.00018 |
| Rv0173 | lprK | 1.14 | 0.09858 |
| Rv0174 | hypothetical protein Rv0174 | 33.36 | 0.16958 |
| Rv0175 | hypothetical protein Rv0175 | 0.79 | 0.00423 |
| Rv0176 | hypothetical protein Rv0176 | 0.66 | 0.00173 |
| Rv0177 | hypothetical protein Rv0177 | 1.34 | 0.19182 |
| Rv0178 | hypothetical protein Rv0178 | 1.16 | 0.33622 |
| Rv0179c | lprO | 1.87 | 0.06484 |
| Rv0180c | hypothetical protein Rv0180c | 3.41 | 0.06454 |
| Rv0181c | hypothetical protein Rv0181c | 1.22 | 0.10798 |
| Rv0182c | sigG | 0.67 | 0.00000 |
| Rv0183 | hypothetical protein Rv0183 | 0.89 | 0.05013 |
| Rv0184 | hypothetical protein Rv0184 | 2.00 | 0.07437 |
| Rv0185 | hypothetical protein Rv0185 | 1.73 | 0.01289 |
| Rv0186 | bglS | 1.07 | 0.23834 |
| Rv0187 | hypothetical protein Rv0187 | 0.78 | 0.00100 |
| Rv0188 | hypothetical protein Rv0188 | 2.65 | 0.00023 |
| Rv0189c | ilvD | 0.90 | 0.02650 |
| Rv0190 | hypothetical protein Rv0190 | 1.02 | 0.46542 |
| Rv0191 | hypothetical protein Rv0191 | 1.04 | 0.20226 |
| Rv0192 | hypothetical protein Rv0192 | 1.21 | 0.20679 |
| Rv0193c | hypothetical protein Rv0193c | 1.44 | 0.10067 |
| Rv0194 | hypothetical protein Rv0194 | 1.04 | 0.40536 |
| Rv0195 | hypothetical protein Rv0195 | 1.27 | 0.24536 |
| Rv0196 | hypothetical protein Rv0196 | 1.07 | 0.23444 |
| Rv0197 | hypothetical protein Rv0197 | 3.14 | 0.00644 |
| Rv0198c | hypothetical protein Rv0198c | 1.22 | 0.02295 |
| Rv0199 | hypothetical protein Rv0199 | 1.50 | 0.14779 |
| Rv0200 | hypothetical protein Rv0200 | 1.12 | 0.24937 |
| Rv0201c | hypothetical protein Rv0201c | 1.06 | 0.35582 |
| Rv0202c | mmpL11 | 0.87 | 0.20441 |
| Rv0203 | hypothetical protein Rv0203 | 1.07 | 0.26790 |
| Rv0204c | hypothetical protein Rv0204c | 2.79 | 0.00217 |
| Rv0205 | hypothetical protein Rv0205 | 1.44 | 0.02955 |
| Rv0206c | mmpL3 | 1.96 | 0.08076 |
| Rv0207c | hypothetical protein Rv0207c | 0.93 | 0.31878 |
| Rv0208c | hypothetical protein Rv0208c | 1.06 | 0.41338 |
| Rv0209 | hypothetical protein Rv0209 | 0.97 | 0.36805 |
| Rv0210 | hypothetical protein Rv0210 | 1.48 | 0.02479 |
| Rv0211 | pckA | 2.58 | 0.00000 |
| Rv0212c | nadR | 0.87 | 0.21020 |
| Rv0213c | hypothetical protein Rv0213c | 1.62 | 0.05259 |
| Rv0214 | fadD4 | 1.38 | 0.07466 |
| Rv0215c | fadE3 | 0.79 | 0.01473 |
| Rv0216 | hypothetical protein Rv0216 | 2.25 | 0.00465 |
| Rv0217c | lipW | 2.69 | 0.04463 |
| Rv0218 | hypothetical protein Rv0218 | 1.39 | 0.00816 |
| Rv0219 | hypothetical protein Rv0219 | 2.36 | 0.01607 |
| Rv0220 | lipC | 0.86 | 0.14786 |
| Rv0221 | hypothetical protein Rv0221 | 1.06 | 0.31824 |
| Rv0222 | echA1 | 1.32 | 0.16352 |
| Rv0223c | hypothetical protein Rv0223c | 3.83 | 0.01541 |
| Rv0224c | hypothetical protein Rv0224c | 0.92 | 0.10697 |
| Rv0225 | hypothetical protein Rv0225 | 2.68 | 0.06529 |
| Rv0226c | hypothetical protein Rv0226c | 1.24 | 0.07132 |
| Rv0227c | hypothetical protein Rv0227c | 1.13 | 0.25885 |
| Rv0228 | hypothetical protein Rv0228 | 3.55 | 0.03895 |
| Rv0229c | hypothetical protein Rv0229c | 2.45 | 0.27827 |
| Rv0230c | hypothetical protein Rv0230c | 1.26 | 0.23287 |
| Rv0231 | fadE4 | 1.88 | 0.04610 |
| Rv0232 | hypothetical protein Rv0232 | 1.74 | 0.18288 |
| Rv0233 | nrdB | 1.05 | 0.42057 |
| Rv0234c | gabD2 | 3.58 | 0.09771 |
| Rv0235c | hypothetical protein Rv0235c | 2.54 | 0.06671 |
| Rv0236c | hypothetical protein Rv0236c | 0.75 | 0.04156 |
| Rv0237 | lpqI | 1.99 | 0.09071 |
| Rv0238 | hypothetical protein Rv0238 | 2.18 | 0.01391 |
| Rv0239 | hypothetical protein Rv0239 | 0.96 | 0.43430 |
| Rv0240 | hypothetical protein Rv0240 | 1.39 | 0.19817 |
| Rv0241c | hypothetical protein Rv0241c | 0.75 | 0.00973 |
| Rv0242c | fabG4 | 0.87 | 0.10981 |
| Rv0243 | fadA2 | 0.66 | 0.00002 |
| Rv0244c | fadE5 | 1.52 | 0.01495 |
| Rv0245 | hypothetical protein Rv0245 | 1.07 | 0.29100 |
| Rv0246 | hypothetical protein Rv0246 | 1.56 | 0.03617 |
| Rv0247c | hypothetical protein Rv0247c | 1.10 | 0.31282 |
| Rv0248c | hypothetical protein Rv0248c | 0.81 | 0.10219 |
| Rv0249c | hypothetical protein Rv0249c | 1.44 | 0.10930 |
| Rv0250c | hypothetical protein Rv0250c | 1.54 | 0.02343 |
| Rv0251c | hsp | 3.85 | 0.00392 |
| Rv0252 | nirB | 1.00 | 0.49233 |
| Rv0253 | nirD | 0.83 | 0.17040 |
| Rv0254c | cobU | 0.73 | 0.00125 |
| Rv0255c | cobQ | 2.24 | 0.00000 |
| Rv0256c | PPE | 1.88 | 0.04945 |
| Rv0257c | hypothetical protein Rv0257c | 2.33 | 0.01242 |
| Rv0258c | hypothetical protein Rv0258c | 2.02 | 0.02580 |
| Rv0259c | hypothetical protein Rv0259c | 2.47 | 0.01552 |
| Rv0260c | hypothetical protein Rv0260c | 2.45 | 0.00602 |
| Rv0261c | narK3 | 2.15 | 0.00747 |
| Rv0262c | hypothetical protein Rv0262c | 2.22 | 0.01359 |
| Rv0263c | hypothetical protein Rv0263c | 1.68 | 0.01253 |
| Rv0264c | hypothetical protein Rv0264c | 1.73 | 0.00913 |
| Rv0265c | fecB2 | 2.33 | 0.01438 |
| Rv0266c | hypothetical protein Rv0266c | 0.96 | 0.34521 |
| Rv0267 | narU | 1.56 | 0.00017 |
| Rv0268c | hypothetical protein Rv0268c | 1.00 | 0.49561 |
| Rv0269c | hypothetical protein Rv0269c | 2.21 | 0.01347 |
| Rv0270 | fadD2 | 0.80 | 0.10075 |
| Rv0271c | fadE6 | 1.20 | 0.09660 |
| Rv0272c | hypothetical protein Rv0272c | 1.13 | 0.09933 |
| Rv0273c | hypothetical protein Rv0273c | 1.76 | 0.02046 |
| Rv0274 | hypothetical protein Rv0274 | 1.11 | 0.25855 |
| Rv0275c | fadD27 | 2.48 | 0.00227 |
| Rv0276 | hypothetical protein Rv0276 | 1.17 | 0.19934 |
| Rv0277c | hypothetical protein Rv0277c | 1.29 | 0.07920 |
| Rv0278c | PE_PGRS | 1.74 | 0.08196 |
| Rv0279c | PE_PGRS | 6.24 | 0.04026 |
| Rv0280 | PPE | 1.55 | 0.33659 |
| Rv0281 | hypothetical protein Rv0281 | 1.54 | 0.03886 |
| Rv0282 | hypothetical protein Rv0282 | 2.24 | 0.00127 |
| Rv0283 | hypothetical protein Rv0283 | 1.15 | 0.09714 |
| Rv0284 | hypothetical protein Rv0284 | 2.01 | 0.00079 |
| Rv0285 | PE | 1.57 | 0.00294 |
| Rv0286 | PPE | 3.09 | 0.03992 |
| Rv0287 | hypothetical protein Rv0287 | 2.20 | 0.00042 |
| Rv0288 | hypothetical protein Rv0288 | 2.85 | 0.00359 |
| Rv0289 | hypothetical protein Rv0289 | 1.32 | 0.00829 |
| Rv0290 | hypothetical protein Rv0290 | 1.22 | 0.09494 |
| Rv0291 | hypothetical protein Rv0291 | 0.83 | 0.00000 |
| Rv0292 | hypothetical protein Rv0292 | 1.32 | 0.06654 |
| Rv0293c | hypothetical protein Rv0293c | 1.20 | 0.01285 |
| Rv0294 | hypothetical protein Rv0294 | 0.80 | 0.01812 |
| Rv0295c | hypothetical protein Rv0295c | 1.43 | 0.09367 |
| Rv0296c | atsG | 1.63 | 0.06785 |
| Rv0297 | PE_PGRS | 1.10 | 0.17676 |
| Rv0298 | hypothetical protein Rv0298 | 0.63 | 0.00013 |
| Rv0299 | hypothetical protein Rv0299 | 0.93 | 0.18312 |
| Rv0300 | hypothetical protein Rv0300 | 1.01 | 0.48617 |
| Rv0301 | hypothetical protein Rv0301 | 1.18 | 0.11050 |
| Rv0302 | hypothetical protein Rv0302 | 0.60 | 0.00000 |
| Rv0303 | hypothetical protein Rv0303 | 2.04 | 0.04198 |
| Rv0304c | PPE | 4.84 | 0.02911 |
| Rv0305c | PPE | 5.90 | 0.06182 |
| Rv0306 | hypothetical protein Rv0306 | 1.62 | 0.05164 |
| Rv0307c | hypothetical protein Rv0307c | 1.23 | 0.00424 |
| Rv0308 | hypothetical protein Rv0308 | 1.23 | 0.03538 |
| Rv0309 | hypothetical protein Rv0309 | 2.13 | 0.00430 |
| Rv0310c | hypothetical protein Rv0310c | 1.01 | 0.43982 |
| Rv0311 | hypothetical protein Rv0311 | 1.87 | 0.02787 |
| Rv0312 | hypothetical protein Rv0312 | 1.29 | 0.15379 |
| Rv0313 | hypothetical protein Rv0313 | 0.98 | 0.33951 |
| Rv0314c | hypothetical protein Rv0314c | 0.54 | 0.00006 |
| Rv0315 | hypothetical protein Rv0315 | 1.60 | 0.06993 |
| Rv0316 | hypothetical protein Rv0316 | 1.88 | 0.04886 |
| Rv0317c | glpQ2 | 1.61 | 0.00336 |
| Rv0318c | hypothetical protein Rv03183 | 0.76 | 0.00536 |
| Rv0319 | pcp | 2.26 | 0.01617 |
| Rv0320 | hypothetical protein Rv0320 | 0.63 | 0.00251 |
| Rv0321 | dcd | 1.88 | 0.01377 |
| Rv0322 | udgA | 1.02 | 0.41133 |
| Rv0323c | hypothetical protein Rv0323c | 0.93 | 0.34427 |
| Rv0324 | hypothetical protein Rv0324 | 2.41 | 0.00592 |
| Rv0325 | hypothetical protein Rv0325 | 2.25 | 0.11709 |
| Rv0326 | hypothetical protein Rv0326 | 1.05 | 0.41048 |
| Rv0327c | hypothetical protein Rv0327c | 0.94 | 0.32114 |
| Rv0328 | hypothetical protein Rv0328 | 0.75 | 0.00054 |
| Rv0329c | hypothetical protein Rv0329c | 1.35 | 0.15773 |
| Rv0330c | hypothetical protein Rv0330c | 2.64 | 0.02259 |
| Rv0331 | hypothetical protein Rv0331 | 0.98 | 0.44778 |
| Rv0332 | hypothetical protein Rv0332 | 0.99 | 0.45237 |
| Rv0333 | hypothetical protein Rv0333 | 1.40 | 0.07186 |
| Rv0334 | rmlA | 2.17 | 0.03880 |
| Rv0335c | PE | 1.33 | 0.12482 |
| Rv0336 | hypothetical protein Rv0336 | 2.42 | 0.00956 |
| Rv0337c | aspC | 1.15 | 0.01907 |
| Rv0338c | hypothetical protein Rv0338c | 1.98 | 0.08029 |
| Rv0339c | hypothetical protein Rv0339c | 2.30 | 0.03348 |
| Rv0340 | hypothetical protein Rv0340 | 1.58 | 0.02116 |
| Rv0341 | hypothetical protein Rv0341 | 0.88 | 0.01978 |
| Rv0342 | hypothetical protein Rv0342 | 1.04 | 0.39340 |
| Rv0343 | hypothetical protein Rv0343 | 1.22 | 0.06952 |
| Rv0344c | lpqJ | 3.69 | 0.02948 |
| Rv0345 | hypothetical protein Rv0345 | 0.86 | 0.12102 |
| Rv0346c | aroP2 | 0.73 | 0.00058 |
| Rv0347 | hypothetical protein Rv0347 | 1.76 | 0.02655 |
| Rv0348 | hypothetical protein Rv0348 | 0.90 | 0.28542 |
| Rv0349 | hypothetical protein Rv0349 | 1.43 | 0.02056 |
| Rv0350 | dnaK | 1.62 | 0.00285 |
| Rv0351 | grpE | 1.31 | 0.02918 |
| Rv0352 | dnaJ | 0.93 | 0.06574 |
| Rv0353 | hspR | 1.53 | 0.04134 |
| Rv0354c | PPE | 4.68 | 0.04355 |
| Rv0355c | PPE | 11.63 | 0.00847 |
| Rv0356c | hypothetical protein Rv0356c | 0.84 | 0.03224 |
| Rv0357c | purA | 1.63 | 0.01636 |
| Rv0358 | hypothetical protein Rv0358 | 1.59 | 0.03809 |
| Rv0359 | hypothetical protein Rv0359 | 1.70 | 0.00969 |
| Rv0360c | hypothetical protein Rv0360c | 0.81 | 0.04112 |
| Rv0361 | hypothetical protein Rv0361 | 5.32 | 0.02577 |
| Rv0362 | mgtE | 0.70 | 0.00000 |
| Rv0363c | fba | 1.42 | 0.03549 |
| Rv0364 | hypothetical protein Rv0364 | 1.06 | 0.35874 |
| Rv0365c | hypothetical protein Rv0365c | 1.17 | 0.09675 |
| Rv0366c | hypothetical protein Rv0366c | 2.05 | 0.02023 |
| Rv0367c | hypothetical protein Rv0367c | 2.05 | 0.00227 |
| Rv0368c | hypothetical protein Rv0368c | 1.70 | 0.08883 |
| Rv0369c | hypothetical protein Rv0369c | 1.22 | 0.03243 |
| Rv0370c | hypothetical protein Rv0370c | 0.61 | 0.00001 |
| Rv0372c | hypothetical protein Rv0372c | 1.29 | 0.02082 |
| Rv0373c | hypothetical protein Rv0373c | 0.81 | 0.05198 |
| Rv0374c | hypothetical protein Rv0374c | 1.63 | 0.01124 |
| Rv0375c | hypothetical protein Rv0375c | 1.12 | 0.19646 |
| Rv0377 | hypothetical protein Rv0377 | 2.74 | 0.03920 |
| Rv0379 | sec | 1.22 | 0.28895 |
| Rv0381c | hypothetical protein Rv0381c | 1.03 | 0.40442 |
| Rv0383c | hypothetical protein Rv0383c | 0.82 | 0.11270 |
| Rv0384c | clpB | 2.59 | 0.28260 |
| Rv0385 | hypothetical protein Rv0385 | 1.67 | 0.00493 |
| Rv0386 | hypothetical protein Rv0386 | 0.97 | 0.37845 |
| Rv0387c | hypothetical protein Rv0387c | 8.09 | 0.00316 |
| Rv0388c | PPE | 3.50 | 0.00551 |
| Rv0389 | purT | 1.96 | 0.00520 |
| Rv0390 | hypothetical protein Rv0390 | 1.05 | 0.31849 |
| Rv0391 | metZ | 1.18 | 0.08861 |
| Rv0392c | hypothetical protein Rv0392c | 1.57 | 0.00059 |
| Rv0393 | hypothetical protein Rv0393 | 1.20 | 0.03221 |
| Rv0394c | hypothetical protein Rv0394c | 1.74 | 0.00239 |
| Rv0395 | hypothetical protein Rv0395 | 2.92 | 0.03912 |
| Rv0396 | hypothetical protein Rv0396 | 1.84 | 0.01146 |
| Rv0397 | hypothetical protein Rv0397 | 1.82 | 0.01544 |
| Rv0398c | hypothetical protein Rv0398c | 1.25 | 0.02453 |
| Rv0399c | lpqK | 2.63 | 0.00613 |
| Rv0400c | fadE7 | 1.07 | 0.06665 |
| Rv0401 | hypothetical protein Rv0401 | 0.95 | 0.33983 |
| Rv0402c | mmpL1 | 1.69 | 0.00454 |
| Rv0403c | mmpS1 | 3.34 | 0.02117 |
| Rv0404 | fadD30 | 0.60 | 0.00000 |
| Rv0405 | pks6 | 0.88 | 0.03039 |
| Rv0406c | hypothetical protein Rv0406c | 2.22 | 0.00812 |
| Rv0407 | hypothetical protein Rv0407 | 2.60 | 0.02748 |
| Rv0408 | pta | 1.11 | 0.22268 |
| Rv0409 | ackA | 0.91 | 0.14080 |
| Rv0410c | pknG | 1.69 | 0.00000 |
| Rv0411c | glnH | 1.81 | 0.00893 |
| Rv0412c | hypothetical protein Rv0412c | 2.49 | 0.00003 |
| Rv0413 | mutT3 | 0.82 | 0.11063 |
| Rv0414c | thiE | 1.86 | 0.00683 |
| Rv0415 | hypothetical protein Rv0415 | 0.85 | 0.09021 |
| Rv0416 | hypothetical protein Rv0416 | 1.13 | 0.00763 |
| Rv0417 | thiG | 1.06 | 0.29521 |
| Rv0418 | lpqL | 1.15 | 0.20553 |
| Rv0419 | lpqM | 3.33 | 0.00422 |
| Rv0420c | hypothetical protein Rv0420c | 1.61 | 0.01670 |
| Rv0421c | hypothetical protein Rv0421c | 1.87 | 0.08628 |
| Rv0422c | thiD | 1.41 | 0.13995 |
| Rv0423c | thiC | 0.81 | 0.00443 |
| Rv0424c | hypothetical protein Rv0424c | 0.63 | 0.00119 |
| Rv0425c | ctpH | 0.89 | 0.00033 |
| Rv0426c | hypothetical protein Rv0426c | 1.21 | 0.09791 |
| Rv0427c | xthA | 1.23 | 0.21266 |
| Rv0428c | hypothetical protein Rv0428c | 5.37 | 0.03284 |
| Rv0429c | def | 1.71 | 0.02653 |
| Rv0430 | hypothetical protein Rv0430 | 0.78 | 0.06340 |
| Rv0431 | hypothetical protein Rv0431 | 2.45 | 0.02703 |
| Rv0432 | sodC | 0.89 | 0.32758 |
| Rv0433 | hypothetical protein Rv0433 | 1.02 | 0.41288 |
| Rv0434 | hypothetical protein Rv0434 | 1.08 | 0.19574 |
| Rv0435c | hypothetical protein Rv0435c | 1.11 | 0.14439 |
| Rv0436c | pssA | 3.92 | 0.01146 |
| Rv0437c | psd | 1.13 | 0.14594 |
| Rv0438c | moaA3 | 1.85 | 0.00754 |
| Rv0439c | hypothetical protein Rv0439c | 1.11 | 0.00538 |
| Rv0440 | groEL2 | 3.57 | 0.00051 |
| Rv0441c | hypothetical protein Rv0441c | 2.93 | 0.00087 |
| Rv0442c | PPE | 2.56 | 0.01579 |
| Rv0443 | hypothetical protein Rv0443 | 1.06 | 0.28757 |
| Rv0444c | hypothetical protein Rv0444c | 1.52 | 0.09453 |
| Rv0445c | sigK | 0.81 | 0.03537 |
| Rv0446c | hypothetical protein Rv0446c | 1.64 | 0.02923 |
| Rv0447c | ufaA1 | 0.94 | 0.26571 |
| Rv0448c | hypothetical protein Rv0448c | 1.24 | 0.00649 |
| Rv0449c | hypothetical protein Rv0449c | 0.86 | 0.00087 |
| Rv0450c | mmpL4 | 1.29 | 0.04406 |
| Rv0451c | mmpS4 | 1.35 | 0.05462 |
| Rv0452 | hypothetical protein Rv0452 | 1.30 | 0.10641 |
| Rv0453 | PPE | 1.77 | 0.06300 |
| Rv0454 | hypothetical protein Rv0454 | 1.97 | 0.01627 |
| Rv0455c | hypothetical protein Rv0455c | 0.99 | 0.47987 |
| Rv0456c | echA2 | 0.82 | 0.09675 |
| Rv0457c | hypothetical protein Rv0457c | 2.51 | 0.06141 |
| Rv0458 | hypothetical protein Rv0458 | 1.64 | 0.11962 |
| Rv0459 | hypothetical protein Rv0459 | 1.01 | 0.45005 |
| Rv0460 | hypothetical protein Rv0460 | 1.26 | 0.03249 |
| Rv0461 | hypothetical protein Rv0461 | 1.19 | 0.04873 |
| Rv0462 | hypothetical protein Rv0462 | 0.98 | 0.45599 |
| Rv0463 | hypothetical protein Rv0463 | 1.41 | 0.00547 |
| Rv0464c | hypothetical protein Rv0464c | 1.56 | 0.04183 |
| Rv0465c | hypothetical protein Rv0465c | 3.60 | 0.00528 |
| Rv0466 | hypothetical protein Rv0466 | 0.94 | 0.28593 |
| Rv0467 | aceA | 1.52 | 0.00428 |
| Rv0468 | fadB2 | 0.85 | 0.00925 |
| Rv0469 | umaA1 | 1.50 | 0.08759 |
| Rv0470c | umaA2 | 1.87 | 0.10337 |
| Rv0471c | hypothetical protein Rv0471c | 3.68 | 0.03200 |
| Rv0472c | hypothetical protein Rv0472c | 2.11 | 0.08836 |
| Rv0473 | hypothetical protein Rv0473 | 2.05 | 0.01862 |
| Rv0474 | hypothetical protein Rv0474 | 1.04 | 0.44613 |
| Rv0475 | hypothetical protein Rv0475 | 0.82 | 0.00632 |
| Rv0476 | hypothetical protein Rv0476 | 0.92 | 0.22391 |
| Rv0477 | hypothetical protein Rv0477 | 1.00 | 0.47248 |
| Rv0478 | deoC | 0.92 | 0.12372 |
| Rv0479c | hypothetical protein Rv0479c | 0.91 | 0.38367 |
| Rv0480c | hypothetical protein Rv0480c | 0.79 | 0.01570 |
| Rv0481c | hypothetical protein Rv0481c | 3.58 | 0.01507 |
| Rv0482 | murB | 1.44 | 0.00020 |
| Rv0483 | hypothetical protein Rv0483 | 2.09 | 0.00509 |
| Rv0484c | hypothetical protein Rv0484c | 1.48 | 0.00653 |
| Rv0485 | hypothetical protein Rv0485 | 1.41 | 0.00000 |
| Rv0486 | hypothetical protein Rv0486 | 0.99 | 0.39639 |
| Rv0487 | hypothetical protein Rv0487 | 1.00 | 0.49272 |
| Rv0488 | hypothetical protein Rv0488 | 1.51 | 0.01040 |
| Rv0489 | gpm | 2.39 | 0.00038 |
| Rv0490 | senX3 | 1.06 | 0.14565 |
| Rv0491 | regX3 | 1.71 | 0.02021 |
| Rv0492c | hypothetical protein Rv0492c | 1.71 | 0.00131 |
| Rv0493c | hypothetical protein Rv0493c | 0.66 | 0.00348 |
| Rv0494 | hypothetical protein Rv0494 | 3.11 | 0.01890 |
| Rv0495c | hypothetical protein Rv0495c | 0.76 | 0.02044 |
| Rv0496 | hypothetical protein Rv0496 | 0.78 | 0.00000 |
| Rv0497 | hypothetical protein Rv0497 | 1.23 | 0.03663 |
| Rv0498 | hypothetical protein Rv0498 | 1.18 | 0.26358 |
| Rv0499 | hypothetical protein Rv0499 | 0.93 | 0.18791 |
| Rv0500 | proC | 0.91 | 0.19831 |
| Rv0501 | galE1 | 0.92 | 0.19813 |
| Rv0502 | hypothetical protein Rv0502 | 1.31 | 0.23169 |
| Rv0503c | cmaA2 | 2.04 | 0.04420 |
| Rv0504c | hypothetical protein Rv0504c | 1.92 | 0.02445 |
| Rv0505c | serB | 2.54 | 0.01103 |
| Rv0506 | mmpS2 | 1.72 | 0.06144 |
| Rv0507 | mmpL2 | 2.15 | 0.02694 |
| Rv0508 | hypothetical protein Rv0508 | 0.87 | 0.28710 |
| Rv0509 | hemA | 0.79 | 0.04491 |
| Rv0510 | hemC | 0.55 | 0.00740 |
| Rv0511 | cysG | 2.02 | 0.02601 |
| Rv0512 | hemB | 0.75 | 0.02125 |
| Rv0513 | hypothetical protein Rv0513 | 0.92 | 0.32960 |
| Rv0514 | hypothetical protein Rv0514 | 1.06 | 0.33463 |
| Rv0515 | hypothetical protein Rv0515 | 3.79 | 0.00602 |
| Rv0516c | hypothetical protein Rv0516c | 0.88 | 0.02097 |
| Rv0517 | hypothetical protein Rv0517 | 0.89 | 0.05898 |
| Rv0518 | hypothetical protein Rv0518 | 1.12 | 0.18740 |
| Rv0519c | hypothetical protein Rv0519c | 2.37 | 0.03368 |
| Rv0520 | hypothetical protein Rv0520 | 1.66 | 0.00455 |
| Rv0521c | hypothetical protein Rv0521c | 0.72 | 0.00007 |
| Rv0522 | gabP | 1.53 | 0.02282 |
| Rv0523c | hypothetical protein Rv0523c | 1.59 | 0.08362 |
| Rv0524 | hemL | 0.60 | 0.00012 |
| Rv0525 | hypothetical protein Rv0525 | 0.96 | 0.36495 |
| Rv0526 | hypothetical protein Rv0526 | 1.58 | 0.17058 |
| Rv0527 | ccsA | 0.79 | 0.00086 |
| Rv0528 | hypothetical protein Rv0528 | 0.83 | 0.05421 |
| Rv0529 | ccsB | 1.08 | 0.22810 |
| Rv0530 | hypothetical protein Rv0530 | 1.44 | 0.00223 |
| Rv0531 | hypothetical protein Rv0531 | 1.22 | 0.16656 |
| Rv0532 | PE_PGRS | 1.32 | 0.11756 |
| Rv0533c | fabH | 1.41 | 0.01893 |
| Rv0534c | menA | 1.18 | 0.27854 |
| Rv0535 | pnp | 2.29 | 0.04354 |
| Rv0536 | galE2 | 1.35 | 0.00401 |
| Rv0537c | hypothetical protein Rv0537c | 2.63 | 0.00160 |
| Rv0538 | hypothetical protein Rv0538 | 2.69 | 0.00167 |
| Rv0539 | hypothetical protein Rv0539 | 1.22 | 0.00486 |
| Rv0540 | hypothetical protein Rv0540 | 1.75 | 0.00001 |
| Rv0541c | hypothetical protein Rv0541c | 1.05 | 0.32493 |
| Rv0542c | menE | 0.83 | 0.23413 |
| Rv0543c | hypothetical protein Rv0543c | 0.99 | 0.46086 |
| Rv0544c | hypothetical protein Rv0544c | 0.97 | 0.41609 |
| Rv0545c | pitA | 0.71 | 0.01103 |
| Rv0546c | hypothetical protein Rv0546c | 1.25 | 0.23549 |
| Rv0547c | hypothetical protein Rv0547c | 1.84 | 0.02729 |
| Rv0548c | menB | 2.91 | 0.02124 |
| Rv0549c | hypothetical protein Rv0549c | 2.07 | 0.00096 |
| Rv0550c | hypothetical protein Rv0550c | 1.07 | 0.26538 |
| Rv0551c | fadD8 | 1.19 | 0.09898 |
| Rv0552 | hypothetical protein Rv0552 | 1.20 | 0.06050 |
| Rv0553 | menC | 0.92 | 0.23410 |
| Rv0554 | bpoC | 0.68 | 0.01874 |
| Rv0555 | menD | 1.83 | 0.03192 |
| Rv0556 | hypothetical protein Rv0556 | 0.90 | 0.31135 |
| Rv0557 | hypothetical protein Rv0557 | 1.01 | 0.44915 |
| Rv0558 | ubiE | 0.81 | 0.00077 |
| Rv0559c | hypothetical protein Rv0559c | 1.06 | 0.16921 |
| Rv0560c | hypothetical protein Rv0560c | 2.65 | 0.02135 |
| Rv0561c | hypothetical protein Rv0561c | 1.54 | 0.00176 |
| Rv0562 | grcC1 | 0.82 | 0.08827 |
| Rv0563 | htpX | 1.01 | 0.47742 |
| Rv0564c | gpdA1 | 0.71 | 0.00000 |
| Rv0565c | hypothetical protein Rv0565c | 2.73 | 0.04709 |
| Rv0566c | hypothetical protein Rv0566c | 1.66 | 0.08557 |
| Rv0567 | hypothetical protein Rv0567 | 1.85 | 0.02441 |
| Rv0568 | hypothetical protein Rv0568 | 0.69 | 0.00006 |
| Rv0569 | hypothetical protein Rv0569 | 4.94 | 0.00729 |
| Rv0570 | nrdZ | 1.96 | 0.00000 |
| Rv0571c | hypothetical protein Rv0571c | 0.93 | 0.16249 |
| Rv0572c | hypothetical protein Rv0572c | 2.17 | 0.00163 |
| Rv0573c | hypothetical protein Rv0573c | 1.93 | 0.00743 |
| Rv0574c | hypothetical protein Rv0574c | 1.85 | 0.00203 |
| Rv0575c | hypothetical protein Rv0575c | 1.49 | 0.01878 |
| Rv0576 | hypothetical protein Rv0576 | 1.21 | 0.17056 |
| Rv0577 | hypothetical protein Rv0577 | 1.24 | 0.09758 |
| Rv0578c | PE_PGRS | 0.91 | 0.15960 |
| Rv0579 | hypothetical protein Rv0579 | 0.93 | 0.16288 |
| Rv0580c | hypothetical protein Rv0580c | 0.92 | 0.21143 |
| Rv0581 | hypothetical protein Rv0581 | 1.13 | 0.13672 |
| Rv0582 | hypothetical protein Rv0582 | 1.09 | 0.25178 |
| Rv0583c | lpqN | 1.09 | 0.30931 |
| Rv0584 | hypothetical protein Rv0584 | 1.16 | 0.23259 |
| Rv0585c | hypothetical protein Rv0585c | 0.84 | 0.04254 |
| Rv0586 | hypothetical protein Rv0586 | 1.32 | 0.02539 |
| Rv0587 | hypothetical protein Rv0587 | 1.25 | 0.12852 |
| Rv0588 | hypothetical protein Rv0588 | 2.32 | 0.00343 |
| Rv0589 | mce2 | 1.72 | 0.02870 |
| Rv0590 | hypothetical protein Rv0590 | 1.08 | 0.27477 |
| Rv0591 | hypothetical protein Rv0591 | 2.93 | 0.02030 |
| Rv0592 | hypothetical protein Rv0592 | 4.76 | 0.00164 |
| Rv0593 | lprL | 1.04 | 0.28300 |
| Rv0594 | hypothetical protein Rv0594 | 1.46 | 0.02538 |
| Rv0595c | hypothetical protein Rv0595c | 1.49 | 0.01260 |
| Rv0596c | hypothetical protein Rv0596c | 0.99 | 0.44744 |
| Rv0597c | hypothetical protein Rv0597c | 1.41 | 0.12338 |
| Rv0598c | hypothetical protein Rv0598c | 2.04 | 0.02385 |
| Rv0599c | hypothetical protein Rv0599c | 1.42 | 0.02601 |
| Rv0600c | hypothetical protein Rv0600c | 1.69 | 0.01625 |
| Rv0601c | hypothetical protein Rv0601c | 4.14 | 0.02187 |
| Rv0602c | tcrA | 1.59 | 0.02480 |
| Rv0603 | hypothetical protein Rv0603 | 1.21 | 0.03274 |
| Rv0604 | lpqO | 1.02 | 0.35319 |
| Rv0605 | hypothetical protein Rv0605 | 1.25 | 0.06168 |
| Rv0606 | hypothetical protein Rv0606 | 0.92 | 0.22309 |
| Rv0607 | hypothetical protein Rv0607 | 1.63 | 0.00186 |
| Rv0608 | hypothetical protein Rv0608 | 1.62 | 0.04827 |
| Rv0609 | hypothetical protein Rv0609 | 1.67 | 0.00273 |
| Rv0610c | hypothetical protein Rv0610c | 1.24 | 0.16771 |
| Rv0611c | hypothetical protein Rv0611c | 1.02 | 0.41560 |
| Rv0612 | hypothetical protein Rv0612 | 0.99 | 0.46182 |
| Rv0613c | hypothetical protein Rv0613c | 1.78 | 0.14770 |
| Rv0614 | hypothetical protein Rv0614 | 0.99 | 0.46783 |
| Rv0615 | hypothetical protein Rv0615 | 1.39 | 0.06441 |
| Rv0616c | hypothetical protein Rv0616c | 1.70 | 0.00321 |
| Rv0617 | hypothetical protein Rv0617 | 4.13 | 0.08337 |
| Rv0618 | galT′ | 1.58 | 0.17496 |
| Rv0619 | galT | 0.87 | 0.04564 |
| Rv0620 | galK | 1.86 | 0.09685 |
| Rv0621 | hypothetical protein Rv0621 | 2.22 | 0.02435 |
| Rv0622 | hypothetical protein Rv0622 | 2.42 | 0.02745 |
| Rv0623 | hypothetical protein Rv0623 | 2.51 | 0.00560 |
| Rv0624 | hypothetical protein Rv0624 | 2.17 | 0.03266 |
| Rv0625c | hypothetical protein Rv0625c | 1.47 | 0.09723 |
| Rv0626 | hypothetical protein Rv0626 | 1.35 | 0.01001 |
| Rv0627 | hypothetical protein Rv0627 | 1.12 | 0.23594 |
| Rv0628c | hypothetical protein Rv0628c | 1.87 | 0.00249 |
| Rv0629c | recD | 0.99 | 0.47070 |
| Rv0630c | recB | 1.72 | 0.00811 |
| Rv0631c | recC | 1.87 | 0.07661 |
| Rv0632c | echA3 | 1.14 | 0.05891 |
| Rv0633c | hypothetical protein Rv0633c | 5.80 | 0.09837 |
| Rv0634c | hypothetical protein Rv0634c | 1.01 | 0.42720 |
| Rv0635 | hypothetical protein Rv0635 | 0.71 | 0.00001 |
| Rv0636 | hypothetical protein Rv0636 | 0.72 | 0.00096 |
| Rv0637 | hypothetical protein Rv0637 | 0.98 | 0.44924 |
| Rv0638 | secE | 1.05 | 0.35305 |
| Rv0639 | nusG | 1.15 | 0.19532 |
| Rv0640 | rplK | 1.01 | 0.43122 |
| Rv0641 | rplA | 0.85 | 0.01986 |
| Rv0642c | mmaA4 | 1.09 | 0.40058 |
| Rv0643c | mmaA3 | 1.87 | 0.04225 |
| Rv0644c | mmaA2 | 0.66 | 0.00051 |
| Rv0645c | mmaA1 | 1.27 | 0.02866 |
| Rv0646c | lipG | 0.84 | 0.01422 |
| Rv0647c | hypothetical protein Rv0647c | 1.03 | 0.38837 |
| Rv0648 | hypothetical protein Rv0648 | 3.08 | 0.00768 |
| Rv0649 | fabD2 | 1.74 | 0.00787 |
| Rv0650 | hypothetical protein Rv0650 | 1.08 | 0.04081 |
| Rv0651 | rplJ | 1.24 | 0.07950 |
| Rv0652 | rplL | 0.75 | 0.05830 |
| Rv0653c | hypothetical protein Rv0653c | 2.52 | 0.01477 |
| Rv0654 | hypothetical protein Rv0654 | 1.22 | 0.02754 |
| Rv0655 | hypothetical protein Rv0655 | 0.78 | 0.17899 |
| Rv0656c | hypothetical protein Rv0656c | 1.00 | 0.48461 |
| Rv0657c | hypothetical protein Rv0657c | 1.43 | 0.05504 |
| Rv0658c | hypothetical protein Rv0658c | 1.44 | 0.14414 |
| Rv0659c | hypothetical protein Rv0659c | 1.18 | 0.20351 |
| Rv0660c | hypothetical protein Rv0660c | 0.99 | 0.47866 |
| Rv0661c | hypothetical protein Rv0661c | 2.28 | 0.05818 |
| Rv0662c | hypothetical protein Rv0662c | 3.15 | 0.07117 |
| Rv0663 | atsD | 1.95 | 0.08954 |
| Rv0664 | hypothetical protein Rv0664 | 0.67 | 0.00050 |
| Rv0665 | hypothetical protein Rv0665 | 1.24 | 0.26561 |
| Rv0666 | hypothetical protein Rv0666 | 0.86 | 0.19477 |
| Rv0667 | rpoB | 0.78 | 0.07167 |
| Rv0668 | rpoC | 0.60 | 0.00017 |
| Rv0669c | hypothetical protein Rv0669c | 1.46 | 0.16201 |
| Rv0670 | end | 2.44 | 0.09477 |
| Rv0671 | lpqP | 2.46 | 0.06178 |
| Rv0672 | fadE8 | 1.60 | 0.12684 |
| Rv0673 | echA4 | 0.88 | 0.08679 |
| Rv0674 | hypothetical protein Rv0674 | 0.93 | 0.35229 |
| Rv0675 | echA5 | 1.42 | 0.07488 |
| Rv0676c | mmpL5 | 2.19 | 0.02616 |
| Rv0677c | mmpS5 | 0.95 | 0.38635 |
| Rv0678 | hypothetical protein Rv0678 | 1.87 | 0.00798 |
| Rv0679c | hypothetical protein Rv0679c | 1.55 | 0.00988 |
| Rv0680c | hypothetical protein Rv0680c | 0.91 | 0.34220 |
| Rv0681 | hypothetical protein Rv0681 | 1.10 | 0.05407 |
| Rv0682 | rpsL | 0.45 | 0.00007 |
| Rv0683 | rpsG | 0.41 | 0.00000 |
| Rv0684 | fusA | 0.57 | 0.00125 |
| Rv0685 | tuf | 1.28 | 0.21090 |
| Rv0686 | hypothetical protein Rv0686 | 1.02 | 0.45035 |
| Rv0687 | hypothetical protein Rv0687 | 2.43 | 0.00365 |
| Rv0688 | hypothetical protein Rv0688 | 0.92 | 0.12749 |
| Rv0689c | hypothetical protein Rv0689c | 1.24 | 0.00873 |
| Rv0690c | hypothetical protein Rv0690c | 1.02 | 0.46873 |
| Rv0691c | hypothetical protein Rv0691c | 2.90 | 0.02105 |
| Rv0692 | hypothetical protein Rv0692 | 0.18 | 0.00000 |
| Rv0693 | pqqE | 0.45 | 0.00004 |
| Rv0694 | lldD1 | 0.47 | 0.00004 |
| Rv0695 | hypothetical protein Rv0695 | 6.87 | 0.13644 |
| Rv0696 | hypothetical protein Rv0696 | 0.44 | 0.00051 |
| Rv0697 | hypothetical protein Rv0697 | 1.43 | 0.08397 |
| Rv0698 | hypothetical protein Rv0698 | 3.22 | 0.01617 |
| Rv0699 | hypothetical protein Rv0699 | 1.07 | 0.32749 |
| Rv0700 | rpsJ | 0.35 | 0.00000 |
| Rv0701 | rplC | 0.45 | 0.00001 |
| Rv0702 | rplD | 0.53 | 0.00391 |
| Rv0703 | rplW | 0.41 | 0.00000 |
| Rv0704 | rplB | 0.39 | 0.00021 |
| Rv0705 | rpsS | 0.72 | 0.08317 |
| Rv0706 | rplV | 0.43 | 0.00003 |
| Rv0707 | rpsC | 0.78 | 0.00000 |
| Rv0708 | rplP | 0.42 | 0.00039 |
| Rv0709 | rpmC | 0.66 | 0.13699 |
| Rv0710 | rpsQ | 0.47 | 0.00095 |
| Rv0711 | atsA | 1.05 | 0.42694 |
| Rv0712 | hypothetical protein Rv0712 | 2.03 | 0.06708 |
| Rv0713 | hypothetical protein Rv0713 | 0.72 | 0.00130 |
| Rv0714 | rplN | 3.01 | 0.06161 |
| Rv0715 | rplX | 0.68 | 0.08267 |
| Rv0716 | rplE | 0.42 | 0.00000 |
| Rv0717 | rpsN | 0.86 | 0.34926 |
| Rv0719 | rplF | 0.86 | 0.33463 |
| Rv0720 | rplR | 1.52 | 0.08874 |
| Rv0721 | rpsE | 0.84 | 0.04743 |
| Rv0722 | rpmD | 0.45 | 0.00004 |
| Rv0723 | rplO | 0.57 | 0.00011 |
| Rv0724 | sppA | 0.98 | 0.42392 |
| Rv0725c | hypothetical protein Rv0725c | 1.73 | 0.07006 |
| Rv0726c | hypothetical protein Rv0726c | 1.18 | 0.01539 |
| Rv0727c | fucA | 2.60 | 0.00824 |
| Rv0728c | hypothetical protein Rv0728c | 1.88 | 0.06336 |
| Rv0729 | xylB | 0.98 | 0.43673 |
| Rv0730 | hypothetical protein Rv0730 | 1.11 | 0.33131 |
| Rv0731c | hypothetical protein Rv0731c | 1.96 | 0.03987 |
| Rv0732 | secY | 1.70 | 0.18136 |
| Rv0733 | adk | 0.62 | 0.00017 |
| Rv0734 | map′ | 0.50 | 0.00001 |
| Rv0735 | sigL | 1.31 | 0.04305 |
| Rv0736 | hypothetical protein Rv0736 | 0.80 | 0.08557 |
| Rv0737 | hypothetical protein Rv0737 | 2.03 | 0.02063 |
| Rv0738 | hypothetical protein Rv0738 | 0.68 | 0.00406 |
| Rv0739 | hypothetical protein Rv0739 | 1.32 | 0.05574 |
| Rv0740 | hypothetical protein Rv0740 | 1.03 | 0.42070 |
| Rv0741 | hypothetical protein Rv0741 | 1.54 | 0.07662 |
| Rv0742 | PE_PGRS | 0.92 | 0.26700 |
| Rv0743c | hypothetical protein Rv0743c | 1.08 | 0.00331 |
| Rv0744c | hypothetical protein Rv0744c | 0.66 | 0.00542 |
| Rv0745 | hypothetical protein Rv0745 | 0.80 | 0.04147 |
| Rv0746 | PE_PGRS | 3.45 | 0.04764 |
| Rv0747 | PE_PGRS | 7.33 | 0.02625 |
| Rv0748 | hypothetical protein Rv0748 | 0.73 | 0.00051 |
| Rv0749 | hypothetical protein Rv0749 | 1.12 | 0.23418 |
| Rv0750 | hypothetical protein Rv0750 | 1.27 | 0.04241 |
| Rv0751c | mmsB | 1.12 | 0.12286 |
| Rv0752c | fadE9 | 0.76 | 0.07426 |
| Rv0753c | mmsA | 1.04 | 0.38964 |
| Rv0754 | PE_PGRS | 1.05 | 0.34685 |
| Rv0755c | PPE | 1.09 | 0.19960 |
| Rv0756c | hypothetical protein Rv0756c | 0.64 | 0.00033 |
| Rv0757 | phoP | 0.87 | 0.02137 |
| Rv0759c | hypothetical protein Rv0759c | 4.38 | 0.04744 |
| Rv0761c | adhB | 1.83 | 0.14165 |
| Rv0763c | hypothetical protein Rv0763c | 1.63 | 0.17092 |
| Rv0764c | hypothetical protein Rv0764c | 0.77 | 0.02008 |
| Rv0765c | hypothetical protein Rv0765c | 1.10 | 0.28745 |
| Rv0766c | hypothetical protein Rv0766c | 1.51 | 0.22874 |
| Rv0767c | hypothetical protein Rv0767c | 0.99 | 0.47086 |
| Rv0769 | hypothetical protein Rv0769 | 1.35 | 0.05923 |
| Rv0770 | hypothetical protein Rv0770 | 1.28 | 0.05356 |
| Rv0771 | hypothetical protein Rv0771 | 1.54 | 0.02230 |
| Rv0772 | purD | 2.61 | 0.00338 |
| Rv0773c | ggtA | 0.91 | 0.12548 |
| Rv0774c | hypothetical protein Rv0774c | 1.75 | 0.02445 |
| Rv0775 | hypothetical protein Rv0775 | 2.11 | 0.02125 |
| Rv0776c | hypothetical protein Rv0776c | 1.34 | 0.00004 |
| Rv0777 | purB | 0.91 | 0.02740 |
| Rv0778 | hypothetical protein Rv0778 | 1.12 | 0.12886 |
| Rv0779c | hypothetical protein Rv0779c | 1.08 | 0.04739 |
| Rv0780 | purC | 1.08 | 0.29474 |
| Rv0781 | ptrBb | 2.19 | 0.04833 |
| Rv0782 | ptrBa | 2.00 | 0.00882 |
| Rv0783c | hypothetical protein Rv0783c | 1.02 | 0.46985 |
| Rv0784 | hypothetical protein Rv0784 | 2.10 | 0.02079 |
| Rv0785 | hypothetical protein Rv0785 | 1.16 | 0.13423 |
| Rv0786c | hypothetical protein Rv0786c | 1.16 | 0.24176 |
| Rv0787 | hypothetical protein Rv0787 | 0.62 | 0.01198 |
| Rv0788 | purQ | 0.93 | 0.23557 |
| Rv0789c | hypothetical protein Rv0789c | 2.83 | 0.03030 |
| Rv0790c | hypothetical protein Rv0790c | 3.99 | 0.00784 |
| Rv0791c | hypothetical protein Rv0791c | 2.29 | 0.00199 |
| Rv0792c | hypothetical protein Rv0792c | 1.85 | 0.00288 |
| Rv0793 | hypothetical protein Rv0793 | 2.02 | 0.02183 |
| Rv0794c | lpdB | 2.28 | 0.00781 |
| Rv0795 | hypothetical protein Rv0795 | 1.50 | 0.10087 |
| Rv0796 | hypothetical protein Rv0796 | 2.33 | 0.02857 |
| Rv0797 | hypothetical protein Rv0797 | 3.01 | 0.01738 |
| Rv0798c | hypothetical protein Rv0798c | 0.89 | 0.13472 |
| Rv0799c | hypothetical protein Rv0799c | 1.49 | 0.10722 |
| Rv0800 | pepC | 0.73 | 0.00999 |
| Rv0801 | hypothetical protein Rv0801 | 1.12 | 0.17970 |
| Rv0802c | hypothetical protein Rv0802c | 1.15 | 0.19254 |
| Rv0803 | purL | 0.98 | 0.43052 |
| Rv0804 | hypothetical protein Rv0804 | 0.89 | 0.20418 |
| Rv0805 | hypothetical protein Rv0805 | 1.28 | 0.14958 |
| Rv0806c | cpsY | 1.37 | 0.00390 |
| Rv0807 | hypothetical protein Rv0807 | 0.98 | 0.37674 |
| Rv0808 | purF | 0.99 | 0.48634 |
| Rv0809 | purM | 0.99 | 0.45253 |
| Rv0810c | hypothetical protein Rv0810c | 0.99 | 0.46943 |
| Rv0811c | hypothetical protein Rv0811c | 1.25 | 0.02402 |
| Rv0812 | pabC | 1.43 | 0.15050 |
| Rv0813c | hypothetical protein Rv0813c | 0.88 | 0.02638 |
| Rv0814c | sseC2 | 0.63 | 0.00116 |
| Rv0815c | cysA2 | 0.91 | 0.24675 |
| Rv0816c | thiX | 2.24 | 0.02116 |
| Rv0817c | hypothetical protein Rv0817c | 0.70 | 0.00001 |
| Rv0818 | hypothetical protein Rv0818 | 1.24 | 0.11793 |
| Rv0819 | hypothetical protein Rv0819 | 0.92 | 0.09184 |
| Rv0820 | phoT | 1.92 | 0.01033 |
| Rv0821c | phoY2 | 0.77 | 0.00167 |
| Rv0822c | hypothetical protein Rv0822c | 1.32 | 0.08487 |
| Rv0823c | hypothetical protein Rv0823c | 3.08 | 0.02703 |
| Rv0824c | desA1 | 1.37 | 0.00725 |
| Rv0825c | hypothetical protein Rv0825c | 0.64 | 0.00000 |
| Rv0826 | hypothetical protein Rv0826 | 1.41 | 0.00080 |
| Rv0827c | hypothetical protein Rv0827c | 1.23 | 0.10806 |
| Rv0828c | hypothetical protein Rv0828c | 3.69 | 0.00251 |
| Rv0829 | hypothetical protein Rv0829 | 1.72 | 0.00547 |
| Rv0830 | hypothetical protein Rv0830 | 1.85 | 0.01080 |
| Rv0831c | hypothetical protein Rv0831c | 1.05 | 0.34167 |
| Rv0832 | PE_PGRS | 1.31 | 0.02592 |
| Rv0833 | PE_PGRS | 2.82 | 0.01917 |
| Rv0834c | PE_PGRS | 2.63 | 0.02795 |
| Rv0835 | lpqQ | 0.90 | 0.18314 |
| Rv0836c | hypothetical protein Rv0836c | 6.74 | 0.00420 |
| Rv0837c | hypothetical protein Rv0837c | 9.84 | 0.02515 |
| Rv0838 | lpqR | 2.10 | 0.01254 |
| Rv0839 | hypothetical protein Rv0839 | 4.67 | 0.01997 |
| Rv0840c | hypothetical protein Rv0840c | 1.17 | 0.22365 |
| Rv0841c | hypothetical protein Rv0841c | 1.72 | 0.02504 |
| Rv0842 | hypothetical protein Rv0842 | 1.48 | 0.02957 |
| Rv0843 | hypothetical protein Rv0843 | 1.72 | 0.02709 |
| Rv0844c | narL | 1.56 | 0.07729 |
| Rv0845 | hypothetical protein Rv0845 | 2.10 | 0.03001 |
| Rv0846c | hypothetical protein Rv0846c | 2.32 | 0.00750 |
| Rv0847 | lpqS | 1.40 | 0.04277 |
| Rv0848 | cysM3 | 3.34 | 0.04213 |
| Rv0849 | hypothetical protein Rv0849 | 2.67 | 0.07392 |
| Rv0850 | hypothetical protein Rv0850 | 3.57 | 0.01719 |
| Rv0851c | hypothetical protein Rv0851c | 1.56 | 0.02427 |
| Rv0852 | fadD16 | 0.97 | 0.40894 |
| Rv0853c | pdc | 1.04 | 0.13693 |
| Rv0854 | hypothetical protein Rv0854 | 0.85 | 0.00549 |
| Rv0855 | far | 0.84 | 0.00346 |
| Rv0856 | hypothetical protein Rv0856 | 1.01 | 0.47509 |
| Rv0857 | hypothetical protein Rv0857 | 1.19 | 0.17231 |
| Rv0858c | hypothetical protein Rv0858c | 1.85 | 0.08286 |
| Rv0859 | fadA | 0.81 | 0.06593 |
| Rv0860 | fadB | 0.70 | 0.04421 |
| Rv0861c | hypothetical protein Rv0861c | 1.02 | 0.39616 |
| Rv0862c | hypothetical protein Rv0862c | 0.68 | 0.00003 |
| Rv0863 | hypothetical protein Rv0863 | 1.00 | 0.49309 |
| Rv0864 | moaC2 | 0.55 | 0.00000 |
| Rv0865 | mog | 1.07 | 0.25703 |
| Rv0866 | moaE2 | 1.03 | 0.36493 |
| Rv0867c | hypothetical protein Rv0867c | 1.05 | 0.33107 |
| Rv0868c | moaD2 | 1.22 | 0.16089 |
| Rv0869c | moaA2 | 1.25 | 0.01537 |
| Rv0870c | hypothetical protein Rv0870c | 1.30 | 0.00001 |
| Rv0871 | cspB | 1.46 | 0.10784 |
| Rv0872c | PE_PGRS | 1.29 | 0.11193 |
| Rv0873 | fadE10 | 2.39 | 0.00705 |
| Rv0874c | hypothetical protein Rv0874c | 1.43 | 0.00117 |
| Rv0875c | hypothetical protein Rv0875c | 1.17 | 0.03838 |
| Rv0876c | hypothetical protein Rv0876c | 1.35 | 0.00320 |
| Rv0877 | hypothetical protein Rv0877 | 0.71 | 0.00021 |
| Rv0878c | PPE | 0.89 | 0.07391 |
| Rv0879c | hypothetical protein Rv0879c | 2.17 | 0.00275 |
| Rv0880 | hypothetical protein Rv0880 | 0.72 | 0.00352 |
| Rv0881 | hypothetical protein Rv0881 | 1.75 | 0.00279 |
| Rv0882 | hypothetical protein Rv0882 | 0.75 | 0.00629 |
| Rv0883c | hypothetical protein Rv0883c | 2.31 | 0.03460 |
| Rv0884c | serC | 1.38 | 0.00980 |
| Rv0885 | hypothetical protein Rv0885 | 3.83 | 0.00000 |
| Rv0886 | fprB | 1.35 | 0.00114 |
| Rv0887c | hypothetical protein Rv0887c | 2.07 | 0.00739 |
| Rv0888 | hypothetical protein Rv0888 | 1.49 | 0.11778 |
| Rv0889c | citA | 0.85 | 0.05160 |
| Rv0890c | hypothetical protein Rv0890c | 1.05 | 0.19575 |
| Rv0891c | hypothetical protein Rv0891c | 1.10 | 0.15599 |
| Rv0892 | hypothetical protein Rv0892 | 2.87 | 0.00238 |
| Rv0893c | hypothetical protein Rv0893c | 1.24 | 0.03573 |
| Rv0894 | hypothetical protein Rv0894 | 2.04 | 0.02818 |
| Rv0895 | hypothetical protein Rv0895 | 1.56 | 0.02756 |
| Rv0896 | gltA2 | 0.58 | 0.00000 |
| Rv0897c | hypothetical protein Rv0897c | 2.13 | 0.00098 |
| Rv0898c | hypothetical protein Rv0898c | 0.78 | 0.00553 |
| Rv0899 | ompA | 1.62 | 0.01324 |
| Rv0900 | hypothetical protein Rv0900 | 1.09 | 0.28061 |
| Rv0901 | hypothetical protein Rv0901 | 1.47 | 0.13657 |
| Rv0902c | hypothetical protein Rv0902c | 1.38 | 0.04965 |
| Rv0903c | hypothetical protein Rv0903c | 1.27 | 0.06684 |
| Rv0904c | accD3 | 2.39 | 0.01308 |
| Rv0905 | echA6 | 1.09 | 0.20956 |
| Rv0906 | hypothetical protein Rv0906 | 1.15 | 0.12609 |
| Rv0907 | hypothetical protein Rv0907 | 0.68 | 0.00203 |
| Rv0908 | ctpE | 0.59 | 0.00255 |
| Rv0909 | hypothetical protein Rv0909 | 0.63 | 0.00010 |
| Rv0910 | hypothetical protein Rv0910 | 0.78 | 0.09756 |
| Rv0911 | hypothetical protein Rv0911 | 1.16 | 0.30033 |
| Rv0912 | hypothetical protein Rv0912 | 1.46 | 0.15219 |
| Rv0913c | hypothetical protein Rv0913c | 1.23 | 0.00412 |
| Rv0914c | hypothetical protein Rv0914c | 1.19 | 0.20338 |
| Rv0915c | PPE | 2.72 | 0.02457 |
| Rv0916c | PE | 1.35 | 0.04300 |
| Rv0917 | betP | 1.43 | 0.09174 |
| Rv0918 | hypothetical protein Rv0918 | 1.54 | 0.00565 |
| Rv0919 | hypothetical protein Rv0919 | 0.88 | 0.16915 |
| Rv0920c | hypothetical protein Rv0920c | 2.10 | 0.06212 |
| Rv0921 | hypothetical protein Rv0921 | 0.70 | 0.00765 |
| Rv0922 | hypothetical protein Rv0922 | 1.11 | 0.30667 |
| Rv0923c | hypothetical protein Rv0923c | 2.41 | 0.00355 |
| Rv0924c | nramp | 1.22 | 0.09940 |
| Rv0925c | hypothetical protein Rv0925c | 3.03 | 0.00707 |
| Rv0926c | hypothetical protein Rv0926c | 0.73 | 0.00015 |
| Rv0927c | hypothetical protein Rv0927c | 1.64 | 0.03314 |
| Rv0928 | phoS2 | 1.15 | 0.17691 |
| Rv0929 | pstC2 | 1.89 | 0.04650 |
| Rv0930 | pstA1 | 1.43 | 0.06777 |
| Rv0931c | pknD | 1.14 | 0.27394 |
| Rv0932c | pstS | 0.61 | 0.00010 |
| Rv0933 | pstB | 1.06 | 0.30190 |
| Rv0934 | phoS1 | 0.88 | 0.31029 |
| Rv0935 | pstC | 1.13 | 0.25955 |
| Rv0936 | pstA2 | 1.84 | 0.09977 |
| Rv0937c | hypothetical protein Rv0937c | 0.95 | 0.30151 |
| Rv0938 | hypothetical protein Rv0938 | 0.94 | 0.34580 |
| Rv0939 | hypothetical protein Rv0939 | 1.38 | 0.03800 |
| Rv0940c | hypothetical protein Rv0940c | 0.86 | 0.01062 |
| Rv0941c | hypothetical protein Rv0941c | 1.05 | 0.35071 |
| Rv0942 | hypothetical protein Rv0942 | 3.27 | 0.00683 |
| Rv0943c | hypothetical protein Rv0943c | 2.18 | 0.06148 |
| Rv0944 | hypothetical protein Rv0944 | 0.82 | 0.06073 |
| Rv0945 | hypothetical protein Rv0945 | 1.00 | 0.48054 |
| Rv0946c | pgi | 1.18 | 0.14165 |
| Rv0947c | hypothetical protein Rv0947c | 1.67 | 0.02110 |
| Rv0948c | hypothetical protein Rv0948c | 0.61 | 0.00540 |
| Rv0949 | uvrD | 1.08 | 0.20092 |
| Rv0950c | hypothetical protein Rv0950c | 1.09 | 0.22888 |
| Rv0951 | sucC | 0.74 | 0.00211 |
| Rv0952 | sucD | 0.68 | 0.00065 |
| Rv0953c | hypothetical protein Rv0953c | 0.71 | 0.00047 |
| Rv0954 | hypothetical protein Rv0954 | 0.92 | 0.27339 |
| Rv0955 | hypothetical protein Rv0955 | 1.28 | 0.16423 |
| Rv0956 | purN | 1.39 | 0.07753 |
| Rv0957 | purH | 1.69 | 0.01060 |
| Rv0958 | hypothetical protein Rv0958 | 1.20 | 0.14256 |
| Rv0959 | hypothetical protein Rv0959 | 0.67 | 0.00134 |
| Rv0960 | hypothetical protein Rv0960 | 0.66 | 0.00002 |
| Rv0961 | hypothetical protein Rv0961 | 2.57 | 0.01257 |
| Rv0962c | hypothetical protein Rv0962c | 2.10 | 0.00013 |
| Rv0963c | hypothetical protein Rv0963c | 5.16 | 0.01500 |
| Rv0964c | hypothetical protein Rv0964c | 1.61 | 0.00177 |
| Rv0965c | hypothetical protein Rv0965c | 1.97 | 0.00226 |
| Rv0966c | hypothetical protein Rv0966c | 4.56 | 0.00169 |
| Rv0967 | hypothetical protein Rv0967 | 2.49 | 0.00646 |
| Rv0968 | hypothetical protein Rv0968 | 26.61 | 0.04204 |
| Rv0969 | ctpV | 2.45 | 0.01023 |
| Rv0970 | hypothetical protein Rv0970 | 22.75 | 0.00880 |
| Rv0971c | echA7 | 1.56 | 0.00093 |
| Rv0973c | accA2 | 1.67 | 0.00683 |
| Rv0974c | accD2 | 2.68 | 0.03225 |
| Rv0975c | fadE13 | 5.02 | 0.03182 |
| Rv0976c | hypothetical protein Rv0976c | 1.19 | 0.17643 |
| Rv0977 | PE_PGRS | 3.99 | 0.01847 |
| Rv0978c | PE_PGRS | 5.12 | 0.00047 |
| Rv0979c | hypothetical protein Rv0979c | 1.19 | 0.05179 |
| Rv0980c | PE_PGRS | 3.35 | 0.00934 |
| Rv0981 | hypothetical protein Rv0981 | 36.33 | 0.01248 |
| Rv0982 | hypothetical protein Rv0982 | 6.44 | 0.00833 |
| Rv0983 | hypothetical protein Rv0983 | 4.58 | 0.04662 |
| Rv0984 | moaB2 | 20.31 | 0.01409 |
| Rv0985c | mscL | 1.02 | 0.46125 |
| Rv0986 | hypothetical protein Rv0986 | 1.84 | 0.04955 |
| Rv0987 | hypothetical protein Rv0987 | 3.76 | 0.02001 |
| Rv0988 | hypothetical protein Rv0988 | 6.97 | 0.00409 |
| Rv0989c | grcC2 | 19.63 | 0.01630 |
| Rv0990c | hypothetical protein Rv0990c | 2.22 | 0.04391 |
| Rv0991c | hypothetical protein Rv0991c | 3.20 | 0.01170 |
| Rv0992c | hypothetical protein Rv0992c | 5.35 | 0.08165 |
| Rv0993 | galU | 1.03 | 0.39319 |
| Rv0994 | moeA | 0.87 | 0.05246 |
| Rv0995 | rimJ | 1.17 | 0.17350 |
| Rv0996 | hypothetical protein Rv0996 | 1.49 | 0.00036 |
| Rv0997 | hypothetical protein Rv0997 | 1.20 | 0.14410 |
| Rv0998 | hypothetical protein Rv0998 | 0.85 | 0.00061 |
| Rv0999 | hypothetical protein Rv0999 | 1.70 | 0.07768 |
| Rv1000 | hypothetical protein Rv1000 | 0.96 | 0.29679 |
| Rv1001 | arcA | 1.29 | 0.01722 |
| Rv1002c | hypothetical protein Rv1002c | 1.40 | 0.23097 |
| Rv1003 | hypothetical protein Rv1003 | 1.13 | 0.27889 |
| Rv1004c | hypothetical protein Rv1004c | 0.75 | 0.00000 |
| Rv1005c | pabB | 0.98 | 0.44325 |
| Rv1006 | hypothetical protein Rv1006 | 0.81 | 0.03776 |
| Rv1007c | metS | 1.13 | 0.27356 |
| Rv1008 | hypothetical protein Rv1008 | 1.10 | 0.31141 |
| Rv1009 | hypothetical protein Rv1009 | 0.79 | 0.00006 |
| Rv1010 | ksgA | 0.88 | 0.22534 |
| Rv1011 | hypothetical protein Rv1011 | 1.56 | 0.01903 |
| Rv1012 | hypothetical protein Rv1012 | 0.79 | 0.02015 |
| Rv1013 | pks16 | 1.51 | 0.06001 |
| Rv1014c | pth | 1.43 | 0.00007 |
| Rv1015c | rplY | 1.47 | 0.05258 |
| Rv1016c | lpqT | 1.16 | 0.06412 |
| Rv1017c | prsA | 0.75 | 0.00000 |
| Rv1018c | glmU | 7.97 | 0.00196 |
| Rv1019 | hypothetical protein Rv1019 | 0.85 | 0.03630 |
| Rv1020 | mfd | 1.48 | 0.00410 |
| Rv1021 | hypothetical protein Rv1021 | 1.64 | 0.08465 |
| Rv1022 | lpqU | 1.12 | 0.30432 |
| Rv1023 | eno | 1.24 | 0.12574 |
| Rv1024 | hypothetical protein Rv1024 | 2.03 | 0.05660 |
| Rv1025 | hypothetical protein Rv1025 | 1.12 | 0.25845 |
| Rv1026 | hypothetical protein Rv1026 | 1.96 | 0.05906 |
| Rv1027c | kdpE | 0.95 | 0.25394 |
| Rv1028c | kdpD | 1.58 | 0.00266 |
| Rv1029 | kdpA | 4.56 | 0.00093 |
| Rv1030 | kdpB | 1.26 | 0.13577 |
| Rv1031 | kdpC | 2.60 | 0.00529 |
| Rv1032c | hypothetical protein Rv1032c | 2.96 | 0.00175 |
| Rv1033c | hypothetical protein Rv1033c | 3.04 | 0.01341 |
| Rv1034c | hypothetical protein Rv1034c | 1.58 | 0.14088 |
| Rv1035c | hypothetical protein Rv1035c | 1.18 | 0.05436 |
| Rv1036c | hypothetical protein Rv1036c | 1.54 | 0.15984 |
| Rv1037c | hypothetical protein Rv1037c | 0.40 | 0.00000 |
| Rv1038c | hypothetical protein Rv1038c | 0.41 | 0.00000 |
| Rv1039c | PPE | 5.41 | 0.01891 |
| Rv1040c | PE | 2.32 | 0.08717 |
| Rv1041c | hypothetical protein Rv1041c | 3.05 | 0.01947 |
| Rv1042c | hypothetical protein Rv1042c | 1.06 | 0.33500 |
| Rv1043c | hypothetical protein Rv1043c | 1.91 | 0.04380 |
| Rv1044 | hypothetical protein Rv1044 | 2.35 | 0.03435 |
| Rv1045 | hypothetical protein Rv1045 | 0.71 | 0.00003 |
| Rv1046c | hypothetical protein Rv1046c | 1.18 | 0.24543 |
| Rv1047 | hypothetical protein Rv1047 | 2.10 | 0.03443 |
| Rv1048c | hypothetical protein Rv1048c | 1.10 | 0.31782 |
| Rv1049 | hypothetical protein Rv1049 | 4.17 | 0.01166 |
| Rv1050 | hypothetical protein Rv1050 | 0.81 | 0.00590 |
| Rv1051c | hypothetical protein Rv1051c | 2.05 | 0.04175 |
| Rv1052 | hypothetical protein Rv1052 | 0.96 | 0.14745 |
| Rv1053c | hypothetical protein Rv1053c | 1.95 | 0.06211 |
| Rv1054 | hypothetical protein Rv1054 | 1.27 | 0.22304 |
| Rv1055 | hypothetical protein Rv1055 | 1.41 | 0.20520 |
| Rv1056 | hypothetical protein Rv1056 | 2.58 | 0.04409 |
| Rv1057 | hypothetical protein Rv1057 | 0.90 | 0.17607 |
| Rv1058 | fadD14 | 2.24 | 0.02797 |
| Rv1059 | hypothetical protein Rv1059 | 0.92 | 0.14302 |
| Rv1060 | hypothetical protein Rv1060 | 1.09 | 0.32719 |
| Rv1061 | hypothetical protein Rv1061 | 1.57 | 0.08810 |
| Rv1062 | hypothetical protein Rv1062 | 1.48 | 0.00011 |
| Rv1063c | hypothetical protein Rv1063c | 1.33 | 0.06210 |
| Rv1064c | lpqV | 1.32 | 0.16695 |
| Rv1065 | hypothetical protein Rv1065 | 0.70 | 0.00106 |
| Rv1066 | hypothetical protein Rv1066 | 1.47 | 0.03102 |
| Rv1067c | PE_PGRS | 1.31 | 0.00400 |
| Rv1068c | PE_PGRS | 1.05 | 0.37683 |
| Rv1069c | hypothetical protein Rv1069c | 1.24 | 0.00937 |
| Rv1070c | echA8 | 2.11 | 0.08960 |
| Rv1071c | echA9 | 1.00 | 0.49670 |
| Rv1072 | hypothetical protein Rv1072 | 0.90 | 0.31109 |
| Rv1073 | hypothetical protein Rv1073 | 2.57 | 0.00000 |
| Rv1074c | fadA3 | 1.70 | 0.16498 |
| Rv1075c | hypothetical protein Rv1075c | 3.04 | 0.00889 |
| Rv1076 | lipU | 1.03 | 0.39071 |
| Rv1077 | cysM2 | 0.91 | 0.21210 |
| Rv1078 | pra | 0.52 | 0.00000 |
| Rv1079 | metB | 0.76 | 0.00959 |
| Rv1080c | greA | 0.66 | 0.00337 |
| Rv1081c | hypothetical protein Rv1081c | 3.34 | 0.03267 |
| Rv1082 | hypothetical protein Rv1082 | 1.21 | 0.16714 |
| Rv1083 | hypothetical protein Rv1083 | 1.84 | 0.06768 |
| Rv1084 | hypothetical protein Rv1084 | 0.71 | 0.00491 |
| Rv1085c | hypothetical protein Rv1085c | 2.67 | 0.01000 |
| Rv1086 | hypothetical protein Rv1086 | 0.66 | 0.00107 |
| Rv1087 | PE_PGRS | 1.75 | 0.02267 |
| Rv1088 | PE | 0.97 | 0.38865 |
| Rv1089 | PE | 1.17 | 0.32466 |
| Rv1090 | hypothetical protein Rv1090 | 0.76 | 0.01283 |
| Rv1091 | PE_PGRS | 1.48 | 0.00927 |
| Rv1092c | coaA | 2.01 | 0.03113 |
| Rv1093 | glyA | 1.81 | 0.17215 |
| Rv1094 | desA2 | 2.49 | 0.22808 |
| Rv1095 | phoH2 | 0.88 | 0.18537 |
| Rv1097c | hypothetical protein Rv1097c | 1.13 | 0.35097 |
| Rv1098c | fum | 5.42 | 0.13753 |
| Rv1099c | hypothetical protein Rv1099c | 2.76 | 0.06091 |
| Rv1100 | hypothetical protein Rv1100 | 1.47 | 0.10802 |
| Rv1101c | hypothetical protein Rv1101c | 1.35 | 0.13170 |
| Rv1102c | hypothetical protein Rv1102c | 2.07 | 0.24745 |
| Rv1103c | hypothetical protein Rv1103c | 1.66 | 0.07487 |
| Rv1104 | hypothetical protein Rv1104 | 1.48 | 0.09307 |
| Rv1105 | hypothetical protein Rv1105 | 1.23 | 0.00353 |
| Rv1106c | hypothetical protein Rv1106c | 1.30 | 0.17897 |
| Rv1107c | xseB | 0.87 | 0.14427 |
| Rv1108c | xseA | 0.83 | 0.14244 |
| Rv1109c | hypothetical protein Rv1109c | 1.12 | 0.30060 |
| Rv1110 | lytB′ | 0.65 | 0.01082 |
| Rv1111c | hypothetical protein Rv1111c | 2.30 | 0.05081 |
| Rv1112 | hypothetical protein Rv1112 | 0.82 | 0.13545 |
| Rv1113 | hypothetical protein Rv1113 | 0.77 | 0.01472 |
| Rv1114 | hypothetical protein Rv1114 | 1.57 | 0.05439 |
| Rv1115 | hypothetical protein Rv1115 | 1.41 | 0.00087 |
| Rv1116 | hypothetical protein Rv1116 | 1.28 | 0.08533 |
| Rv1117 | hypothetical protein Rv1117 | 1.05 | 0.15858 |
| Rv1118c | hypothetical protein Rv1118c | 1.19 | 0.18449 |
| Rv1119c | hypothetical protein Rv1119c | 1.15 | 0.10672 |
| Rv1120c | hypothetical protein Rv1120c | 0.70 | 0.00174 |
| Rv1121 | zwf | 1.33 | 0.04588 |
| Rv1122 | gnd2 | 0.67 | 0.01574 |
| Rv1123c | bpoB | 3.01 | 0.02140 |
| Rv1124 | ephC | 0.95 | 0.38794 |
| Rv1125 | hypothetical protein Rv1125 | 1.72 | 0.01284 |
| Rv1126c | hypothetical protein Rv1126c | 2.00 | 0.03184 |
| Rv1127c | ppdK | 0.99 | 0.48109 |
| Rv1128c | hypothetical protein Rv1128c | 3.40 | 0.00264 |
| Rv1129c | hypothetical protein Rv1129c | 1.75 | 0.02576 |
| Rv1130 | hypothetical protein Rv1130 | 5.76 | 0.01903 |
| Rv1131 | gltA1 | 4.46 | 0.00449 |
| Rv1132 | hypothetical protein Rv1132 | 1.66 | 0.07321 |
| Rv1133c | metE | 0.57 | 0.00000 |
| Rv1134 | hypothetical protein Rv1134 | 0.54 | 0.00002 |
| Rv1135c | PPE | 1.24 | 0.02337 |
| Rv1136 | hypothetical protein Rv1136 | 0.70 | 0.00061 |
| Rv1137c | hypothetical protein Rv1137c | 3.32 | 0.02465 |
| Rv1138c | hypothetical protein Rv1138c | 2.01 | 0.06195 |
| Rv1139c | hypothetical protein Rv1139c | 3.94 | 0.00254 |
| Rv1140 | hypothetical protein Rv1140 | 2.49 | 0.06003 |
| Rv1141c | echA11 | 1.79 | 0.04200 |
| Rv1143 | mcr | 1.69 | 0.11368 |
| Rv1145 | hypothetical protein Rv1145 | 1.22 | 0.15221 |
| Rv1147 | hypothetical protein Rv1147 | 0.72 | 0.00003 |
| Rv1149 | hypothetical protein Rv1149 | 1.32 | 0.09472 |
| Rv1150 | hypothetical protein Rv1150 | 0.96 | 0.36701 |
| Rv1151c | hypothetical protein Rv1151c | 2.94 | 0.06813 |
| Rv1152 | hypothetical protein Rv1152 | 0.28 | 0.29003 |
| Rv1153c | omt | 1.63 | 0.04357 |
| Rv1154c | hypothetical protein Rv1154c | 1.62 | 0.00029 |
| Rv1155 | hypothetical protein Rv1155 | 1.44 | 0.08357 |
| Rv1156 | hypothetical protein Rv1156 | 1.00 | 0.49335 |
| Rv1157c | hypothetical protein Rv1157c | 1.33 | 0.00877 |
| Rv1158c | hypothetical protein Rv1158c | 1.73 | 0.03002 |
| Rv1159 | hypothetical protein Rv1159 | 0.97 | 0.38022 |
| Rv1160 | mutT2 | 1.47 | 0.00049 |
| Rv1161 | narG | 1.19 | 0.00647 |
| Rv1162 | narH | 1.11 | 0.24754 |
| Rv1163 | narJ | 1.28 | 0.00227 |
| Rv1164 | narI | 1.55 | 0.02080 |
| Rv1165 | hypothetical protein Rv1165 | 1.51 | 0.03391 |
| Rv1166 | lpqW | 1.21 | 0.17597 |
| Rv1167c | hypothetical protein Rv1167c | 1.82 | 0.00601 |
| Rv1168c | PPE | 1.36 | 0.05962 |
| Rv1169c | PE | 2.82 | 0.00373 |
| Rv1170 | hypothetical protein Rv1170 | 4.49 | 0.01103 |
| Rv1171 | hypothetical protein Rv1171 | 1.27 | 0.09437 |
| Rv1172c | PE | 1.47 | 0.02067 |
| Rv1173 | hypothetical protein Rv1173 | 0.69 | 0.00000 |
| Rv1174c | hypothetical protein Rv1174c | 0.74 | 0.00000 |
| Rv1175c | fadH | 1.18 | 0.09299 |
| Rv1176c | hypothetical protein Rv1176c | 1.21 | 0.07698 |
| Rv1177 | fdxC | 1.04 | 0.34657 |
| Rv1178 | hypothetical protein Rv1178 | 0.92 | 0.32601 |
| Rv1179c | hypothetical protein Rv1179c | 2.30 | 0.01039 |
| Rv1180 | pks3 | 0.66 | 0.00000 |
| Rv1181 | pks4 | 1.18 | 0.17140 |
| Rv1182 | papA3 | 0.85 | 0.14339 |
| Rv1183 | mmpL10 | 2.14 | 0.01295 |
| Rv1184c | hypothetical protein Rv1184c | 1.00 | 0.49361 |
| Rv1185c | fadD21 | 0.83 | 0.09483 |
| Rv1186c | hypothetical protein Rv1186c | 0.99 | 0.40699 |
| Rv1187 | rocA | 4.67 | 0.01807 |
| Rv1188 | hypothetical protein Rv1188 | 1.33 | 0.04688 |
| Rv1189 | sigI | 1.56 | 0.03997 |
| Rv1190 | hypothetical protein Rv1190 | 1.96 | 0.01655 |
| Rv1191 | hypothetical protein Rv1191 | 1.20 | 0.02625 |
| Rv1192 | hypothetical protein Rv1192 | 2.36 | 0.02835 |
| Rv1193 | fadD36 | 1.00 | 0.48571 |
| Rv1194c | hypothetical protein Rv1194c | 0.90 | 0.07512 |
| Rv1195 | PE | 0.74 | 0.04791 |
| Rv1196 | PPE | 0.53 | 0.00023 |
| Rv1197 | hypothetical protein Rv1197 | 0.47 | 0.00000 |
| Rv1198 | hypothetical protein Rv1198 | 0.41 | 0.00000 |
| Rv1199c | hypothetical protein Rv1199c | 2.05 | 0.01200 |
| Rv1200 | hypothetical protein Rv1200 | 0.86 | 0.10216 |
| Rv1201c | hypothetical protein Rv1201c | 0.85 | 0.00295 |
| Rv1202 | dapE | 1.64 | 0.00574 |
| Rv1203c | hypothetical protein Rv1203c | 1.42 | 0.04351 |
| Rv1204c | hypothetical protein Rv1204c | 4.22 | 0.00519 |
| Rv1205 | hypothetical protein Rv1205 | 0.87 | 0.00676 |
| Rv1206 | fadD6 | 1.32 | 0.00251 |
| Rv1207 | folP2 | 0.82 | 0.00957 |
| Rv1208 | hypothetical protein Rv1208 | 1.18 | 0.11495 |
| Rv1209 | hypothetical protein Rv1209 | 1.14 | 0.22783 |
| Rv1210 | tagA | 1.40 | 0.05103 |
| Rv1211 | hypothetical protein Rv1211 | 0.72 | 0.00000 |
| Rv1212c | hypothetical protein Rv1212c | 1.05 | 0.15968 |
| Rv1213 | glgC | 1.57 | 0.00927 |
| Rv1214c | PE | 1.79 | 0.00808 |
| Rv1215c | hypothetical protein Rv1215c | 1.64 | 0.01316 |
| Rv1216c | hypothetical protein Rv1216c | 1.05 | 0.42413 |
| Rv1217c | hypothetical protein Rv1217c | 0.93 | 0.31566 |
| Rv1218c | hypothetical protein Rv1218c | 1.86 | 0.00245 |
| Rv1219c | hypothetical protein Rv1219c | 1.12 | 0.06240 |
| Rv1220c | hypothetical protein Rv1220c | 1.15 | 0.11186 |
| Rv1221 | sigE | 1.68 | 0.00275 |
| Rv1222 | hypothetical protein Rv1222 | 1.45 | 0.00326 |
| Rv1223 | htrA | 1.01 | 0.47247 |
| Rv1224 | hypothetical protein Rv1224 | 1.09 | 0.11084 |
| Rv1225c | hypothetical protein Rv1225c | 2.54 | 0.02950 |
| Rv1226c | hypothetical protein Rv1226c | 1.30 | 0.18667 |
| Rv1227c | hypothetical protein Rv1227c | 2.31 | 0.02707 |
| Rv1228 | lpqX | 1.01 | 0.45531 |
| Rv1229c | mrp | 2.76 | 0.02849 |
| Rv1230c | hypothetical protein Rv1230c | 1.11 | 0.18723 |
| Rv1231c | hypothetical protein Rv1231c | 1.58 | 0.00735 |
| Rv1232c | hypothetical protein Rv1232c | 1.23 | 0.06505 |
| Rv1233c | hypothetical protein Rv1233c | 1.07 | 0.37875 |
| Rv1234 | hypothetical protein Rv1234 | 0.98 | 0.25551 |
| Rv1235 | lpqY | 1.26 | 0.02925 |
| Rv1236 | sugA | 1.15 | 0.18038 |
| Rv1237 | sugB | 1.27 | 0.19436 |
| Rv1238 | sugC | 0.62 | 0.00000 |
| Rv1239c | corA | 1.05 | 0.14669 |
| Rv1240 | mdh | 1.06 | 0.38997 |
| Rv1241 | hypothetical protein Rv1241 | 1.11 | 0.27651 |
| Rv1242 | hypothetical protein Rv1242 | 1.14 | 0.20161 |
| Rv1243c | PE_PGRS | 0.97 | 0.27560 |
| Rv1244 | lpqZ | 1.01 | 0.47731 |
| Rv1245c | hypothetical protein Rv1245c | 1.68 | 0.00556 |
| Rv1246c | hypothetical protein Rv1246c | 1.20 | 0.05708 |
| Rv1247c | hypothetical protein Rv1247c | 1.34 | 0.01653 |
| Rv1248c | sucA | 1.14 | 0.27861 |
| Rv1249c | hypothetical protein Rv1249c | 1.17 | 0.08872 |
| Rv1250 | hypothetical protein Rv1250 | 0.87 | 0.19155 |
| Rv1251c | hypothetical protein Rv1251c | 1.72 | 0.00000 |
| Rv1252c | lprE | 1.16 | 0.06028 |
| Rv1253 | deaD | 1.13 | 0.15285 |
| Rv1254 | hypothetical protein Rv1254 | 0.91 | 0.10389 |
| Rv1255c | hypothetical protein Rv1255c | 1.44 | 0.00089 |
| Rv1256c | hypothetical protein Rv1256c | 1.01 | 0.46500 |
| Rv1257c | hypothetical protein Rv1257c | 1.38 | 0.00046 |
| Rv1258c | hypothetical protein Rv1258c | 1.47 | 0.00003 |
| Rv1259 | hypothetical protein Rv1259 | 1.30 | 0.00045 |
| Rv1260 | hypothetical protein Rv1260 | 1.42 | 0.05211 |
| Rv1261c | hypothetical protein Rv1261c | 1.06 | 0.27062 |
| Rv1262c | hypothetical protein Rv1262c | 0.95 | 0.31631 |
| Rv1263 | amiB2 | 1.13 | 0.04430 |
| Rv1264 | hypothetical protein Rv1264 | 1.10 | 0.24502 |
| Rv1265 | hypothetical protein Rv1265 | 1.29 | 0.06979 |
| Rv1266c | pknH | 0.61 | 0.00053 |
| Rv1267c | embR | 1.17 | 0.04495 |
| Rv1268c | hypothetical protein Rv1268c | 1.32 | 0.03004 |
| Rv1269c | hypothetical protein Rv1269c | 1.00 | 0.48181 |
| Rv1270c | lprA | 1.18 | 0.05115 |
| Rv1271c | hypothetical protein Rv1271c | 1.50 | 0.00208 |
| Rv1272c | hypothetical protein Rv1272c | 1.01 | 0.47595 |
| Rv1273c | hypothetical protein Rv1273c | 1.44 | 0.06701 |
| Rv1274 | lprB | 0.81 | 0.06916 |
| Rv1275 | lprC | 1.23 | 0.08088 |
| Rv1276c | hypothetical protein Rv1276c | 0.92 | 0.01917 |
| Rv1277 | hypothetical protein Rv1277 | 2.09 | 0.00743 |
| Rv1278 | hypothetical protein Rv1278 | 1.55 | 0.00072 |
| Rv1279 | hypothetical protein Rv1279 | 0.94 | 0.34059 |
| Rv1280c | oppA | 4.13 | 0.09223 |
| Rv1281c | oppD | 1.32 | 0.14671 |
| Rv1282c | oppC | 1.27 | 0.00402 |
| Rv1283c | oppB | 1.52 | 0.08792 |
| Rv1285 | cysD | 1.30 | 0.07983 |
| Rv1286 | cysN | 1.96 | 0.01479 |
| Rv1287 | hypothetical protein Rv1287 | 1.46 | 0.01965 |
| Rv1288 | hypothetical protein Rv1288 | 1.72 | 0.02510 |
| Rv1289 | hypothetical protein Rv1289 | 1.13 | 0.26606 |
| Rv1290c | hypothetical protein Rv1290c | 2.18 | 0.02815 |
| Rv1291c | hypothetical protein Rv1291c | 2.28 | 0.01389 |
| Rv1292 | argS | 1.30 | 0.16101 |
| Rv1293 | lysA | 1.23 | 0.19155 |
| Rv1294 | thrA | 1.01 | 0.46399 |
| Rv1295 | thrC | 1.43 | 0.13946 |
| Rv1296 | thrB | 1.08 | 0.31812 |
| Rv1297 | rho | 0.50 | 0.00088 |
| Rv1298 | rpmE | 0.67 | 0.02560 |
| Rv1299 | prfA | 0.69 | 0.21114 |
| Rv1300 | hemK | 0.88 | 0.13282 |
| Rv1301 | hypothetical protein Rv1301 | 1.27 | 0.08049 |
| Rv1302 | rfe | 2.52 | 0.00000 |
| Rv1303 | hypothetical protein Rv1303 | 0.84 | 0.00231 |
| Rv1304 | atpB | 0.30 | 0.00000 |
| Rv1305 | atpE | 0.58 | 0.00000 |
| Rv1306 | atpF | 0.30 | 0.00000 |
| Rv1307 | atpH | 0.75 | 0.05475 |
| Rv1308 | atpA | 0.45 | 0.00007 |
| Rv1309 | atpG | 0.74 | 0.00000 |
| Rv1310 | atpD | 0.26 | 0.00000 |
| Rv1311 | atpC | 0.36 | 0.00000 |
| Rv1312 | hypothetical protein Rv1312 | 0.49 | 0.00000 |
| Rv1313c | hypothetical protein Rv1313c | 2.95 | 0.01266 |
| Rv1314c | hypothetical protein Rv1314c | 0.93 | 0.14013 |
| Rv1315 | murA | 1.16 | 0.18391 |
| Rv1316c | ogt | 1.43 | 0.00668 |
| Rv1317c | alkA | 3.54 | 0.02047 |
| Rv1318c | hypothetical protein Rv1318c | 0.73 | 0.00922 |
| Rv1319c | hypothetical protein Rv1319c | 1.90 | 0.04069 |
| Rv1320c | hypothetical protein Rv1320c | 0.97 | 0.36934 |
| Rv1321 | hypothetical protein Rv1321 | 1.03 | 0.37147 |
| Rv1323 | fadA4 | 0.89 | 0.05672 |
| Rv1324 | hypothetical protein Rv1324 | 0.75 | 0.17967 |
| Rv1325c | PE_PGRS | 1.20 | 0.10731 |
| Rv1326c | glgB | 1.22 | 0.06709 |
| Rv1327c | hypothetical protein Rv1327c | 1.14 | 0.05132 |
| Rv1329c | dinG | 2.22 | 0.05022 |
| Rv1330c | hypothetical protein Rv1330c | 1.34 | 0.08264 |
| Rv1331 | hypothetical protein Rv1331 | 0.94 | 0.27341 |
| Rv1332 | hypothetical protein Rv1332 | 1.24 | 0.16027 |
| Rv1333 | hypothetical protein Rv1333 | 0.91 | 0.16613 |
| Rv1334 | hypothetical protein Rv1334 | 1.05 | 0.40203 |
| Rv1335 | hypothetical protein Rv1335 | 1.07 | 0.31108 |
| Rv1336 | cysM | 1.25 | 0.21789 |
| Rv1337 | hypothetical protein Rv1337 | 1.07 | 0.01368 |
| Rv1338 | murI | 0.97 | 0.43099 |
| Rv1339 | hypothetical protein Rv1339 | 0.99 | 0.48134 |
| Rv1340 | rphA | 0.81 | 0.02853 |
| Rv1341 | hypothetical protein Rv1341 | 0.93 | 0.32458 |
| Rv1342c | pks14 | 1.15 | 0.10015 |
| Rv1343c | hypothetical protein Rv1343c | 1.81 | 0.00018 |
| Rv1344 | hypothetical protein Rv1344 | 1.05 | 0.26597 |
| Rv1345 | fadD33 | 0.83 | 0.00082 |
| Rv1346 | fadE14 | 1.25 | 0.02857 |
| Rv1347c | hypothetical protein Rv1347c | 1.16 | 0.06139 |
| Rv1348 | hypothetical protein Rv1348 | 1.16 | 0.06511 |
| Rv1349 | hypothetical protein Rv1349 | 1.07 | 0.30428 |
| Rv1350 | fabG2 | 1.40 | 0.00094 |
| Rv1351 | hypothetical protein Rv1351 | 0.99 | 0.43174 |
| Rv1352 | hypothetical protein Rv1352 | 1.47 | 0.07423 |
| Rv1353c | hypothetical protein Rv1353c | 1.76 | 0.11361 |
| Rv1354c | hypothetical protein Rv1354c | 1.24 | 0.03439 |
| Rv1355c | moeY | 1.43 | 0.10703 |
| Rv1356c | hypothetical protein Rv1356c | 1.53 | 0.01321 |
| Rv1357c | hypothetical protein Rv1357c | 1.41 | 0.03337 |
| Rv1358 | hypothetical protein Rv1358 | 1.04 | 0.43616 |
| Rv1359 | hypothetical protein Rv1359 | 1.11 | 0.23799 |
| Rv1360 | hypothetical protein Rv1360 | 0.79 | 0.08190 |
| Rv1361c | PPE | 1.01 | 0.49071 |
| Rv1362c | hypothetical protein Rv1362c | 0.82 | 0.00038 |
| Rv1363c | hypothetical protein Rv1363c | 0.97 | 0.36222 |
| Rv1364c | rsbU | 1.12 | 0.06520 |
| Rv1365c | hypothetical protein Rv1365c | 1.64 | 0.00945 |
| Rv1366 | hypothetical protein Rv1366 | 1.39 | 0.01767 |
| Rv1367c | hypothetical protein Rv1367c | 2.36 | 0.00200 |
| Rv1368 | lprF | 1.09 | 0.15258 |
| Rv1369c | hypothetical protein Rv1369c | 1.93 | 0.01780 |
| Rv1370c | hypothetical protein Rv1370c | 1.49 | 0.10347 |
| Rv1371 | hypothetical protein Rv1371 | 1.41 | 0.02361 |
| Rv1372 | pks18 | 1.75 | 0.01549 |
| Rv1373 | hypothetical protein Rv1373 | 1.76 | 0.01780 |
| Rv1374c | hypothetical protein Rv1374c | 1.14 | 0.29068 |
| Rv1375 | hypothetical protein Rv1375 | 2.30 | 0.00037 |
| Rv1376 | hypothetical protein Rv1376 | 1.22 | 0.13627 |
| Rv1377c | hypothetical protein Rv1377c | 1.09 | 0.21699 |
| Rv1378c | hypothetical protein Rv1378c | 1.41 | 0.09196 |
| Rv1379 | pyrR | 1.06 | 0.24224 |
| Rv1380 | pyrB | 0.88 | 0.07050 |
| Rv1382 | hypothetical protein Rv1382 | 0.72 | 0.00562 |
| Rv1383 | carA | 2.56 | 0.01031 |
| Rv1384 | carB | 0.74 | 0.00491 |
| Rv1385 | pyrF | 0.95 | 0.15507 |
| Rv1386 | PE | 0.95 | 0.38321 |
| Rv1387 | PPE | 1.04 | 0.45197 |
| Rv1388 | mIHF | 0.72 | 0.00414 |
| Rv1389 | gmk | 1.29 | 0.07955 |
| Rv1390 | hypothetical protein Rv1390 | 0.77 | 0.00195 |
| Rv1392 | metK | 0.96 | 0.41771 |
| Rv1393c | hypothetical protein Rv1393c | 3.53 | 0.00615 |
| Rv1394c | hypothetical protein Rv1394c | 2.38 | 0.00061 |
| Rv1395 | hypothetical protein Rv1395 | 1.87 | 0.05974 |
| Rv1396c | PE_PGRS | 1.03 | 0.44535 |
| Rv1397c | hypothetical protein Rv1397c | 1.84 | 0.00257 |
| Rv1398c | hypothetical protein Rv1398c | 1.06 | 0.40189 |
| Rv1399c | lipH | 0.95 | 0.19086 |
| Rv1400c | lipI | 1.44 | 0.12702 |
| Rv1401 | hypothetical protein Rv1401 | 0.92 | 0.17814 |
| Rv1402 | priA | 1.05 | 0.20788 |
| Rv1403c | hypothetical protein Rv1403c | 0.86 | 0.04401 |
| Rv1404 | hypothetical protein Rv1404 | 0.99 | 0.47058 |
| Rv1405c | hypothetical protein Rv1405c | 2.90 | 0.00092 |
| Rv1406 | fmt | 2.96 | 0.00365 |
| Rv1407 | fmu | 1.37 | 0.00116 |
| Rv1408 | rpe | 5.94 | 0.03224 |
| Rv1409 | ribG | 1.34 | 0.00805 |
| Rv1410c | hypothetical protein Rv1410c | 0.93 | 0.35595 |
| Rv1411c | lprG | 0.89 | 0.06531 |
| Rv1412 | ribC | 0.94 | 0.30926 |
| Rv1413 | hypothetical protein Rv1413 | 1.20 | 0.09545 |
| Rv1414 | hypothetical protein Rv1414 | 2.10 | 0.07565 |
| Rv1415 | ribA2 | 0.98 | 0.32606 |
| Rv1416 | ribH | 0.93 | 0.35412 |
| Rv1417 | hypothetical protein Rv1417 | 1.17 | 0.19517 |
| Rv1418 | lprH | 0.81 | 0.01369 |
| Rv1419 | hypothetical protein Rv1419 | 2.85 | 0.01007 |
| Rv1420 | uvrC | 1.74 | 0.09725 |
| Rv1421 | hypothetical protein Rv1421 | 1.07 | 0.06854 |
| Rv1422 | hypothetical protein Rv1422 | 1.58 | 0.12463 |
| Rv1423 | hypothetical protein Rv1423 | 1.03 | 0.38849 |
| Rv1424c | hypothetical protein Rv1424c | 1.12 | 0.24556 |
| Rv1425 | hypothetical protein Rv1425 | 1.93 | 0.11914 |
| Rv1426c | lipO | 0.76 | 0.00775 |
| Rv1427c | fadD12 | 1.35 | 0.03093 |
| Rv1428c | hypothetical protein Rv1428c | 2.71 | 0.09460 |
| Rv1429 | hypothetical protein Rv1429 | 1.95 | 0.06563 |
| Rv1430 | PE | 1.24 | 0.06888 |
| Rv1431 | hypothetical protein Rv1431 | 1.60 | 0.14703 |
| Rv1432 | hypothetical protein Rv1432 | 4.53 | 0.06103 |
| Rv1433 | hypothetical protein Rv1433 | 1.28 | 0.13343 |
| Rv1434 | hypothetical protein Rv1434 | 1.57 | 0.00499 |
| Rv1435c | hypothetical protein Rv1435c | 1.10 | 0.39899 |
| Rv1436 | gap | 1.17 | 0.36478 |
| Rv1437 | pgk | 0.84 | 0.22806 |
| Rv1438 | tpi | 0.63 | 0.00108 |
| Rv1439c | hypothetical protein Rv1439c | 1.41 | 0.08386 |
| Rv1440 | secG | 0.88 | 0.22239 |
| Rv1441c | PE_PGRS | 1.30 | 0.04780 |
| Rv1442 | bisC | 1.07 | 0.33727 |
| Rv1443c | hypothetical protein Rv1443c | 1.31 | 0.02496 |
| Rv1444c | hypothetical protein Rv1444c | 0.71 | 0.00054 |
| Rv1445c | devB | 1.20 | 0.00567 |
| Rv1446c | opcA | 0.96 | 0.36465 |
| Rv1447c | zwf2 | 1.13 | 0.01429 |
| Rv1448c | tal | 0.35 | 0.00000 |
| Rv1449c | tkt | 1.02 | 0.43725 |
| Rv1450c | PE_PGRS | 0.95 | 0.38156 |
| Rv1451 | ctaB | 0.78 | 0.00015 |
| Rv1452c | PE_PGRS | 1.76 | 0.17083 |
| Rv1453 | hypothetical protein Rv1453 | 1.06 | 0.25315 |
| Rv1454c | qor | 0.67 | 0.00017 |
| Rv1455 | hypothetical protein Rv1455 | 1.34 | 0.00155 |
| Rv1456c | hypothetical protein Rv1456c | 1.01 | 0.47327 |
| Rv1457c | hypothetical protein Rv1457c | 1.17 | 0.07969 |
| Rv1458c | hypothetical protein Rv1458c | 0.89 | 0.05392 |
| Rv1459c | hypothetical protein Rv1459c | 1.62 | 0.00064 |
| Rv1460 | hypothetical protein Rv1460 | 1.87 | 0.00000 |
| Rv1461 | hypothetical protein Rv1461 | 1.28 | 0.00738 |
| Rv1462 | hypothetical protein Rv1462 | 1.76 | 0.00003 |
| Rv1463 | hypothetical protein Rv1463 | 1.09 | 0.12217 |
| Rv1464 | hypothetical protein Rv1464 | 1.54 | 0.00016 |
| Rv1465 | hypothetical protein Rv1465 | 1.43 | 0.00655 |
| Rv1466 | hypothetical protein Rv1466 | 0.80 | 0.01458 |
| Rv1467c | fadE15 | 1.30 | 0.01125 |
| Rv1468c | PE_PGRS | 0.82 | 0.07307 |
| Rv1469 | ctpD | 1.60 | 0.00323 |
| Rv1470 | trxA | 0.92 | 0.00542 |
| Rv1471 | trxB | 2.70 | 0.01803 |
| Rv1472 | echA12 | 0.83 | 0.00431 |
| Rv1473 | hypothetical protein Rv1473 | 1.79 | 0.02413 |
| Rv1474c | hypothetical protein Rv1474c | 0.64 | 0.00002 |
| Rv1475c | acn | 2.27 | 0.05720 |
| Rv1476 | hypothetical protein Rv1476 | 1.02 | 0.46786 |
| Rv1477 | hypothetical protein Rv1477 | 1.33 | 0.20057 |
| Rv1479 | moxR | 1.18 | 0.29454 |
| Rv1480 | hypothetical protein Rv1480 | 1.59 | 0.34899 |
| Rv1481 | hypothetical protein Rv1481 | 1.05 | 0.40667 |
| Rv1483 | fabG1 | 1.51 | 0.13917 |
| Rv1485 | hemZ | 1.95 | 0.00820 |
| Rv1487 | hypothetical protein Rv1487 | 0.92 | 0.19144 |
| Rv1488 | hypothetical protein Rv1488 | 2.33 | 0.06373 |
| Rv1489c | hypothetical protein Rv1489c | 2.45 | 0.07586 |
| Rv1490 | hypothetical protein Rv1490 | 3.09 | 0.01141 |
| Rv1491c | hypothetical protein Rv1491c | 1.39 | 0.03761 |
| Rv1492 | mutA | 1.64 | 0.04838 |
| Rv1493 | mutB | 2.38 | 0.00126 |
| Rv1494 | hypothetical protein Rv1494 | 0.98 | 0.34713 |
| Rv1495 | hypothetical protein Rv1495 | 1.52 | 0.03408 |
| Rv1496 | hypothetical protein Rv1496 | 1.45 | 0.06861 |
| Rv1497 | lipL | 1.56 | 0.10899 |
| Rv1498c | hypothetical protein Rv1498c | 1.41 | 0.05947 |
| Rv1499 | hypothetical protein Rv1499 | 2.72 | 0.00337 |
| Rv1500 | hypothetical protein Rv1500 | 1.39 | 0.09154 |
| Rv1501 | hypothetical protein Rv1501 | 1.52 | 0.03859 |
| Rv1502 | hypothetical protein Rv1502 | 0.74 | 0.00006 |
| Rv1503c | hypothetical protein Rv1503c | 1.03 | 0.40129 |
| Rv1504c | hypothetical protein Rv1504c | 1.09 | 0.25132 |
| Rv1505c | hypothetical protein Rv1505c | 1.05 | 0.18422 |
| Rv1506c | hypothetical protein Rv1506c | 1.05 | 0.36955 |
| Rv1507c | hypothetical protein Rv1507c | 1.10 | 0.27823 |
| Rv1508c | hypothetical protein Rv1508c | 1.11 | 0.17632 |
| Rv1509 | hypothetical protein Rv1509 | 1.24 | 0.10388 |
| Rv1510 | hypothetical protein Rv1510 | 0.80 | 0.00004 |
| Rv1511 | gmdA | 1.12 | 0.05207 |
| Rv1512 | epiA | 0.99 | 0.48572 |
| Rv1513 | hypothetical protein Rv1513 | 2.04 | 0.00987 |
| Rv1514c | hypothetical protein Rv1514c | 0.63 | 0.00016 |
| Rv1515c | hypothetical protein Rv1515c | 2.90 | 0.01081 |
| Rv1516c | hypothetical protein Rv1516c | 1.80 | 0.03344 |
| Rv1517 | hypothetical protein Rv1517 | 2.41 | 0.00441 |
| Rv1518 | hypothetical protein Rv1518 | 0.93 | 0.19993 |
| Rv1519 | hypothetical protein Rv1519 | 1.65 | 0.00684 |
| Rv1520 | hypothetical protein Rv1520 | 0.72 | 0.00001 |
| Rv1521 | fadD25 | 1.97 | 0.06446 |
| Rv1522c | mmpL12 | 1.59 | 0.10291 |
| Rv1523 | hypothetical protein Rv1523 | 1.70 | 0.09471 |
| Rv1524 | hypothetical protein Rv1524 | 2.02 | 0.03413 |
| Rv1525 | wbbl2 | 0.77 | 0.00335 |
| Rv1526c | hypothetical protein Rv1526c | 2.13 | 0.05358 |
| Rv1527c | pks5 | 1.96 | 0.01846 |
| Rv1528c | papA4 | 3.20 | 0.29593 |
| Rv1529 | fadD24 | 1.68 | 0.00347 |
| Rv1530 | adh | 0.66 | 0.00065 |
| Rv1531 | hypothetical protein Rv1531 | 0.81 | 0.03967 |
| Rv1532c | hypothetical protein Rv1532c | 14.25 | 0.03868 |
| Rv1533 | hypothetical protein Rv1533 | 1.10 | 0.24718 |
| Rv1534 | hypothetical protein Rv1534 | 0.66 | 0.00051 |
| Rv1535 | hypothetical protein Rv1535 | 0.70 | 0.00278 |
| Rv1536 | ileS | 0.66 | 0.01394 |
| Rv1537 | dinX | 0.96 | 0.39509 |
| Rv1538c | ansA | 3.33 | 0.00319 |
| Rv1539 | lspA | 1.09 | 0.22576 |
| Rv1540 | hypothetical protein Rv1540 | 1.90 | 0.00068 |
| Rv1541c | lprI | 0.80 | 0.00016 |
| Rv1542c | glbN | 1.17 | 0.04215 |
| Rv1543 | hypothetical protein Rv1543 | 0.92 | 0.05148 |
| Rv1544 | hypothetical protein Rv1544 | 0.97 | 0.38289 |
| Rv1545 | hypothetical protein Rv1545 | 0.98 | 0.32444 |
| Rv1546 | hypothetical protein Rv1546 | 0.96 | 0.35012 |
| Rv1547 | dnaE1 | 1.20 | 0.18489 |
| Rv1548c | PPE | 1.32 | 0.00097 |
| Rv1549 | fadD11′ | 1.65 | 0.00969 |
| Rv1550 | fadD11 | 1.06 | 0.31705 |
| Rv1551 | plsB1 | 0.72 | 0.00368 |
| Rv1552 | frdA | 2.51 | 0.02553 |
| Rv1553 | frdB | 1.00 | 0.49240 |
| Rv1554 | frdC | 1.61 | 0.01975 |
| Rv1555 | frdD | 1.01 | 0.45856 |
| Rv1556 | hypothetical protein Rv1556 | 2.40 | 0.00747 |
| Rv1557 | mmpL6 | 1.00 | 0.48533 |
| Rv1558 | hypothetical protein Rv1558 | 1.32 | 0.04460 |
| Rv1559 | ilvA | 1.51 | 0.03460 |
| Rv1560 | hypothetical protein Rv1560 | 1.45 | 0.03285 |
| Rv1561 | hypothetical protein Rv1561 | 2.32 | 0.02333 |
| Rv1562c | glgZ | 1.60 | 0.03181 |
| Rv1563c | glgY | 1.40 | 0.10954 |
| Rv1564c | glgX | 1.31 | 0.06004 |
| Rv1565c | hypothetical protein Rv1565c | 3.06 | 0.01050 |
| Rv1566c | hypothetical protein Rv1566c | 0.97 | 0.39000 |
| Rv1567c | hypothetical protein Rv1567c | 1.00 | 0.48824 |
| Rv1568 | bioA | 1.33 | 0.03251 |
| Rv1569 | bioF | 1.67 | 0.06271 |
| Rv1570 | bioD | 1.87 | 0.00145 |
| Rv1571 | hypothetical protein Rv1571 | 1.07 | 0.27120 |
| Rv1572c | hypothetical protein Rv1572c | 2.94 | 0.00927 |
| Rv1573 | hypothetical protein Rv1573 | 1.40 | 0.15465 |
| Rv1574 | hypothetical protein Rv1574 | 1.38 | 0.07829 |
| Rv1575 | hypothetical protein Rv1575 | 3.37 | 0.01664 |
| Rv1576c | hypothetical protein Rv1576c | 0.75 | 0.04077 |
| Rv1577c | hypothetical protein Rv1577c | 2.87 | 0.02350 |
| Rv1578c | hypothetical protein Rv1578c | 1.53 | 0.08781 |
| Rv1579c | hypothetical protein Rv1579c | 1.08 | 0.10273 |
| Rv1580c | hypothetical protein Rv1580c | 1.53 | 0.10338 |
| Rv1581c | hypothetical protein Rv1581c | 4.28 | 0.03061 |
| Rv1582c | hypothetical protein Rv1582c | 3.00 | 0.02166 |
| Rv1583c | hypothetical protein Rv1583c | 0.85 | 0.00371 |
| Rv1584c | hypothetical protein Rv1584c | 1.16 | 0.23402 |
| Rv1585c | hypothetical protein Rv1585c | 1.83 | 0.00988 |
| Rv1586c | hypothetical protein Rv1586c | 1.87 | 0.02238 |
| Rv1587c | hypothetical protein Rv1587c | 8.46 | 0.01030 |
| Rv1588c | hypothetical protein Rv1588c | 1.76 | 0.00222 |
| Rv1589 | bioB | 0.80 | 0.00245 |
| Rv1590 | hypothetical protein Rv1590 | 1.13 | 0.07794 |
| Rv1591 | hypothetical protein Rv1591 | 1.54 | 0.04048 |
| Rv1592c | hypothetical protein Rv1592c | 2.39 | 0.00072 |
| Rv1593c | hypothetical protein Rv1593c | 1.10 | 0.31019 |
| Rv1594 | nadA | 1.03 | 0.43886 |
| Rv1595 | nadB | 0.74 | 0.00352 |
| Rv1596 | nadC | 0.88 | 0.17879 |
| Rv1597 | hypothetical protein Rv1597 | 1.00 | 0.49264 |
| Rv1598c | hypothetical protein Rv1598c | 0.92 | 0.21420 |
| Rv1599 | hisD | 1.25 | 0.02885 |
| Rv1600 | hisC | 1.79 | 0.00001 |
| Rv1601 | hisB | 1.32 | 0.06025 |
| Rv1602 | hisH | 1.48 | 0.02802 |
| Rv1603 | hisA | 0.98 | 0.44595 |
| Rv1604 | impA | 0.97 | 0.44701 |
| Rv1605 | hisF | 1.41 | 0.08652 |
| Rv1606 | hisI2 | 0.76 | 0.05123 |
| Rv1607 | chaA | 0.78 | 0.00389 |
| Rv1608c | bcpB | 1.66 | 0.01811 |
| Rv1609 | trpE | 1.01 | 0.45955 |
| Rv1610 | hypothetical protein Rv1610 | 1.01 | 0.45364 |
| Rv1611 | trpC | 0.82 | 0.10784 |
| Rv1612 | trpB | 1.51 | 0.15330 |
| Rv1613 | trpA | 1.03 | 0.45141 |
| Rv1614 | lgt | 0.70 | 0.01885 |
| Rv1615 | hypothetical protein Rv1615 | 1.01 | 0.48417 |
| Rv1616 | hypothetical protein Rv1616 | 1.51 | 0.08484 |
| Rv1617 | pykA | 1.15 | 0.27259 |
| Rv1618 | tesB1 | 0.79 | 0.07842 |
| Rv1619 | hypothetical protein Rv1619 | 1.47 | 0.08159 |
| Rv1620c | cydC | 1.85 | 0.12167 |
| Rv1621c | cydD | 2.00 | 0.05786 |
| Rv1622c | cydB | 2.03 | 0.07699 |
| Rv1623c | appC | 1.92 | 0.02903 |
| Rv1624c | hypothetical protein Rv1624c | 2.67 | 0.01634 |
| Rv1625c | hypothetical protein Rv1625c | 1.51 | 0.14636 |
| Rv1626 | hypothetical protein Rv1626 | 0.57 | 0.00001 |
| Rv1627c | hypothetical protein Rv1627c | 1.61 | 0.09058 |
| Rv1628c | hypothetical protein Rv1628c | 0.90 | 0.33297 |
| Rv1629 | polA | 1.21 | 0.16223 |
| Rv1630 | rpsA | 0.69 | 0.03663 |
| Rv1631 | hypothetical protein Rv1631 | 0.63 | 0.00000 |
| Rv1632c | hypothetical protein Rv1632c | 1.81 | 0.09798 |
| Rv1633 | uvrB | 1.66 | 0.00683 |
| Rv1634 | hypothetical protein Rv1634 | 1.22 | 0.00981 |
| Rv1635c | hypothetical protein Rv1635c | 1.08 | 0.15147 |
| Rv1636 | hypothetical protein Rv1636 | 1.54 | 0.00004 |
| Rv1637c | hypothetical protein Rv1637c | 1.27 | 0.00018 |
| Rv1638 | uvrA | 2.24 | 0.00000 |
| Rv1639c | hypothetical protein Rv1639c | 0.81 | 0.02847 |
| Rv1640c | lysX | 1.51 | 0.09300 |
| Rv1641 | infC | 0.74 | 0.00007 |
| Rv1642 | rpmI | 0.57 | 0.05666 |
| Rv1643 | rplT | 0.61 | 0.00778 |
| Rv1644 | tsnR | 0.78 | 0.01645 |
| Rv1645c | hypothetical protein Rv1645c | 1.18 | 0.24164 |
| Rv1646 | PE | 1.27 | 0.27730 |
| Rv1647 | hypothetical protein Rv1647 | 1.13 | 0.15421 |
| Rv1648 | hypothetical protein Rv1648 | 0.83 | 0.16213 |
| Rv1649 | pheS | 1.84 | 0.00194 |
| Rv1650 | pheT | 0.94 | 0.37187 |
| Rv1651c | PE_PGRS | 1.94 | 0.01701 |
| Rv1652 | argC | 0.83 | 0.07586 |
| Rv1653 | argJ | 1.14 | 0.13383 |
| Rv1654 | argB | 1.02 | 0.35719 |
| Rv1655 | argD | 1.46 | 0.00414 |
| Rv1656 | argF | 1.40 | 0.00287 |
| Rv1657 | argR | 3.04 | 0.02277 |
| Rv1658 | argG | 5.07 | 0.01503 |
| Rv1659 | argH | 2.01 | 0.05138 |
| Rv1660 | pks10 | 4.30 | 0.03167 |
| Rv1661 | pks7 | 0.68 | 0.00501 |
| Rv1662 | pks8 | 1.03 | 0.44321 |
| Rv1663 | pks17 | 1.15 | 0.03195 |
| Rv1664 | pks9 | 1.55 | 0.01526 |
| Rv1665 | pks11 | 1.66 | 0.08933 |
| Rv1666c | hypothetical protein Rv1666c | 3.58 | 0.13938 |
| Rv1667c | hypothetical protein Rv1667c | 0.97 | 0.39557 |
| Rv1669 | hypothetical protein Rv1669 | 0.87 | 0.04204 |
| Rv1670 | hypothetical protein Rv1670 | 1.58 | 0.06119 |
| Rv1671 | hypothetical protein Rv1671 | 0.97 | 0.28377 |
| Rv1672c | hypothetical protein Rv1672c | 1.25 | 0.04618 |
| Rv1673c | hypothetical protein Rv1673c | 2.66 | 0.03339 |
| Rv1674c | hypothetical protein Rv1674c | 1.61 | 0.00289 |
| Rv1675c | hypothetical protein Rv1675c | 5.90 | 0.04301 |
| Rv1676 | hypothetical protein Rv1676 | 0.58 | 0.00000 |
| Rv1677 | dsbF | 1.22 | 0.32562 |
| Rv1678 | hypothetical protein Rv1678 | 0.85 | 0.16465 |
| Rv1679 | fadE16 | 1.30 | 0.24356 |
| Rv1680 | hypothetical protein Rv1680 | 1.25 | 0.18781 |
| Rv1681 | moeX | 3.65 | 0.01577 |
| Rv1682 | hypothetical protein Rv1682 | 0.90 | 0.29064 |
| Rv1683 | hypothetical protein Rv1683 | 1.44 | 0.01226 |
| Rv1684 | hypothetical protein Rv1684 | 1.32 | 0.16142 |
| Rv1685c | hypothetical protein Rv1685c | 1.65 | 0.01999 |
| Rv1686c | hypothetical protein Rv1686c | 0.84 | 0.21445 |
| Rv1687c | hypothetical protein Rv1687c | 1.99 | 0.00002 |
| Rv1688 | hypothetical protein Rv1688 | 1.65 | 0.04256 |
| Rv1689 | tyrS | 1.06 | 0.28292 |
| Rv1690 | lprJ | 1.03 | 0.45452 |
| Rv1691 | hypothetical protein Rv1691 | 1.15 | 0.04402 |
| Rv1692 | hypothetical protein Rv1692 | 1.03 | 0.36762 |
| Rv1693 | hypothetical protein Rv1693 | 1.13 | 0.12706 |
| Rv1694 | tlyA | 0.90 | 0.19435 |
| Rv1695 | hypothetical protein Rv1695 | 1.11 | 0.28026 |
| Rv1696 | recN | 0.65 | 0.00029 |
| Rv1697 | hypothetical protein Rv1697 | 2.28 | 0.02200 |
| Rv1698 | hypothetical protein Rv1698 | 0.95 | 0.40141 |
| Rv1699 | pyrG | 1.57 | 0.00434 |
| Rv1700 | hypothetical protein Rv1700 | 0.67 | 0.00114 |
| Rv1701 | hypothetical protein Rv1701 | 1.80 | 0.00100 |
| Rv1702c | hypothetical protein Rv1702c | 2.68 | 0.00226 |
| Rv1703c | hypothetical protein Rv1703c | 1.56 | 0.16497 |
| Rv1704c | cycA | 0.75 | 0.02002 |
| Rv1705c | PPE | 1.78 | 0.01907 |
| Rv1706c | PPE | 0.59 | 0.00274 |
| Rv1707 | hypothetical protein Rv1707 | 1.73 | 0.03950 |
| Rv1708 | hypothetical protein Rv1708 | 0.82 | 0.11542 |
| Rv1709 | hypothetical protein Rv1709 | 1.52 | 0.06575 |
| Rv1710 | hypothetical protein Rv1710 | 1.61 | 0.11954 |
| Rv1711 | hypothetical protein Rv1711 | 0.95 | 0.08753 |
| Rv1712 | cmk | 0.97 | 0.45943 |
| Rv1713 | hypothetical protein Rv1713 | 0.99 | 0.46990 |
| Rv1714 | hypothetical protein Rv1714 | 1.94 | 0.20206 |
| Rv1715 | fadB3 | 1.36 | 0.01365 |
| Rv1716 | hypothetical protein Rv1716 | 0.65 | 0.00049 |
| Rv1717 | hypothetical protein Rv1717 | 1.34 | 0.02055 |
| Rv1718 | hypothetical protein Rv1718 | 0.82 | 0.01172 |
| Rv1719 | hypothetical protein Rv1719 | 1.09 | 0.23389 |
| Rv1720c | hypothetical protein Rv1720c | 0.99 | 0.43602 |
| Rv1721c | hypothetical protein Rv1721c | 1.22 | 0.23990 |
| Rv1722 | hypothetical protein Rv1722 | 3.03 | 0.01070 |
| Rv1723 | hypothetical protein Rv1723 | 3.19 | 0.06086 |
| Rv1724c | hypothetical protein Rv1724c | 1.73 | 0.05508 |
| Rv1725c | hypothetical protein Rv1725c | 2.11 | 0.02778 |
| Rv1726 | hypothetical protein Rv1726 | 1.35 | 0.03454 |
| Rv1727 | hypothetical protein Rv1727 | 1.12 | 0.08564 |
| Rv1728c | hypothetical protein Rv1728c | 1.08 | 0.31481 |
| Rv1729c | hypothetical protein Rv1729c | 1.60 | 0.12238 |
| Rv1730c | hypothetical protein Rv1730c | 3.54 | 0.00274 |
| Rv1731 | gabD1 | 0.80 | 0.00159 |
| Rv1732c | hypothetical protein Rv1732c | 1.40 | 0.00000 |
| Rv1733c | hypothetical protein Rv1733c | 3.03 | 0.00426 |
| Rv1734c | hypothetical protein Rv1734c | 0.95 | 0.38479 |
| Rv1735c | hypothetical protein Rv1735c | 1.01 | 0.44572 |
| Rv1736c | narX | 1.23 | 0.11838 |
| Rv1737c | narK2 | 1.03 | 0.40641 |
| Rv1738 | hypothetical protein Rv1738 | 1.28 | 0.18728 |
| Rv1739c | hypothetical protein Rv1739c | 1.23 | 0.16163 |
| Rv1740 | hypothetical protein Rv1740 | 1.08 | 0.29251 |
| Rv1741 | hypothetical protein Rv1741 | 1.30 | 0.01158 |
| Rv1742 | hypothetical protein Rv1742 | 5.42 | 0.03402 |
| Rv1743 | pknE | 1.31 | 0.06227 |
| Rv1744c | hypothetical protein Rv1744c | 0.98 | 0.46906 |
| Rv1745c | hypothetical protein Rv1745c | 2.02 | 0.02135 |
| Rv1746 | pknF | 1.29 | 0.00312 |
| Rv1747 | hypothetical protein Rv1747 | 0.97 | 0.43449 |
| Rv1748 | hypothetical protein Rv1748 | 1.20 | 0.03823 |
| Rv1749c | hypothetical protein Rv1749c | 1.15 | 0.00036 |
| Rv1750c | fadD1 | 1.32 | 0.00909 |
| Rv1751 | hypothetical protein Rv1751 | 0.87 | 0.01325 |
| Rv1752 | hypothetical protein Rv1752 | 5.71 | 0.00428 |
| Rv1753c | PPE | 7.30 | 0.00541 |
| Rv1754c | hypothetical protein Rv1754c | 1.51 | 0.18247 |
| Rv1755c | plcD | 1.04 | 0.22674 |
| Rv1756c | hypothetical protein Rv1756c | 2.28 | 0.09355 |
| Rv1757c | hypothetical protein Rv1757c | 3.14 | 0.05771 |
| Rv1758 | hypothetical protein Rv1758 | 0.76 | 0.00431 |
| Rv1759c | PE_PGRS(wag22) | 3.03 | 0.00598 |
| Rv1760 | hypothetical protein Rv1760 | 1.16 | 0.14968 |
| Rv1761c | hypothetical protein Rv1761c | 1.55 | 0.03119 |
| Rv1762c | hypothetical protein Rv1762c | 1.12 | 0.32369 |
| Rv1763 | hypothetical protein Rv1763 | 2.03 | 0.10003 |
| Rv1764 | hypothetical protein Rv1764 | 1.42 | 0.23067 |
| Rv1765c | hypothetical protein Rv1765c | 2.01 | 0.28177 |
| Rv1766 | hypothetical protein Rv1766 | 0.75 | 0.00008 |
| Rv1767 | hypothetical protein Rv1767 | 4.45 | 0.08185 |
| Rv1768 | PE_PGRS | 1.56 | 0.10717 |
| Rv1770 | hypothetical protein Rv1770 | 0.86 | 0.03927 |
| Rv1771 | hypothetical protein Rv1771 | 1.45 | 0.21240 |
| Rv1772 | hypothetical protein Rv1772 | 0.50 | 0.00007 |
| Rv1774 | hypothetical protein Rv1774 | 1.84 | 0.03025 |
| Rv1775 | hypothetical protein Rv1775 | 1.68 | 0.16443 |
| Rv1776c | hypothetical protein Rv1776c | 0.75 | 0.04044 |
| Rv1777 | hypothetical protein Rv1777 | 2.86 | 0.03024 |
| Rv1778c | hypothetical protein Rv1778c | 1.49 | 0.00977 |
| Rv1779c | hypothetical protein Rv1779c | 1.68 | 0.03426 |
| Rv1780 | hypothetical protein Rv1780 | 1.51 | 0.05890 |
| Rv1781c | hypothetical protein Rv1781c | 0.93 | 0.26262 |
| Rv1782 | hypothetical protein Rv1782 | 2.32 | 0.00023 |
| Rv1783 | hypothetical protein Rv1783 | 1.68 | 0.00249 |
| Rv1784 | hypothetical protein Rv1784 | 1.16 | 0.20960 |
| Rv1785c | hypothetical protein Rv1785c | 1.06 | 0.37190 |
| Rv1786 | hypothetical protein Rv1786 | 1.18 | 0.18164 |
| Rv1787 | PPE | 5.90 | 0.10000 |
| Rv1788 | PE | 2.60 | 0.09177 |
| Rv1789 | PPE | 2.01 | 0.05061 |
| Rv1790 | PPE | 1.60 | 0.00689 |
| Rv1791 | PE | 1.64 | 0.04554 |
| Rv1792 | hypothetical protein Rv1792 | 0.50 | 0.00000 |
| Rv1793 | hypothetical protein Rv1793 | 0.62 | 0.01066 |
| Rv1794 | hypothetical protein Rv1794 | 0.76 | 0.01059 |
| Rv1795 | hypothetical protein Rv1795 | 1.09 | 0.26501 |
| Rv1796 | hypothetical protein Rv1796 | 1.51 | 0.01291 |
| Rv1797 | hypothetical protein Rv1797 | 0.86 | 0.14025 |
| Rv1798 | hypothetical protein Rv1798 | 0.96 | 0.41037 |
| Rv1799 | lppT | 1.33 | 0.04441 |
| Rv1800 | PPE | 3.02 | 0.04637 |
| Rv1801 | PPE | 2.79 | 0.00524 |
| Rv1802 | PPE | 1.94 | 0.06717 |
| Rv1803c | PE_PGRS | 1.40 | 0.08648 |
| Rv1804c | hypothetical protein Rv1804c | 1.26 | 0.10120 |
| Rv1805c | hypothetical protein Rv1805c | 2.22 | 0.04238 |
| Rv1806 | PE | 3.24 | 0.01184 |
| Rv1807 | PPE | 1.95 | 0.00514 |
| Rv1808 | PPE | 2.85 | 0.00340 |
| Rv1809 | PPE | 2.94 | 0.00516 |
| Rv1810 | hypothetical protein Rv1810 | 2.00 | 0.03038 |
| Rv1811 | mgtC | 3.02 | 0.02923 |
| Rv1812c | hypothetical protein Rv1812c | 0.97 | 0.43417 |
| Rv1814 | hypothetical protein Rv1814 | 2.49 | 0.09120 |
| Rv1815 | hypothetical protein Rv1815 | 3.13 | 0.07426 |
| Rv1816 | hypothetical protein Rv1816 | 1.02 | 0.45055 |
| Rv1818c | PE_PGRS | 0.74 | 0.01793 |
| Rv1819c | hypothetical protein Rv1819c | 1.78 | 0.03700 |
| Rv1820 | ilvG | 0.88 | 0.07671 |
| Rv1821 | secA2 | 1.14 | 0.43425 |
| Rv1822 | pgsA2 | 1.49 | 0.02925 |
| Rv1823 | hypothetical protein Rv1823 | 3.20 | 0.12384 |
| Rv1824 | hypothetical protein Rv1824 | 1.53 | 0.04701 |
| Rv1825 | hypothetical protein Rv1825 | 3.85 | 0.00250 |
| Rv1826 | gcvH | 0.79 | 0.00590 |
| Rv1827 | hypothetical protein Rv1827 | 0.79 | 0.06946 |
| Rv1828 | hypothetical protein Rv1828 | 0.86 | 0.07848 |
| Rv1829 | hypothetical protein Rv1829 | 1.30 | 0.15301 |
| Rv1830 | hypothetical protein Rv1830 | 1.03 | 0.45623 |
| Rv1831 | hypothetical protein Rv1831 | 1.71 | 0.00635 |
| Rv1832 | gcvB | 1.62 | 0.00709 |
| Rv1833c | hypothetical protein Rv1833c | 1.13 | 0.22309 |
| Rv1834 | hypothetical protein Rv1834 | 1.03 | 0.37847 |
| Rv1835c | hypothetical protein Rv1835c | 1.45 | 0.02225 |
| Rv1836c | hypothetical protein Rv1836c | 0.55 | 0.00000 |
| Rv1837c | glcB | 1.14 | 0.15116 |
| Rv1838c | hypothetical protein Rv1838c | 0.69 | 0.00033 |
| Rv1839c | hypothetical protein Rv1839c | 1.34 | 0.02154 |
| Rv1840c | PE_PGRS | 1.20 | 0.17434 |
| Rv1841c | hypothetical protein Rv1841c | 1.34 | 0.04746 |
| Rv1842c | hypothetical protein Rv1842c | 0.63 | 0.00016 |
| Rv1843c | guaB1 | 0.78 | 0.00005 |
| Rv1844c | gnd | 0.98 | 0.46663 |
| Rv1845c | hypothetical protein Rv1845c | 1.11 | 0.13530 |
| Rv1846c | hypothetical protein Rv1846c | 0.81 | 0.17315 |
| Rv1847 | hypothetical protein Rv1847 | 1.13 | 0.15175 |
| Rv1848 | ureA | 0.87 | 0.08625 |
| Rv1849 | ureB | 1.34 | 0.11255 |
| Rv1850 | ureC | 1.32 | 0.23137 |
| Rv1851 | ureF | 1.24 | 0.03955 |
| Rv1852 | ureG | 1.03 | 0.23952 |
| Rv1853 | ureD | 1.09 | 0.06865 |
| Rv1854c | ndh | 1.53 | 0.05141 |
| Rv1855c | hypothetical protein Rv1855c | 2.52 | 0.01409 |
| Rv1856c | hypothetical protein Rv1856c | 1.61 | 0.22693 |
| Rv1857 | modA | 1.04 | 0.38319 |
| Rv1858 | modB | 0.92 | 0.09089 |
| Rv1859 | modC | 1.51 | 0.02802 |
| Rv1860 | modD | 0.72 | 0.00852 |
| Rv1861 | hypothetical protein Rv1861 | 0.91 | 0.14070 |
| Rv1862 | adhA | 1.48 | 0.43981 |
| Rv1863c | hypothetical protein Rv1863c | 1.38 | 0.16088 |
| Rv1865c | hypothetical protein Rv1865c | 1.40 | 0.19903 |
| Rv1867 | hypothetical protein Rv1867 | 1.96 | 0.08716 |
| Rv1868 | hypothetical protein Rv1868 | 1.36 | 0.43810 |
| Rv1869c | hypothetical protein Rv1869c | 1.21 | 0.30395 |
| Rv1870c | hypothetical protein Rv1870c | 1.02 | 0.48150 |
| Rv1871c | hypothetical protein Rv1871c | 0.45 | 0.00018 |
| Rv1872c | lldD2 | 0.41 | 0.00000 |
| Rv1873 | hypothetical protein Rv1873 | 1.99 | 0.00876 |
| Rv1874 | hypothetical protein Rv1874 | 0.81 | 0.02937 |
| Rv1875 | hypothetical protein Rv1875 | 1.36 | 0.01282 |
| Rv1876 | bfrA | 2.97 | 0.05670 |
| Rv1877 | hypothetical protein Rv1877 | 1.87 | 0.05747 |
| Rv1878 | glnA3 | 0.91 | 0.22621 |
| Rv1879 | hypothetical protein Rv1879 | 0.82 | 0.02111 |
| Rv1880c | hypothetical protein Rv1880c | 0.73 | 0.00045 |
| Rv1881c | lppE | 1.69 | 0.08253 |
| Rv1882c | hypothetical protein Rv1882c | 1.33 | 0.20662 |
| Rv1883c | hypothetical protein Rv1883c | 1.08 | 0.33208 |
| Rv1884c | hypothetical protein Rv1884c | 0.52 | 0.00000 |
| Rv1885c | hypothetical protein Rv1885c | 1.11 | 0.24029 |
| Rv1886c | fbpB | 0.44 | 0.00005 |
| Rv1887 | hypothetical protein Rv1887 | 0.76 | 0.04701 |
| Rv1888c | hypothetical protein Rv1888c | 1.43 | 0.05233 |
| Rv1889c | hypothetical protein Rv1889c | 1.42 | 0.00457 |
| Rv1890c | hypothetical protein Rv1890c | 0.75 | 0.03183 |
| Rv1891 | hypothetical protein Rv1891 | 1.08 | 0.27583 |
| Rv1892 | hypothetical protein Rv1892 | 0.78 | 0.14377 |
| Rv1893 | hypothetical protein Rv1893 | 1.49 | 0.00035 |
| Rv1894c | hypothetical protein Rv1894c | 2.57 | 0.02893 |
| Rv1895 | hypothetical protein Rv1895 | 1.11 | 0.22002 |
| Rv1896c | hypothetical protein Rv1896c | 1.54 | 0.11087 |
| Rv1897c | hypothetical protein Rv1897c | 1.97 | 0.00413 |
| Rv1898 | hypothetical protein Rv1898 | 0.63 | 0.00226 |
| Rv1899c | lppD | 1.90 | 0.00929 |
| Rv1900c | lipJ | 1.42 | 0.16436 |
| Rv1901 | cinA | 2.77 | 0.01932 |
| Rv1902c | nanT | 1.14 | 0.22817 |
| Rv1903 | hypothetical protein Rv1903 | 1.99 | 0.01177 |
| Rv1904 | hypothetical protein Rv1904 | 0.72 | 0.03529 |
| Rv1905c | aao | 1.70 | 0.00147 |
| Rv1906c | hypothetical protein Rv1906c | 0.67 | 0.00734 |
| Rv1907c | hypothetical protein Rv1907c | 5.64 | 0.00357 |
| Rv1908c | katG | 3.17 | 0.02620 |
| Rv1909c | furA | 2.80 | 0.00351 |
| Rv1910c | hypothetical protein Rv1910c | 1.25 | 0.06789 |
| Rv1911c | lppC | 1.32 | 0.13293 |
| Rv1912c | fadB5 | 1.21 | 0.03685 |
| Rv1913 | hypothetical protein Rv1913 | 1.21 | 0.14228 |
| Rv1914c | hypothetical protein Rv1914c | 0.57 | 0.00008 |
| Rv1915 | aceAa | 0.85 | 0.03945 |
| Rv1916 | aceAb | 0.74 | 0.03592 |
| Rv1917c | PPE | 1.91 | 0.02588 |
| Rv1918c | PPE | 1.70 | 0.05546 |
| Rv1919c | hypothetical protein Rv1919c | 1.01 | 0.47858 |
| Rv1920 | hypothetical protein Rv1920 | 0.99 | 0.47475 |
| Rv1921c | lppF | 1.31 | 0.10504 |
| Rv1922 | hypothetical protein Rv1922 | 1.87 | 0.02998 |
| Rv1923 | lipD | 1.64 | 0.01848 |
| Rv1924c | hypothetical protein Rv1924c | 1.73 | 0.00044 |
| Rv1925 | fadD31 | 0.75 | 0.00053 |
| Rv1926c | hypothetical protein Rv1926c | 1.22 | 0.06210 |
| Rv1927 | hypothetical protein Rv1927 | 0.83 | 0.14015 |
| Rv1928c | hypothetical protein Rv1928c | 1.32 | 0.00527 |
| Rv1929c | hypothetical protein Rv1929c | 1.08 | 0.08757 |
| Rv1930c | hypothetical protein Rv1930c | 2.04 | 0.00077 |
| Rv1931c | hypothetical protein Rv1931c | 1.55 | 0.05625 |
| Rv1932 | tpx | 1.03 | 0.38933 |
| Rv1933c | fadE18 | 1.40 | 0.01154 |
| Rv1934c | fadE17 | 1.28 | 0.17671 |
| Rv1935c | echA13 | 1.41 | 0.03903 |
| Rv1936 | hypothetical protein Rv1936 | 3.23 | 0.00639 |
| Rv1937 | hypothetical protein Rv1937 | 1.05 | 0.28489 |
| Rv1938 | ephB | 1.52 | 0.01770 |
| Rv1939 | hypothetical protein Rv1939 | 1.64 | 0.01204 |
| Rv1940 | ribA | 0.86 | 0.21619 |
| Rv1941 | hypothetical protein Rv1941 | 1.72 | 0.01180 |
| Rv1942c | hypothetical protein Rv1942c | 3.05 | 0.02583 |
| Rv1943c | hypothetical protein Rv1943c | 1.18 | 0.26553 |
| Rv1944c | hypothetical protein Rv1944c | 1.17 | 0.08913 |
| Rv1945 | hypothetical protein Rv1945 | 5.83 | 0.02865 |
| Rv1946c | lppG | 0.96 | 0.37767 |
| Rv1947 | hypothetical protein Rv1947 | 1.80 | 0.07874 |
| Rv1948c | hypothetical protein Rv1948c | 0.95 | 0.35248 |
| Rv1949c | hypothetical protein Rv1949c | 1.99 | 0.02128 |
| Rv1950c | hypothetical protein Rv1950c | 1.19 | 0.18547 |
| Rv1951c | hypothetical protein Rv1951c | 1.88 | 0.02981 |
| Rv1952 | hypothetical protein Rv1952 | 1.30 | 0.16240 |
| Rv1953 | hypothetical protein Rv1953 | 2.41 | 0.02054 |
| Rv1954c | hypothetical protein Rv1954c | 2.93 | 0.00651 |
| Rv1955 | hypothetical protein Rv1955 | 2.92 | 0.02145 |
| Rv1956 | hypothetical protein Rv1956 | 2.26 | 0.00089 |
| Rv1957 | hypothetical protein Rv1957 | 1.22 | 0.16863 |
| Rv1958c | hypothetical protein Rv1958c | 0.68 | 0.00016 |
| Rv1959c | hypothetical protein Rv1959c | 1.08 | 0.34005 |
| Rv1960c | hypothetical protein Rv1960c | 1.12 | 0.18595 |
| Rv1961 | hypothetical protein Rv1961 | 1.49 | 0.10692 |
| Rv1962c | hypothetical protein Rv1962c | 0.70 | 0.00000 |
| Rv1963c | hypothetical protein Rv1963c | 0.95 | 0.39421 |
| Rv1964 | hypothetical protein Rv1964 | 0.81 | 0.00697 |
| Rv1965 | hypothetical protein Rv1965 | 1.33 | 0.15855 |
| Rv1966 | mce3 | 1.71 | 0.05221 |
| Rv1967 | hypothetical protein Rv1967 | 1.71 | 0.09890 |
| Rv1968 | hypothetical protein Rv1968 | 1.82 | 0.07973 |
| Rv1969 | hypothetical protein Rv1969 | 2.10 | 0.02570 |
| Rv1970 | lprM | 3.14 | 0.00621 |
| Rv1971 | hypothetical protein Rv1971 | 2.06 | 0.05069 |
| Rv1972 | hypothetical protein Rv1972 | 3.15 | 0.00108 |
| Rv1973 | hypothetical protein Rv1973 | 1.22 | 0.00674 |
| Rv1974 | hypothetical protein Rv1974 | 2.91 | 0.02246 |
| Rv1975 | hypothetical protein Rv1975 | 3.71 | 0.01994 |
| Rv1976c | hypothetical protein Rv1976c | 1.77 | 0.02485 |
| Rv1977 | hypothetical protein Rv1977 | 0.85 | 0.03215 |
| Rv1978 | hypothetical protein Rv1978 | 1.57 | 0.01344 |
| Rv1979c | hypothetical protein Rv1979c | 0.94 | 0.30727 |
| Rv1980c | mpt64 | 0.95 | 0.35869 |
| Rv1981c | nrdF | 3.47 | 0.00570 |
| Rv1982c | hypothetical protein Rv1982c | 1.41 | 0.03835 |
| Rv1983 | PE_PGRS | 3.62 | 0.02044 |
| Rv1984c | hypothetical protein Rv1984c | 3.67 | 0.00897 |
| Rv1985c | hypothetical protein Rv1985c | 1.59 | 0.01003 |
| Rv1986 | hypothetical protein Rv1986 | 3.82 | 0.00338 |
| Rv1987 | hypothetical protein Rv1987 | 2.22 | 0.02092 |
| Rv1988 | hypothetical protein Rv1988 | 2.86 | 0.00518 |
| Rv1989c | hypothetical protein Rv1989c | 1.50 | 0.00343 |
| Rv1990c | hypothetical protein Rv1990c | 2.72 | 0.00014 |
| Rv1991c | hypothetical protein Rv1991c | 1.30 | 0.05558 |
| Rv1992c | ctpG | 2.31 | 0.00000 |
| Rv1993c | hypothetical protein Rv1993c | 1.66 | 0.00047 |
| Rv1994c | hypothetical protein Rv1994c | 0.97 | 0.39773 |
| Rv1995 | hypothetical protein Rv1995 | 7.14 | 0.00904 |
| Rv1996 | hypothetical protein Rv1996 | 1.60 | 0.07980 |
| Rv1997 | ctpF | 13.16 | 0.00700 |
| Rv1998c | hypothetical protein Rv1998c | 1.31 | 0.02228 |
| Rv1999c | hypothetical protein Rv1999c | 4.42 | 0.01590 |
| Rv2000 | hypothetical protein Rv2000 | 1.10 | 0.07829 |
| Rv2001 | hypothetical protein Rv2001 | 3.48 | 0.01670 |
| Rv2002 | fabG3 | 1.04 | 0.36926 |
| Rv2003c | hypothetical protein Rv2003c | 1.73 | 0.00464 |
| Rv2004c | hypothetical protein Rv2004c | 1.41 | 0.02812 |
| Rv2005c | hypothetical protein Rv2005c | 2.22 | 0.00366 |
| Rv2006 | otsB | 1.73 | 0.03839 |
| Rv2007c | fdxA | 3.87 | 0.06076 |
| Rv2008c | hypothetical protein Rv2008c | 1.50 | 0.02231 |
| Rv2009 | hypothetical protein Rv2009 | 1.45 | 0.04126 |
| Rv2010 | hypothetical protein Rv2010 | 0.67 | 0.00049 |
| Rv2011c | hypothetical protein Rv2011c | 1.42 | 0.09925 |
| Rv2012 | hypothetical protein Rv2012 | 1.10 | 0.27344 |
| Rv2013 | hypothetical protein Rv2013 | 1.25 | 0.01183 |
| Rv2014 | hypothetical protein Rv2014 | 0.77 | 0.00096 |
| Rv2015c | hypothetical protein Rv2015c | 2.21 | 0.00001 |
| Rv2016 | hypothetical protein Rv2016 | 1.13 | 0.13223 |
| Rv2017 | hypothetical protein Rv2017 | 4.92 | 0.01282 |
| Rv2018 | hypothetical protein Rv2018 | 2.30 | 0.00000 |
| Rv2019 | hypothetical protein Rv2019 | 1.36 | 0.03636 |
| Rv2020c | hypothetical protein Rv2020c | 1.40 | 0.01749 |
| Rv2021c | hypothetical protein Rv2021c | 1.99 | 0.00452 |
| Rv2022c | hypothetical protein Rv2022c | 1.12 | 0.02442 |
| Rv2023c | hypothetical protein Rv2023c | 1.93 | 0.04334 |
| Rv2024c | hypothetical protein Rv2024c | 2.44 | 0.10083 |
| Rv2025c | hypothetical protein Rv2025c | 1.06 | 0.11663 |
| Rv2026c | hypothetical protein Rv2026c | 3.45 | 0.00170 |
| Rv2027c | hypothetical protein Rv2027c | 3.11 | 0.02450 |
| Rv2028c | hypothetical protein Rv2028c | 1.30 | 0.06884 |
| Rv2029c | pfkB | 1.54 | 0.00627 |
| Rv2030c | hypothetical protein Rv2030c | 7.79 | 0.00003 |
| Rv2031c | hspX | 24.96 | 0.00885 |
| Rv2032 | hypothetical protein Rv2032 | 5.46 | 0.01284 |
| Rv2033c | hypothetical protein Rv2033c | 1.81 | 0.00285 |
| Rv2034 | hypothetical protein Rv2034 | 1.73 | 0.00021 |
| Rv2035 | hypothetical protein Rv2035 | 1.17 | 0.11416 |
| Rv2036 | hypothetical protein Rv2036 | 0.88 | 0.17905 |
| Rv2037c | hypothetical protein Rv2037c | 1.02 | 0.43047 |
| Rv2038c | hypothetical protein Rv2038c | 0.64 | 0.00000 |
| Rv2039c | hypothetical protein Rv2039c | 1.39 | 0.00814 |
| Rv2040c | hypothetical protein Rv2040c | 1.44 | 0.02980 |
| Rv2041c | hypothetical protein Rv2041c | 1.38 | 0.02204 |
| Rv2042c | hypothetical protein Rv2042c | 1.33 | 0.05580 |
| Rv2043c | pncA | 1.54 | 0.00005 |
| Rv2044c | hypothetical protein Rv2044c | 0.99 | 0.47669 |
| Rv2045c | lipT | 1.16 | 0.24791 |
| Rv2046 | lppI | 0.91 | 0.24654 |
| Rv2047c | hypothetical protein Rv2047c | 1.26 | 0.00251 |
| Rv2048c | pks12 | 0.77 | 0.03189 |
| Rv2049c | hypothetical protein Rv2049c | 1.19 | 0.04441 |
| Rv2050 | hypothetical protein Rv2050 | 1.77 | 0.01998 |
| Rv2051c | hypothetical protein Rv2051c | 1.32 | 0.00002 |
| Rv2052c | hypothetical protein Rv2052c | 1.05 | 0.34550 |
| Rv2053c | hypothetical protein Rv2053c | 0.92 | 0.18854 |
| Rv2054 | hypothetical protein Rv2054 | 0.85 | 0.13340 |
| Rv2055c | rpsR2 | 2.58 | 0.00272 |
| Rv2056c | rpsN2 | 4.29 | 0.00000 |
| Rv2057c | rpmG | 5.92 | 0.00130 |
| Rv2058c | rpmB2 | 1.95 | 0.02776 |
| Rv2059 | hypothetical protein Rv2059 | 2.62 | 0.00032 |
| Rv2060 | hypothetical protein Rv2060 | 1.13 | 0.10411 |
| Rv2061c | hypothetical protein Rv2061c | 0.71 | 0.02102 |
| Rv2062c | cobN | 1.99 | 0.08279 |
| Rv2063c | hypothetical protein Rv2063c | 1.45 | 0.02477 |
| Rv2064 | cobG | 1.03 | 0.38870 |
| Rv2065 | cobH | 1.13 | 0.01515 |
| Rv2066 | cobI | 1.70 | 0.00109 |
| Rv2067c | hypothetical protein Rv2067c | 2.18 | 0.04175 |
| Rv2068c | blaC | 2.00 | 0.00127 |
| Rv2069 | sigC | 2.23 | 0.00382 |
| Rv2070c | cobK | 0.93 | 0.39080 |
| Rv2071c | cobM | 1.36 | 0.00580 |
| Rv2072c | cobL | 2.11 | 0.00442 |
| Rv2073c | hypothetical protein Rv2073c | 1.09 | 0.28462 |
| Rv2074 | hypothetical protein Rv2074 | 0.54 | 0.00001 |
| Rv2075c | hypothetical protein Rv2075c | 1.72 | 0.00273 |
| Rv2076c | hypothetical protein Rv2076c | 0.85 | 0.13633 |
| Rv2077c | hypothetical protein Rv2077c | 0.99 | 0.46615 |
| Rv2078 | hypothetical protein Rv2078 | 0.89 | 0.10992 |
| Rv2079 | hypothetical protein Rv2079 | 1.16 | 0.12628 |
| Rv2080 | lppJ | 3.28 | 0.03239 |
| Rv2081c | hypothetical protein Rv2081c | 1.36 | 0.02098 |
| Rv2082 | hypothetical protein Rv2082 | 0.80 | 0.17407 |
| Rv2083 | hypothetical protein Rv2083 | 1.10 | 0.17312 |
| Rv2084 | hypothetical protein Rv2084 | 0.89 | 0.04539 |
| Rv2085 | hypothetical protein Rv2085 | 1.04 | 0.37481 |
| Rv2086 | hypothetical protein Rv2086 | 1.18 | 0.21983 |
| Rv2087 | hypothetical protein Rv2087 | 1.51 | 0.02896 |
| Rv2088 | pknJ | 1.03 | 0.37533 |
| Rv2089c | pepE | 4.39 | 0.00486 |
| Rv2090 | hypothetical protein Rv2090 | 0.83 | 0.04229 |
| Rv2091c | hypothetical protein Rv2091c | 1.02 | 0.45201 |
| Rv2092c | helY | 0.95 | 0.33445 |
| Rv2093c | hypothetical protein Rv2093c | 1.82 | 0.04539 |
| Rv2094c | hypothetical protein Rv2094c | 0.63 | 0.00072 |
| Rv2095c | hypothetical protein Rv2095c | 2.10 | 0.05750 |
| Rv2096c | hypothetical protein Rv2096c | 0.62 | 0.00000 |
| Rv2097c | hypothetical protein Rv2097c | 1.73 | 0.07454 |
| Rv2098c | PE_PGRS | 1.16 | 0.25685 |
| Rv2099c | PE | 2.14 | 0.01290 |
| Rv2100 | hypothetical protein Rv2100 | 1.33 | 0.01697 |
| Rv2101 | helZ | 1.30 | 0.26045 |
| Rv2102 | hypothetical protein Rv2102 | 0.79 | 0.02918 |
| Rv2103c | hypothetical protein Rv2103c | 2.12 | 0.09583 |
| Rv2104c | hypothetical protein Rv2104c | 1.06 | 0.25429 |
| Rv2105 | hypothetical protein Rv2105 | 1.00 | 0.49971 |
| Rv2106 | hypothetical protein Rv2106 | 2.12 | 0.09068 |
| Rv2107 | PE | 1.29 | 0.10681 |
| Rv2108 | PPE | 1.06 | 0.37623 |
| Rv2109c | prcA | 1.61 | 0.20944 |
| Rv2110c | prcB | 0.59 | 0.00007 |
| Rv2111c | hypothetical protein Rv2111c | 1.21 | 0.28126 |
| Rv2112c | hypothetical protein Rv2112c | 0.70 | 0.00124 |
| Rv2113 | hypothetical protein Rv2113 | 2.06 | 0.00327 |
| Rv2114 | hypothetical protein Rv2114 | 2.06 | 0.00298 |
| Rv2115c | hypothetical protein Rv2115c | 1.46 | 0.00432 |
| Rv2116 | lppK | 1.50 | 0.00927 |
| Rv2117 | hypothetical protein Rv2117 | 1.11 | 0.03952 |
| Rv2118c | hypothetical protein Rv2118c | 0.85 | 0.13389 |
| Rv2119 | hypothetical protein Rv2119 | 1.25 | 0.01859 |
| Rv2120c | hypothetical protein Rv2120c | 0.95 | 0.34665 |
| Rv2121c | hisG | 1.73 | 0.07733 |
| Rv2122c | hisI | 3.39 | 0.00297 |
| Rv2123 | PPE | 10.48 | 0.02159 |
| Rv2124c | metH | 1.38 | 0.10374 |
| Rv2125 | hypothetical protein Rv2125 | 1.43 | 0.06030 |
| Rv2126c | PE_PGRS | 1.24 | 0.00004 |
| Rv2127 | ansP | 0.77 | 0.01430 |
| Rv2128 | hypothetical protein Rv2128 | 0.85 | 0.01835 |
| Rv2129c | hypothetical protein Rv2129c | 1.84 | 0.00636 |
| Rv2130c | cysS2 | 1.94 | 0.05262 |
| Rv2131c | cysQ | 2.43 | 0.00606 |
| Rv2132 | hypothetical protein Rv2132 | 1.27 | 0.00663 |
| Rv2133c | hypothetical protein Rv2133c | 1.65 | 0.02642 |
| Rv2134c | hypothetical protein Rv2134c | 1.14 | 0.15478 |
| Rv2135c | hypothetical protein Rv2135c | 1.43 | 0.06341 |
| Rv2136c | hypothetical protein Rv2136c | 2.06 | 0.01659 |
| Rv2137c | hypothetical protein Rv2137c | 0.77 | 0.16604 |
| Rv2138 | lppL | 1.08 | 0.40877 |
| Rv2139 | pyrD | 1.34 | 0.11151 |
| Rv2140c | hypothetical protein Rv2140c | 1.34 | 0.12137 |
| Rv2141c | dapE2 | 1.93 | 0.00837 |
| Rv2142c | hypothetical protein Rv2142c | 4.80 | 0.02637 |
| Rv2143 | hypothetical protein Rv2143 | 2.76 | 0.01108 |
| Rv2144c | hypothetical protein Rv2144c | 0.71 | 0.00108 |
| Rv2146c | hypothetical protein Rv2146c | 1.65 | 0.02026 |
| Rv2147c | hypothetical protein Rv2147c | 1.61 | 0.02241 |
| Rv2148c | hypothetical protein Rv2148c | 0.91 | 0.22570 |
| Rv2149c | yfiH | 3.71 | 0.05042 |
| Rv2150c | ftsZ | 1.87 | 0.16620 |
| Rv2151c | ftsQ | 1.74 | 0.16848 |
| Rv2152c | murC | 0.85 | 0.20588 |
| Rv2153c | murG | 1.00 | 0.49542 |
| Rv2154c | ftsW | 1.24 | 0.28155 |
| Rv2155c | murD | 2.12 | 0.05573 |
| Rv2156c | murX | 0.76 | 0.00194 |
| Rv2158c | murE | 1.45 | 0.05226 |
| Rv2159c | hypothetical protein Rv2159c | 1.12 | 0.30984 |
| Rv2160c | hypothetical protein Rv2160c | 0.84 | 0.30152 |
| Rv2161c | hypothetical protein Rv2161c | 1.07 | 0.35411 |
| Rv2162c | PE_PGRS | 1.55 | 0.03271 |
| Rv2163c | pbpB | 1.26 | 0.04704 |
| Rv2164c | hypothetical protein Rv2164c | 1.16 | 0.25628 |
| Rv2165c | hypothetical protein Rv2165c | 1.32 | 0.07172 |
| Rv2166c | hypothetical protein Rv2166c | 3.00 | 0.00290 |
| Rv2167c | hypothetical protein Rv2167c | 3.26 | 0.05412 |
| Rv2168c | hypothetical protein Rv2168c | 1.58 | 0.10968 |
| Rv2169c | hypothetical protein Rv2169c | 1.01 | 0.48696 |
| Rv2170 | hypothetical protein Rv2170 | 3.91 | 0.00324 |
| Rv2171 | lppM | 0.76 | 0.00129 |
| Rv2172c | hypothetical protein Rv2172c | 1.18 | 0.13342 |
| Rv2173 | idsA2 | 3.88 | 0.00346 |
| Rv2174 | hypothetical protein Rv2174 | 1.42 | 0.04928 |
| Rv2175c | hypothetical protein Rv2175c | 1.83 | 0.00962 |
| Rv2176 | pknL | 1.82 | 0.00256 |
| Rv2177c | hypothetical protein Rv2177c | 2.90 | 0.03053 |
| Rv2178c | aroG | 1.17 | 0.07775 |
| Rv2179c | hypothetical protein Rv2179c | 1.47 | 0.13985 |
| Rv2180c | hypothetical protein Rv2180c | 1.91 | 0.00023 |
| Rv2181 | hypothetical protein Rv2181 | 2.03 | 0.05073 |
| Rv2182c | hypothetical protein Rv2182c | 1.60 | 0.00007 |
| Rv2183c | hypothetical protein Rv2183c | 1.22 | 0.04687 |
| Rv2184c | hypothetical protein Rv2184c | 0.81 | 0.09024 |
| Rv2185c | hypothetical protein Rv2185c | 1.81 | 0.04347 |
| Rv2186c | hypothetical protein Rv2186c | 1.55 | 0.09628 |
| Rv2187 | fadD15 | 1.47 | 0.05234 |
| Rv2188c | hypothetical protein Rv2188c | 0.74 | 0.00198 |
| Rv2189c | hypothetical protein Rv2189c | 0.87 | 0.00011 |
| Rv2190c | hypothetical protein Rv2190c | 0.51 | 0.00013 |
| Rv2191 | hypothetical protein Rv2191 | 1.01 | 0.46720 |
| Rv2192c | trpD | 1.49 | 0.16577 |
| Rv2193 | ctaE | 0.77 | 0.00253 |
| Rv2194 | qcrC | 1.97 | 0.15118 |
| Rv2195 | qcrA | 0.51 | 0.00001 |
| Rv2196 | qcrB | 0.57 | 0.01581 |
| Rv2197c | hypothetical protein Rv2197c | 0.84 | 0.10335 |
| Rv2198c | mmpS3 | 0.78 | 0.09545 |
| Rv2199c | hypothetical protein Rv2199c | 1.09 | 0.41037 |
| Rv2200c | ctaC | 0.70 | 0.05223 |
| Rv2201 | asnB | 1.49 | 0.12230 |
| Rv2202c | cbhK | 0.84 | 0.00216 |
| Rv2203 | hypothetical protein Rv2203 | 1.55 | 0.18089 |
| Rv2204c | hypothetical protein Rv2204c | 0.58 | 0.00252 |
| Rv2205c | hypothetical protein Rv2205c | 1.28 | 0.12324 |
| Rv2206 | hypothetical protein Rv2206 | 0.73 | 0.01190 |
| Rv2207 | cobT | 0.98 | 0.47511 |
| Rv2208 | cobS | 2.51 | 0.11411 |
| Rv2209 | hypothetical protein Rv2209 | 1.00 | 0.49853 |
| Rv2210c | ilvE | 0.75 | 0.00497 |
| Rv2211c | gcvT | 0.69 | 0.00009 |
| Rv2212 | hypothetical protein Rv2212 | 1.88 | 0.01167 |
| Rv2213 | pepB | 1.67 | 0.01534 |
| Rv2214c | ephD | 0.65 | 0.00018 |
| Rv2215 | sucB | 2.09 | 0.01180 |
| Rv2216 | hypothetical protein Rv2216 | 0.92 | 0.33884 |
| Rv2217 | lipB | 1.59 | 0.06818 |
| Rv2218 | lipA | 1.08 | 0.28106 |
| Rv2219 | hypothetical protein Rv2219 | 0.87 | 0.00341 |
| Rv2220 | glnA1 | 0.61 | 0.00022 |
| Rv2221c | glnE | 1.23 | 0.08313 |
| Rv2222c | glnA2 | 0.72 | 0.01469 |
| Rv2223c | hypothetical protein Rv2223c | 2.38 | 0.01647 |
| Rv2224c | hypothetical protein Rv2224c | 0.64 | 0.00091 |
| Rv2225 | panB | 2.10 | 0.00003 |
| Rv2226 | hypothetical protein Rv2226 | 0.89 | 0.10499 |
| Rv2227 | hypothetical protein Rv2227 | 2.54 | 0.00390 |
| Rv2228c | hypothetical protein Rv2228c | 0.90 | 0.32675 |
| Rv2229c | hypothetical protein Rv2229c | 1.64 | 0.04398 |
| Rv2230c | hypothetical protein Rv2230c | 1.00 | 0.49350 |
| Rv2231c | cobC | 1.18 | 0.05200 |
| Rv2232 | hypothetical protein Rv2232 | 2.66 | 0.03427 |
| Rv2233 | hypothetical protein Rv2233 | 1.42 | 0.08668 |
| Rv2234 | ptpA | 1.23 | 0.17460 |
| Rv2235 | hypothetical protein Rv2235 | 2.90 | 0.03888 |
| Rv2236c | cobD | 0.89 | 0.04695 |
| Rv2237 | hypothetical protein Rv2237 | 0.85 | 0.03769 |
| Rv2238c | ahpE | 0.90 | 0.30819 |
| Rv2239c | hypothetical protein Rv2239c | 1.09 | 0.21265 |
| Rv2240c | hypothetical protein Rv2240c | 1.25 | 0.10737 |
| Rv2241 | aceE | 0.88 | 0.08244 |
| Rv2242 | hypothetical protein Rv2242 | 1.28 | 0.21860 |
| Rv2243 | fabD | 1.12 | 0.17272 |
| Rv2244 | acpM | 0.81 | 0.24418 |
| Rv2245 | kasA | 0.96 | 0.43917 |
| Rv2246 | kasB | 0.67 | 0.01915 |
| Rv2247 | accD6 | 1.98 | 0.06107 |
| Rv2248 | hypothetical protein Rv2248 | 2.84 | 0.17042 |
| Rv2249c | glpD1 | 1.64 | 0.00080 |
| Rv2250c | hypothetical protein Rv2250c | 0.82 | 0.08353 |
| Rv2251 | hypothetical protein Rv2251 | 0.81 | 0.00138 |
| Rv2252 | hypothetical protein Rv2252 | 1.00 | 0.49595 |
| Rv2253 | hypothetical protein Rv2253 | 1.21 | 0.22088 |
| Rv2254c | hypothetical protein Rv2254c | 0.90 | 0.15366 |
| Rv2255c | hypothetical protein Rv2255c | 1.00 | 0.49233 |
| Rv2256c | hypothetical protein Rv2256c | 1.34 | 0.11402 |
| Rv2257c | hypothetical protein Rv2257c | 1.97 | 0.05799 |
| Rv2258c | hypothetical protein Rv2258c | 1.23 | 0.26464 |
| Rv2259 | adhE2 | 0.99 | 0.46523 |
| Rv2260 | hypothetical protein Rv2260 | 0.68 | 0.00035 |
| Rv2261c | hypothetical protein Rv2261c | 0.95 | 0.31390 |
| Rv2262c | hypothetical protein Rv2262c | 0.90 | 0.17111 |
| Rv2263 | hypothetical protein Rv2263 | 0.93 | 0.32922 |
| Rv2264c | hypothetical protein Rv2264c | 1.28 | 0.07714 |
| Rv2265 | hypothetical protein Rv2265 | 1.56 | 0.05317 |
| Rv2266 | hypothetical protein Rv2266 | 1.42 | 0.18137 |
| Rv2267c | hypothetical protein Rv2267c | 0.94 | 0.33382 |
| Rv2268c | hypothetical protein Rv2268c | 1.34 | 0.05949 |
| Rv2269c | hypothetical protein Rv2269c | 3.13 | 0.00298 |
| Rv2270 | lppN | 0.61 | 0.00129 |
| Rv2271 | hypothetical protein Rv2271 | 0.85 | 0.09975 |
| Rv2272 | hypothetical protein Rv2272 | 1.32 | 0.19302 |
| Rv2273 | hypothetical protein Rv2273 | 5.05 | 0.00943 |
| Rv2274c | hypothetical protein Rv2274c | 1.51 | 0.13481 |
| Rv2275 | hypothetical protein Rv2275 | 4.27 | 0.01797 |
| Rv2276 | hypothetical protein Rv2276 | 0.78 | 0.00703 |
| Rv2277c | hypothetical protein Rv2277c | 1.39 | 0.00685 |
| Rv2278 | hypothetical protein Rv2278 | 1.06 | 0.41954 |
| Rv2279 | hypothetical protein Rv2279 | 2.43 | 0.01660 |
| Rv2280 | hypothetical protein Rv2280 | 0.52 | 0.00010 |
| Rv2281 | pitB | 1.30 | 0.00000 |
| Rv2282c | hypothetical protein Rv2282c | 0.98 | 0.41009 |
| Rv2283 | hypothetical protein Rv2283 | 1.13 | 0.00252 |
| Rv2284 | lipM | 1.91 | 0.05263 |
| Rv2285 | hypothetical protein Rv2285 | 1.36 | 0.00317 |
| Rv2286c | hypothetical protein Rv2286c | 0.66 | 0.00070 |
| Rv2287 | yjcE | 1.29 | 0.00542 |
| Rv2288 | hypothetical protein Rv2288 | 0.93 | 0.27581 |
| Rv2289 | cdh | 1.77 | 0.04718 |
| Rv2290 | lppO | 4.63 | 0.03736 |
| Rv2291 | sseB | 0.94 | 0.35218 |
| Rv2292c | hypothetical protein Rv2292c | 2.19 | 0.03059 |
| Rv2293c | hypothetical protein Rv2293c | 3.66 | 0.02575 |
| Rv2294 | hypothetical protein Rv2294 | 0.80 | 0.15275 |
| Rv2295 | hypothetical protein Rv2295 | 8.17 | 0.05320 |
| Rv2296 | hypothetical protein Rv2296 | 0.61 | 0.00008 |
| Rv2297 | hypothetical protein Rv2297 | 1.71 | 0.15563 |
| Rv2298 | hypothetical protein Rv2298 | 0.75 | 0.02024 |
| Rv2299c | htpG | 3.20 | 0.05972 |
| Rv2300c | hypothetical protein Rv2300c | 0.87 | 0.22693 |
| Rv2301 | hypothetical protein Rv2301 | 1.16 | 0.35753 |
| Rv2302 | hypothetical protein Rv2302 | 0.80 | 0.15004 |
| Rv2303c | hypothetical protein Rv2303c | 2.66 | 0.03136 |
| Rv2304c | hypothetical protein Rv2304c | 1.09 | 0.32832 |
| Rv2305 | hypothetical protein Rv2305 | 1.23 | 0.04246 |
| Rv2306c | hypothetical protein Rv2306c | 3.33 | 0.00169 |
| Rv2307c | hypothetical protein Rv2307c | 1.17 | 0.13350 |
| Rv2308 | hypothetical protein Rv2308 | 1.42 | 0.01864 |
| Rv2309c | hypothetical protein Rv2309c | 1.18 | 0.20067 |
| Rv2310 | hypothetical protein Rv2310 | 1.48 | 0.00001 |
| Rv2311 | hypothetical protein Rv2311 | 0.81 | 0.01061 |
| Rv2312 | hypothetical protein Rv2312 | 1.18 | 0.08105 |
| Rv2313c | hypothetical protein Rv2313c | 0.78 | 0.00283 |
| Rv2314c | hypothetical protein Rv2314c | 1.61 | 0.00972 |
| Rv2315c | hypothetical protein Rv2315c | 0.74 | 0.01168 |
| Rv2316 | uspA | 2.22 | 0.01590 |
| Rv2317 | uspE | 0.76 | 0.02787 |
| Rv2318 | uspC | 1.97 | 0.01498 |
| Rv2319c | hypothetical protein Rv2319c | 1.74 | 0.08537 |
| Rv2320c | rocE | 1.03 | 0.39593 |
| Rv2321c | rocD2 | 1.17 | 0.01397 |
| Rv2322c | rocD1 | 2.12 | 0.00958 |
| Rv2323c | hypothetical protein Rv2323c | 4.78 | 0.03378 |
| Rv2324 | hypothetical protein Rv2324 | 1.08 | 0.31364 |
| Rv2325c | hypothetical protein Rv2325c | 0.88 | 0.09908 |
| Rv2326c | hypothetical protein Rv2326c | 2.00 | 0.00796 |
| Rv2327 | hypothetical protein Rv2327 | 1.84 | 0.01394 |
| Rv2328 | PE | 2.70 | 0.04551 |
| Rv2329c | narK1 | 1.89 | 0.04617 |
| Rv2330c | lppP | 2.02 | 0.00449 |
| Rv2331 | hypothetical protein Rv2331 | 0.99 | 0.46469 |
| Rv2332 | mez | 1.04 | 0.29871 |
| Rv2333c | hypothetical protein Rv2333c | 1.40 | 0.12314 |
| Rv2334 | cysK | 1.12 | 0.38225 |
| Rv2335 | cysE | 1.32 | 0.17968 |
| Rv2336 | hypothetical protein Rv2336 | 1.31 | 0.12011 |
| Rv2337c | hypothetical protein Rv2337c | 2.01 | 0.02652 |
| Rv2338c | moeW | 2.00 | 0.04685 |
| Rv2339 | mmpL9 | 1.42 | 0.10401 |
| Rv2340c | PE | 0.93 | 0.22982 |
| Rv2341 | lppQ | 1.14 | 0.26260 |
| Rv2342 | hypothetical protein Rv2342 | 1.29 | 0.11778 |
| Rv2343c | dnaG | 2.74 | 0.00073 |
| Rv2344c | dgt | 1.49 | 0.21042 |
| Rv2345 | hypothetical protein Rv2345 | 2.16 | 0.08295 |
| Rv2346c | hypothetical protein Rv2346c | 0.51 | 0.00000 |
| Rv2347c | hypothetical protein Rv2347c | 0.49 | 0.00000 |
| Rv2348c | hypothetical protein Rv2348c | 0.51 | 0.00068 |
| Rv2349c | plcC | 2.17 | 0.08134 |
| Rv2350c | plcB | 0.85 | 0.08433 |
| Rv2351c | plcA | 3.54 | 0.02280 |
| Rv2352c | PPE | 0.76 | 0.01017 |
| Rv2353c | PPE | 1.75 | 0.08025 |
| Rv2354 | hypothetical protein Rv2354 | 1.57 | 0.10415 |
| Rv2355 | hypothetical protein Rv2355 | 2.48 | 0.09685 |
| Rv2356c | PPE | 0.84 | 0.12558 |
| Rv2357c | glyS | 0.90 | 0.18071 |
| Rv2358 | hypothetical protein Rv2358 | 1.84 | 0.01134 |
| Rv2359 | furB | 0.91 | 0.03397 |
| Rv2360c | hypothetical protein Rv2360c | 1.20 | 0.00271 |
| Rv2361c | hypothetical protein Rv2361c | 1.35 | 0.08310 |
| Rv2362c | hypothetical protein Rv2362c | 1.20 | 0.03519 |
| Rv2363 | amiA2 | 1.69 | 0.13858 |
| Rv2364c | bex | 0.99 | 0.41490 |
| Rv2365c | hypothetical protein Rv2365c | 1.10 | 0.01891 |
| Rv2366c | hypothetical protein Rv2366c | 0.79 | 0.04180 |
| Rv2367c | hypothetical protein Rv2367c | 1.19 | 0.15605 |
| Rv2368c | phoH | 1.32 | 0.01032 |
| Rv2369c | hypothetical protein Rv2369c | 1.37 | 0.00409 |
| Rv2370c | hypothetical protein Rv2370c | 3.95 | 0.00516 |
| Rv2371 | PE | 0.90 | 0.02037 |
| Rv2372c | hypothetical protein Rv2372c | 1.56 | 0.00838 |
| Rv2373c | dnaJ2 | 1.87 | 0.00143 |
| Rv2374c | hrcA | 1.23 | 0.08721 |
| Rv2375 | hypothetical protein Rv2375 | 1.85 | 0.01592 |
| Rv2376c | hypothetical protein Rv2376c | 0.52 | 0.00002 |
| Rv2377c | mbtH | 2.12 | 0.03560 |
| Rv2378c | mbtG | 2.06 | 0.00147 |
| Rv2379c | mbtF | 1.51 | 0.00028 |
| Rv2380c | mbtE | 3.99 | 0.00720 |
| Rv2381c | mbtD | 3.15 | 0.05137 |
| Rv2382c | mbtC | 4.27 | 0.01205 |
| Rv2383c | mbtB | 5.04 | 0.01804 |
| Rv2384 | mbtA | 1.68 | 0.02517 |
| Rv2385 | lipK | 3.53 | 0.04556 |
| Rv2386c | trpE2 | 1.18 | 0.10213 |
| Rv2387 | hypothetical protein Rv2387 | 1.26 | 0.04676 |
| Rv2388c | hemN | 0.63 | 0.00000 |
| Rv2389c | hypothetical protein Rv2389c | 1.26 | 0.19292 |
| Rv2390c | hypothetical protein Rv2390c | 0.86 | 0.00305 |
| Rv2391 | nirA | 0.34 | 0.00000 |
| Rv2392 | cysH | 0.46 | 0.00156 |
| Rv2393 | hypothetical protein Rv2393 | 0.49 | 0.00512 |
| Rv2394 | ggtB | 0.71 | 0.06190 |
| Rv2395 | hypothetical protein Rv2395 | 1.04 | 0.42273 |
| Rv2396 | PE_PGRS | 0.68 | 0.00058 |
| Rv2397c | cysA | 1.70 | 0.12695 |
| Rv2398c | cysW | 0.71 | 0.00210 |
| Rv2399c | cysT | 3.75 | 0.05881 |
| Rv2400c | subI | 1.23 | 0.14022 |
| Rv2401 | hypothetical protein Rv2401 | 1.33 | 0.03746 |
| Rv2402 | hypothetical protein Rv2402 | 1.04 | 0.38583 |
| Rv2403c | lppR | 1.12 | 0.00500 |
| Rv2404c | lepA | 1.58 | 0.02695 |
| Rv2405 | hypothetical protein Rv2405 | 1.10 | 0.23855 |
| Rv2406c | hypothetical protein Rv2406c | 0.98 | 0.44630 |
| Rv2407 | hypothetical protein Rv2407 | 3.69 | 0.10612 |
| Rv2408 | PE | 1.27 | 0.00000 |
| Rv2409c | hypothetical protein Rv2409c | 1.10 | 0.20552 |
| Rv2410c | hypothetical protein Rv2410c | 1.13 | 0.18418 |
| Rv2411c | hypothetical protein Rv2411c | 2.46 | 0.00174 |
| Rv2412 | rpsT | 1.01 | 0.47209 |
| Rv2413c | hypothetical protein Rv2413c | 1.02 | 0.29491 |
| Rv2414c | hypothetical protein Rv2414c | 0.95 | 0.32546 |
| Rv2415c | hypothetical protein Rv2415c | 2.46 | 0.03389 |
| Rv2416c | hypothetical protein Rv2416c | 1.49 | 0.00002 |
| Rv2417c | hypothetical protein Rv2417c | 0.88 | 0.12899 |
| Rv2418c | hypothetical protein Rv2418c | 1.01 | 0.47714 |
| Rv2419c | hypothetical protein Rv2419c | 0.98 | 0.41793 |
| Rv2420c | hypothetical protein Rv2420c | 0.67 | 0.00120 |
| Rv2421c | hypothetical protein Rv2421c | 4.66 | 0.01912 |
| Rv2422 | hypothetical protein Rv2422 | 0.90 | 0.06529 |
| Rv2423 | hypothetical protein Rv2423 | 0.74 | 0.01182 |
| Rv2424c | hypothetical protein Rv2424c | 1.95 | 0.02106 |
| Rv2425c | hypothetical protein Rv2425c | 0.91 | 0.20372 |
| Rv2426c | hypothetical protein Rv2426c | 0.82 | 0.00654 |
| Rv2427c | proA | 1.87 | 0.05161 |
| Rv2428 | ahpC | 1.11 | 0.31339 |
| Rv2429 | ahpD | 3.46 | 0.00157 |
| Rv2430c | PPE | 1.84 | 0.14131 |
| Rv2431c | PE | 0.80 | 0.00434 |
| Rv2432c | hypothetical protein Rv2432c | 0.62 | 0.00028 |
| Rv2433c | hypothetical protein Rv2433c | 1.03 | 0.37910 |
| Rv2434c | hypothetical protein Rv2434c | 1.61 | 0.05569 |
| Rv2435c | hypothetical protein Rv2435c | 1.03 | 0.40852 |
| Rv2436 | rbsK | 1.31 | 0.06404 |
| Rv2437 | hypothetical protein Rv2437 | 2.18 | 0.00133 |
| Rv2438c | hypothetical protein Rv2438c | 1.06 | 0.39475 |
| Rv2439c | proB | 1.48 | 0.22648 |
| Rv2440c | obg | 0.59 | 0.00008 |
| Rv2441c | rpmA | 0.83 | 0.16086 |
| Rv2442c | rplU | 0.63 | 0.00288 |
| Rv2443 | dctA | 1.02 | 0.44358 |
| Rv2444c | rne | 0.63 | 0.00264 |
| Rv2445c | ndkA | 0.83 | 0.25375 |
| Rv2446c | hypothetical protein Rv2446c | 0.68 | 0.00096 |
| Rv2447c | folC | 0.81 | 0.06509 |
| Rv2448c | valS | 1.09 | 0.15899 |
| Rv2449c | hypothetical protein Rv2449c | 0.97 | 0.30339 |
| Rv2450c | hypothetical protein Rv2450c | 1.24 | 0.19442 |
| Rv2451 | hypothetical protein Rv2451 | 2.98 | 0.01746 |
| Rv2452c | hypothetical protein Rv2452c | 2.34 | 0.02241 |
| Rv2453c | hypothetical protein Rv2453c | 1.35 | 0.01570 |
| Rv2454c | hypothetical protein Rv2454c | 1.68 | 0.00003 |
| Rv2455c | hypothetical protein Rv2455c | 2.38 | 0.00000 |
| Rv2456c | hypothetical protein Rv2456c | 0.78 | 0.06663 |
| Rv2457c | clpX | 0.78 | 0.08205 |
| Rv2458 | hypothetical protein Rv2458 | 2.45 | 0.03179 |
| Rv2459 | hypothetical protein Rv2459 | 0.92 | 0.29479 |
| Rv2460c | clpP2 | 1.10 | 0.36087 |
| Rv2461c | clpP | 1.08 | 0.07240 |
| Rv2462c | tig | 0.74 | 0.00913 |
| Rv2463 | lipP | 1.03 | 0.40134 |
| Rv2464c | hypothetical protein Rv2464c | 1.21 | 0.06317 |
| Rv2465c | rpi | 1.01 | 0.44818 |
| Rv2466c | hypothetical protein Rv2466c | 0.66 | 0.00213 |
| Rv2467 | pepD | 1.85 | 0.02781 |
| Rv2468c | hypothetical protein Rv2468c | 0.98 | 0.46265 |
| Rv2469c | hypothetical protein Rv2469c | 0.92 | 0.26635 |
| Rv2470 | glbO | 0.79 | 0.00010 |
| Rv2471 | hypothetical protein Rv2471 | 1.50 | 0.00057 |
| Rv2472 | hypothetical protein Rv2472 | 1.24 | 0.26904 |
| Rv2473 | hypothetical protein Rv2473 | 3.01 | 0.02369 |
| Rv2474c | hypothetical protein Rv2474c | 0.66 | 0.00026 |
| Rv2475c | hypothetical protein Rv2475c | 2.41 | 0.04336 |
| Rv2476c | hypothetical protein Rv2476c | 1.07 | 0.33697 |
| Rv2477c | hypothetical protein Rv2477c | 1.03 | 0.44447 |
| Rv2478c | hypothetical protein Rv2478c | 0.81 | 0.00146 |
| Rv2479c | hypothetical protein Rv2479c | 2.26 | 0.05002 |
| Rv2480c | hypothetical protein Rv2480c | 1.01 | 0.46523 |
| Rv2481c | hypothetical protein Rv2481c | 2.29 | 0.05958 |
| Rv2482c | plsB2 | 1.05 | 0.42399 |
| Rv2483c | hypothetical protein Rv2483c | 1.68 | 0.02160 |
| Rv2484c | hypothetical protein Rv2484c | 0.77 | 0.04231 |
| Rv2485c | lipQ | 0.97 | 0.45586 |
| Rv2486 | echA14 | 1.09 | 0.32693 |
| Rv2487c | PE_PGRS | 1.12 | 0.14068 |
| Rv2488c | hypothetical protein Rv2488c | 1.21 | 0.15385 |
| Rv2489c | hypothetical protein Rv2489c | 1.51 | 0.07173 |
| Rv2490c | PE_PGRS | 0.65 | 0.00508 |
| Rv2491 | hypothetical protein Rv2491 | 2.70 | 0.05925 |
| Rv2492 | hypothetical protein Rv2492 | 0.73 | 0.00001 |
| Rv2493 | hypothetical protein Rv2493 | 1.52 | 0.11098 |
| Rv2494 | hypothetical protein Rv2494 | 2.33 | 0.03838 |
| Rv2495c | pdhC | 1.01 | 0.48192 |
| Rv2496c | pdhB | 1.28 | 0.02409 |
| Rv2497c | pdhA | 2.04 | 0.00079 |
| Rv2498c | citE | 1.19 | 0.07162 |
| Rv2499c | hypothetical protein Rv2499c | 1.55 | 0.00371 |
| Rv2500c | fadE19 | 1.32 | 0.00009 |
| Rv2501c | accA1 | 0.94 | 0.36257 |
| Rv2502c | accD1 | 1.24 | 0.03456 |
| Rv2503c | scoB | 1.15 | 0.00225 |
| Rv2504c | scoA | 1.75 | 0.01215 |
| Rv2505c | fadD35 | 1.32 | 0.04651 |
| Rv2506 | hypothetical protein Rv2506 | 1.28 | 0.13916 |
| Rv2507 | hypothetical protein Rv2507 | 0.94 | 0.20747 |
| Rv2508c | hypothetical protein Rv2508c | 0.99 | 0.48723 |
| Rv2509 | hypothetical protein Rv2509 | 1.03 | 0.41555 |
| Rv2510c | hypothetical protein Rv2510c | 0.95 | 0.36428 |
| Rv2511 | hypothetical protein Rv2511 | 2.55 | 0.01770 |
| Rv2512c | hypothetical protein Rv2512c | 2.26 | 0.00053 |
| Rv2513 | hypothetical protein Rv2513 | 0.87 | 0.00268 |
| Rv2514c | hypothetical protein Rv2514c | 1.07 | 0.33966 |
| Rv2515c | hypothetical protein Rv2515c | 4.26 | 0.03532 |
| Rv2516c | hypothetical protein Rv2516c | 1.12 | 0.12309 |
| Rv2517c | hypothetical protein Rv2517c | 1.89 | 0.00006 |
| Rv2518c | lppS | 1.07 | 0.31373 |
| Rv2519 | PE | 2.54 | 0.06324 |
| Rv2520c | hypothetical protein Rv2520c | 1.70 | 0.01040 |
| Rv2521 | bcp | 0.88 | 0.11628 |
| Rv2522c | hypothetical protein Rv2522c | 0.63 | 0.00000 |
| Rv2523c | acpS | 1.44 | 0.00631 |
| Rv2524c | fas | 4.50 | 0.00737 |
| Rv2525c | hypothetical protein Rv2525c | 4.15 | 0.02559 |
| Rv2526 | hypothetical protein Rv2526 | 1.31 | 0.01486 |
| Rv2527 | hypothetical protein Rv2527 | 3.04 | 0.02814 |
| Rv2528c | mrr | 0.85 | 0.19141 |
| Rv2529 | hypothetical protein Rv2529 | 3.07 | 0.03856 |
| Rv2530c | hypothetical protein Rv2530c | 1.16 | 0.15802 |
| Rv2531c | adi | 5.34 | 0.00500 |
| Rv2532c | hypothetical protein Rv2532c | 0.90 | 0.14199 |
| Rv2533c | nusB | 4.01 | 0.22260 |
| Rv2534c | efp | 1.04 | 0.42008 |
| Rv2536 | hypothetical protein Rv2536 | 1.07 | 0.38716 |
| Rv2538c | aroB | 0.82 | 0.02476 |
| Rv2539c | aroK | 1.11 | 0.48058 |
| Rv2540c | aroF | 1.56 | 0.11445 |
| Rv2541 | hypothetical protein Rv2541 | 0.14 | 0.37531 |
| Rv2542 | hypothetical protein Rv2542 | 0.74 | 0.00185 |
| Rv2543 | lppA | 2.46 | 0.05829 |
| Rv2544 | lppB | 1.39 | 0.17298 |
| Rv2545 | hypothetical protein Rv2545 | 1.95 | 0.05670 |
| Rv2546 | hypothetical protein Rv2546 | 1.32 | 0.02845 |
| Rv2547 | hypothetical protein Rv2547 | 1.73 | 0.01097 |
| Rv2548 | hypothetical protein Rv2548 | 1.22 | 0.09934 |
| Rv2549c | hypothetical protein Rv2549c | 1.34 | 0.13585 |
| Rv2550c | hypothetical protein Rv2550c | 1.01 | 0.46317 |
| Rv2551c | hypothetical protein Rv2551c | 0.70 | 0.00005 |
| Rv2552c | aroE | 1.17 | 0.19946 |
| Rv2553c | hypothetical protein Rv2553c | 1.74 | 0.06274 |
| Rv2554c | hypothetical protein Rv2554c | 0.99 | 0.47562 |
| Rv2555c | alaS | 0.96 | 0.33294 |
| Rv2556c | hypothetical protein Rv2556c | 0.74 | 0.04046 |
| Rv2557 | hypothetical protein Rv2557 | 1.16 | 0.20327 |
| Rv2558 | hypothetical protein Rv2558 | 0.87 | 0.24229 |
| Rv2559c | hypothetical protein Rv2559c | 1.15 | 0.07933 |
| Rv2560 | hypothetical protein Rv2560 | 1.12 | 0.22725 |
| Rv2561 | hypothetical protein Rv2561 | 3.38 | 0.02275 |
| Rv2562 | hypothetical protein Rv2562 | 1.12 | 0.30580 |
| Rv2563 | hypothetical protein Rv2563 | 2.52 | 0.01629 |
| Rv2564 | glnQ | 0.89 | 0.11184 |
| Rv2565 | hypothetical protein Rv2565 | 1.14 | 0.10470 |
| Rv2566 | hypothetical protein Rv2566 | 2.39 | 0.00290 |
| Rv2567 | hypothetical protein Rv2567 | 2.29 | 0.05235 |
| Rv2568c | hypothetical protein Rv2568c | 1.86 | 0.04863 |
| Rv2569c | hypothetical protein Rv2569c | 2.02 | 0.04431 |
| Rv2570 | hypothetical protein Rv2570 | 1.30 | 0.05926 |
| Rv2571c | hypothetical protein Rv2571c | 1.83 | 0.00807 |
| Rv2572c | aspS | 0.91 | 0.37332 |
| Rv2573 | hypothetical protein Rv2573 | 1.19 | 0.03423 |
| Rv2574 | hypothetical protein Rv2574 | 1.00 | 0.49967 |
| Rv2575 | hypothetical protein Rv2575 | 0.97 | 0.37682 |
| Rv2576c | hypothetical protein Rv2576c | 0.88 | 0.23992 |
| Rv2577 | hypothetical protein Rv2577 | 3.41 | 0.00799 |
| Rv2578c | hypothetical protein Rv2578c | 1.66 | 0.06933 |
| Rv2579 | linB | 2.14 | 0.01950 |
| Rv2580c | hisS | 0.87 | 0.19858 |
| Rv2581c | hypothetical protein Rv2581c | 0.95 | 0.27868 |
| Rv2582 | ppiB | 0.96 | 0.42187 |
| Rv2583c | relA | 1.89 | 0.01739 |
| Rv2584c | apt | 0.58 | 0.00007 |
| Rv2585c | hypothetical protein Rv2585c | 2.28 | 0.07308 |
| Rv2586c | secF | 0.75 | 0.04265 |
| Rv2588c | hypothetical protein Rv2588c | 0.99 | 0.49151 |
| Rv2589 | gabT | 2.19 | 0.07390 |
| Rv2590 | fadD9 | 0.79 | 0.04982 |
| Rv2591 | PE_PGRS | 1.38 | 0.18442 |
| Rv2592c | ruvB | 0.68 | 0.00136 |
| Rv2593c | ruvA | 1.01 | 0.47946 |
| Rv2594c | ruvC | 1.08 | 0.11605 |
| Rv2595 | hypothetical protein Rv2595 | 1.01 | 0.44767 |
| Rv2596 | hypothetical protein Rv2596 | 2.14 | 0.04759 |
| Rv2597 | hypothetical protein Rv2597 | 0.85 | 0.00318 |
| Rv2598 | hypothetical protein Rv2598 | 1.15 | 0.05166 |
| Rv2599 | hypothetical protein Rv2599 | 1.71 | 0.01203 |
| Rv2600 | hypothetical protein Rv2600 | 1.10 | 0.33636 |
| Rv2601 | speE | 2.37 | 0.00130 |
| Rv2602 | hypothetical protein Rv2602 | 0.82 | 0.05292 |
| Rv2603c | hypothetical protein Rv2603c | 1.43 | 0.01820 |
| Rv2604c | hypothetical protein Rv2604c | 0.64 | 0.00000 |
| Rv2605c | tesB2 | 0.97 | 0.39860 |
| Rv2606c | hypothetical protein Rv2606c | 1.14 | 0.07462 |
| Rv2607 | pdxH | 1.11 | 0.13425 |
| Rv2608 | PPE | 1.50 | 0.08568 |
| Rv2609c | hypothetical protein Rv2609c | 1.22 | 0.00444 |
| Rv2610c | hypothetical protein Rv2610c | 1.07 | 0.37386 |
| Rv2611c | hypothetical protein Rv2611c | 0.92 | 0.19731 |
| Rv2612c | pgsA | 0.76 | 0.04050 |
| Rv2613c | hypothetical protein Rv2613c | 1.30 | 0.09674 |
| Rv2614c | thrS | 0.61 | 0.00174 |
| Rv2615c | PE_PGRS | 3.25 | 0.01623 |
| Rv2616 | hypothetical protein Rv2616 | 0.71 | 0.00089 |
| Rv2617c | hypothetical protein Rv2617c | 3.10 | 0.00068 |
| Rv2618 | hypothetical protein Rv2618 | 0.94 | 0.15990 |
| Rv2619c | hypothetical protein Rv2619c | 1.65 | 0.00041 |
| Rv2620c | hypothetical protein Rv2620c | 0.71 | 0.03213 |
| Rv2621c | hypothetical protein Rv2621c | 2.29 | 0.00882 |
| Rv2622 | hypothetical protein Rv2622 | 1.47 | 0.01244 |
| Rv2623 | hypothetical protein Rv2623 | 25.84 | 0.00024 |
| Rv2624c | hypothetical protein Rv2624c | 2.29 | 0.04052 |
| Rv2625c | hypothetical protein Rv2625c | 1.76 | 0.01152 |
| Rv2626c | hypothetical protein Rv2626c | 14.58 | 0.00012 |
| Rv2627c | hypothetical protein Rv2627c | 7.97 | 0.00799 |
| Rv2628 | hypothetical protein Rv2628 | 8.36 | 0.00407 |
| Rv2629 | hypothetical protein Rv2629 | 1.18 | 0.21118 |
| Rv2630 | hypothetical protein Rv2630 | 2.34 | 0.02136 |
| Rv2631 | hypothetical protein Rv2631 | 2.08 | 0.00128 |
| Rv2632c | hypothetical protein Rv2632c | 0.74 | 0.01290 |
| Rv2633c | hypothetical protein Rv2633c | 1.47 | 0.12951 |
| Rv2634c | PE_PGRS | 0.89 | 0.21565 |
| Rv2635 | hypothetical protein Rv2635 | 0.89 | 0.04697 |
| Rv2636 | hypothetical protein Rv2636 | 0.91 | 0.09629 |
| Rv2637 | dedA | 0.70 | 0.00018 |
| Rv2638 | hypothetical protein Rv2638 | 0.94 | 0.35381 |
| Rv2639c | hypothetical protein Rv2639c | 1.00 | 0.45208 |
| Rv2640c | hypothetical protein Rv2640c | 2.21 | 0.02820 |
| Rv2641 | hypothetical protein Rv2641 | 1.43 | 0.00027 |
| Rv2642 | hypothetical protein Rv2642 | 1.27 | 0.08078 |
| Rv2643 | arsC | 3.46 | 0.00125 |
| Rv2644c | hypothetical protein Rv2644c | 1.20 | 0.22872 |
| Rv2645 | hypothetical protein Rv2645 | 1.37 | 0.00000 |
| Rv2646 | hypothetical protein Rv2646 | 1.82 | 0.04165 |
| Rv2647 | hypothetical protein Rv2647 | 0.93 | 0.08640 |
| Rv2648 | hypothetical protein Rv2648 | 1.80 | 0.15314 |
| Rv2649 | hypothetical protein Rv2649 | 2.71 | 0.05367 |
| Rv2650c | hypothetical protein Rv2650c | 2.57 | 0.01063 |
| Rv2651c | hypothetical protein Rv2651c | 1.90 | 0.00020 |
| Rv2652c | hypothetical protein Rv2652c | 1.84 | 0.03276 |
| Rv2653c | hypothetical protein Rv2653c | 2.05 | 0.02308 |
| Rv2654c | hypothetical protein Rv2654c | 0.82 | 0.01710 |
| Rv2655c | hypothetical protein Rv2655c | 1.74 | 0.00618 |
| Rv2656c | hypothetical protein Rv2656c | 0.94 | 0.03462 |
| Rv2657c | hypothetical protein Rv2657c | 2.80 | 0.00818 |
| Rv2658c | hypothetical protein Rv2658c | 1.24 | 0.00571 |
| Rv2659c | hypothetical protein Rv2659c | 3.28 | 0.00297 |
| Rv2660c | hypothetical protein Rv2660c | 3.34 | 0.00552 |
| Rv2661c | hypothetical protein Rv2661c | 1.32 | 0.00658 |
| Rv2662 | hypothetical protein Rv2662 | 3.86 | 0.04735 |
| Rv2663 | hypothetical protein Rv2663 | 2.34 | 0.01569 |
| Rv2664 | hypothetical protein Rv2664 | 0.95 | 0.13426 |
| Rv2665 | hypothetical protein Rv2665 | 1.07 | 0.23519 |
| Rv2666 | hypothetical protein Rv2666 | 1.29 | 0.16892 |
| Rv2667 | clpX′ | 0.92 | 0.21150 |
| Rv2668 | hypothetical protein Rv2668 | 0.93 | 0.37019 |
| Rv2669 | hypothetical protein Rv2669 | 1.90 | 0.00877 |
| Rv2670c | hypothetical protein Rv2670c | 0.86 | 0.08957 |
| Rv2671 | ribD | 1.62 | 0.00021 |
| Rv2672 | hypothetical protein Rv2672 | 1.41 | 0.09911 |
| Rv2673 | hypothetical protein Rv2673 | 1.13 | 0.01703 |
| Rv2674 | hypothetical protein Rv2674 | 1.22 | 0.23894 |
| Rv2675c | hypothetical protein Rv2675c | 0.84 | 0.01330 |
| Rv2676c | hypothetical protein Rv2676c | 1.29 | 0.12083 |
| Rv2677c | hemY′ | 0.79 | 0.02954 |
| Rv2678c | hemE | 0.69 | 0.00322 |
| Rv2679 | echA15 | 0.88 | 0.08346 |
| Rv2680 | hypothetical protein Rv2680 | 0.70 | 0.00974 |
| Rv2681 | hypothetical protein Rv2681 | 1.03 | 0.46070 |
| Rv2682c | dxs | 0.79 | 0.07711 |
| Rv2683 | hypothetical protein Rv2683 | 0.90 | 0.17132 |
| Rv2684 | arsA | 0.91 | 0.35179 |
| Rv2685 | arsB | 1.15 | 0.17026 |
| Rv2686c | hypothetical protein Rv2686c | 0.91 | 0.24549 |
| Rv2687c | hypothetical protein Rv2687c | 0.77 | 0.02458 |
| Rv2688c | hypothetical protein Rv2688c | 1.73 | 0.03285 |
| Rv2689c | hypothetical protein Rv2689c | 1.62 | 0.07349 |
| Rv2690c | hypothetical protein Rv2690c | 1.24 | 0.09009 |
| Rv2691 | trkA | 1.12 | 0.12281 |
| Rv2692 | trkB | 1.40 | 0.02287 |
| Rv2693c | hypothetical protein Rv2693c | 0.87 | 0.11034 |
| Rv2694c | hypothetical protein Rv2694c | 1.48 | 0.02250 |
| Rv2695 | hypothetical protein Rv2695 | 1.31 | 0.00005 |
| Rv2696c | hypothetical protein Rv2696c | 0.83 | 0.06041 |
| Rv2697c | dut | 1.19 | 0.00390 |
| Rv2698 | hypothetical protein Rv2698 | 1.60 | 0.00039 |
| Rv2699c | hypothetical protein Rv2699c | 1.57 | 0.00158 |
| Rv2700 | hypothetical protein Rv2700 | 2.16 | 0.00881 |
| Rv2701c | suhB | 1.08 | 0.26093 |
| Rv2702 | ppgK | 1.23 | 0.10523 |
| Rv2703 | sigA | 0.91 | 0.18600 |
| Rv2704 | hypothetical protein Rv2704 | 0.95 | 0.25417 |
| Rv2705c | hypothetical protein Rv2705c | 1.97 | 0.04122 |
| Rv2706c | hypothetical protein Rv2706c | 2.23 | 0.00106 |
| Rv2707 | hypothetical protein Rv2707 | 3.10 | 0.00283 |
| Rv2708c | hypothetical protein Rv2708c | 0.78 | 0.01348 |
| Rv2709 | hypothetical protein Rv2709 | 1.40 | 0.05816 |
| Rv2710 | sigB | 4.70 | 0.00001 |
| Rv2711 | ideR | 1.78 | 0.00193 |
| Rv2712c | hypothetical protein Rv2712c | 2.39 | 0.00592 |
| Rv2713 | hypothetical protein Rv2713 | 2.67 | 0.00817 |
| Rv2714 | hypothetical protein Rv2714 | 1.68 | 0.00788 |
| Rv2715 | hypothetical protein Rv2715 | 1.53 | 0.05209 |
| Rv2716 | hypothetical protein Rv2716 | 2.81 | 0.04132 |
| Rv2717c | hypothetical protein Rv2717c | 1.66 | 0.05810 |
| Rv2718c | hypothetical protein Rv2718c | 1.25 | 0.14938 |
| Rv2719c | hypothetical protein Rv2719c | 1.66 | 0.06060 |
| Rv2720 | lexA | 1.21 | 0.21206 |
| Rv2721c | hypothetical protein Rv2721c | 1.37 | 0.16370 |
| Rv2722 | hypothetical protein Rv2722 | 1.56 | 0.03751 |
| Rv2723 | hypothetical protein Rv2723 | 2.04 | 0.03459 |
| Rv2724c | fadE20 | 0.82 | 0.02523 |
| Rv2725c | hflX | 2.05 | 0.00697 |
| Rv2726c | dapF | 0.69 | 0.00000 |
| Rv2727c | miaA | 2.98 | 0.04376 |
| Rv2728c | hypothetical protein Rv2728c | 0.86 | 0.11802 |
| Rv2729c | hypothetical protein Rv2729c | 2.45 | 0.01198 |
| Rv2730 | hypothetical protein Rv2730 | 1.26 | 0.17416 |
| Rv2731 | hypothetical protein Rv2731 | 0.80 | 0.00194 |
| Rv2732c | hypothetical protein Rv2732c | 0.77 | 0.05528 |
| Rv2733c | hypothetical protein Rv2733c | 1.20 | 0.19110 |
| Rv2734 | hypothetical protein Rv2734 | 1.09 | 0.04926 |
| Rv2735c | hypothetical protein Rv2735c | 3.82 | 0.05487 |
| Rv2736c | recX | 1.20 | 0.05063 |
| Rv2737c | recA | 1.22 | 0.08417 |
| Rv2738c | hypothetical protein Rv2738c | 1.11 | 0.23716 |
| Rv2739c | hypothetical protein Rv2739c | 1.52 | 0.02031 |
| Rv2740 | hypothetical protein Rv2740 | 0.96 | 0.41676 |
| Rv2741 | PE_PGRS | 1.46 | 0.00056 |
| Rv2742c | hypothetical protein Rv2742c | 1.56 | 0.00000 |
| Rv2743c | hypothetical protein Rv2743c | 1.43 | 0.00001 |
| Rv2744c | 35kd_ag | 3.33 | 0.00012 |
| Rv2745c | hypothetical protein Rv2745c | 1.12 | 0.22706 |
| Rv2746c | pgsA3 | 0.91 | 0.10923 |
| Rv2747 | hypothetical protein Rv2747 | 1.17 | 0.07512 |
| Rv2748c | ftsK | 0.93 | 0.31136 |
| Rv2749 | hypothetical protein Rv2749 | 1.84 | 0.01074 |
| Rv2750 | hypothetical protein Rv2750 | 1.54 | 0.04071 |
| Rv2751 | hypothetical protein Rv2751 | 1.04 | 0.35378 |
| Rv2752c | hypothetical protein Rv2752c | 0.90 | 0.15851 |
| Rv2753c | dapA | 1.36 | 0.10906 |
| Rv2754c | hypothetical protein Rv2754c | 1.26 | 0.11460 |
| Rv2755c | hsdS′ | 2.78 | 0.02927 |
| Rv2756c | hsdM | 1.28 | 0.10720 |
| Rv2757c | hypothetical protein Rv2757c | 3.10 | 0.00981 |
| Rv2758c | hypothetical protein Rv2758c | 1.00 | 0.49506 |
| Rv2759c | hypothetical protein Rv2759c | 1.87 | 0.01170 |
| Rv2760c | hypothetical protein Rv2760c | 1.10 | 0.22730 |
| Rv2761c | hypothetical protein Rv2761c | 1.34 | 0.07453 |
| Rv2762c | hypothetical protein Rv2762c | 0.93 | 0.13938 |
| Rv2763c | dfrA | 1.32 | 0.14250 |
| Rv2764c | thyA | 1.22 | 0.16879 |
| Rv2765 | hypothetical protein Rv2765 | 3.19 | 0.01922 |
| Rv2766c | fabG5 | 1.16 | 0.23026 |
| Rv2767c | hypothetical protein Rv2767c | 2.88 | 0.05569 |
| Rv2768c | PPE | 0.85 | 0.03167 |
| Rv2769c | PE | 3.06 | 0.04184 |
| Rv2770c | PPE | 1.80 | 0.12298 |
| Rv2771c | hypothetical protein Rv2771c | 0.92 | 0.19053 |
| Rv2772c | hypothetical protein Rv2772c | 0.69 | 0.00381 |
| Rv2773c | dapB | 0.97 | 0.41073 |
| Rv2774c | hypothetical protein Rv2774c | 0.73 | 0.00199 |
| Rv2775 | hypothetical protein Rv2775 | 1.02 | 0.45759 |
| Rv2776c | hypothetical protein Rv2776c | 0.85 | 0.09592 |
| Rv2777c | hypothetical protein Rv2777c | 2.47 | 0.02311 |
| Rv2778c | hypothetical protein Rv2778c | 0.62 | 0.00029 |
| Rv2779c | hypothetical protein Rv2779c | 1.56 | 0.11593 |
| Rv2780 | ald | 2.43 | 0.00147 |
| Rv2781c | hypothetical protein Rv2781c | 1.00 | 0.45641 |
| Rv2782c | pepR | 0.92 | 0.41579 |
| Rv2783c | gpsI | 0.58 | 0.00062 |
| Rv2784c | lppU | 1.21 | 0.20832 |
| Rv2785c | rpsO | 0.76 | 0.03661 |
| Rv2786c | ribF | 1.08 | 0.22086 |
| Rv2787 | hypothetical protein Rv2787 | 2.24 | 0.00825 |
| Rv2788 | sirR | 1.02 | 0.44998 |
| Rv2789c | fadE21 | 1.25 | 0.02695 |
| Rv2790c | ltp1 | 2.55 | 0.03996 |
| Rv2791c | hypothetical protein Rv2791c | 1.45 | 0.00001 |
| Rv2792c | hypothetical protein Rv2792c | 1.51 | 0.01051 |
| Rv2793c | truB | 1.36 | 0.14355 |
| Rv2794c | hypothetical protein Rv2794c | 0.98 | 0.46795 |
| Rv2795c | hypothetical protein Rv2795c | 1.63 | 0.06238 |
| Rv2796c | lppV | 1.04 | 0.40070 |
| Rv2797c | hypothetical protein Rv2797c | 0.82 | 0.04301 |
| Rv2798c | hypothetical protein Rv2798c | 0.62 | 0.00042 |
| Rv2799 | hypothetical protein Rv2799 | 1.52 | 0.03443 |
| Rv2800 | hypothetical protein Rv2800 | 1.08 | 0.16426 |
| Rv2801c | hypothetical protein Rv2801c | 1.31 | 0.04550 |
| Rv2802c | hypothetical protein Rv2802c | 0.97 | 0.41322 |
| Rv2803c | hypothetical protein Rv2803c | 0.89 | 0.18622 |
| Rv2804c | hypothetical protein Rv2804c | 1.51 | 0.02500 |
| Rv2805 | hypothetical protein Rv2805 | 1.49 | 0.03138 |
| Rv2806 | hypothetical protein Rv2806 | 1.18 | 0.20381 |
| Rv2807 | hypothetical protein Rv2807 | 1.99 | 0.04213 |
| Rv2808 | hypothetical protein Rv2808 | 1.70 | 0.14601 |
| Rv2809 | hypothetical protein Rv2809 | 1.02 | 0.34900 |
| Rv2810c | hypothetical protein Rv2810c | 2.26 | 0.01307 |
| Rv2811 | hypothetical protein Rv2811 | 1.23 | 0.00000 |
| Rv2812 | hypothetical protein Rv2812 | 0.85 | 0.07788 |
| Rv2813 | hypothetical protein Rv2813 | 2.56 | 0.01615 |
| Rv2814c | hypothetical protein Rv2814c | 1.85 | 0.12120 |
| Rv2815c | hypothetical protein Rv2815c | 2.18 | 0.05931 |
| Rv2816c | hypothetical protein Rv2816c | 0.62 | 0.00022 |
| Rv2817c | hypothetical protein Rv2817c | 1.54 | 0.06478 |
| Rv2818c | hypothetical protein Rv2818c | 0.80 | 0.08176 |
| Rv2819c | hypothetical protein Rv2819c | 1.56 | 0.05724 |
| Rv2820c | hypothetical protein Rv2820c | 2.35 | 0.06612 |
| Rv2821c | hypothetical protein Rv2821c | 0.98 | 0.44563 |
| Rv2822c | hypothetical protein Rv2822c | 0.99 | 0.47564 |
| Rv2823c | hypothetical protein Rv2823c | 1.18 | 0.26622 |
| Rv2824c | hypothetical protein Rv2824c | 0.73 | 0.01090 |
| Rv2825c | hypothetical protein Rv2825c | 1.27 | 0.14124 |
| Rv2826c | hypothetical protein Rv2826c | 1.43 | 0.13033 |
| Rv2827c | hypothetical protein Rv2827c | 1.88 | 0.04334 |
| Rv2828c | hypothetical protein Rv2828c | 1.16 | 0.23712 |
| Rv2829c | hypothetical protein Rv2829c | 3.05 | 0.05554 |
| Rv2830c | hypothetical protein Rv2830c | 0.71 | 0.00576 |
| Rv2831 | echA16 | 1.38 | 0.19407 |
| Rv2832c | ugpC | 0.94 | 0.15426 |
| Rv2833c | ugpB | 3.79 | 0.00664 |
| Rv2834c | ugpE | 2.15 | 0.03886 |
| Rv2835c | ugpA | 2.42 | 0.00299 |
| Rv2836c | dinF | 0.81 | 0.13231 |
| Rv2837c | hypothetical protein Rv2837c | 1.31 | 0.06924 |
| Rv2838c | rbfA | 1.53 | 0.11850 |
| Rv2839c | infB | 1.42 | 0.00025 |
| Rv2840c | hypothetical protein Rv2840c | 0.57 | 0.00452 |
| Rv2841c | nusA | 1.55 | 0.00994 |
| Rv2842c | hypothetical protein Rv2842c | 1.55 | 0.01074 |
| Rv2843 | hypothetical protein Rv2843 | 2.17 | 0.00430 |
| Rv2844 | hypothetical protein Rv2844 | 1.40 | 0.17267 |
| Rv2845c | proS | 1.89 | 0.00775 |
| Rv2846c | efpA | 0.54 | 0.00000 |
| Rv2847c | cysG2 | 1.14 | 0.19560 |
| Rv2848c | cobB | 1.08 | 0.28867 |
| Rv2849c | cobA | 0.85 | 0.15270 |
| Rv2850c | hypothetical protein Rv2850c | 0.58 | 0.00103 |
| Rv2851c | hypothetical protein Rv2851c | 1.55 | 0.06739 |
| Rv2852c | hypothetical protein Rv2852c | 0.57 | 0.00007 |
| Rv2853 | PE_PGRS | 0.98 | 0.45544 |
| Rv2854 | hypothetical protein Rv2854 | 1.71 | 0.02749 |
| Rv2855 | gorA | 1.33 | 0.19985 |
| Rv2856 | nicT | 1.51 | 0.00958 |
| Rv2857c | hypothetical protein Rv2857c | 1.30 | 0.09789 |
| Rv2858c | aldC | 0.94 | 0.38765 |
| Rv2859c | hypothetical protein Rv2859c | 0.96 | 0.40136 |
| Rv2860c | glnA4 | 1.31 | 0.16975 |
| Rv2861c | map | 1.18 | 0.19417 |
| Rv2862c | hypothetical protein Rv2862c | 0.61 | 0.00000 |
| Rv2863 | hypothetical protein Rv2863 | 2.02 | 0.00791 |
| Rv2864c | hypothetical protein Rv2864c | 0.76 | 0.00810 |
| Rv2865 | hypothetical protein Rv2865 | 1.59 | 0.00505 |
| Rv2866 | hypothetical protein Rv2866 | 0.61 | 0.00000 |
| Rv2867c | hypothetical protein Rv2867c | 1.11 | 0.18368 |
| Rv2868c | gcpE | 0.71 | 0.00226 |
| Rv2869c | hypothetical protein Rv2869c | 2.01 | 0.07171 |
| Rv2870c | hypothetical protein Rv2870c | 1.37 | 0.12454 |
| Rv2871 | hypothetical protein Rv2871 | 1.24 | 0.00339 |
| Rv2872 | hypothetical protein Rv2872 | 1.61 | 0.06172 |
| Rv2873 | mpt83 | 1.82 | 0.00939 |
| Rv2874 | hypothetical protein Rv2874 | 0.78 | 0.01449 |
| Rv2875 | mpt70 | 1.93 | 0.08411 |
| Rv2876 | hypothetical protein Rv2876 | 0.94 | 0.27901 |
| Rv2877c | hypothetical protein Rv2877c | 2.73 | 0.07693 |
| Rv2878c | mpt53 | 0.78 | 0.00399 |
| Rv2879c | hypothetical protein Rv2879c | 0.61 | 0.00019 |
| Rv2880c | hypothetical protein Rv2880c | 0.86 | 0.10168 |
| Rv2881c | cdsA | 0.90 | 0.15601 |
| Rv2882c | frr | 0.90 | 0.27609 |
| Rv2883c | pyrH | 1.19 | 0.25302 |
| Rv2884 | hypothetical protein Rv2884 | 1.10 | 0.18425 |
| Rv2885c | hypothetical protein Rv2885c | 0.90 | 0.02155 |
| Rv2886c | hypothetical protein Rv2886c | 1.19 | 0.28009 |
| Rv2887 | hypothetical protein Rv2887 | 2.82 | 0.00877 |
| Rv2888c | amiC | 0.95 | 0.30218 |
| Rv2889c | tsf | 1.07 | 0.32403 |
| Rv2890c | rpsB | 0.98 | 0.43303 |
| Rv2891 | hypothetical protein Rv2891 | 0.93 | 0.12329 |
| Rv2892c | PPE | 1.59 | 0.12745 |
| Rv2893 | hypothetical protein Rv2893 | 2.30 | 0.01855 |
| Rv2894c | xerC | 1.59 | 0.07031 |
| Rv2895c | viuB | 0.84 | 0.07260 |
| Rv2896c | hypothetical protein Rv2896c | 1.52 | 0.00767 |
| Rv2897c | hypothetical protein Rv2897c | 1.32 | 0.01111 |
| Rv2898c | hypothetical protein Rv2898c | 0.91 | 0.15658 |
| Rv2899c | fdhD | 1.69 | 0.01986 |
| Rv2900c | fdhF | 0.70 | 0.04645 |
| Rv2901c | hypothetical protein Rv2901c | 0.78 | 0.06429 |
| Rv2902c | rnhB | 0.73 | 0.03273 |
| Rv2903c | lepB | 1.02 | 0.45146 |
| Rv2904c | rplS | 0.79 | 0.05684 |
| Rv2905 | lppW | 1.28 | 0.19870 |
| Rv2906c | trmD | 0.66 | 0.00076 |
| Rv2907c | rimM | 2.58 | 0.02823 |
| Rv2908c | hypothetical protein Rv2908c | 0.88 | 0.25865 |
| Rv2909c | rpsP | 0.97 | 0.37422 |
| Rv2910c | hypothetical protein Rv2910c | 0.88 | 0.23535 |
| Rv2911 | dacB | 1.84 | 0.01735 |
| Rv2912c | hypothetical protein Rv2912c | 0.88 | 0.23840 |
| Rv2913c | hypothetical protein Rv2913c | 1.53 | 0.00577 |
| Rv2914c | pknI | 1.96 | 0.00829 |
| Rv2915c | hypothetical protein Rv2915c | 2.08 | 0.02931 |
| Rv2916c | ffh | 1.13 | 0.23057 |
| Rv2917 | hypothetical protein Rv2917 | 4.41 | 0.06488 |
| Rv2918c | glnD | 0.72 | 0.00736 |
| Rv2919c | glnB | 0.75 | 0.00082 |
| Rv2920c | amt | 0.69 | 0.01380 |
| Rv2921c | ftsY | 1.23 | 0.27260 |
| Rv2922c | smc | 0.73 | 0.13845 |
| Rv2923c | hypothetical protein Rv2923c | 1.25 | 0.28096 |
| Rv2924c | fpg | 0.92 | 0.22400 |
| Rv2925c | rnc | 1.19 | 0.23938 |
| Rv2926c | hypothetical protein Rv2926c | 1.44 | 0.14914 |
| Rv2927c | hypothetical protein Rv2927c | 1.02 | 0.45406 |
| Rv2928 | tesA | 1.88 | 0.14310 |
| Rv2929 | hypothetical protein Rv2929 | 1.71 | 0.13655 |
| Rv2930 | fadD26 | 1.95 | 0.00748 |
| Rv2931 | ppsA | 3.67 | 0.02082 |
| Rv2932 | ppsB | 2.33 | 0.00823 |
| Rv2933 | ppsC | 3.09 | 0.02651 |
| Rv2934 | ppsD | 0.99 | 0.46482 |
| Rv2935 | ppsE | 1.49 | 0.04733 |
| Rv2936 | drrA | 0.96 | 0.29835 |
| Rv2937 | drrB | 2.38 | 0.04492 |
| Rv2938 | drrC | 1.34 | 0.11321 |
| Rv2939 | papA5 | 0.95 | 0.29644 |
| Rv2940c | mas | 0.86 | 0.07588 |
| Rv2941 | fadD28 | 1.67 | 0.01862 |
| Rv2942 | mmpL7 | 1.28 | 0.19932 |
| Rv2943 | hypothetical protein Rv2943 | 0.90 | 0.12105 |
| Rv2944 | hypothetical protein Rv2944 | 2.09 | 0.02764 |
| Rv2945c | lppX | 1.76 | 0.05843 |
| Rv2946c | pks1 | 0.94 | 0.38253 |
| Rv2947c | pks15 | 1.25 | 0.16500 |
| Rv2948c | fadD22 | 0.98 | 0.45365 |
| Rv2949c | hypothetical protein Rv2949c | 1.02 | 0.46775 |
| Rv2950c | fadD29 | 1.21 | 0.24613 |
| Rv2951c | hypothetical protein Rv2951c | 1.01 | 0.49143 |
| Rv2952 | hypothetical protein Rv2952 | 0.79 | 0.01388 |
| Rv2953 | hypothetical protein Rv2953 | 0.98 | 0.43151 |
| Rv2954c | hypothetical protein Rv2954c | 0.77 | 0.08123 |
| Rv2955c | hypothetical protein Rv2955c | 0.92 | 0.21665 |
| Rv2956 | hypothetical protein Rv2956 | 0.69 | 0.00086 |
| Rv2957 | hypothetical protein Rv2957 | 1.18 | 0.08778 |
| Rv2958c | hypothetical protein Rv2958c | 4.23 | 0.03139 |
| Rv2959c | hypothetical protein Rv2959c | 2.39 | 0.06953 |
| Rv2960c | hypothetical protein Rv2960c | 1.51 | 0.14371 |
| Rv2961 | hypothetical protein Rv2961 | 1.83 | 0.03294 |
| Rv2962c | hypothetical protein Rv2962c | 4.25 | 0.02576 |
| Rv2963 | hypothetical protein Rv2963 | 3.50 | 0.00842 |
| Rv2964 | purU | 1.01 | 0.42498 |
| Rv2965c | kdtB | 2.68 | 0.08690 |
| Rv2966c | hypothetical protein Rv2966c | 0.93 | 0.36524 |
| Rv2967c | pca | 1.63 | 0.15393 |
| Rv2968c | hypothetical protein Rv2968c | 1.55 | 0.14923 |
| Rv2969c | hypothetical protein Rv2969c | 0.91 | 0.32370 |
| Rv2970c | lipN | 0.94 | 0.43046 |
| Rv2971 | hypothetical protein Rv2971 | 2.69 | 0.07165 |
| Rv2972c | hypothetical protein Rv2972c | 0.89 | 0.07015 |
| Rv2973c | recG | 2.20 | 0.10229 |
| Rv2974c | hypothetical protein Rv2974c | 0.85 | 0.07257 |
| Rv2975c | hypothetical protein Rv2975c | 1.28 | 0.20301 |
| Rv2976c | ung | 0.87 | 0.11130 |
| Rv2977c | thiL | 0.83 | 0.12441 |
| Rv2978c | hypothetical protein Rv2978c | 1.04 | 0.38454 |
| Rv2979c | hypothetical protein Rv2979c | 2.85 | 0.00135 |
| Rv2980 | hypothetical protein Rv2980 | 1.37 | 0.10657 |
| Rv2981c | ddlA | 1.09 | 0.13693 |
| Rv2982c | gpdA2 | 0.98 | 0.45988 |
| Rv2983 | hypothetical protein Rv2983 | 1.87 | 0.00436 |
| Rv2984 | ppk | 1.16 | 0.28773 |
| Rv2985 | mutT1 | 3.00 | 0.02448 |
| Rv2986c | hupB | 0.44 | 0.00000 |
| Rv2987c | leuD | 1.09 | 0.35772 |
| Rv2988c | leuC | 0.75 | 0.03309 |
| Rv2989 | hypothetical protein Rv2989 | 0.74 | 0.01923 |
| Rv2990c | hypothetical protein Rv2990c | 1.13 | 0.28565 |
| Rv2991 | hypothetical protein Rv2991 | 1.22 | 0.03607 |
| Rv2992c | gltS | 1.31 | 0.20774 |
| Rv2993c | hypothetical protein Rv2993c | 1.14 | 0.21598 |
| Rv2994 | hypothetical protein Rv2994 | 0.79 | 0.05130 |
| Rv2995c | leuB | 1.63 | 0.05390 |
| Rv2996c | serA | 0.97 | 0.40744 |
| Rv2997 | hypothetical protein Rv2997 | 1.78 | 0.01605 |
| Rv2998 | hypothetical protein Rv2998 | 0.74 | 0.03967 |
| Rv2999 | lppY | 2.78 | 0.01537 |
| Rv3000 | hypothetical protein Rv3000 | 1.09 | 0.27597 |
| Rv3001c | ilvC | 0.86 | 0.09312 |
| Rv3002c | ilvN | 0.53 | 0.00000 |
| Rv3003c | ilvB | 0.91 | 0.28990 |
| Rv3004 | hypothetical protein Rv3004 | 0.88 | 0.11012 |
| Rv3005c | hypothetical protein Rv3005c | 2.12 | 0.06439 |
| Rv3006 | lppZ | 0.72 | 0.00859 |
| Rv3007c | hypothetical protein Rv3007c | 1.21 | 0.01678 |
| Rv3008 | hypothetical protein Rv3008 | 1.48 | 0.09143 |
| Rv3009c | gatB | 3.75 | 0.01614 |
| Rv3010c | pfkA | 1.82 | 0.09395 |
| Rv3011c | gatA | 1.54 | 0.16158 |
| Rv3012c | gatC | 1.09 | 0.31638 |
| Rv3013 | hypothetical protein Rv3013 | 1.82 | 0.12998 |
| Rv3014c | ligA | 0.79 | 0.04331 |
| Rv3015c | hypothetical protein Rv3015c | 0.66 | 0.00027 |
| Rv3016 | lpqA | 1.86 | 0.01411 |
| Rv3017c | hypothetical protein Rv3017c | 1.23 | 0.25383 |
| Rv3018c | PPE | 1.88 | 0.03773 |
| Rv3019c | hypothetical protein Rv3019c | 1.56 | 0.05829 |
| Rv3020c | PE | 1.10 | 0.19035 |
| Rv3021c | PPE | 2.43 | 0.00313 |
| Rv3022c | PPE | 0.80 | 0.07372 |
| Rv3023c | hypothetical protein Rv3023c | 3.28 | 0.04450 |
| Rv3024c | hypothetical protein Rv3024c | 0.92 | 0.32289 |
| Rv3025c | hypothetical protein Rv3025c | 0.89 | 0.00403 |
| Rv3026c | hypothetical protein Rv3026c | 0.87 | 0.20926 |
| Rv3027c | hypothetical protein Rv3027c | 1.10 | 0.28932 |
| Rv3028c | fixB | 0.66 | 0.01470 |
| Rv3029c | fixA | 1.23 | 0.18982 |
| Rv3030 | hypothetical protein Rv3030 | 0.57 | 0.00001 |
| Rv3031 | hypothetical protein Rv3031 | 1.19 | 0.23868 |
| Rv3032 | hypothetical protein Rv3032 | 0.77 | 0.01324 |
| Rv3033 | hypothetical protein Rv3033 | 1.21 | 0.01755 |
| Rv3034c | hypothetical protein Rv3034c | 1.40 | 0.12390 |
| Rv3035 | hypothetical protein Rv3035 | 1.14 | 0.11637 |
| Rv3036c | hypothetical protein Rv3036c | 2.34 | 0.01882 |
| Rv3037c | hypothetical protein Rv3037c | 1.23 | 0.06564 |
| Rv3038c | hypothetical protein Rv3038c | 1.30 | 0.25881 |
| Rv3039c | echA17 | 1.33 | 0.01724 |
| Rv3040c | hypothetical protein Rv3040c | 0.78 | 0.04927 |
| Rv3041c | hypothetical protein Rv3041c | 1.87 | 0.04990 |
| Rv3042c | serB2 | 0.72 | 0.01309 |
| Rv3043c | ctaD | 1.28 | 0.00482 |
| Rv3044 | fecB | 0.69 | 0.00215 |
| Rv3045 | adhC | 1.01 | 0.46223 |
| Rv3046c | hypothetical protein Rv3046c | 0.82 | 0.08426 |
| Rv3047c | hypothetical protein Rv3047c | 2.04 | 0.00430 |
| Rv3048c | nrdG | 2.90 | 0.00654 |
| Rv3049c | hypothetical protein Rv3049c | 1.17 | 0.16164 |
| Rv3050c | hypothetical protein Rv3050c | 1.17 | 0.29772 |
| Rv3051c | nrdE | 0.79 | 0.10716 |
| Rv3052c | nrdI | 0.97 | 0.36992 |
| Rv3053c | nrdH | 1.17 | 0.12744 |
| Rv3054c | hypothetical protein Rv3054c | 1.23 | 0.02114 |
| Rv3055 | hypothetical protein Rv3055 | 2.13 | 0.02698 |
| Rv3056 | dinP | 2.31 | 0.04737 |
| Rv3057c | hypothetical protein Rv3057c | 0.89 | 0.12510 |
| Rv3058c | hypothetical protein Rv3058c | 1.64 | 0.03907 |
| Rv3059 | hypothetical protein Rv3059 | 1.90 | 0.11130 |
| Rv3060c | hypothetical protein Rv3060c | 0.64 | 0.00000 |
| Rv3061c | fadE22 | 1.12 | 0.26670 |
| Rv3062 | ligB | 1.15 | 0.25663 |
| Rv3063 | cstA | 0.66 | 0.00000 |
| Rv3064c | hypothetical protein Rv3064c | 1.11 | 0.33230 |
| Rv3065 | emrE | 0.73 | 0.00091 |
| Rv3066 | hypothetical protein Rv3066 | 2.08 | 0.08153 |
| Rv3067 | hypothetical protein Rv3067 | 1.83 | 0.12702 |
| Rv3068c | pgmA | 0.77 | 0.06616 |
| Rv3069 | hypothetical protein Rv3069 | 2.29 | 0.07630 |
| Rv3070 | hypothetical protein Rv3070 | 0.67 | 0.00004 |
| Rv3071 | hypothetical protein Rv3071 | 1.24 | 0.08371 |
| Rv3073c | hypothetical protein Rv3073c | 1.11 | 0.13040 |
| Rv3074 | hypothetical protein Rv3074 | 2.92 | 0.00570 |
| Rv3075c | hypothetical protein Rv3075c | 0.94 | 0.27367 |
| Rv3076 | hypothetical protein Rv3076 | 3.96 | 0.00300 |
| Rv3077 | atsF | 1.12 | 0.21159 |
| Rv3078 | hypothetical protein Rv3078 | 1.69 | 0.00198 |
| Rv3079c | hypothetical protein Rv3079c | 0.95 | 0.17004 |
| Rv3080c | pknK | 2.52 | 0.00015 |
| Rv3081 | hypothetical protein Rv3081 | 0.89 | 0.05345 |
| Rv3082c | virS | 3.39 | 0.00380 |
| Rv3083 | hypothetical protein Rv3083 | 1.04 | 0.18967 |
| Rv3084 | lipR | 1.31 | 0.00421 |
| Rv3085 | hypothetical protein Rv3085 | 1.49 | 0.03231 |
| Rv3086 | adhD | 0.89 | 0.13343 |
| Rv3087 | hypothetical protein Rv3087 | 1.07 | 0.23857 |
| Rv3088 | hypothetical protein Rv3088 | 2.14 | 0.00087 |
| Rv3089 | fadD13 | 1.24 | 0.04200 |
| Rv3090 | hypothetical protein Rv3090 | 1.93 | 0.02318 |
| Rv3091 | hypothetical protein Rv3091 | 1.86 | 0.02699 |
| Rv3092c | hypothetical protein Rv3092c | 1.19 | 0.13738 |
| Rv3093c | hypothetical protein Rv3093c | 0.99 | 0.41246 |
| Rv3094c | hypothetical protein Rv3094c | 1.07 | 0.33902 |
| Rv3095 | hypothetical protein Rv3095 | 1.02 | 0.40385 |
| Rv3096 | hypothetical protein Rv3096 | 1.22 | 0.22713 |
| Rv3097c | PE | 4.93 | 0.06727 |
| Rv3098c | hypothetical protein Rv3098c | 1.17 | 0.17963 |
| Rv3099c | hypothetical protein Rv3099c | 1.43 | 0.02539 |
| Rv3100c | smpB | 1.26 | 0.16847 |
| Rv3101c | ftsX | 2.24 | 0.03022 |
| Rv3102c | ftsE | 1.22 | 0.01337 |
| Rv3103c | hypothetical protein Rv3103c | 1.15 | 0.00028 |
| Rv3104c | hypothetical protein Rv3104c | 1.64 | 0.03335 |
| Rv3105c | prfB | 2.93 | 0.00136 |
| Rv3106 | fprA | 1.22 | 0.14203 |
| Rv3107c | hypothetical protein Rv3107c | 1.79 | 0.00389 |
| Rv3108 | hypothetical protein Rv3108 | 1.62 | 0.02983 |
| Rv3109 | moaA | 0.81 | 0.00032 |
| Rv3110 | moaB | 3.37 | 0.05826 |
| Rv3111 | moaC | 0.96 | 0.42261 |
| Rv3112 | moaD | 1.23 | 0.27165 |
| Rv3113 | hypothetical protein Rv3113 | 2.07 | 0.10127 |
| Rv3114 | hypothetical protein Rv3114 | 1.41 | 0.10431 |
| Rv3115 | hypothetical protein Rv3115 | 3.32 | 0.04543 |
| Rv3116 | moeB | 0.83 | 0.17244 |
| Rv3117 | cysA3 | 0.85 | 0.22256 |
| Rv3118 | sseC | 0.51 | 0.00011 |
| Rv3119 | moaE | 1.08 | 0.40147 |
| Rv3120 | hypothetical protein Rv3120 | 2.36 | 0.09363 |
| Rv3121 | hypothetical protein Rv3121 | 2.21 | 0.03634 |
| Rv3122 | hypothetical protein Rv3122 | 0.87 | 0.00090 |
| Rv3123 | hypothetical protein Rv3123 | 1.88 | 0.05108 |
| Rv3124 | hypothetical protein Rv3124 | 1.92 | 0.01620 |
| Rv3125c | PPE | 1.60 | 0.01793 |
| Rv3126c | hypothetical protein Rv3126c | 2.16 | 0.00012 |
| Rv3127 | hypothetical protein Rv3127 | 9.49 | 0.02020 |
| Rv3128c | hypothetical protein Rv3128c | 3.18 | 0.00420 |
| Rv3129 | hypothetical protein Rv3129 | 2.12 | 0.04078 |
| Rv3130c | hypothetical protein Rv3130c | 15.36 | 0.00037 |
| Rv3131 | hypothetical protein Rv3131 | 11.36 | 0.00080 |
| Rv3132c | hypothetical protein Rv3132c | 4.83 | 0.00134 |
| Rv3133c | hypothetical protein Rv3133c | 2.13 | 0.00748 |
| Rv3134c | hypothetical protein Rv3134c | 7.06 | 0.00688 |
| Rv3135 | PPE | 1.49 | 0.06951 |
| Rv3136 | PPE | 0.86 | 0.12183 |
| Rv3137 | hypothetical protein Rv3137 | 2.09 | 0.01570 |
| Rv3138 | pflA | 1.93 | 0.00899 |
| Rv3139 | fadE24 | 5.80 | 0.00421 |
| Rv3140 | fadE23 | 3.54 | 0.01414 |
| Rv3141 | fadB4 | 1.09 | 0.17330 |
| Rv3142c | hypothetical protein Rv3142c | 1.13 | 0.20360 |
| Rv3143 | hypothetical protein Rv3143 | 1.88 | 0.02475 |
| Rv3144c | PPE | 1.05 | 0.19138 |
| Rv3145 | nuoA | 1.13 | 0.31117 |
| Rv3146 | nuoB | 0.53 | 0.00273 |
| Rv3147 | nuoC | 1.62 | 0.13189 |
| Rv3148 | nuoD | 0.45 | 0.00003 |
| Rv3149 | nuoE | 1.43 | 0.11004 |
| Rv3150 | nuoF | 0.78 | 0.21139 |
| Rv3151 | nuoG | 1.09 | 0.32037 |
| Rv3152 | nuoH | 1.79 | 0.09022 |
| Rv3153 | nuoI | 0.88 | 0.28299 |
| Rv3154 | nuoJ | 0.62 | 0.00001 |
| Rv3155 | nuoK | 1.18 | 0.26070 |
| Rv3156 | nuoL | 0.66 | 0.00191 |
| Rv3157 | nuoM | 2.23 | 0.13427 |
| Rv3158 | nuoN | 0.51 | 0.00000 |
| Rv3159c | PPE | 0.91 | 0.15166 |
| Rv3160c | hypothetical protein Rv3160c | 1.12 | 0.30677 |
| Rv3161c | hypothetical protein Rv3161c | 1.95 | 0.05539 |
| Rv3162c | hypothetical protein Rv3162c | 0.77 | 0.06955 |
| Rv3163c | hypothetical protein Rv3163c | 2.42 | 0.03986 |
| Rv3164c | moxR3 | 1.30 | 0.01173 |
| Rv3165c | hypothetical protein Rv3165c | 3.24 | 0.06883 |
| Rv3166c | hypothetical protein Rv3166c | 1.08 | 0.00332 |
| Rv3167c | hypothetical protein Rv3167c | 1.08 | 0.34129 |
| Rv3168 | hypothetical protein Rv3168 | 0.71 | 0.00000 |
| Rv3169 | hypothetical protein Rv3169 | 1.02 | 0.37200 |
| Rv3170 | hypothetical protein Rv3170 | 1.79 | 0.04150 |
| Rv3171c | hpx | 0.85 | 0.03078 |
| Rv3172c | hypothetical protein Rv3172c | 1.67 | 0.07897 |
| Rv3173c | hypothetical protein Rv3173c | 1.85 | 0.04692 |
| Rv3174 | hypothetical protein Rv3174 | 1.18 | 0.23323 |
| Rv3175 | hypothetical protein Rv3175 | 1.70 | 0.00516 |
| Rv3176c | lipS | 1.51 | 0.11063 |
| Rv3177 | hypothetical protein Rv3177 | 1.73 | 0.04845 |
| Rv3178 | hypothetical protein Rv3178 | 3.45 | 0.02179 |
| Rv3179 | hypothetical protein Rv3179 | 1.25 | 0.01069 |
| Rv3180c | hypothetical protein Rv3180c | 3.29 | 0.01208 |
| Rv3181c | hypothetical protein Rv3181c | 1.53 | 0.00068 |
| Rv3182 | hypothetical protein Rv3182 | 2.74 | 0.06192 |
| Rv3183 | hypothetical protein Rv3183 | 1.62 | 0.04730 |
| Rv3184 | hypothetical protein Rv3184 | 1.36 | 0.24449 |
| Rv3185 | hypothetical protein Rv3185 | 2.78 | 0.05989 |
| Rv3186 | hypothetical protein Rv3186 | 1.38 | 0.20911 |
| Rv3187 | hypothetical protein Rv3187 | 2.64 | 0.05540 |
| Rv3188 | hypothetical protein Rv3188 | 1.49 | 0.00984 |
| Rv3189 | hypothetical protein Rv3189 | 2.18 | 0.00400 |
| Rv3190c | hypothetical protein Rv3190c | 1.13 | 0.30350 |
| Rv3191c | hypothetical protein Rv3191c | 4.75 | 0.02392 |
| Rv3192 | hypothetical protein Rv3192 | 1.47 | 0.01042 |
| Rv3193c | hypothetical protein Rv3193c | 1.40 | 0.05735 |
| Rv3194c | hypothetical protein Rv3194c | 1.58 | 0.09728 |
| Rv3195 | hypothetical protein Rv3195 | 2.94 | 0.03687 |
| Rv3196 | hypothetical protein Rv3196 | 0.79 | 0.05091 |
| Rv3197 | hypothetical protein Rv3197 | 1.51 | 0.05102 |
| Rv3198c | uvrD2 | 0.81 | 0.07929 |
| Rv3199c | hypothetical protein Rv3199c | 1.72 | 0.04179 |
| Rv3200c | hypothetical protein Rv3200c | 0.81 | 0.10355 |
| Rv3201c | hypothetical protein Rv3201c | 1.05 | 0.28054 |
| Rv3202c | hypothetical protein Rv3202c | 0.67 | 0.01692 |
| Rv3203 | lipV | 1.29 | 0.01124 |
| Rv3204 | hypothetical protein Rv3204 | 1.64 | 0.09539 |
| Rv3205c | hypothetical protein Rv3205c | 0.99 | 0.46895 |
| Rv3206c | moeZ | 0.98 | 0.44412 |
| Rv3207c | hypothetical protein Rv3207c | 4.00 | 0.08772 |
| Rv3208 | hypothetical protein Rv3208 | 0.95 | 0.36123 |
| Rv3209 | hypothetical protein Rv3209 | 0.99 | 0.47169 |
| Rv3210c | hypothetical protein Rv3210c | 1.04 | 0.43912 |
| Rv3211 | rhlE | 0.70 | 0.04024 |
| Rv3212 | hypothetical protein Rv3212 | 0.68 | 0.03008 |
| Rv3213c | hypothetical protein Rv3213c | 1.49 | 0.19765 |
| Rv3214 | entD | 0.89 | 0.20269 |
| Rv3215 | entC | 1.57 | 0.13877 |
| Rv3216 | hypothetical protein Rv3216 | 0.74 | 0.00472 |
| Rv3217c | hypothetical protein Rv3217c | 1.07 | 0.28830 |
| Rv3218 | hypothetical protein Rv3218 | 1.52 | 0.01068 |
| Rv3219 | whiB1 | 0.91 | 0.02113 |
| Rv3220c | hypothetical protein Rv3220c | 0.65 | 0.00339 |
| Rv3221c | hypothetical protein Rv3221c | 0.82 | 0.01296 |
| Rv3222c | hypothetical protein Rv3222c | 1.54 | 0.00029 |
| Rv3223c | sigH | 3.41 | 0.00429 |
| Rv3224 | hypothetical protein Rv3224 | 1.15 | 0.21521 |
| Rv3225c | hypothetical protein Rv3225c | 1.64 | 0.04300 |
| Rv3226c | hypothetical protein Rv3226c | 1.43 | 0.00087 |
| Rv3227 | aroA | 2.78 | 0.02827 |
| Rv3228 | hypothetical protein Rv3228 | 1.79 | 0.06263 |
| Rv3229c | desA3 | 1.28 | 0.18746 |
| Rv3230c | hypothetical protein Rv3230c | 1.90 | 0.04881 |
| Rv3231c | hypothetical protein Rv3231c | 3.60 | 0.01542 |
| Rv3232c | pvdS | 0.71 | 0.01664 |
| Rv3233c | hypothetical protein Rv3233c | 1.27 | 0.08863 |
| Rv3234c | hypothetical protein Rv3234c | 1.13 | 0.37662 |
| Rv3235 | hypothetical protein Rv3235 | 4.08 | 0.02502 |
| Rv3236c | kefB | 1.04 | 0.39997 |
| Rv3237c | hypothetical protein Rv3237c | 0.90 | 0.11136 |
| Rv3238c | hypothetical protein Rv3238c | 0.61 | 0.00087 |
| Rv3239c | hypothetical protein Rv3239c | 1.21 | 0.13929 |
| Rv3240c | secA | 0.83 | 0.09786 |
| Rv3241c | hypothetical protein Rv3241c | 1.79 | 0.01956 |
| Rv3242c | hypothetical protein Rv3242c | 1.20 | 0.19643 |
| Rv3243c | hypothetical protein Rv3243c | 1.26 | 0.05160 |
| Rv3244c | lpqB | 1.05 | 0.39932 |
| Rv3245c | mtrB | 3.56 | 0.03339 |
| Rv3246c | mtrA | 1.11 | 0.30173 |
| Rv3247c | tmk | 1.25 | 0.04148 |
| Rv3248c | sahH | 1.07 | 0.37392 |
| Rv3249c | hypothetical protein Rv3249c | 1.55 | 0.10356 |
| Rv3250c | rubB | 0.88 | 0.00721 |
| Rv3251c | rubA | 1.48 | 0.07865 |
| Rv3252c | hypothetical protein Rv3252c | 2.78 | 0.06568 |
| Rv3253c | hypothetical protein Rv3253c | 0.95 | 0.38700 |
| Rv3254 | hypothetical protein Rv3254 | 0.88 | 0.25035 |
| Rv3255c | manA | 1.11 | 0.31437 |
| Rv3256c | hypothetical protein Rv3256c | 0.76 | 0.07567 |
| Rv3257c | pmmA | 0.86 | 0.00053 |
| Rv3258c | hypothetical protein Rv3258c | 0.69 | 0.00884 |
| Rv3259 | hypothetical protein Rv3259 | 1.04 | 0.42999 |
| Rv3260c | whiB2 | 0.62 | 0.00049 |
| Rv3261 | hypothetical protein Rv3261 | 1.95 | 0.14297 |
| Rv3262 | hypothetical protein Rv3262 | 0.83 | 0.03386 |
| Rv3263 | hypothetical protein Rv3263 | 1.51 | 0.07391 |
| Rv3264c | rmlA2 | 0.64 | 0.01531 |
| Rv3265c | wbbL | 2.12 | 0.02831 |
| Rv3266c | rmlD | 1.73 | 0.03624 |
| Rv3267 | hypothetical protein Rv3267 | 1.93 | 0.07271 |
| Rv3268 | hypothetical protein Rv3268 | 2.06 | 0.01737 |
| Rv3269 | hypothetical protein Rv3269 | 1.27 | 0.11489 |
| Rv3270 | ctpC | 2.35 | 0.00081 |
| Rv3271c | hypothetical protein Rv3271c | 1.47 | 0.04208 |
| Rv3272 | hypothetical protein Rv3272 | 1.72 | 0.08938 |
| Rv3273 | hypothetical protein Rv3273 | 1.18 | 0.29007 |
| Rv3274c | fadE25 | 1.16 | 0.09228 |
| Rv3275c | purE | 1.40 | 0.04458 |
| Rv3276c | purK | 2.11 | 0.04597 |
| Rv3277 | hypothetical protein Rv3277 | 1.38 | 0.02454 |
| Rv3278c | hypothetical protein Rv3278c | 0.94 | 0.40517 |
| Rv3279c | birA | 3.07 | 0.04098 |
| Rv3280 | accD5 | 1.95 | 0.07278 |
| Rv3281 | hypothetical protein Rv3281 | 0.57 | 0.00192 |
| Rv3282 | hypothetical protein Rv3282 | 2.23 | 0.02989 |
| Rv3283 | sseA | 0.92 | 0.23093 |
| Rv3284 | hypothetical protein Rv3284 | 1.15 | 0.18177 |
| Rv3285 | accA3 | 1.25 | 0.14719 |
| Rv3286c | sigF | 1.02 | 0.45768 |
| Rv3287c | rsbW | 4.58 | 0.00051 |
| Rv3288c | hypothetical protein Rv3288c | 1.83 | 0.01907 |
| Rv3289c | hypothetical protein Rv3289c | 1.94 | 0.03139 |
| Rv3290c | lat | 5.59 | 0.00864 |
| Rv3291c | hypothetical protein Rv3291c | 1.70 | 0.00502 |
| Rv3292 | hypothetical protein Rv3292 | 1.57 | 0.00251 |
| Rv3293 | aldB | 1.17 | 0.00186 |
| Rv3294 | hypothetical protein Rv3294 | 0.70 | 0.00000 |
| Rv3295 | hypothetical protein Rv3295 | 0.97 | 0.45574 |
| Rv3296 | lhr | 1.74 | 0.00218 |
| Rv3297 | nei | 2.54 | 0.01338 |
| Rv3298c | lpqC | 1.04 | 0.37722 |
| Rv3299c | atsB | 1.06 | 0.33981 |
| Rv3300c | hypothetical protein Rv3300c | 1.52 | 0.16456 |
| Rv3301c | phoY1 | 1.82 | 0.14106 |
| Rv3302c | glpD2 | 0.82 | 0.16515 |
| Rv3303c | lpdA | 2.34 | 0.08355 |
| Rv3304 | hypothetical protein Rv3304 | 0.71 | 0.00048 |
| Rv3305c | amiA | 3.60 | 0.08888 |
| Rv3306c | amiB | 0.53 | 0.00000 |
| Rv3307 | deoD | 1.62 | 0.20954 |
| Rv3308 | pmmB | 0.45 | 0.00000 |
| Rv3309c | upp | 0.90 | 0.31359 |
| Rv3310 | hypothetical protein Rv3310 | 1.56 | 0.15190 |
| Rv3311 | hypothetical protein Rv3311 | 1.12 | 0.33212 |
| Rv3312c | hypothetical protein Rv3312c | 0.77 | 0.06475 |
| Rv3313c | add | 1.70 | 0.00390 |
| Rv3314c | deoA | 1.73 | 0.03139 |
| Rv3315c | cdd | 0.82 | 0.08961 |
| Rv3316 | sdhC | 1.25 | 0.17277 |
| Rv3317 | sdhD | 0.72 | 0.00012 |
| Rv3318 | sdhA | 1.54 | 0.01649 |
| Rv3319 | sdhB | 1.58 | 0.04031 |
| Rv3320c | hypothetical protein Rv3320c | 2.92 | 0.01743 |
| Rv3321c | hypothetical protein Rv3321c | 0.91 | 0.27034 |
| Rv3322c | moaE3 | 4.89 | 0.02068 |
| Rv3323c | gphA | 1.40 | 0.15141 |
| Rv3324c | moaC3 | 1.85 | 0.01741 |
| Rv3325 | hypothetical protein Rv3325 | 1.95 | 0.09245 |
| Rv3326 | hypothetical protein Rv3326 | 2.07 | 0.02164 |
| Rv3327 | hypothetical protein Rv3327 | 0.79 | 0.01080 |
| Rv3328c | sigJ | 1.21 | 0.27673 |
| Rv3329 | hypothetical protein Rv3329 | 1.32 | 0.13551 |
| Rv3330 | hypothetical protein Rv3330 | 2.38 | 0.04316 |
| Rv3331 | sugI | 1.66 | 0.01716 |
| Rv3332 | nagA | 1.77 | 0.00217 |
| Rv3333c | hypothetical protein Rv3333c | 4.95 | 0.01583 |
| Rv3334 | hypothetical protein Rv3334 | 3.31 | 0.00023 |
| Rv3335c | hypothetical protein Rv3335c | 3.01 | 0.00970 |
| Rv3336c | trpS | 0.80 | 0.00518 |
| Rv3337 | hypothetical protein Rv3337 | 1.52 | 0.00128 |
| Rv3338 | hypothetical protein Rv3338 | 0.87 | 0.22290 |
| Rv3339c | icd1 | 0.84 | 0.02092 |
| Rv3340 | metC | 2.77 | 0.00067 |
| Rv3341 | metA | 1.14 | 0.23739 |
| Rv3342 | hypothetical protein Rv3342 | 1.75 | 0.08017 |
| Rv3343c | PPE | 8.09 | 0.05291 |
| Rv3344c | PE_PGRS | 2.16 | 0.14594 |
| Rv3345c | PE_PGRS | 5.18 | 0.09996 |
| Rv3346c | hypothetical protein Rv3346c | 1.15 | 0.25223 |
| Rv3347c | PPE | 13.09 | 0.01320 |
| Rv3348 | hypothetical protein Rv3348 | 1.06 | 0.37443 |
| Rv3349c | hypothetical protein Rv3349c | 13.96 | 0.03784 |
| Rv3350c | PPE | 1.53 | 0.11423 |
| Rv3351c | hypothetical protein Rv3351c | 2.95 | 0.08169 |
| Rv3352c | hypothetical protein Rv3352c | 0.96 | 0.32326 |
| Rv3353c | hypothetical protein Rv3353c | 1.74 | 0.05641 |
| Rv3354 | hypothetical protein Rv3354 | 0.98 | 0.43085 |
| Rv3355c | hypothetical protein Rv3355c | 2.83 | 0.09095 |
| Rv3356c | folD | 1.63 | 0.11369 |
| Rv3357 | hypothetical protein Rv3357 | 1.80 | 0.11759 |
| Rv3358 | hypothetical protein Rv3358 | 1.54 | 0.08361 |
| Rv3359 | hypothetical protein Rv3359 | 1.36 | 0.17407 |
| Rv3360 | hypothetical protein Rv3360 | 1.30 | 0.05231 |
| Rv3361c | hypothetical protein Rv3361c | 1.40 | 0.12656 |
| Rv3362c | hypothetical protein Rv3362c | 0.95 | 0.25962 |
| Rv3363c | hypothetical protein Rv3363c | 2.08 | 0.05007 |
| Rv3364c | hypothetical protein Rv3364c | 0.78 | 0.03620 |
| Rv3365c | hypothetical protein Rv3365c | 1.54 | 0.13777 |
| Rv3366 | spoU | 0.74 | 0.00015 |
| Rv3367 | PE_PGRS | 1.02 | 0.30467 |
| Rv3368c | hypothetical protein Rv3368c | 1.20 | 0.25124 |
| Rv3369 | hypothetical protein Rv3369 | 0.94 | 0.23774 |
| Rv3370c | dnaE2 | 3.56 | 0.01869 |
| Rv3371 | hypothetical protein Rv3371 | 1.89 | 0.02063 |
| Rv3372 | otsB2 | 1.11 | 0.34111 |
| Rv3373 | echA18 | 2.02 | 0.03648 |
| Rv3374 | echA18′ | 0.97 | 0.44299 |
| Rv3375 | amiD | 1.73 | 0.00352 |
| Rv3376 | hypothetical protein Rv3376 | 0.63 | 0.00262 |
| Rv3377c | hypothetical protein Rv3377c | 2.29 | 0.01137 |
| Rv3378c | hypothetical protein Rv3378c | 0.97 | 0.45047 |
| Rv3379c | hypothetical protein Rv3379c | 1.44 | 0.02013 |
| Rv3380c | hypothetical protein Rv3380c | 1.86 | 0.10723 |
| Rv3381c | hypothetical protein Rv3381c | 1.51 | 0.14524 |
| Rv3382c | lytB | 0.83 | 0.02359 |
| Rv3383c | idsB | 1.20 | 0.09936 |
| Rv3384c | hypothetical protein Rv3384c | 2.24 | 0.05665 |
| Rv3385c | hypothetical protein Rv3385c | 1.42 | 0.00014 |
| Rv3386 | hypothetical protein Rv3386 | 0.76 | 0.11586 |
| Rv3387 | hypothetical protein Rv3387 | 2.69 | 0.03112 |
| Rv3388 | PE_PGRS | 0.84 | 0.07700 |
| Rv3389c | hypothetical protein Rv3389c | 1.91 | 0.04137 |
| Rv3390 | lpqD | 1.38 | 0.20881 |
| Rv3391 | acrA1 | 1.91 | 0.06617 |
| Rv3392c | cmaA1 | 1.51 | 0.12682 |
| Rv3393 | iunH | 1.39 | 0.08824 |
| Rv3394c | hypothetical protein Rv3394c | 0.82 | 0.09902 |
| Rv3395c | hypothetical protein Rv3395c | 1.39 | 0.05278 |
| Rv3396c | guaA | 1.46 | 0.05905 |
| Rv3397c | phyA | 1.38 | 0.07370 |
| Rv3398c | idsA | 5.25 | 0.13070 |
| Rv3399 | hypothetical protein Rv3399 | 1.10 | 0.20706 |
| Rv3401 | hypothetical protein Rv3401 | 1.90 | 0.18283 |
| Rv3402c | hypothetical protein Rv3402c | 1.86 | 0.29686 |
| Rv3403c | hypothetical protein Rv3403c | 2.50 | 0.05817 |
| Rv3404c | hypothetical protein Rv3404c | 0.00 | 0.14325 |
| Rv3405c | hypothetical protein Rv3405c | 1.18 | 0.30598 |
| Rv3406 | hypothetical protein Rv3406 | 2.57 | 0.15256 |
| Rv3407 | hypothetical protein Rv3407 | 0.56 | 0.01913 |
| Rv3408 | hypothetical protein Rv3408 | 0.70 | 0.03316 |
| Rv3409c | choD | 1.03 | 0.41306 |
| Rv3410c | guaB3 | 1.41 | 0.12393 |
| Rv3411c | guaB2 | 1.16 | 0.10913 |
| Rv3412 | hypothetical protein Rv3412 | 1.26 | 0.11522 |
| Rv3413c | hypothetical protein Rv3413c | 1.64 | 0.09733 |
| Rv3414c | sigD | 0.88 | 0.03625 |
| Rv3415c | hypothetical protein Rv3415c | 3.75 | 0.02823 |
| Rv3416 | whiB3 | 1.10 | 0.25742 |
| Rv3417c | groEL1 | 1.15 | 0.21246 |
| Rv3418c | groES | 1.68 | 0.00423 |
| Rv3419c | gcp | 0.99 | 0.43361 |
| Rv3420c | rimI | 1.43 | 0.10245 |
| Rv3421c | hypothetical protein Rv3421c | 1.10 | 0.20211 |
| Rv3422c | hypothetical protein Rv3422c | 2.67 | 0.08006 |
| Rv3423c | alr | 1.08 | 0.25623 |
| Rv3424c | hypothetical protein Rv3424c | 1.43 | 0.06883 |
| Rv3425 | PPE | 1.45 | 0.09891 |
| Rv3426 | PPE | 1.70 | 0.11033 |
| Rv3427c | hypothetical protein Rv3427c | 1.26 | 0.00633 |
| Rv3428c | hypothetical protein Rv3428c | 1.67 | 0.01236 |
| Rv3429 | PPE | 1.80 | 0.03086 |
| Rv3430c | hypothetical protein Rv3430c | 1.95 | 0.03363 |
| Rv3431c | hypothetical protein Rv3431c | 1.11 | 0.05691 |
| Rv3432c | gadB | 0.83 | 0.08441 |
| Rv3433c | hypothetical protein Rv3433c | 1.90 | 0.03983 |
| Rv3434c | hypothetical protein Rv3434c | 0.77 | 0.00000 |
| Rv3435c | hypothetical protein Rv3435c | 1.60 | 0.16144 |
| Rv3436c | glmS | 0.80 | 0.01351 |
| Rv3437 | hypothetical protein Rv3437 | 0.97 | 0.42478 |
| Rv3438 | hypothetical protein Rv3438 | 2.53 | 0.05972 |
| Rv3439c | hypothetical protein Rv3439c | 0.99 | 0.41805 |
| Rv3440c | hypothetical protein Rv3440c | 0.65 | 0.00038 |
| Rv3441c | mrsA | 1.32 | 0.22656 |
| Rv3442c | rpsI | 0.66 | 0.01022 |
| Rv3443c | rplM | 0.84 | 0.20824 |
| Rv3444c | hypothetical protein Rv3444c | 0.79 | 0.00019 |
| Rv3445c | hypothetical protein Rv3445c | 1.31 | 0.26960 |
| Rv3446c | hypothetical protein Rv3446c | 1.17 | 0.19650 |
| Rv3447c | hypothetical protein Rv3447c | 1.26 | 0.15794 |
| Rv3448 | hypothetical protein Rv3448 | 1.65 | 0.09235 |
| Rv3449 | hypothetical protein Rv3449 | 2.47 | 0.08011 |
| Rv3450c | hypothetical protein Rv3450c | 0.71 | 0.00004 |
| Rv3451 | hypothetical protein Rv3451 | 2.43 | 0.04513 |
| Rv3452 | hypothetical protein Rv3452 | 0.78 | 0.03341 |
| Rv3453 | hypothetical protein Rv3453 | 2.38 | 0.05151 |
| Rv3454 | hypothetical protein Rv3454 | 1.39 | 0.07555 |
| Rv3455c | truA | 1.45 | 0.08907 |
| Rv3456c | rplQ | 0.43 | 0.00000 |
| Rv3457c | rpoA | 0.67 | 0.00000 |
| Rv3458c | rpsD | 0.65 | 0.00082 |
| Rv3459c | rpsK | 0.71 | 0.00019 |
| Rv3460c | rpsM | 0.95 | 0.21113 |
| Rv3461c | rpmJ | 0.56 | 0.00000 |
| Rv3462c | infA | 0.51 | 0.00003 |
| Rv3463 | hypothetical protein Rv3463 | 1.58 | 0.01285 |
| Rv3464 | rmlB | 2.77 | 0.03653 |
| Rv3465 | rmlC | 0.91 | 0.28413 |
| Rv3466 | hypothetical protein Rv3466 | 1.49 | 0.00003 |
| Rv3467 | hypothetical protein Rv3467 | 1.58 | 0.06779 |
| Rv3468c | rmlB3 | 3.22 | 0.00320 |
| Rv3469c | mhpE | 3.29 | 0.03261 |
| Rv3470c | ilvB2 | 2.84 | 0.03006 |
| Rv3471c | hypothetical protein Rv3471c | 3.21 | 0.01319 |
| Rv3472 | hypothetical protein Rv3472 | 0.92 | 0.28306 |
| Rv3473c | bpoA | 1.67 | 0.00568 |
| Rv3474 | hypothetical protein Rv3474 | 1.39 | 0.09240 |
| Rv3475 | hypothetical protein Rv3475 | 3.49 | 0.04337 |
| Rv3476c | kgtP | 2.01 | 0.05901 |
| Rv3477 | PE | 0.66 | 0.07147 |
| Rv3478 | PPE | 0.58 | 0.00266 |
| Rv3479 | hypothetical protein Rv3479 | 1.31 | 0.14416 |
| Rv3480c | hypothetical protein Rv3480c | 1.09 | 0.25646 |
| Rv3481c | hypothetical protein Rv3481c | 1.39 | 0.11569 |
| Rv3482c | hypothetical protein Rv3482c | 0.63 | 0.00014 |
| Rv3483c | hypothetical protein Rv3483c | 0.98 | 0.40316 |
| Rv3484 | cpsA | 0.75 | 0.00362 |
| Rv3485c | hypothetical protein Rv3485c | 1.40 | 0.04493 |
| Rv3486 | hypothetical protein Rv3486 | 0.68 | 0.00007 |
| Rv3487c | lipF | 0.96 | 0.44290 |
| Rv3488 | hypothetical protein Rv3488 | 1.18 | 0.30826 |
| Rv3489 | hypothetical protein Rv3489 | 0.77 | 0.02537 |
| Rv3490 | otsA | 1.85 | 0.06368 |
| Rv3491 | hypothetical protein Rv3491 | 1.28 | 0.17228 |
| Rv3492c | hypothetical protein Rv3492c | 0.74 | 0.02637 |
| Rv3493c | hypothetical protein Rv3493c | 1.53 | 0.18149 |
| Rv3494c | hypothetical protein Rv3494c | 0.78 | 0.08215 |
| Rv3495c | lprN | 1.99 | 0.12899 |
| Rv3496c | hypothetical protein Rv3496c | 3.12 | 0.08886 |
| Rv3497c | hypothetical protein Rv3497c | 2.47 | 0.05149 |
| Rv3498c | hypothetical protein Rv3498c | 0.98 | 0.43550 |
| Rv3499c | mce4 | 1.24 | 0.25204 |
| Rv3500c | hypothetical protein Rv3500c | 1.27 | 0.25918 |
| Rv3501c | hypothetical protein Rv3501c | 1.37 | 0.21523 |
| Rv3502c | hypothetical protein Rv3502c | 0.88 | 0.06191 |
| Rv3503c | fdxD | 1.87 | 0.04600 |
| Rv3504 | fadE26 | 1.63 | 0.00109 |
| Rv3505 | fadE27 | 1.32 | 0.05415 |
| Rv3506 | fadD17 | 1.45 | 0.02500 |
| Rv3507 | PE_PGRS | 1.07 | 0.37104 |
| Rv3508 | PE_PGRS | 0.97 | 0.39733 |
| Rv3509c | ilvX | 0.91 | 0.08815 |
| Rv3510c | hypothetical protein Rv3510c | 1.19 | 0.17412 |
| Rv3511 | PE_PGRS | 1.18 | 0.01052 |
| Rv3512 | PE_PGRS | 1.15 | 0.00312 |
| Rv3513c | fadD18 | 1.68 | 0.01578 |
| Rv3514 | PE_PGRS | 0.99 | 0.47621 |
| Rv3515c | fadD19 | 3.12 | 0.00910 |
| Rv3516 | echA19 | 1.49 | 0.06493 |
| Rv3517 | hypothetical protein Rv3517 | 1.14 | 0.01839 |
| Rv3518c | hypothetical protein Rv3518c | 1.41 | 0.02979 |
| Rv3519 | hypothetical protein Rv3519 | 1.15 | 0.16900 |
| Rv3520c | hypothetical protein Rv3520c | 0.95 | 0.32820 |
| Rv3521 | hypothetical protein Rv3521 | 2.88 | 0.00874 |
| Rv3522 | hypothetical protein Rv3522 | 1.17 | 0.11270 |
| Rv3523 | hypothetical protein Rv3523 | 1.23 | 0.11106 |
| Rv3524 | hypothetical protein Rv3524 | 1.71 | 0.02327 |
| Rv3525c | hypothetical protein Rv3525c | 1.25 | 0.05067 |
| Rv3526 | hypothetical protein Rv3526 | 1.24 | 0.00376 |
| Rv3527 | hypothetical protein Rv3527 | 2.48 | 0.05851 |
| Rv3528c | hypothetical protein Rv3528c | 1.09 | 0.31711 |
| Rv3529c | hypothetical protein Rv3529c | 1.52 | 0.00916 |
| Rv3530c | hypothetical protein Rv3530c | 2.27 | 0.02883 |
| Rv3531c | hypothetical protein Rv3531c | 2.39 | 0.00294 |
| Rv3532 | PPE | 2.94 | 0.01497 |
| Rv3533c | PPE | 1.57 | 0.08755 |
| Rv3534c | hypothetical protein Rv3534c | 1.54 | 0.00177 |
| Rv3535c | hypothetical protein Rv3535c | 1.30 | 0.01288 |
| Rv3536c | hypothetical protein Rv3536c | 0.76 | 0.02299 |
| Rv3537 | hypothetical protein Rv3537 | 1.75 | 0.00060 |
| Rv3538 | ufaA2 | 2.08 | 0.00280 |
| Rv3539 | PE | 7.61 | 0.00599 |
| Rv3540c | ltp2 | 1.05 | 0.40774 |
| Rv3541c | hypothetical protein Rv3541c | 1.19 | 0.19158 |
| Rv3542c | hypothetical protein Rv3542c | 2.26 | 0.04832 |
| Rv3543c | fadE29 | 2.40 | 0.06398 |
| Rv3544c | fadE28 | 2.63 | 0.06079 |
| Rv3545c | hypothetical protein Rv3545c | 1.12 | 0.05221 |
| Rv3546 | fadA5 | 1.49 | 0.16829 |
| Rv3547 | hypothetical protein Rv3547 | 1.11 | 0.20915 |
| Rv3548c | hypothetical protein Rv3548c | 0.85 | 0.01333 |
| Rv3549c | hypothetical protein Rv3549c | 2.35 | 0.03833 |
| Rv3550 | echA20 | 0.94 | 0.27249 |
| Rv3551 | hypothetical protein Rv3551 | 2.10 | 0.08295 |
| Rv3552 | hypothetical protein Rv3552 | 1.18 | 0.27238 |
| Rv3553 | hypothetical protein Rv3553 | 1.39 | 0.06305 |
| Rv3554 | fdxB | 0.93 | 0.28609 |
| Rv3555c | hypothetical protein Rv3555c | 1.53 | 0.04160 |
| Rv3556c | fadA6 | 0.92 | 0.29713 |
| Rv3557c | hypothetical protein Rv3557c | 1.86 | 0.01990 |
| Rv3558 | PPE | 1.61 | 0.07843 |
| Rv3559c | hypothetical protein Rv3559c | 1.63 | 0.02191 |
| Rv3560c | fadE30 | 2.07 | 0.05771 |
| Rv3561 | fadD3 | 1.68 | 0.08003 |
| Rv3562 | fadE31 | 1.06 | 0.20402 |
| Rv3563 | fadE32 | 1.17 | 0.09331 |
| Rv3564 | fadE33 | 1.40 | 0.07759 |
| Rv3565 | aspB | 1.02 | 0.43176 |
| Rv3566c | nhoA | 1.16 | 0.33618 |
| Rv3567c | hypothetical protein Rv3567c | 2.22 | 0.04362 |
| Rv3568c | hypothetical protein Rv3568c | 1.33 | 0.14612 |
| Rv3569c | hypothetical protein Rv3569c | 0.91 | 0.24673 |
| Rv3570c | hypothetical protein Rv3570c | 0.69 | 0.00009 |
| Rv3571 | hypothetical protein Rv3571 | 2.19 | 0.01085 |
| Rv3572 | hypothetical protein Rv3572 | 2.84 | 0.04517 |
| Rv3573c | fadE34 | 1.45 | 0.06980 |
| Rv3574 | hypothetical protein Rv3574 | 1.03 | 0.39857 |
| Rv3575c | hypothetical protein Rv3575c | 3.19 | 0.01175 |
| Rv3576 | pknM | 0.66 | 0.00027 |
| Rv3577 | hypothetical protein Rv3577 | 1.48 | 0.09430 |
| Rv3578 | arsB2 | 0.71 | 0.00056 |
| Rv3579c | hypothetical protein Rv3579c | 0.95 | 0.37384 |
| Rv3580c | cysS | 1.17 | 0.10740 |
| Rv3581c | hypothetical protein Rv3581c | 1.03 | 0.37106 |
| Rv3582c | hypothetical protein Rv3582c | 1.18 | 0.16538 |
| Rv3583c | hypothetical protein Rv3583c | 0.61 | 0.00001 |
| Rv3584 | lpqE | 0.42 | 0.00000 |
| Rv3585 | radA | 1.05 | 0.24700 |
| Rv3586 | hypothetical protein Rv3586 | 0.91 | 0.20970 |
| Rv3587c | hypothetical protein Rv3587c | 2.20 | 0.02322 |
| Rv3588c | hypothetical protein Rv3588c | 0.81 | 0.03836 |
| Rv3589 | mutY | 1.75 | 0.11301 |
| Rv3590c | PE_PGRS | 3.59 | 0.15730 |
| Rv3591c | hypothetical protein Rv3591c | 1.79 | 0.05653 |
| Rv3592 | hypothetical protein Rv3592 | 0.52 | 0.00000 |
| Rv3593 | lpqF | 0.63 | 0.00000 |
| Rv3594 | hypothetical protein Rv3594 | 0.75 | 0.00221 |
| Rv3595c | PE_PGRS | 1.28 | 0.13962 |
| Rv3596c | clpC | 0.66 | 0.01833 |
| Rv3597c | lsr2 | 1.31 | 0.11830 |
| Rv3598c | lysS | 1.16 | 0.23713 |
| Rv3599c | hypothetical protein Rv3599c | 3.26 | 0.07093 |
| Rv3600c | hypothetical protein Rv3600c | 1.01 | 0.45779 |
| Rv3601c | panD | 1.77 | 0.00105 |
| Rv3602c | panC | 1.72 | 0.02963 |
| Rv3603c | hypothetical protein Rv3603c | 1.76 | 0.00002 |
| Rv3604c | hypothetical protein Rv3604c | 1.89 | 0.00952 |
| Rv3605c | hypothetical protein Rv3605c | 0.96 | 0.35437 |
| Rv3606c | folK | 1.05 | 0.40125 |
| Rv3607c | folX | 1.08 | 0.14688 |
| Rv3608c | folP | 1.39 | 0.03600 |
| Rv3609c | folE | 0.92 | 0.31578 |
| Rv3610c | ftsH | 1.40 | 0.02413 |
| Rv3611 | hypothetical protein Rv3611 | 1.59 | 0.02714 |
| Rv3612c | hypothetical protein Rv3612c | 0.76 | 0.09531 |
| Rv3613c | hypothetical protein Rv3613c | 1.83 | 0.01818 |
| Rv3614c | hypothetical protein Rv3614c | 0.99 | 0.48969 |
| Rv3615c | hypothetical protein Rv3615c | 2.02 | 0.15176 |
| Rv3616c | hypothetical protein Rv3616c | 0.94 | 0.45148 |
| Rv3617 | ephA | 1.68 | 0.00189 |
| Rv3618 | hypothetical protein Rv3618 | 0.51 | 0.00061 |
| Rv3619c | hypothetical protein Rv3619c | 0.48 | 0.00000 |
| Rv3620c | hypothetical protein Rv3620c | 0.30 | 0.00000 |
| Rv3621c | PPE | 1.06 | 0.40476 |
| Rv3622c | PE | 1.87 | 0.06139 |
| Rv3623 | lpqG | 1.32 | 0.05084 |
| Rv3624c | hpt | 0.62 | 0.00074 |
| Rv3625c | mesJ | 1.10 | 0.22637 |
| Rv3626c | hypothetical protein Rv3626c | 1.32 | 0.15538 |
| Rv3627c | hypothetical protein Rv3627c | 1.68 | 0.00193 |
| Rv3628 | ppa | 0.89 | 0.27729 |
| Rv3629c | hypothetical protein Rv3629c | 1.21 | 0.16293 |
| Rv3630 | hypothetical protein Rv3630 | 0.83 | 0.15263 |
| Rv3631 | hypothetical protein Rv3631 | 0.90 | 0.13164 |
| Rv3632 | hypothetical protein Rv3632 | 1.19 | 0.32258 |
| Rv3633 | hypothetical protein Rv3633 | 1.07 | 0.32767 |
| Rv3634c | rmlB2 | 0.81 | 0.00737 |
| Rv3635 | hypothetical protein Rv3635 | 1.48 | 0.04311 |
| Rv3636 | hypothetical protein Rv3636 | 0.65 | 0.00014 |
| Rv3637 | hypothetical protein Rv3637 | 3.61 | 0.04899 |
| Rv3638 | hypothetical protein Rv3638 | 1.18 | 0.23884 |
| Rv3639c | hypothetical protein Rv3639c | 5.06 | 0.01837 |
| Rv3640c | hypothetical protein Rv3640c | 0.69 | 0.00002 |
| Rv3641c | fic | 1.09 | 0.35147 |
| Rv3642c | hypothetical protein Rv3642c | 1.07 | 0.35853 |
| Rv3643 | hypothetical protein Rv3643 | 3.58 | 0.03715 |
| Rv3644c | hypothetical protein Rv3644c | 1.53 | 0.14032 |
| Rv3645 | hypothetical protein Rv3645 | 0.93 | 0.25380 |
| Rv3646c | topA | 1.35 | 0.19349 |
| Rv3647c | hypothetical protein Rv3647c | 0.82 | 0.05926 |
| Rv3648c | cspA | 0.13 | 0.00000 |
| Rv3649 | hypothetical protein Rv3649 | 1.73 | 0.02063 |
| Rv3650 | PE | 4.61 | 0.00783 |
| Rv3651 | hypothetical protein Rv3651 | 3.17 | 0.00384 |
| Rv3652 | PE_PGRS | 1.74 | 0.00051 |
| Rv3653 | PE_PGRS | 1.22 | 0.00002 |
| Rv3654c | hypothetical protein Rv3654c | 1.07 | 0.27791 |
| Rv3655c | hypothetical protein Rv3655c | 3.64 | 0.03178 |
| Rv3656c | hypothetical protein Rv3656c | 1.06 | 0.26561 |
| Rv3657c | hypothetical protein Rv3657c | 1.12 | 0.08130 |
| Rv3658c | hypothetical protein Rv3658c | 1.38 | 0.05158 |
| Rv3659c | trbB | 1.00 | 0.49922 |
| Rv3660c | hypothetical protein Rv3660c | 2.03 | 0.00035 |
| Rv3661 | hypothetical protein Rv3661 | 2.32 | 0.02382 |
| Rv3662c | hypothetical protein Rv3662c | 1.42 | 0.00002 |
| Rv3663c | dppD | 2.95 | 0.05715 |
| Rv3664c | dppC | 1.49 | 0.04136 |
| Rv3665c | dppB | 3.39 | 0.01285 |
| Rv3666c | dppA | 0.89 | 0.07753 |
| Rv3667 | acs | 2.02 | 0.02356 |
| Rv3668c | hypothetical protein Rv3668c | 1.71 | 0.00422 |
| Rv3669 | hypothetical protein Rv3669 | 3.04 | 0.00450 |
| Rv3670 | ephE | 2.72 | 0.06882 |
| Rv3671c | hypothetical protein Rv3671c | 1.90 | 0.04611 |
| Rv3672c | hypothetical protein Rv3672c | 1.03 | 0.39211 |
| Rv3673c | hypothetical protein Rv3673c | 1.51 | 0.09442 |
| Rv3674c | nth | 1.12 | 0.21819 |
| Rv3675 | hypothetical protein Rv3675 | 1.31 | 0.04044 |
| Rv3676 | hypothetical protein Rv3676 | 1.18 | 0.29455 |
| Rv3677c | hypothetical protein Rv3677c | 0.72 | 0.00374 |
| Rv3678c | hypothetical protein Rv3678c | 0.57 | 0.00044 |
| Rv3679 | hypothetical protein Rv3679 | 1.50 | 0.10581 |
| Rv3680 | hypothetical protein Rv3680 | 0.74 | 0.03538 |
| Rv3681c | whiB4 | 1.92 | 0.02947 |
| Rv3682 | ponA′ | 1.21 | 0.33829 |
| Rv3683 | hypothetical protein Rv3683 | 1.35 | 0.05818 |
| Rv3684 | hypothetical protein Rv3684 | 0.87 | 0.20142 |
| Rv3685c | hypothetical protein Rv3685c | 0.86 | 0.05680 |
| Rv3686c | hypothetical protein Rv3686c | 0.56 | 0.01248 |
| Rv3687c | hypothetical protein Rv3687c | 1.76 | 0.14789 |
| Rv3688c | hypothetical protein Rv3688c | 0.53 | 0.00000 |
| Rv3689 | hypothetical protein Rv3689 | 2.74 | 0.07155 |
| Rv3690 | hypothetical protein Rv3690 | 0.87 | 0.21707 |
| Rv3691 | hypothetical protein Rv3691 | 1.03 | 0.43509 |
| Rv3692 | moxR2 | 1.07 | 0.40076 |
| Rv3693 | hypothetical protein Rv3693 | 0.96 | 0.43273 |
| Rv3694c | hypothetical protein Rv3694c | 0.76 | 0.00513 |
| Rv3695 | hypothetical protein Rv3695 | 2.19 | 0.09410 |
| Rv3696c | glpK | 0.70 | 0.00927 |
| Rv3697c | hypothetical protein Rv3697c | 3.27 | 0.04006 |
| Rv3698 | hypothetical protein Rv3698 | 2.62 | 0.00835 |
| Rv3699 | hypothetical protein Rv3699 | 1.85 | 0.01924 |
| Rv3700c | hypothetical protein Rv3700c | 0.86 | 0.11903 |
| Rv3701c | hypothetical protein Rv3701c | 1.11 | 0.25300 |
| Rv3702c | hypothetical protein Rv3702c | 2.31 | 0.01081 |
| Rv3703c | hypothetical protein Rv3703c | 0.89 | 0.06791 |
| Rv3704c | gshA | 1.67 | 0.00713 |
| Rv3705c | hypothetical protein Rv3705c | 1.20 | 0.20596 |
| Rv3706c | hypothetical protein Rv3706c | 6.86 | 0.01995 |
| Rv3707c | hypothetical protein Rv3707c | 1.29 | 0.07234 |
| Rv3708c | asd | 1.46 | 0.10380 |
| Rv3709c | ask | 1.42 | 0.04555 |
| Rv3710 | leuA | 1.23 | 0.15218 |
| Rv3711c | dnaQ | 0.95 | 0.37342 |
| Rv3712 | hypothetical protein Rv3712 | 1.55 | 0.01620 |
| Rv3713 | cobQ2 | 1.71 | 0.06423 |
| Rv3714c | hypothetical protein Rv3714c | 1.30 | 0.00000 |
| Rv3715c | recR | 1.05 | 0.30667 |
| Rv3716c | hypothetical protein Rv3716c | 1.25 | 0.10267 |
| Rv3717 | hypothetical protein Rv3717 | 1.35 | 0.11525 |
| Rv3718c | hypothetical protein Rv3718c | 0.91 | 0.27036 |
| Rv3719 | hypothetical protein Rv3719 | 1.11 | 0.27339 |
| Rv3720 | hypothetical protein Rv3720 | 1.40 | 0.08770 |
| Rv3721c | dnaZX | 3.14 | 0.02265 |
| Rv3722c | hypothetical protein Rv3722c | 0.62 | 0.00015 |
| Rv3723 | hypothetical protein Rv3723 | 1.47 | 0.02267 |
| Rv3724 | hypothetical protein Rv3724 | 1.23 | 0.18655 |
| Rv3725 | hypothetical protein Rv3725 | 0.98 | 0.38003 |
| Rv3726 | hypothetical protein Rv3726 | 0.73 | 0.00451 |
| Rv3727 | hypothetical protein Rv3727 | 1.09 | 0.30819 |
| Rv3728 | hypothetical protein Rv3728 | 1.16 | 0.23619 |
| Rv3729 | hypothetical protein Rv3729 | 1.60 | 0.00009 |
| Rv3730c | hypothetical protein Rv3730c | 1.10 | 0.11314 |
| Rv3731 | ligC | 1.97 | 0.03330 |
| Rv3732 | hypothetical protein Rv3732 | 0.96 | 0.38275 |
| Rv3733c | hypothetical protein Rv3733c | 2.89 | 0.09991 |
| Rv3734c | hypothetical protein Rv3734c | 0.57 | 0.00009 |
| Rv3735 | hypothetical protein Rv3735 | 1.38 | 0.20893 |
| Rv3736 | hypothetical protein Rv3736 | 0.98 | 0.45520 |
| Rv3737 | hypothetical protein Rv3737 | 4.66 | 0.06929 |
| Rv3738c | PPE | 2.51 | 0.10593 |
| Rv3739c | PPE | 1.83 | 0.14086 |
| Rv3740c | hypothetical protein Rv3740c | 1.18 | 0.31662 |
| Rv3741c | hypothetical protein Rv3741c | 1.84 | 0.05691 |
| Rv3742c | hypothetical protein Rv3742c | 0.83 | 0.07679 |
| Rv3743c | hypothetical protein Rv3743c | 1.35 | 0.18649 |
| Rv3744 | hypothetical protein Rv3744 | 1.10 | 0.33378 |
| Rv3745c | hypothetical protein Rv3745c | 1.94 | 0.02418 |
| Rv3746c | PE | 2.03 | 0.06670 |
| Rv3747 | hypothetical protein Rv3747 | 1.64 | 0.01591 |
| Rv3748 | hypothetical protein Rv3748 | 0.55 | 0.00021 |
| Rv3749c | hypothetical protein Rv3749c | 0.85 | 0.16329 |
| Rv3750c | hypothetical protein Rv3750c | 0.91 | 0.31656 |
| Rv3751 | hypothetical protein Rv3751 | 1.28 | 0.05386 |
| Rv3752c | hypothetical protein Rv3752c | 1.44 | 0.07038 |
| Rv3753c | hypothetical protein Rv3753c | 1.65 | 0.10543 |
| Rv3754 | tyrA | 1.58 | 0.02878 |
| Rv3755c | hypothetical protein Rv3755c | 1.12 | 0.30586 |
| Rv3756c | proZ | 1.00 | 0.49466 |
| Rv3757c | proW | 1.10 | 0.13266 |
| Rv3758c | proV | 0.70 | 0.02999 |
| Rv3759c | proX | 1.03 | 0.39772 |
| Rv3760 | hypothetical protein Rv3760 | 1.92 | 0.03904 |
| Rv3761c | fadE36 | 0.97 | 0.25071 |
| Rv3762c | hypothetical protein Rv3762c | 0.67 | 0.00016 |
| Rv3763 | lpqH | 0.99 | 0.42272 |
| Rv3764c | hypothetical protein Rv3764c | 0.84 | 0.19358 |
| Rv3765c | hypothetical protein Rv3765c | 1.24 | 0.01218 |
| Rv3766 | hypothetical protein Rv3766 | 0.63 | 0.00698 |
| Rv3767c | hypothetical protein Rv3767c | 1.20 | 0.12487 |
| Rv3768 | hypothetical protein Rv3768 | 1.13 | 0.13500 |
| Rv3769 | hypothetical protein Rv3769 | 1.26 | 0.25175 |
| Rv3770c | hypothetical protein Rv3770c | 1.82 | 0.10614 |
| Rv3771c | hypothetical protein Rv3771c | 1.41 | 0.11011 |
| Rv3772 | hisC2 | 1.18 | 0.21807 |
| Rv3773c | hypothetical protein Rv3773c | 0.69 | 0.00037 |
| Rv3774 | echA21 | 0.45 | 0.00001 |
| Rv3775 | lipE | 1.74 | 0.05544 |
| Rv3776 | hypothetical protein Rv3776 | 0.67 | 0.00182 |
| Rv3777 | hypothetical protein Rv3777 | 1.07 | 0.17981 |
| Rv3778c | hypothetical protein Rv3778c | 1.24 | 0.12034 |
| Rv3779 | hypothetical protein Rv3779 | 1.89 | 0.05477 |
| Rv3780 | hypothetical protein Rv3780 | 1.34 | 0.23921 |
| Rv3781 | hypothetical protein Rv3781 | 0.86 | 0.29741 |
| Rv3782 | rfbE | 1.13 | 0.37191 |
| Rv3783 | hypothetical protein Rv3783 | 2.23 | 0.09856 |
| Rv3784 | epiB | 1.01 | 0.48809 |
| Rv3785 | hypothetical protein Rv3785 | 1.78 | 0.08031 |
| Rv3786c | hypothetical protein Rv3786c | 1.07 | 0.38296 |
| Rv3787c | hypothetical protein Rv3787c | 3.09 | 0.06985 |
| Rv3788 | hypothetical protein Rv3788 | 1.84 | 0.09341 |
| Rv3789 | hypothetical protein Rv3789 | 6.83 | 0.04745 |
| Rv3790 | hypothetical protein Rv3790 | 0.92 | 0.34925 |
| Rv3791 | hypothetical protein Rv3791 | 0.96 | 0.45374 |
| Rv3792 | hypothetical protein Rv3792 | 1.02 | 0.46276 |
| Rv3793 | embC | 0.83 | 0.10456 |
| Rv3794 | embA | 1.09 | 0.34581 |
| Rv3795 | embB | 1.08 | 0.23968 |
| Rv3796 | atsH | 2.15 | 0.01247 |
| Rv3797 | fadE35 | 1.38 | 0.02072 |
| Rv3798 | hypothetical protein Rv3798 | 1.64 | 0.10882 |
| Rv3799c | accD4 | 0.69 | 0.00000 |
| Rv3800c | pks13 | 1.10 | 0.28087 |
| Rv3801c | fadD32 | 1.23 | 0.03936 |
| Rv3802c | hypothetical protein Rv3802c | 1.34 | 0.09435 |
| Rv3803c | fbpC1 | 1.50 | 0.02447 |
| Rv3804c | fbpA | 0.28 | 0.00000 |
| Rv3805c | hypothetical protein Rv3805c | 1.12 | 0.13868 |
| Rv3806c | hypothetical protein Rv3806c | 2.53 | 0.08033 |
| Rv3807c | hypothetical protein Rv3807c | 1.28 | 0.11475 |
| Rv3808c | hypothetical protein Rv3808c | 1.17 | 0.35706 |
| Rv3809c | glf | 1.20 | 0.13034 |
| Rv3810 | pirG | 0.89 | 0.32587 |
| Rv3811 | csp | 2.03 | 0.00508 |
| Rv3812 | PE_PGRS | 1.19 | 0.21438 |
| Rv3813c | hypothetical protein Rv3813c | 1.35 | 0.08188 |
| Rv3814c | hypothetical protein Rv3814c | 0.71 | 0.02389 |
| Rv3815c | hypothetical protein Rv3815c | 1.23 | 0.08452 |
| Rv3816c | hypothetical protein Rv3816c | 1.95 | 0.07802 |
| Rv3817 | hypothetical protein Rv3817 | 1.54 | 0.09835 |
| Rv3818 | hypothetical protein Rv3818 | 0.71 | 0.00001 |
| Rv3819 | hypothetical protein Rv3819 | 4.71 | 0.03191 |
| Rv3820c | papA2 | 3.07 | 0.05938 |
| Rv3821 | hypothetical protein Rv3821 | 0.85 | 0.00799 |
| Rv3822 | hypothetical protein Rv3822 | 2.47 | 0.11809 |
| Rv3823c | mmpL8 | 0.79 | 0.00802 |
| Rv3824c | papA1 | 0.96 | 0.43143 |
| Rv3825c | pks2 | 1.74 | 0.13457 |
| Rv3826 | fadD23 | 1.14 | 0.37656 |
| Rv3827c | hypothetical protein Rv3827c | 1.30 | 0.06918 |
| Rv3828c | hypothetical protein Rv3828c | 0.65 | 0.00324 |
| Rv3829c | hypothetical protein Rv3829c | 3.49 | 0.02860 |
| Rv3830c | hypothetical protein Rv3830c | 0.77 | 0.04417 |
| Rv3831 | hypothetical protein Rv3831 | 2.93 | 0.05827 |
| Rv3832c | hypothetical protein Rv3832c | 1.13 | 0.34945 |
| Rv3833 | hypothetical protein Rv3833 | 2.46 | 0.05261 |
| Rv3834c | serS | 0.81 | 0.10145 |
| Rv3835 | hypothetical protein Rv3835 | 1.25 | 0.24312 |
| Rv3836 | hypothetical protein Rv3836 | 1.66 | 0.13811 |
| Rv3837c | hypothetical protein Rv3837c | 1.14 | 0.27424 |
| Rv3838c | pheA | 2.24 | 0.08402 |
| Rv3839 | hypothetical protein Rv3839 | 2.64 | 0.00157 |
| Rv3840 | hypothetical protein Rv3840 | 2.51 | 0.05356 |
| Rv3841 | bfrB | 0.42 | 0.00000 |
| Rv3842c | glpQ1 | 1.50 | 0.02774 |
| Rv3843c | hypothetical protein Rv3843c | 0.79 | 0.04803 |
| Rv3844 | hypothetical protein Rv3844 | 1.30 | 0.06297 |
| Rv3845 | hypothetical protein Rv3845 | 5.03 | 0.02550 |
| Rv3846 | sodA | 1.63 | 0.00106 |
| Rv3847 | hypothetical protein Rv3847 | 1.19 | 0.20626 |
| Rv3848 | hypothetical protein Rv3848 | 2.90 | 0.00173 |
| Rv3849 | hypothetical protein Rv3849 | 0.58 | 0.00000 |
| Rv3850 | hypothetical protein Rv3850 | 1.04 | 0.40426 |
| Rv3851 | hypothetical protein Rv3851 | 0.82 | 0.00094 |
| Rv3852 | hns | 0.87 | 0.11172 |
| Rv3853 | menG | 0.81 | 0.01107 |
| Rv3854c | hypothetical protein Rv3854c | 1.93 | 0.01334 |
| Rv3855 | hypothetical protein Rv3855 | 1.47 | 0.00918 |
| Rv3856c | hypothetical protein Rv3856c | 1.28 | 0.15202 |
| Rv3857c | hypothetical protein Rv3857c | 1.56 | 0.03614 |
| Rv3858c | gltD | 1.03 | 0.43810 |
| Rv3859c | gltB | 1.09 | 0.13995 |
| Rv3860 | hypothetical protein Rv3860 | 0.96 | 0.13322 |
| Rv3861 | hypothetical protein Rv3861 | 2.25 | 0.03422 |
| Rv3862c | hypothetical protein Rv3862c | 5.31 | 0.00028 |
| Rv3863 | hypothetical protein Rv3863 | 1.60 | 0.01024 |
| Rv3864 | hypothetical protein Rv3864 | 1.13 | 0.22402 |
| Rv3865 | hypothetical protein Rv3865 | 1.00 | 0.47226 |
| Rv3866 | hypothetical protein Rv3866 | 0.87 | 0.00351 |
| Rv3867 | hypothetical protein Rv3867 | 0.97 | 0.35508 |
| Rv3868 | hypothetical protein Rv3868 | 1.10 | 0.38791 |
| Rv3869 | hypothetical protein Rv3869 | 1.81 | 0.04253 |
| Rv3870 | hypothetical protein Rv3870 | 0.95 | 0.06023 |
| Rv3871 | hypothetical protein Rv3871 | 1.22 | 0.07421 |
| Rv3872 | PE | 1.05 | 0.24763 |
| Rv3873 | PPE | 1.99 | 0.00982 |
| Rv3874 | hypothetical protein Rv3874 | 0.40 | 0.00002 |
| Rv3875 | esat6 | 0.60 | 0.00598 |
| Rv3876 | hypothetical protein Rv3876 | 0.89 | 0.29202 |
| Rv3877 | hypothetical protein Rv3877 | 1.72 | 0.11673 |
| Rv3878 | hypothetical protein Rv3878 | 1.91 | 0.02637 |
| Rv3879c | hypothetical protein Rv3879c | 2.21 | 0.05255 |
| Rv3880c | hypothetical protein Rv3880c | 1.35 | 0.22837 |
| Rv3881c | hypothetical protein Rv3881c | 0.72 | 0.01638 |
| Rv3882c | hypothetical protein Rv3882c | 1.07 | 0.41036 |
| Rv3883c | hypothetical protein Rv3883c | 0.83 | 0.24373 |
| Rv3884c | hypothetical protein Rv3884c | 0.66 | 0.00021 |
| Rv3885c | hypothetical protein Rv3885c | 0.85 | 0.05166 |
| Rv3886c | hypothetical protein Rv3886c | 3.06 | 0.04876 |
| Rv3887c | hypothetical protein Rv3887c | 1.30 | 0.19037 |
| Rv3888c | hypothetical protein Rv3888c | 0.58 | 0.00001 |
| Rv3889c | hypothetical protein Rv3889c | 0.94 | 0.11708 |
| Rv3890c | hypothetical protein Rv3890c | 1.32 | 0.00049 |
| Rv3891c | hypothetical protein Rv3891c | 0.85 | 0.00616 |
| Rv3892c | PPE | 1.21 | 0.20402 |
| Rv3893c | PE | 1.46 | 0.01413 |
| Rv3894c | hypothetical protein Rv3894c | 5.20 | 0.00170 |
| Rv3895c | hypothetical protein Rv3895c | 1.46 | 0.06505 |
| Rv3896c | hypothetical protein Rv3896c | 2.18 | 0.00674 |
| Rv3897c | hypothetical protein Rv3897c | 1.06 | 0.30032 |
| Rv3898c | hypothetical protein Rv3898c | 2.18 | 0.00067 |
| Rv3899c | hypothetical protein Rv3899c | 1.43 | 0.07891 |
| Rv3900c | hypothetical protein Rv3900c | 2.05 | 0.00275 |
| Rv3901c | hypothetical protein Rv3901c | 0.81 | 0.01548 |
| Rv3902c | hypothetical protein Rv3902c | 3.63 | 0.22916 |
| Rv3903c | hypothetical protein Rv3903c | 0.83 | 0.07736 |
| Rv3904c | hypothetical protein Rv3904c | 1.23 | 0.16778 |
| Rv3905c | hypothetical protein Rv3905c | 1.06 | 0.33299 |
| Rv3906c | hypothetical protein Rv3906c | 0.98 | 0.43633 |
| Rv3907c | pcnA | 1.56 | 0.02738 |
| Rv3908 | hypothetical protein Rv3908 | 1.27 | 0.11454 |
| Rv3909 | hypothetical protein Rv3909 | 1.36 | 0.07030 |
| Rv3910 | hypothetical protein Rv3910 | 0.92 | 0.18306 |
| Rv3911 | sigM | 2.49 | 0.03446 |
| Rv3912 | hypothetical protein Rv3912 | 2.68 | 0.00309 |
| Rv3913 | trxB2 | 1.72 | 0.04598 |
| Rv3914 | trxC | 1.80 | 0.10738 |
| Rv3915 | cwlM | 0.99 | 0.47181 |
| Rv3916c | hypothetical protein Rv3916c | 0.76 | 0.05702 |
| Rv3917c | parA | 1.64 | 0.00630 |
| Rv3918c | parB | 3.24 | 0.09025 |
| Rv3919c | gid | 1.05 | 0.43638 |
| Rv3920c | hypothetical protein Rv3920c | 0.65 | 0.00038 |
| Rv3921c | hypothetical protein Rv3921c | 0.79 | 0.14341 |
| Rv3922c | hypothetical protein Rv3922c | 0.49 | 0.00004 |
| Rv3923c | rnpA | 1.01 | 0.48559 |
| Rv3924c | rpmH | 0.66 | 0.00954 |

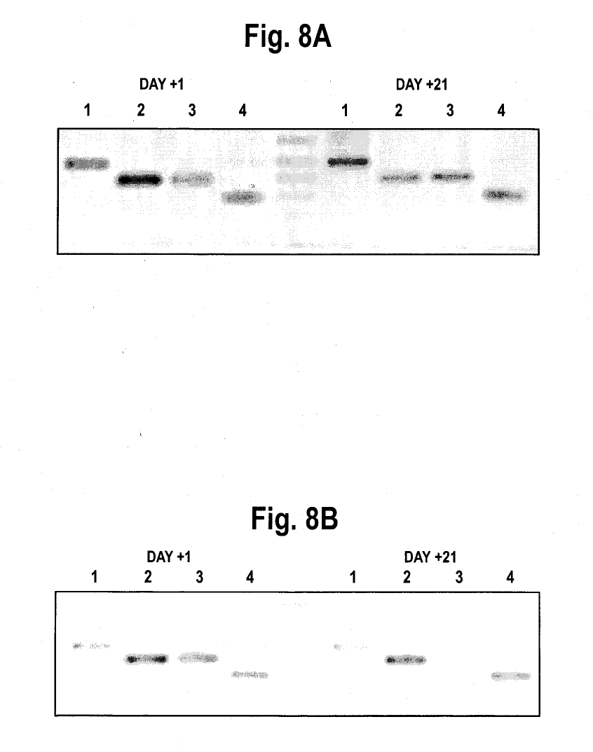

Each pool was generated by mixing an equal volume of pure culture from each mutant to ensure equal representation. Individual pools were then encapsulated into separate hollow fibers and these were either implanted subcutaneously into mice or incubated in vitro. Hollow fiber contents were recovered on day 1 (input pool) and day 21 (output pool) after infection. PCR amplification of the junction sites between the transposons and the adjacent chromosomal DNA in the pooled genomic DNA from recovered mutants was performed at each time point to determine the presence or absence of each mutant. Of the 100 mutants tested, only that containing a transposon insertion in gene MT2660 (Rv2583c,rel_{Mtb}) was present in the in vivo input pool, but absent in the in vivo output pool (FIG. 8b). In contrast, this transposon mutant was readily detectable in both input and output pools in vitro (FIG. 8a). Mice were also infected intravenously with pools representing the same 100 transposon mutants. In concordance with findings from the hollow fiber model in vivo, only the rel_{Mtb}-deficient mutant was found to have significantly decreased survival.

M. tuberculosis deficient in Rel_{Mtb}, an enzyme responsible for the synthesis and hydrolysis of hyperphosphorylated guanine nucleotides involved in the stringent response, has been shown to be significantly attenuated compared to wild-type in the tissues of mice 38 weeks after aerosol infection. Dahl, J. L., et al. Proc Natl Acad Sci USA 100: 10026-10031 (2003). The growth phenotype of the rel_{Mtb} transposon insertion mutant (rel_{Mtb}::Tn), which carries a kanamycin resistance marker, was evaluated in the hollow fiber model in vivo. In order to ensure that each strain was exposed to identical microenvironment conditions, a 1:1 mixture of rel_{Mtb}::Tn and wild-type strain M. tuberculosis CDC 1551 was encapsulated in hollow fibers and either incubated in liquid broth in vitro or implanted subcutaneously in mice. Twenty-one days later, hollow fibers were recovered and their contents were diluted and plated on antibiotic-free and kanamycin-containing plates to assess CFU counts for each hollow, fiber-encapsulated strain. There was no growth deficit of rel_{Mtb}::Tn when incubated in vitro, as both hollow fiber-encapsulated wild-type and rel_{Mtb}::Tn bacilli grew equally after 21 days of incubation (FIG. 8c). However, whereas hollow-fiber encapsulated wild-type bacilli implanted into mice demonstrated reduced growth as compared to those incubated in vitro after 21 days (growth ˜1.5 log for in vivo wild-type vs. ˜3 log₁₀ for in vitro wild-type), hollow fiber-encapsulated rel_{Mtb}::Tn showed markedly reduced survival as compared to wild-type bacilli implanted in vivo (FIG. 8d). Significantly, the decreased survival of the rel_{Mtb}-deficient mutant became apparent as early as 21 days after hollow fiber implantation, as compared to several months using the standard murine aerosol infection model. Dahl, J. L., supra. No significant change in survival compared to wild-type in a M. tuberculosis mutant containing a transposon-insertion in an unrelated gene (MT2749, Rv2675) was observed, suggesting that the presence of the transposon insertion alone does not confer a survival disadvantage in the hollow fiber model in vivo.

Gene Expression of Extracellular M. tuberculosis within Granulomas in Mice

The adaptive response of tubercle bacilli in granulomatous lesions in vivo was determined by studying their global gene expression (for complete gene expression profile, see Table 2). Significantly, the exclusion of host cells and containment of bacilli by the hollow fibers renders whole genome microarray analysis feasible. Hollow fibers containing M. tuberculosis were implanted subcutaneously into mice and retrieved 10 days later. Hollow fiber contents were recovered, immediately snap-frozen, and bacilli from 30-40 implanted fibers were pooled to yield sufficient RNA for analysis. Hollow fiber-encapsulated M. tuberculosis gene expression was compared to that of log-phase in vitro-grown M. tuberculosis. Significant differential gene regulation was defined by both a (i) ≧2-fold change in gene expression as compared to control samples and (ii) p-value <0.01.

In vivo hollow fiber-encapsulated M. tuberculosis demonstrates significant induction of ˜260 genes, including several key regulatory genes (see Table 2). Using the strict criteria outlined above, significant upregulation of Rv3133c (dosR) and 20 other members of the recently described dosR regulon (Voskuil, supra), including hspX, Rv2623c, and Rv2626 (Table 3), was observed, suggesting that the adaptive response of hollow fiber-encapsulated M. tuberculosis in vivo is similar to that of bacilli exposed to low concentrations of nitric oxide in vitro.

TABLE 3
Significantly upregulated dosR regulon genes in the hollow fiber
model in vivo by microarray analysis

| | | | | P- |
|---|---|---|---|---|
| Rv No. | Gene | Protein Function | Fold upregulation | value |
| 80 | | HP | 2.9 | 0.0009 |
| 569 | | CHP | 4.9 | 0.007 |
| 570 | nrdZ | Ribonucleotide | 2 | 6.17E−07 |
| | | reductase cl. II | | |
| 572c | | HP | 2.2 | 0.002 |
| 1733c | | CHP | 3 | 0.004 |
| 1997 | ctpF | Cation transport ATPase | 13.2 | 0.007 |
| 2005c | | CHP-USPA motif | 2.2 | 0.003 |
| 2030c | | CHP | 7.8 | 3.30E−05 |
| 2031c | acr | α-crystallin | 25 | 0.009 |
| 2623 | | CHP-USPA motif | 25.8 | 0.0002 |
| 2626c | | CHP | 14.6 | 0.0001 |
| 2627c | | CHP | 8 | 0.008 |
| 2628 | | HP | 8.4 | 0.004 |
| 2631 | | HP | 2.1 | 0.001 |
| 3126c | | HP | 2.2 | 0.0001 |
| 3128c | | CHP | 3.2 | 0.004 |
| 3130c | | CHP | 15.4 | 0.0003 |
| 3131 | | CHP | 11.4 | 0.0008 |
| 3132c | | Sensor histidine kinase | 4.8 | 0.001 |
| 3133c | dosR | 2-comp. response reg. | 2.1 | 0.007 |
| 3134c | | CHP-USPA motif | 7.1 | 0.007 |

HP = hypothetical protein
CHP = conserved hypothetical protein

The M. tuberculosis hspX gene encodes α-crystallin, a member of the small heat shock protein family with chaperone activity (Yuan, Y., et al. J Bacteriol 178:4484-4492 (1996)), which is powerfully induced under hypoxic conditions (Sherman, supra), and in lung specimens obtained from patients with active tuberculosis disease. Timm, J., et al. Proc Natl Acad Sci USA 100:14321-14326 (2003). Significantly increased expression of hspX, Rv2623c, and Rv2626 has been shown by real time RT-PCR after the onset of Th1-mediated immunity in the mouse model of tuberculosis (Shi, supra) and in various models of latency in vitro. Sherman, supra, Betts, supra, Voskuil, supra, Shi, supra. In addition, upregulation of 12 additional genes of the dosR regulon which fulfilled one, but not both criteria listed above was also observed (see Table 2). Hollow fiber-encapsulated M. tuberculosis in vivo also demonstrated significant upregulation of sigB, sigC, and sigH, which belong to a family of alternative RNA polymerase sigma factors shown to coordinate gene regulation in response to environmental conditions in M. tuberculosis and other bacterial species. Haldenwang, W. G. Tuber Lung Dis 78:175-183 (1995), Gomez, et al., Tuber Lung Dis, 78:175-183 (1997). Significant upregulation of dnaE2, which encodes a DNA polymerase in M. tuberculosis and in other organisms, and which has been shown to be upregulated by several DNA damaging agents and during infection of mice, was detected, contributing to in vivo survival and the emergence of drug resistance. Boshoff, H. I., et al. Cell 113:183-193 (2003). In addition, hollow fiber-encapsulated M. tuberculosis in mice demonstrated significant induction of many other genes recently found to be upregulated in the multibacillary model of murine tuberculosis (Talaat, supra), including RV0967, Rv0970, Rv0978c (PE-PGRS 17), Rv0980c (PE-PGRS 18), Rv982 (mprB), and Rv0988.

Consistent with evidence that M. tuberculosis might switch to C2 carbon sources such as fatty acids during in vivo persistence (McKinney, J. D., et al. Nature 406:735-738 (2000)), significant upregulation of several genes encoding enzymes involved in lipid degradation, including fadD10 (Rv0099), fadD19 (Rv3515c), fadD34 (Rv0035), fadE1 (Rv013 1 c), fadE10 (Rv0873), and fadE24 (Rv3139) was also detected. In addition, significantly increased expression of pckA (Rv0211) was observed, suggesting limited availability of glucose within artificial granulomas in mice. De novo synthesis of glucose-6-phosphate in mycobacteria is achieved through the action of phosphoenolpyruvate carboxykinase (PckA), which converts oxaloacetate to phosphoenolpyruvate, thus diverting carbon derived from β-oxidation of fatty acids into gluconeogenesis. Tuckman, D., et al. J Bacteriol 179:2724-2730 (1997). Increased gene expression of pckA has been reported in the mouse model of tuberculosis (Timm, supra), and disruption of pckA in M. bovis produced attenuated strains in animal models of infection. Liu, K., et al. Microbiology 149:1829-1835 (2003), Collins, D. M., et al. Microbiology 148:3019-3027 (2002). In addition, a 1.5-fold induction of Rv0467 (aceA) (p=0.004) was found, which encodes isocitrate lyase, the first enzyme of the glyoxylate cycle, which is strongly induced in mouse lungs (Timm, supra) and is required for long-term persistence of M. tuberculosis in mice. McKinney, supra.

Consistent with the hypothesis that hollow fiber-encapsulated bacilli in vivo display a reduced replication rate, significantly decreased expression of certain genes encoding ribosomal RNA binding proteins, including Rv0701 (rplC), Rv0703 (rplW), Rv0704 (rplB), Rv0706 (rplV), Rv0708 (rplP), Rv0710 (rplQ), and Rv0716 (rplE) was observed. In agreement with the luciferase assay data suggesting that these bacilli have decreased intracellular energy stores (FIG. 4b), significant downregulation of several genes encoding components of ATP synthase, including Rv1304 (atpB), Rv1306 (atpF), Rv1308 (atpA), Rv1310 (atpD), and Rv1311 (atpC) was found. A complete list of significantly downregulated genes in hollow fiber-encapsulated M. tuberculosis implanted into mice can be found in Table 2. Differential regulation of a subset of 13 genes identified by microarray analysis was confirmed using quantitative real-time RT-PCR (Table 4).

TABLE 4
Confirmation of differential regulation of a subset of genes by
quantitative real-time RT-PCR

| Rv No. | Gene | Protein Function | Fold induction |
|---|---|---|---|
| 1997 | | HP | 32 |
| 2031c | acr | α-crystallin | 294.1 |
| 2623 | | CHP-USPA motif | 512 |
| 2626c | | CHP | 68.6 |
| 2710 | sigB | RNA polymerase sigma | 42.2 |
| | | factor | |
| 2744c | | 35 kd_Ag | 4.3 |
| 3130c | | CHP | 168.9 |
| 3131 | | CHP | 274.3 |
| 3133c | dosR | 2-comp. response regulator | 78.8 |
| 3223c | sigH | RNA polymerase sigma | 17.1 |
| | | factor | |
| 3370c | dnaE2 | DNA polymerase III | 9.8 |
| 3477 | | PE | −4.3 |
| 3648c | cspA | prob. cold shock protein | −7 |

HP = hypothetical protein
CHP = conserved hypothetical protein

Discussion

The hollow fiber encapsulation/implantation technique provides a means to establish a paucibacillary infection with M. tuberculosis in which the bacilli are readily recoverable from infected animals for further analysis. In this model of infection, the tubercle bacilli rapidly enter an altered physiologic state characterized by stationary-state CFU counts and decreased metabolic activity. These bacilli are more susceptible to the antituberculous drug rifampin than they are to isoniazid, consistent with the antibiotic susceptibility profile of persistent bacilli in animal chemotherapy models (Lecoeur, supra, Dickinson, supra) and in human latent tuberculosis infection. Am Rev Respir Dis 145:36-41 (1992), Jasmer, supra, Fox, supra. In addition, expression of rel_{Mtb}, a gene which is essential for long-term persistence in the mouse model of chronic tuberculosis (Dahl, supra), is also necessary for short-term bacillary persistence in the hollow fiber granuloma model in vivo, suggesting a common adaptive strategy of M. tuberculosis in these two models of infection. These results demonstrate induction of dosR (Rv3133c) and 20 other members of the dosR regulon believed to mediate the transition into dormancy, and that rel_{Mtb} is required for M. tuberculosis survival during extracellular persistence within host granulomas. Interestingly, the dormancy phenotype of extracellular M. tuberculosis within host granulomas appears to be immune-mediated and interferon-gamma-dependent.

In this example, the whole-genome transcriptional profile of extracellular M. tuberculosis within granulomas in mice is presented. In vivo hollow fiber-encapsulated M. tuberculosis demonstrates significant induction of several key regulatory genes, including Rv3133c (dosR), as well as 20 other genes of the recently described dosR regulon (Voskuil, supra), the sigma factor genes sigB, sigC, and sigH, the DNA polymerase-encoding dnaE2, and many other genes which were found to be significantly upregulated in the mouse model of pulmonary tuberculosis. Talaat, supra. Increased expression of genes involved in lipid metabolism suggests depletion of glucose and the importance of fatty acids as a primary carbon source for extracellular M. tuberculosis in artificial granulomas in mice, which corroborates previous data in the mouse model of chronic tuberculosis. McKinney, supra. Consistent with the hypothesis that these organisms demonstrate an altered physiologic state characterized by reduced replication and metabolism, significant downregulation of genes encoding key ribosomal RNA binding proteins, and ATP synthase, respectively, was detected. A subset of 13 genes identified by microarray analysis was confirmed to be significantly differentially regulated by quantitative real-time RT-PCR. The common transcriptional profile observed lends further support to the hypothesis that the altered physiologic state of hollow fiber-encapsulated bacilli in vivo is similar to that of persistent bacilli in other models of latency.

It is important to note that although the artificial subcutaneous granulomas surrounding hollow fibers containing M. tuberculosis resemble those formed in mouse lungs after aerosol infection with the same pathogen, granulomas in mice differ significantly from those in humans. In human infection, the granuloma is composed of a central core of macrophages, including multinucleated giant cells, surrounded by macrophages and lymphocytes, including CD4 and CD8 T cells, and B cells. Randhawa, P. S. Pathology 22:153-155 (1990). Although individual components of mouse granulomas are similar to those in humans, the architecture of the mouse granuloma is better characterized as a loose collection of activated and epithelioid macrophages and lymphocytic clusters. Flynn, J. L., and J. Chan. Animal models of tuberculosis. In Tuberculosis. G. S M, editor. Lippicott Williams & Wilkins, Philadelphia. 237-250 (2004). Unlike in human granulomas, multinucleated giant cells are absent in mouse granulomas, and necrosis and caseation are rarely observed. Id. Despite structural differences between granulomas in mice and humans, however, their function is likely to be similar, with respect to containment of infection and creation of a localized environment for the immune response to kill organisms.

Animal models of tuberculosis have shown that M. tuberculosis is an intracellular pathogen, residing within host macrophages. Flynn, supra. However, the precise location of persistent bacilli during human latent tuberculosis infection remains elusive. Specifically, autopsy data of persons who died of non-tuberculosis-related causes have demonstrated that tubercle bacilli may be found outside lung granulomas, in normal-appearing lung tissue (Opie E L, A. J. Arch Pathol Lab Med 4:1-21 (1927)), and in non-macrophage cell types, including alveolar epithelial cells. Hemandez-Pando, R., et al. Lancet 356:2133-2138 (2000). In addition, bacilli residing extracellularly in the caseous material of lung granulomas and cavities may represent an important reservoir of persistent organisms during human latent tuberculosis infection. Grosset, supra. Mouse-implanted hollow fibers containing M. tuberculosis induce splenomegaly and the accumulation of host inflammatory cells, including macrophages and lymphocytes, leading to the formation of artificial granulomas around the fibers. The precise microenvironment conditions prevailing within subcutaneously-implanted hollow fibers are unclear, but may include hypoxia, nutrient starvation, and soluble immunologic factors (e.g., diffusible nitric oxide) secreted by surrounding host immune cells. Diminished ability to contain the growth of hollow fiber-encapsulated M. tuberculosis by IFNγ-deficient mice, as well as significant induction of the dosR regulon by these bacilli suggests that diffusible nitric oxide and/or hypoxia may contribute to the microenvironment experienced by infra-fiber bacilli in vivo. The basis for mouse spleen enlargement and recruitment of host immune cells to the tissues surrounding the hollow fibers is unknown, but may involve diffusion of M. tuberculosis-secreted soluble factors across the hollow fiber membranes.

As used herein, the terms “about” and “approximately” when referring to a numerical value shall have their plain and ordinary meanings to one of ordinary skill in the art. The amount of broadening from the strict numerical boundary depends upon the criticality of the particular element at issue. Thus, as a general matter, “about” or “approximately” broaden the numerical value, yet cannot be given a precise limit. For example, in some cases, “about” or “approximately” may mean ±5%, or ±10%, or ±20%, or ±30%, or ±100% depending on the relevant technology and the effects the variances will elicit.

A number of embodiments of the invention have been described. Nevertheless, it will be understood that various modifications may be made without departing from the spirit and scope of the invention. Accordingly, other embodiments are within the scope of the following claims, and as various changes can be made to the above compositions, formulations, combinations, methods, and processes without departing from the scope of the invention. It is intended that all matter contained in the above description be interpreted as illustrative and not in a limiting sense.

All publications cited herein are hereby incorporated by reference to the same extent as if each publication were individually and specifically indicated to be incorporated by reference and were set forth in its entirety herein.

We claim:

1. A method for evaluating cellular changes in vivo in response to administration of a drug or drugs of interest, comprising:
a) encapsulating cells of interest in a hollow fiber;
b) implanting the hollow fiber into an animal;
c) administering the drug or drugs of interest to the animal;
d) isolating the cells from the hollow fiber; and
e) evaluating the transcriptional profiles of the cells.

2. The method of claim 1, wherein the cells are isolated from a human.

3. The method of claim 1, wherein the cells are selected from the group consisting of: stem cells, peripheral blood cells, lymphoid cells, hepatocytes, bone marrow-derived cells, skin biopsies, broncho-alveolar lavage washings, breast tissue cells, kidney cells, oral, urethral, vaginal, cervical, or gastric, or intestinal mucosal cells or mucosal biopsies, reproductive cells, adipose cells, nerve or stromal cells, bone or synovial cells, or combinations thereof.

4. The method of claim 1, wherein the drug or drugs is selected from the group consisting of: analgesics, anesthetics, anti-acne agents, antibiotics, anticholinergics, anticoagulants, anticonvulsants, antidiabetic agents, antidyskinetics, antifibrotic agents, antifungal agents, anti-glaucoma agents, anti-infectives, anti-inflammatory compounds, antimicrobial compounds, antineoplastics, antiParkinsonian agents, antirheumatic agents, antiosteoporotics, antiseptics, antisporatics, antithrombotics, antivirals, appetitite stimulants, bacteriostatics, biologicals, blood modifiers, bone metabolism regulators, calcium regulators, cardioprotective agents, cardiovascular agents, central nervous system stimulants, cholinesterase inhibitors, contraceptives, cystic fibrosis agents, deodorants, detoxifying agnets, diagnostics, disinfectants, dietary supplements, dopamine receptor agonists, enzymes, erectile dysfunction agents, fertility agents, gastrointestinal agents, gout agents, hormones, hypnotics, immunomodulators, immunosuppressives, keratolytics, mast cell stabilizers, migraine agents, motion sickness agents, multiple sclerosis treatments, muscle relaxants, nasal preparations, nucleoside analogs, obesity agents, opthalmic agents, osteoporosis agents, parasympatholytics, parasympathomimetics, prostaglandins, psychotherapeutic agents, respiratory agents, sclerosing and anti-sclerosing agents, sedatives, skin and mucous membrane agents, smoking cessation agents, sympatholytics, ultraviolet screening agents, urinary tract agents, vaginal agents, vasodilators, or combinations thereof.

5. The method of claim 1, wherein the transcriptional profiles are correlated with specific cellular toxicity profiles.

6. The method of claim 1, wherein the drug or drugs is administered for a period of from about 1 hour to about 30 days.

7. The method of claim 1, wherein the transcriptional profiles are evaluated using microarray analysis.

8. A method for evaluating cellular changes in a microorganism in vivo, comprising:
a) encapsulating one or more microorganisms in a hollow fiber;
b) implanting the hollow fiber into an animal;
c) isolating the microorganism from the hollow fiber; and
e) evaluating the transcriptional profiles of the microorganism.

9. The method of claim 8, wherein the microorganism is deficient in a specific gene.

10. The method of claim 8, wherein the microorganism is evaluated during latency.

11. The method of claim 8, wherein the microorganism is isolated from the hollow fiber after a period of from about 1 hour to about 30 days.

12. The method of claim 8, wherein the microorganism is selected from the group consisting of: Mycobacterium species including M. tuberculosis, Staphylococcal species including Staphylococcus aureus, Streptococcus pneumoniae, Streptococcus pyogenes, other pathogenic Streptococcal species, including Enterococci, Haemophilus species, Moraxella species, Neisseria species, Legionella species, Listeria species, Chlamydia species, Mycoplasma species, Pseudomonas species, Escherichia coli, Klebsiella species, Enterobacter species, Serratia species, Acinetobacter species, Xanthomonas species, Stenotrophomonas, Borrelia species, Treponemal species, Nocardia species, Actinomycete species, Bacteroides species, Clostridial species including C. difficile, Peptostreptococci, Bacillus species, Francisella species, Yersinia species, Candida species, including Candida albicans, Histoplasma species, Cryptococcus species, Aspergillus species, Blastomycosis species, viruses within appropriate cellular carriers including HIV, smallpox virus (Variola), hepatitis A, B, C, D, and E viruses, influenza viruses, rhinoviruses, adenoviruses, coxsackie viruses, parainfluenza viruses, poliovirus, measles virus, Varicella virus, Herpesviruses including HSV-1 and HSV-2, Cytomegalovirus (CMV), Noroviruses, and parasitic species including Plasmodia species, Giardia species, Toxoplasma species, Schistosoma species, Trypanosoma species, and Leishmania species.

13. The method of claim 8, wherein the transcriptional profiles are evaluated using microarray analysis.

14. A method for evaluating cellular changes in a microorganism in vivo in response to administration of a drug or drugs of interest, comprising:
a) encapsulating one or more microorganisms in a hollow fiber;
b) implanting the hollow fiber into an animal;
c) administering the drug or drugs of interest to the animal;
d) isolating the microorganism from the hollow fiber; and
e) evaluating the transcriptional profiles of the microorganism.

15. The method of claim 14, wherein the drug or drugs is an antibacterial, antibiotic, antiviral agent, antiparasitic agent, or anti-fungal agent or combination thereof.

16. The method of claim 14, wherein the drug or drugs is administered for a period of from about 1 hour to about 30 days.

17. The method of claim 14 wherein the microorganism is deficient in a specific gene.

18. The method of claim 14, wherein the microorganism is evaluated during latency.

19. The method of claim 14, wherein the microorganism is selected from the group consisting of: Mycobacterium species including M. tuberculosis, Staphylococcal species including Staphylococcus aureus, Streptococcus pneumoniae, Streptococcus pyogenes, other pathogenic Streptococcal species, including Enterococci, Haemophilus species, Moraxella species, Neisseria species, Legionella species, Listeria species, Chlamydia species, Mycoplasma species, Pseudomonas species, Escherichia coli, Klebsiella species, Enterobacter species, Serratia species, Acinetobacter species, Xanthomonas species, Stenotrophomonas, Borrelia species, Treponemal species, Nocardia species, Actinomycete species, Bacteroides species, Clostridial species including C. difficile, Peptostreptococci, Bacillus species, Francisella species, Yersinia species, Candida species, including Candida albicans, Histoplasma species, Cryptococcus species, Aspergillus species, Blastomycosis species, viruses within appropriate cellular carriers including HIV, smallpox virus (Variola), hepatitis A, B, C, D, and E viruses, influenza viruses, rhinoviruses, adenoviruses, coxsackie viruses, parainfluenza viruses, poliovirus, measles virus, Varicella virus, Herpesviruses including HSV-1 and HSV-2, Cytomegalovirus (CMV), Noroviruses, and parasitic species including Plasmodia species, Giardia species, Toxoplasma species, Schistosoma species, Trypanosoma species, and Leishmania species.

20. The method of claim 14, wherein the transcriptional profiles are evaluated using microarray analysis.

21. A vaccine comprising at least one hollow fiber comprising at least one microorganism.

22. The vaccine of claim 21, wherein the microorganism is attenuated.

23. The vaccine of claim 21, wherein the microorganism is Mycobacterium tuberculosis.

24. A method of vaccinating an animal, comprising: encapsulating one or more microorganisms in a hollow fiber and implanting the hollow fiber into the animal.

25. The method of claim 24, wherein the microorganism is attenuated.

26. The method of claim 24, wherein the microorganism is Mycobacterium tuberculosis.

27. The method of claim 24, wherein the animal is a human.
